SEQUENCE LISTING (953 sequences)

SEQ ID NO: 1 - DNA, 120 bp, synthetic construct
  source 1..120: mol_type = other DNA
tggaggaaccagctgcaaatcacttttttgctctgtaaatttggaagtgtcatgggtgtctgtgggttatttaaaagaaattataacaattttgctaaaccattacaatgttaggtcttt

SEQ ID NO: 2 - DNA, 120 bp, synthetic construct; identical to SEQ ID NO: 1
  source 1..120: mol_type = other DNA

SEQ ID NO: 3 - DNA, 120 bp, synthetic construct; identical to SEQ ID NO: 1
  source 1..120: mol_type = other DNA

SEQ ID NO: 4 - DNA, 79 bp, synthetic construct
  source 1..79: mol_type = other DNA
aggagggctcctggggccgatgggtccagaagaagttccagaaatacattggtttcgccccatgcatcttccatggtcg

SEQ ID NO: 5 - DNA, 79 bp, synthetic construct; identical to SEQ ID NO: 4
  source 1..79: mol_type = other DNA

SEQ ID NO: 6 - DNA, 20 bp, synthetic construct
  source 1..20: mol_type = other DNA
atgggtgtctgtgggttatt

SEQ ID NO: 7 - DNA, 19 bp, synthetic construct
  source 1..19: mol_type = other DNA
tgtgggttatttaaaagaa

SEQ ID NO: 8 - DNA, 20 bp, synthetic construct
  source 1..20: mol_type = other DNA
gggttatttaaaagaaatta

SEQ ID NO: 9 - DNA, 20 bp, synthetic construct
  source 1..20: mol_type = other DNA
tgggtccagaagaagttcca

SEQ ID NO: 10 - DNA, 20 bp, synthetic construct
  source 1..20: mol_type = other DNA
gggtccagaagaagttccag

SEQ ID NO: 11 - RNA, 20 nt, synthetic construct
  source 1..20: mol_type = other RNA
tataacccacagacacccat

SEQ ID NO: 12 - RNA, 20 nt, synthetic construct
  source 1..20: mol_type = other RNA
tttcttttaaataacccaca

SEQ ID NO: 13 - RNA, 20 nt, synthetic construct
  source 1..20: mol_type = other RNA
taatttcttttaaataaccc

SEQ ID NO: 14 - RNA, 20 nt, synthetic construct
  source 1..20: mol_type = other RNA
tggaacttcttctggaccca

SEQ ID NO: 15 - RNA, 20 nt, synthetic construct
  source 1..20: mol_type = other RNA
ttggaacttcttctggaccc

SEQ ID NO: 16 - RNA, 18 nt, synthetic construct
  source 1..18: mol_type = other RNA
gggtgtctgtgggttata

SEQ ID NO: 17 - RNA, 18 nt, synthetic construct
  source 1..18: mol_type = other RNA
tgggttatttaaaagaaa

SEQ ID NO: 18 - RNA, 18 nt, synthetic construct
  source 1..18: mol_type = other RNA
gttatttaaaagaaatta

SEQ ID NO: 19 - RNA, 18 nt, synthetic construct
  source 1..18: mol_type = other RNA
ggtccagaagaagttcca

SEQ ID NO: 20 - RNA, 18 nt, synthetic construct
  source 1..18: mol_type = other RNA
gtccagaagaagttccaa

SEQ ID NO: 21 - RNA, 20 nt, synthetic construct
  source 1..20: mol_type = other RNA
atgggtgtctgtgggttata

SEQ ID NO: 22 - RNA, 20 nt, synthetic construct
  source 1..20: mol_type = other RNA
tgtgggttatttaaaagaaa

SEQ ID NO: 23 - RNA, 20 nt, synthetic construct
  source 1..20: mol_type = other RNA
gggttatttaaaagaaatta

SEQ ID NO: 24 - RNA, 20 nt, synthetic construct
  source 1..20: mol_type = other RNA
tgggtccagaagaagttcca

SEQ ID NO: 25 - RNA, 20 nt, synthetic construct
  source 1..20: mol_type = other RNA
gggtccagaagaagttccaa

SEQ ID NO: 26 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tataacccacagacacccat

SEQ ID NO: 27 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tttcttttaaataacccaca

SEQ ID NO: 28 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
taatttcttttaaataaccc

SEQ ID NO: 29 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tggaacttcttctggaccca

SEQ ID NO: 30 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
ttggaacttcttctggaccc

SEQ ID NO: 31 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gggtgtctgtgggttata

SEQ ID NO: 32 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
tgggttatttaaaagaaa

SEQ ID NO: 33 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gttatttaaaagaaatta

SEQ ID NO: 34 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
ggtccagaagaagttcca

SEQ ID NO: 35 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gtccagaagaagttccaa

SEQ ID NO: 36 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
atgggtgtctgtgggttata

SEQ ID NO: 37 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
tgtgggttatttaaaagaaa

SEQ ID NO: 38 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
gggttatttaaaagaaatta

SEQ ID NO: 39 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
tgggtccagaagaagttcca

SEQ ID NO: 40 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
gggtccagaagaagttccaa

SEQ ID NO: 41 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tataacccacagacacccat

SEQ ID NO: 42 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tttcttttaaataacccaca

SEQ ID NO: 43 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
taatttcttttaaataaccc

SEQ ID NO: 44 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
tggaacttcttctggaccca

SEQ ID NO: 45 - RNA, 20 nt, synthetic construct
  source 1..20: mol_type = other RNA
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-Fluoro nucleotides
  modified_base order(1..3,13..20): mod_base = OTHER; note = Phosphorothioate internucleotide linkage
ttggaacttcttctggaccc

SEQ ID NO: 46 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gggtgtctgtgggttata

SEQ ID NO: 47 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
tgggttatttaaaagaaa

SEQ ID NO: 48 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gttatttaaaagaaatta

SEQ ID NO: 49 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
ggtccagaagaagttcca

SEQ ID NO: 50 - RNA, 18 nt, synthetic construct
  source 1..18: mol_type = other RNA
  modified_base order(1..7,11,13..18): mod_base = OTHER; note = 2-Prime-O-methyl nucleotides
  modified_base order(8..10,12): mod_base = OTHER; note = 2-Prime-Fluoro nucleotides
  modified_base order(1..3,16..18): mod_base = OTHER; note = Phosphorothioate internucleotide linkage
gtccagaagaagttccaa

SEQ ID NO: 51 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
atgggtgtctgtgggttata

SEQ ID NO: 52 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
tgtgggttatttaaaagaaa

SEQ ID NO: 53 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
gggttatttaaaagaaatta

SEQ ID NO: 54 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
tgggtccagaagaagttcca

SEQ ID NO: 55 - RNA, 20 nt, synthetic construct
  source 1..20: mol_type = other RNA
  modified_base order(1..9,13,15..20): mod_base = OTHER; note = 2-Prime-O-methyl nucleotides
  modified_base order(10..12,14): mod_base = OTHER; note = 2-Prime-Fluoro nucleotides
  modified_base order(1..3,18..20): mod_base = OTHER; note = Phosphorothioate internucleotide linkage
gggtccagaagaagttccaa

SEQ ID NO: 56 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-Fluoro nucleotides
  modified_base order(1..3,14..21): mod_base = OTHER; note = Phosphorothioate internucleotide linkage
ttgacattgtactcggcggtt

SEQ ID NO: 57 - RNA, 16 nt, synthetic construct
  source 1..16: mol_type = other RNA
  modified_base order(1..3,5,7,9,11..13,15,16): mod_base = OTHER; note = 2-Prime-O-methyl nucleotides
  modified_base order(4,6,8,10,14): mod_base = OTHER; note = 2-Prime-Fluoro nucleotides
  modified_base order(1..3,14..16): mod_base = OTHER; note = Phosphorothioate internucleotide linkage
ccgagtacaatgtcaa

SEQ ID NO: 58 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-Fluoro nucleotides
  modified_base order(1..3,14..21): mod_base = OTHER; note = Phosphorothioate internucleotide linkage
taaaggtcgaatttgccaatt

SEQ ID NO: 59 - RNA, 16 nt, synthetic construct
  source 1..16: mol_type = other RNA
  modified_base order(1..3,5,7,9,11..13,15,16): mod_base = OTHER; note = 2-Prime-O-methyl nucleotides
  modified_base order(4,6,8,10,14): mod_base = OTHER; note = 2-Prime-Fluoro nucleotides
  modified_base order(1..3,14..16): mod_base = OTHER; note = Phosphorothioate internucleotide linkage
gcaaattcgaccttta

SEQ ID NO: 60 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-Fluoro nucleotides
  modified_base order(1..3,14..21): mod_base = OTHER; note = Phosphorothioate internucleotide linkage
tgcaatttgaatcatttcttt

SEQ ID NO: 61 - RNA, 16 nt, synthetic construct
  source 1..16: mol_type = other RNA
  modified_base order(1..3,5,7,9,11..13,15,16): mod_base = OTHER; note = 2-Prime-O-methyl nucleotides
  modified_base order(4,6,8,10,14): mod_base = OTHER; note = 2-Prime-Fluoro nucleotides
  modified_base order(1..3,14..16): mod_base = OTHER; note = Phosphorothioate internucleotide linkage
aatgattcaaattgca

SEQ ID NO: 62 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-Fluoro nucleotides
  modified_base order(1..3,14..21): mod_base = OTHER; note = Phosphorothioate internucleotide linkage
tagcaatttgaatcatttctt

SEQ ID NO: 63 - RNA, 16 nt, synthetic construct
  source 1..16: mol_type = other RNA
  modified_base order(1..3,5,7,9,11..13,15,16): mod_base = OTHER; note = 2-Prime-O-methyl nucleotides
  modified_base order(4,6,8,10,14): mod_base = OTHER; note = 2-Prime-Fluoro nucleotides
  modified_base order(1..3,14..16): mod_base = OTHER; note = Phosphorothioate internucleotide linkage
atgattcaaattgcta

SEQ ID NO: 64 - DNA, 45 bp, synthetic construct
  source 1..45: mol_type = other DNA
ctggcacgcacgtcctccgccgagtacaatgtcaacaacctggtg

SEQ ID NO: 65 - DNA, 45 bp, synthetic construct
  source 1..45: mol_type = other DNA
ggtcgcttcctggaaattggcaaattcgacctttctcagaaccac

SEQ ID NO: 66 - DNA, 45 bp, synthetic construct
  source 1..45: mol_type = other DNA
ctgtgtttgtttttcaagaaatgattcaaattgctgcttggattt

SEQ ID NO: 67 - DNA, 45 bp, synthetic construct
  source 1..45: mol_type = other DNA
tgtgtttgtttttcaagaaatgattcaaattgctgcttggatttt

SEQ ID NO: 68 - DNA, 45 bp, synthetic construct
  source 1..45: mol_type = other DNA
gaatgtgtatctatttgagtctggaaataactaatgtgtttgata

SEQ ID NO: 69 - DNA, 45 bp, synthetic construct
  source 1..45: mol_type = other DNA
agaaaaaatacaatttctcactttgcatttagtcaaaagaaaaaa

SEQ ID NO: 70 - DNA, 45 bp, synthetic construct
  source 1..45: mol_type = other DNA
ctttatagcaaaatgaaagagaacatgaaatgcttctttctcagt

SEQ ID NO: 71 - DNA, 45 bp, synthetic construct
  source 1..45: mol_type = other DNA
aacatgaaatgcttctttctcagtttattggttgaatgtgtatct

SEQ ID NO: 72 - DNA, 45 bp, synthetic construct
  source 1..45: mol_type = other DNA
cctgtcaccatcatctcagtcatcaatcatccctctgatgtaatg

SEQ ID NO: 73 - DNA, 45 bp, synthetic construct
  source 1..45: mol_type = other DNA
gttacagcttctacctacgcaataagagtttctaattatgatatc

SEQ ID NO: 74 - DNA, 45 bp, Homo sapiens
  source 1..45: mol_type = other DNA
ataattagtttagtttgtggcttcatggaaactccctgtaaacta

SEQ ID NO: 75 - DNA, 45 bp, Homo sapiens
  source 1..45: mol_type = other DNA
gtgtatctatttgagtctggaaataactaatgtgtttgataatta

SEQ ID NO: 76 - DNA, 45 bp, Homo sapiens
  source 1..45: mol_type = other DNA
ttgtgattatctttttgtggtgtgaataaatcttttatcttgaat

SEQ ID NO: 77 - DNA, 45 bp, Homo sapiens
  source 1..45: mol_type = other DNA
ctatgttcattctatagaagaaatgcaaactatcactgtatttta

SEQ ID NO: 78 - DNA, 45 bp, Homo sapiens
  source 1..45: mol_type = other DNA
tatctatttgagtctggaaataactaatgtgtttgataattagtt

SEQ ID NO: 79 - DNA, 45 bp, Homo sapiens
  source 1..45: mol_type = other DNA
aagcggaaagccaatgatgagagcaatgagcattccgatgtgatt

SEQ ID NO: 80 - DNA, 45 bp, Homo sapiens
  source 1..45: mol_type = other DNA
tcattctatagaagaaatgcaaactatcactgtattttaatattt

SEQ ID NO: 81 - DNA, 45 bp, Homo sapiens
  source 1..45: mol_type = other DNA
ggaagaagataaacacctgaaatttcgtatttctcatgaattaga

SEQ ID NO: 82 - DNA, 45 bp, Homo sapiens
  source 1..45: mol_type = other DNA
gaatgtaataagaatttggtggtgtcaattgcttatttgttttcc

SEQ ID NO: 83 - DNA, 45 bp, Homo sapiens
  source 1..45: mol_type = other DNA
ataaatcttttatcttgaatgtaataagaatttggtggtgtcaat

SEQ ID NO: 84 - DNA, 45 bp, Homo sapiens
  source 1..45: mol_type = other DNA
acagccatgaatttcacagccatgaagatatgctggttgtagacc

SEQ ID NO: 85 - DNA, 45 bp, Homo sapiens
  source 1..45: mol_type = other DNA
tgtatctatttgagtctggaaataactaatgtgtttgataattag

SEQ ID NO: 86 - DNA, 45 bp, Homo sapiens
  source 1..45: mol_type = other DNA
taaactaaaagcttcagggttatgtctatgttcattctatagaag

SEQ ID NO: 87 - DNA, 45 bp, Homo sapiens
  source 1..45: mol_type = other DNA
ggagaaaaaatacaatttctcactttgcatttagtcaaaagaaaa

SEQ ID NO: 88 - DNA, 45 bp, Homo sapiens
  source 1..45: mol_type = other DNA
gctgccttgggggtcactgcaattagactgcttaatgaagacatt

SEQ ID NO: 89 - DNA, 45 bp, Homo sapiens
  source 1..45: mol_type = other DNA
tatagcaaaatgaaagagaacatgaaatgcttctttctcagttta

SEQ ID NO: 90 - DNA, 45 bp, Homo sapiens
  source 1..45: mol_type = other DNA
tttaatatttgttattctctcatgaatagaaatttatgtagaagc

SEQ ID NO: 91 - DNA, 45 bp, Homo sapiens
  source 1..45: mol_type = other DNA
atctatttgagtctggaaataactaatgtgtttgataattagttt

SEQ ID NO: 92 - DNA, 45 bp, Homo sapiens
  source 1..45: mol_type = other DNA
acctgaaatttcgtatttctcatgaattagatagtgcatcttctg

SEQ ID NO: 93 - DNA, 45 bp, Homo sapiens
  source 1..45: mol_type = other DNA
atgttcattctatagaagaaatgcaaactatcactgtattttaat

SEQ ID NO: 94 - DNA, 45 bp, Homo sapiens
  source 1..45: mol_type = other DNA
cgatgtgattgatagtcaggaactttccaaagtcagccgtgaatt

SEQ ID NO: 95 - DNA, 45 bp, Homo sapiens
  source 1..45: mol_type = other DNA
aaagtaaggaagaagataaacacctgaaatttcgtatttctcatg

SEQ ID NO: 96 - DNA, 45 bp, Homo sapiens
  source 1..45: mol_type = other DNA
gtttattggttgaatgtgtatctatttgagtctggaaataactaa

SEQ ID NO: 97 - DNA, 45 bp, Homo sapiens
  source 1..45: mol_type = other DNA
gacagccaggactccattgactcgaacgactctgatgatgtagat

SEQ ID NO: 98 - DNA, 45 bp, Homo sapiens
  source 1..45: mol_type = other DNA
gttattctctcatgaatagaaatttatgtagaagcaaacaaaata

SEQ ID NO: 99 - DNA, 45 bp, Homo sapiens
  source 1..45: mol_type = other DNA
aatgctgtgtcctctgaagaaaccaatgactttaaacaagagacc

SEQ ID NO: 100 - DNA, 45 bp, Homo sapiens
  source 1..45: mol_type = other DNA
agcttggacaaaaaggcacacagagttcaattccagttgaacaga

SEQ ID NO: 101 - DNA, 45 bp, Homo sapiens
  source 1..45: mol_type = other DNA
attctatagaagaaatgcaaactatcactgtattttaatatttgt

SEQ ID NO: 102 - DNA, 45 bp, Homo sapiens
  source 1..45: mol_type = other DNA
tgatgtagatgacactgatgattctcaccagtctgatgagtctca

SEQ ID NO: 103 - DNA, 45 bp, Homo sapiens
  source 1..45: mol_type = other DNA
tccaacgaaagccatgaccacatggatgatatggatgatgaagat

SEQ ID NO: 104 - DNA, 45 bp, Homo sapiens
  source 1..45: mol_type = other DNA
aattaaaaggagaaaaaatacaatttctcactttgcatttagtca

SEQ ID NO: 105 - DNA, 45 bp, Homo sapiens
  source 1..45: mol_type = other DNA
aaataactaatgtgtttgataattagtttagtttgtggcttcatg

SEQ ID NO: 106 - DNA, 45 bp, Homo sapiens
  source 1..45: mol_type = other DNA
aaatcttttatcttgaatgtaataagaatttggtggtgtcaattg

SEQ ID NO: 107 - DNA, 45 bp, Homo sapiens
  source 1..45: mol_type = other DNA
attgcttatttgttttcccacggttgtccagcaattaataaaaca

SEQ ID NO: 108 - DNA, 45 bp, Homo sapiens
  source 1..45: mol_type = other DNA
gagcattccgatgtgattgatagtcaggaactttccaaagtcagc

SEQ ID NO: 109 - DNA, 45 bp, Homo sapiens
  source 1..45: mol_type = other DNA
gtaaactaaaagcttcagggttatgtctatgttcattctatagaa

SEQ ID NO: 110 - DNA, 45 bp, Homo sapiens
  source 1..45: mol_type = other DNA
aaactatcactgtattttaatatttgttattctctcatgaataga

SEQ ID NO: 111 - DNA, 45 bp, Homo sapiens
  source 1..45: mol_type = other DNA
gagagcaatgagcattccgatgtgattgatagtcaggaactttcc

SEQ ID NO: 112 - DNA, 45 bp, Homo sapiens
  source 1..45: mol_type = other DNA
gatgattctcaccagtctgatgagtctcaccattctgatgaatct

SEQ ID NO: 113 - DNA, 45 bp, Homo sapiens
  source 1..45: mol_type = other DNA
aataaatcttttatcttgaatgtaataagaatttggtggtgtcaa

SEQ ID NO: 114 - DNA, 45 bp, Homo sapiens
  source 1..45: mol_type = other DNA
tcctctgaagaaaccaatgactttaaacaagagacccttccaagt

SEQ ID NO: 115 - DNA, 45 bp, Homo sapiens
  source 1..45: mol_type = other DNA
atttgttattctctcatgaatagaaatttatgtagaagcaaacaa

SEQ ID NO: 116 - DNA, 45 bp, Homo sapiens
  source 1..45: mol_type = other DNA
acatgaaatgcttctttctcagtttattggttgaatgtgtatcta

SEQ ID NO: 117 - DNA, 45 bp, Homo sapiens
  source 1..45: mol_type = other DNA
tcttttatcttgaatgtaataagaatttggtggtgtcaattgctt

SEQ ID NO: 118 - RNA, 20 nt, synthetic construct
  source 1..20: mol_type = other RNA
tgtggcttcatggaaactcc

SEQ ID NO: 119 - RNA, 20 nt, synthetic construct
  source 1..20: mol_type = other RNA
tctggaaataactaatgtgt

SEQ ID NO: 120 - RNA, 20 nt, synthetic construct
  source 1..20: mol_type = other RNA
tgagtctggaaataactaat

SEQ ID NO: 121 - RNA, 20 nt, synthetic construct
  source 1..20: mol_type = other RNA
tgtggtgtgaataaatcttt

SEQ ID NO: 122 - RNA, 20 nt, synthetic construct
  source 1..20: mol_type = other RNA
agaagaaatgcaaactatca

SEQ ID NO: 123 - RNA, 20 nt, synthetic construct
  source 1..20: mol_type = other RNA
ggaaataactaatgtgtttg

SEQ ID NO: 124 - RNA, 20 nt, synthetic construct
  source 1..20: mol_type = other RNA
gatgagagcaatgagcattc

SEQ ID NO: 125 - RNA, 20 nt, synthetic construct
  source 1..20: mol_type = other RNA
aatgcaaactatcactgtat

SEQ ID NO: 126 - RNA, 20 nt, synthetic construct
  source 1..20: mol_type = other RNA
cctgaaatttcgtatttctc

SEQ ID NO: 127 - RNA, 20 nt, synthetic construct
  source 1..20: mol_type = other RNA
ttggtggtgtcaattgctta

SEQ ID NO: 128 - RNA, 20 nt, synthetic construct
  source 1..20: mol_type = other RNA
tgaatgtaataagaatttgg

SEQ ID NO: 129 - RNA, 20 nt, synthetic construct
  source 1..20: mol_type = other RNA
tctcactttgcatttagtca

SEQ ID NO: 130 - RNA, 20 nt, synthetic construct
  source 1..20: mol_type = other RNA
acagccatgaagatatgctg

SEQ ID NO: 131 - RNA, 20 nt, synthetic construct
  source 1..20: mol_type = other RNA
ctggaaataactaatgtgtt

SEQ ID NO: 132 - RNA, 20 nt, synthetic construct
  source 1..20: mol_type = other RNA
agggttatgtctatgttcat

SEQ ID NO: 133 - RNA, 20 nt, synthetic construct
  source 1..20: mol_type = other RNA
aaagagaacatgaaatgctt

SEQ ID NO: 134 - RNA, 20 nt, synthetic construct
  source 1..20: mol_type = other RNA
tttctcactttgcatttagt

SEQ ID NO: 135 - RNA, 20 nt, synthetic construct
  source 1..20: mol_type = other RNA
actgcaattagactgcttaa

SEQ ID NO: 136 - RNA, 20 nt, synthetic construct
  source 1..20: mol_type = other RNA
gagaacatgaaatgcttctt

SEQ ID NO: 137 - RNA, 20 nt, synthetic construct
  source 1..20: mol_type = other RNA
tctctcatgaatagaaattt

SEQ ID NO: 138 - RNA, 20 nt, synthetic construct
  source 1..20: mol_type = other RNA
tttctcagtttattggttga

SEQ ID NO: 139 - RNA, 20 nt, synthetic construct
  source 1..20: mol_type = other RNA
gaaataactaatgtgtttga

SEQ ID NO: 140 - RNA, 20 nt, synthetic construct
  source 1..20: mol_type = other RNA
tttctcatgaattagatagt

SEQ ID NO: 141 - RNA, 20 nt, synthetic construct
  source 1..20: mol_type = other RNA
aagaaatgcaaactatcact

SEQ ID NO: 142 - RNA, 20 nt, synthetic construct
  source 1..20: mol_type = other RNA
tcaggaactttccaaagtca

SEQ ID NO: 143 - RNA, 20 nt, synthetic construct
  source 1..20: mol_type = other RNA
ataaacacctgaaatttcgt

SEQ ID NO: 144 - RNA, 20 nt, synthetic construct
  source 1..20: mol_type = other RNA
gtgtatctatttgagtctgg

SEQ ID NO: 145 - RNA, 20 nt, synthetic construct
  source 1..20: mol_type = other RNA
attgactcgaacgactctga

SEQ ID NO: 146 - RNA, 20 nt, synthetic construct
  source 1..20: mol_type = other RNA
atagaaatttatgtagaagc

SEQ ID NO: 147 - RNA, 20 nt, synthetic construct
  source 1..20: mol_type = other RNA
gaagaaaccaatgactttaa

SEQ ID NO: 148 - RNA, 20 nt, synthetic construct
  source 1..20: mol_type = other RNA
gcacacagagttcaattcca

SEQ ID NO: 149 - RNA, 20 nt, synthetic construct
  source 1..20: mol_type = other RNA
tgcaaactatcactgtattt

SEQ ID NO: 150 - RNA, 20 nt, synthetic construct
  source 1..20: mol_type = other RNA
tgatgattctcaccagtctg

SEQ ID NO: 151 - RNA, 20 nt, synthetic construct
  source 1..20: mol_type = other RNA
gaccacatggatgatatgga

SEQ ID NO: 152 - RNA, 20 nt, synthetic construct
  source 1..20: mol_type = other RNA
aaatacaatttctcactttg

SEQ ID NO: 153 - RNA, 20 nt, synthetic construct
  source 1..20: mol_type = other RNA
ttgataattagtttagtttg

SEQ ID NO: 154 - RNA, 20 nt, synthetic construct
  source 1..20: mol_type = other RNA
aatgtaataagaatttggtg

SEQ ID NO: 155 - RNA, 20 nt, synthetic construct
  source 1..20: mol_type = other RNA
tcccacggttgtccagcaat

SEQ ID NO: 156 - RNA, 20 nt, synthetic construct
  source 1..20: mol_type = other RNA
attgatagtcaggaactttc

SEQ ID NO: 157 - RNA, 20 nt, synthetic construct
  source 1..20: mol_type = other RNA
cagggttatgtctatgttca

SEQ ID NO: 158 - RNA, 20 nt, synthetic construct
  source 1..20: mol_type = other RNA
tttaatatttgttattctct

SEQ ID NO: 159 - RNA, 20 nt, synthetic construct
  source 1..20: mol_type = other RNA
tccgatgtgattgatagtca

SEQ ID NO: 160 - RNA, 20 nt, synthetic construct
  source 1..20: mol_type = other RNA
tctgatgagtctcaccattc

SEQ ID NO: 161 - RNA, 20 nt, synthetic construct
  source 1..20: mol_type = other RNA
ttgaatgtaataagaatttg

SEQ ID NO: 162 - RNA, 20 nt, synthetic construct
  source 1..20: mol_type = other RNA
aatgactttaaacaagagac

SEQ ID NO: 163 - RNA, 20 nt, synthetic construct
  source 1..20: mol_type = other RNA
atgaatagaaatttatgtag

SEQ ID NO: 164 - RNA, 20 nt, synthetic construct
  source 1..20: mol_type = other RNA
ttctcagtttattggttgaa

SEQ ID NO: 165 - RNA, 20 nt, synthetic construct
  source 1..20: mol_type = other RNA
gtaataagaatttggtggtg

SEQ ID NO: 166 - RNA, 20 nt, synthetic construct
  source 1..20: mol_type = other RNA
ggagtttccatgaagccaca

SEQ ID NO: 167 - RNA, 20 nt, synthetic construct
  source 1..20: mol_type = other RNA
acacattagttatttccaga

SEQ ID NO: 168 - RNA, 20 nt, synthetic construct
  source 1..20: mol_type = other RNA
attagttatttccagactca

SEQ ID NO: 169 - RNA, 20 nt, synthetic construct
  source 1..20: mol_type = other RNA
aaagatttattcacaccaca

SEQ ID NO: 170 - RNA, 20 nt, synthetic construct
  source 1..20: mol_type = other RNA
tgatagtttgcatttcttct

SEQ ID NO: 171 - RNA, 20 nt, synthetic construct
  source 1..20: mol_type = other RNA
caaacacattagttatttcc

SEQ ID NO: 172 - RNA, 20 nt, synthetic construct
  source 1..20: mol_type = other RNA
gaatgctcattgctctcatc

SEQ ID NO: 173 - RNA, 20 nt, synthetic construct
  source 1..20: mol_type = other RNA
atacagtgatagtttgcatt

SEQ ID NO: 174 - RNA, 20 nt, synthetic construct
  source 1..20: mol_type = other RNA
gagaaatacgaaatttcagg

SEQ ID NO: 175 - RNA, 20 nt, synthetic construct
  source 1..20: mol_type = other RNA
taagcaattgacaccaccaa

SEQ ID NO: 176 - RNA, 20 nt, synthetic construct
  source 1..20: mol_type = other RNA
ccaaattcttattacattca

SEQ ID NO: 177 - RNA, 20 nt, synthetic construct
  source 1..20: mol_type = other RNA
tgactaaatgcaaagtgaga

SEQ ID NO: 178 - RNA, 20 nt, synthetic construct
  source 1..20: mol_type = other RNA
cagcatatcttcatggctgt

SEQ ID NO: 179 - RNA, 20 nt, synthetic construct
  source 1..20: mol_type = other RNA
aacacattagttatttccag

SEQ ID NO: 180 - RNA, 20 nt, synthetic construct
  source 1..20: mol_type = other RNA
atgaacatagacataaccct

SEQ ID NO: 181 - RNA, 20 nt, synthetic construct
  source 1..20: mol_type = other RNA
aagcatttcatgttctcttt

SEQ ID NO: 182 - RNA, 20 nt, synthetic construct
  source 1..20: mol_type = other RNA
actaaatgcaaagtgagaaa

SEQ ID NO: 183 - RNA, 20 nt, synthetic construct
  source 1..20: mol_type = other RNA
ttaagcagtctaattgcagt

SEQ ID NO: 184 - RNA, 20 nt, synthetic construct
  source 1..20: mol_type = other RNA
aagaagcatttcatgttctc

SEQ ID NO: 185 - RNA, 20 nt, synthetic construct
  source 1..20: mol_type = other RNA
aaatttctattcatgagaga

SEQ ID NO: 186 - RNA, 20 nt, synthetic construct
  source 1..20: mol_type = other RNA
tcaaccaataaactgagaaa

SEQ ID NO: 187 - RNA, 20 nt, synthetic construct
  source 1..20: mol_type = other RNA
tcaaacacattagttatttc

SEQ ID NO: 188 - RNA, 20 nt, synthetic construct
  source 1..20: mol_type = other RNA
actatctaattcatgagaaa

SEQ ID NO: 189 - RNA, 20 nt, synthetic construct
  source 1..20: mol_type = other RNA
agtgatagtttgcatttctt

SEQ ID NO: 190 - RNA, 20 nt, synthetic construct
  source 1..20: mol_type = other RNA
tgactttggaaagttcctga

SEQ ID NO: 191 - RNA, 20 nt, synthetic construct
  source 1..20: mol_type = other RNA
acgaaatttcaggtgtttat

SEQ ID NO: 192 - RNA, 20 nt, synthetic construct
  source 1..20: mol_type = other RNA
ccagactcaaatagatacac

SEQ ID NO: 193 - RNA, 20 nt, synthetic construct
  source 1..20: mol_type = other RNA
tcagagtcgttcgagtcaat

SEQ ID NO: 194 - RNA, 20 nt, synthetic construct
  source 1..20: mol_type = other RNA
gcttctacataaatttctat

SEQ ID NO: 195 - RNA, 20 nt, synthetic construct
  source 1..20: mol_type = other RNA
ttaaagtcattggtttcttc

SEQ ID NO: 196 - RNA, 20 nt, synthetic construct
  source 1..20: mol_type = other RNA
tggaattgaactctgtgtgc

SEQ ID NO: 197 - RNA, 20 nt, synthetic construct
  source 1..20: mol_type = other RNA
aaatacagtgatagtttgca

SEQ ID NO: 198 - RNA, 20 nt, synthetic construct
  source 1..20: mol_type = other RNA
cagactggtgagaatcatca

SEQ ID NO: 199 - RNA, 20 nt, synthetic construct
  source 1..20: mol_type = other RNA
tccatatcatccatgtggtc

SEQ ID NO: 200 - RNA, 20 nt, synthetic construct
  source 1..20: mol_type = other RNA
caaagtgagaaattgtattt

SEQ ID NO: 201 - RNA, 20 nt, synthetic construct
  source 1..20: mol_type = other RNA
caaactaaactaattatcaa

SEQ ID NO: 202 - RNA, 20 nt, synthetic construct
  source 1..20: mol_type = other RNA
caccaaattcttattacatt

SEQ ID NO: 203 - RNA, 20 nt, synthetic construct
  source 1..20: mol_type = other RNA
attgctggacaaccgtggga

SEQ ID NO: 204 - RNA, 20 nt, synthetic construct
  source 1..20: mol_type = other RNA
gaaagttcctgactatcaat

SEQ ID NO: 205 - RNA, 20 nt, synthetic construct
  source 1..20: mol_type = other RNA
tgaacatagacataaccctg

SEQ ID NO: 206 - RNA, 20 nt, synthetic construct
  source 1..20: mol_type = other RNA
agagaataacaaatattaaa

SEQ ID NO: 207 - RNA, 20 nt, synthetic construct
  source 1..20: mol_type = other RNA
tgactatcaatcacatcgga

SEQ ID NO: 208 - RNA, 20 nt, synthetic construct
  source 1..20: mol_type = other RNA
gaatggtgagactcatcaga

SEQ ID NO: 209 - RNA, 20 nt, synthetic construct
  source 1..20: mol_type = other RNA
caaattcttattacattcaa

SEQ ID NO: 210 - RNA, 20 nt, synthetic construct
  source 1..20: mol_type = other RNA
gtctcttgtttaaagtcatt

SEQ ID NO: 211 - RNA, 20 nt, synthetic construct
  source 1..20: mol_type = other RNA
ctacataaatttctattcat

SEQ ID NO: 212 - RNA, 20 nt, synthetic construct
  source 1..20: mol_type = other RNA
ttcaaccaataaactgagaa

SEQ ID NO: 213 - RNA, 20 nt, synthetic construct
  source 1..20: mol_type = other RNA
caccaccaaattcttattac

SEQ ID NO: 214 - DNA, 45 bp, Mus musculus
  source 1..45: mol_type = other DNA
agtgttagtgagggttaagcaggaatactaactgctcatttctca

SEQ ID NO: 215 - DNA, 45 bp, Mus musculus
  source 1..45: mol_type = other DNA
tctgatgagaccgtcactgctagtacacaagcagacactttcact

SEQ ID NO: 216 - DNA, 45 bp, Mus musculus
  source 1..45: mol_type = other DNA
aagaaaaggatgactttaagcaagaaactcttccaagcaattcca

SEQ ID NO: 217 - DNA, 45 bp, Mus musculus
  source 1..45: mol_type = other DNA
ctatacccttttagttttataattagcatatattttgttgtgatt

SEQ ID NO: 218 - DNA, 45 bp, Mus musculus
  source 1..45: mol_type = other DNA
atgaataaaatgtatgtagaaacaagcaaatttactgaaacaagc

SEQ ID NO: 219 - DNA, 45 bp, Mus musculus
  source 1..45: mol_type = other DNA
ggaagatgataggtatctgaaattccgaatttctcatgaattaga

SEQ ID NO: 220 - DNA, 45 bp, Mus musculus
  source 1..45: mol_type = other DNA
ctggatgaaccaagtctggaaacacacagacttgagcattccaaa

SEQ ID NO: 221 - DNA, 45 bp, Mus musculus
  source 1..45: mol_type = other DNA
acaacaacggaaagggcagccatgagtcaagtcagctggatgaac

SEQ ID NO: 222 - DNA, 45 bp, Mus musculus
  source 1..45: mol_type = other DNA
tatgaataaaatgtatgtagaaacaagcaaatttactgaaacaag

SEQ ID NO: 223 - DNA, 45 bp, Mus musculus
  source 1..45: mol_type = other DNA
gagtctaaggagtccctcgatgtcatccctgttgcccagcttctg

SEQ ID NO: 224 - DNA, 45 bp, Mus musculus
  source 1..45: mol_type = other DNA
ctgaagaaaaggatgactttaagcaagaaactcttccaagcaatt

SEQ ID NO: 225 - DNA, 45 bp, Mus musculus
  source 1..45: mol_type = other DNA
gaagcagaatctccttgcgccacagaatgctgtgtcctctgaaga

SEQ ID NO: 226 - DNA, 45 bp, Mus musculus
  source 1..45: mol_type = other DNA
gtaaccaaaagcttctgcactttgcttctgttcttcctgtacaag

SEQ ID NO: 227 - DNA, 45 bp, Mus musculus
  source 1..45: mol_type = other DNA
tggatatatgtatgtagagaaagagaggtaatattttgggctctt

SEQ ID NO: 228 - DNA, 45 bp, Mus musculus
  source 1..45: mol_type = other DNA
ggactcggatgaatctgacgaatctcaccattcggatgagtctga

SEQ ID NO: 229 - DNA, 45 bp, Mus musculus
  source 1..45: mol_type = other DNA
agagaggtaatattttgggctcttagcttagtctgttgtttcatg

SEQ ID NO: 230 - DNA, 45 bp, Mus musculus
  source 1..45: mol_type = other DNA
atttgcttttgcctgtttggcattgcctcctccctcccggtgaaa

SEQ ID NO: 231 - DNA, 45 bp, Mus musculus
  source 1..45: mol_type = other DNA
gtggactcggatgaatctgacgaatctcaccattcggatgagtct

SEQ ID NO: 232 - DNA, 45 bp, Mus musculus
  source 1..45: mol_type = other DNA
gaaacaagcaaatttactgaaacaagcagaattaaaagagaaact

SEQ ID NO: 233 - DNA, 45 bp, Mus musculus
  source 1..45: mol_type = other DNA
ggatgtgatcgatagtcaagcaagttccaaagccagcctggaaca

SEQ ID NO: 234 - DNA, 45 bp, Mus musculus
  source 1..45: mol_type = other DNA
gagagttcatcttctgaggtcaactaaagaagaggcaaaaacaca

SEQ ID NO: 235 - DNA, 45 bp, Mus musculus
  source 1..45: mol_type = other DNA
aaggaagatgataggtatctgaaattccgaatttctcatgaatta

SEQ ID NO: 236 - DNA, 45 bp, Mus musculus
  source 1..45: mol_type = other DNA
ctaaagaagaggcaaaaacacagttccttactttgcatttagtaa

SEQ ID NO: 237 - DNA, 45 bp, Mus musculus
  source 1..45: mol_type = other DNA
ggactaactacgaccatgagattggcagtgatttgcttttgcctg

SEQ ID NO: 238 - DNA, 45 bp, Mus musculus
  source 1..45: mol_type = other DNA
cttagcttagtctgttgtttcatgcaaacaccgttgtaaccaaaa

SEQ ID NO: 239 - DNA, 45 bp, Mus musculus
  source 1..45: mol_type = other DNA
gaccgtcactgctagtacacaagcagacactttcactccaatcgt

SEQ ID NO: 240 - DNA, 45 bp, Mus musculus
  source 1..45: mol_type = other DNA
aagtttcacagccacaaggacaagctagtcctagaccctaagagt

SEQ ID NO: 241 - DNA, 45 bp, Mus musculus
  source 1..45: mol_type = other DNA
gaggcaaaaacacagttccttactttgcatttagtaaaaacaaga

SEQ ID NO: 242 - DNA, 45 bp, Mus musculus
  source 1..45: mol_type = other DNA
ttctgttcttcctgtacaagaaatgcaaacggccactgcatttta

SEQ ID NO: 243 - DNA, 45 bp, Mus musculus
  source 1..45: mol_type = other DNA
ggcttatggactgaggtcaaagtctaggagtttccaggtttctga

SEQ ID NO: 244 - DNA, 45 bp, Mus musculus
  source 1..45: mol_type = other DNA
agaaaaggatgactttaagcaagaaactcttccaagcaattccaa

SEQ ID NO: 245 - DNA, 45 bp, Mus musculus
  source 1..45: mol_type = other DNA
gaataaaatgtatgtagaaacaagcaaatttactgaaacaagcag

SEQ ID NO: 246 - DNA, 45 bp, Mus musculus
  source 1..45: mol_type = other DNA
gctcttagcttagtctgttgtttcatgcaaacaccgttgtaacca

SEQ ID NO: 247 - DNA, 45 bp, Mus musculus
  source 1..45: mol_type = other DNA
gtaatattttgggctcttagcttagtctgttgtttcatgcaaaca

SEQ ID NO: 248 - DNA, 45 bp, Mus musculus
  source 1..45: mol_type = other DNA
gactgaggtcaaagtctaggagtttccaggtttctgatgaacagt

SEQ ID NO: 249 - DNA, 45 bp, Mus musculus
  source 1..45: mol_type = other DNA
tccaatgaaagccatgaccacatggacgacgatgatgacgatgat

SEQ ID NO: 250 - DNA, 45 bp, Mus musculus
  source 1..45: mol_type = other DNA
taaagaagaggcaaaaacacagttccttactttgcatttagtaaa

SEQ ID NO: 251 - DNA, 45 bp, Mus musculus
  source 1..45: mol_type = other DNA
ttttatgaataaaatgtatgtagaaacaagcaaatttactgaaac

SEQ ID NO: 252 - DNA, 45 bp, Mus musculus
  source 1..45: mol_type = other DNA
taggtatctgaaattccgaatttctcatgaattagagagttcatc

SEQ ID NO: 253 - DNA, 45 bp, Mus musculus
  source 1..45: mol_type = other DNA
gaaattccgaatttctcatgaattagagagttcatcttctgaggt

SEQ ID NO: 254 - DNA, 45 bp, Mus musculus
  source 1..45: mol_type = other DNA
ttagcttagtctgttgtttcatgcaaacaccgttgtaaccaaaag

SEQ ID NO: 255 - DNA, 45 bp, Mus musculus
  source 1..45: mol_type = other DNA
taaaagagaaactgtaacagtctatatcactatacccttttagtt

SEQ ID NO: 256 - DNA, 45 bp, Mus musculus
  source 1..45: mol_type = other DNA
agaaacaagcaaatttactgaaacaagcagaattaaaagagaaac

SEQ ID NO: 257 - DNA, 45 bp, Mus musculus
  source 1..45: mol_type = other DNA
tcttcctgtacaagaaatgcaaacggccactgcattttaatgatt

SEQ ID NO: 258 - DNA, 45 bp, Mus musculus
  source 1..45: mol_type = other DNA
gtccccaacggccgaggtgatagcttggcttatggactgaggtca

SEQ ID NO: 259 - DNA, 45 bp, Mus musculus
  source 1..45: mol_type = other DNA
ttggcttatggactgaggtcaaagtctaggagtttccaggtttct

SEQ ID NO: 260 - DNA, 45 bp, Mus musculus
  source 1..45: mol_type = other DNA
taagagtaaggaagatgataggtatctgaaattccgaatttctca

SEQ ID NO: 261 - DNA, 45 bp, Mus musculus
  source 1..45: mol_type = other DNA
taatattttgggctcttagcttagtctgttgtttcatgcaaacac

SEQ ID NO: 262 - RNA, 20 nt, synthetic construct
  source 1..20: mol_type = other RNA
taagcaggaatactaactgc

SEQ ID NO: 263 - RNA, 20 nt, synthetic construct
  source 1..20: mol_type = other RNA
actgctagtacacaagcaga

SEQ ID NO: 264 - RNA, 20 nt, synthetic construct
  source 1..20: mol_type = other RNA
ttaagcaagaaactcttcca

SEQ ID NO: 265 - RNA, 20 nt, synthetic construct
  source 1..20: mol_type = other RNA
tttataattagcatatattt

SEQ ID NO: 266 - RNA, 20 nt, synthetic construct
  source 1..20: mol_type = other RNA
gtagaaacaagcaaatttac

SEQ ID NO: 267 - RNA, 20 nt, synthetic construct
  source 1..20: mol_type = other RNA
tctgaaattccgaatttctc

SEQ ID NO: 268 - RNA, 20 nt, synthetic construct
  source 1..20: mol_type = other RNA
ctggaaacacacagacttga

SEQ ID NO: 269 - RNA, 20 nt, synthetic construct
  source 1..20: mol_type = other RNA
gcagccatgagtcaagtcag

SEQ ID NO: 270 - RNA, 20 nt, synthetic construct
  source 1..20: mol_type = other RNA
tgtagaaacaagcaaattta

SEQ ID NO: 271 - RNA, 20 nt, synthetic construct
  source 1..20: mol_type = other RNA
ctcgatgtcatccctgttgc

SEQ ID NO: 272 - RNA, 20 nt, synthetic construct
  source 1..20: mol_type = other RNA
actttaagcaagaaactctt

SEQ ID NO: 273 - RNA, 20 nt, synthetic construct
  source 1..20: mol_type = other RNA
tgcgccacagaatgctgtgt

SEQ ID NO: 274 - RNA, 20 nt, synthetic construct
  source 1..20: mol_type = other RNA
tgcactttgcttctgttctt

SEQ ID NO: 275 - RNA, 20 nt, synthetic construct
  source 1..20: mol_type = other RNA
agagaaagagaggtaatatt

SEQ ID NO: 276 - RNA, 20 nt, synthetic construct
  source 1..20: mol_type = other RNA
tgacgaatctcaccattcgg

SEQ ID NO: 277 - RNA, 20 nt, synthetic construct
  source 1..20: mol_type = other RNA
tgggctcttagcttagtctg

SEQ ID NO: 278 - RNA, 20 nt, synthetic construct
  source 1..20: mol_type = other RNA
tttggcattgcctcctccct

SEQ ID NO: 279 - RNA, 20 nt, synthetic construct
  source 1..20: mol_type = other RNA
tctgacgaatctcaccattc

SEQ ID NO: 280 - RNA, 20 nt, synthetic construct
  source 1..20: mol_type = other RNA
actgaaacaagcagaattaa

SEQ ID NO: 281 - RNA, 20 nt, synthetic construct
  source 1..20: mol_type = other RNA
tcaagcaagttccaaagcca

SEQ ID NO: 282 - RNA, 20 nt, synthetic construct
  source 1..20: mol_type = other RNA
gaggtcaactaaagaagagg

SEQ ID NO: 283 - RNA, 20 nt, synthetic construct
  source 1..20: mol_type = other RNA
tatctgaaattccgaatttc

SEQ ID NO: 284 - RNA, 20 nt, synthetic construct
  source 1..20: mol_type = other RNA
aaacacagttccttactttg

SEQ ID NO: 285 - RNA, 20 nt, synthetic construct
  source 1..20: mol_type = other RNA
atgagattggcagtgatttg

SEQ ID NO: 286 - RNA, 20 nt, synthetic construct
  source 1..20: mol_type = other RNA
tgtttcatgcaaacaccgtt

SEQ ID NO: 287 - RNA, 20 nt, synthetic construct
  source 1..20: mol_type = other RNA
tacacaagcagacactttca

SEQ ID NO: 288 - RNA, 20 nt, synthetic construct
  source 1..20: mol_type = other RNA
aaggacaagctagtcctaga

SEQ ID NO: 289 - RNA, 20 nt, synthetic construct
  source 1..20: mol_type = other RNA
ttccttactttgcatttagt

SEQ ID NO: 290 - RNA, 20 nt, synthetic construct
  source 1..20: mol_type = other RNA
acaagaaatgcaaacggcca

SEQ ID NO: 291 - RNA, 20 nt, synthetic construct
  source 1..20: mol_type = other RNA
gtcaaagtctaggagtttcc

SEQ ID NO: 292 - RNA, 20 nt, synthetic construct
  source 1..20: mol_type = other RNA
taagcaagaaactcttccaa

SEQ ID NO: 293 - RNA, 20 nt, synthetic construct
  source 1..20: mol_type = other RNA
agaaacaagcaaatttactg

SEQ ID NO: 294 - RNA, 20 nt, synthetic construct
  source 1..20: mol_type = other RNA
tgttgtttcatgcaaacacc

SEQ ID NO: 295 - RNA, 20 nt, synthetic construct
  source 1..20: mol_type = other RNA
cttagcttagtctgttgttt

SEQ ID NO: 296 - RNA, 20 nt, synthetic construct
  source 1..20: mol_type = other RNA
ctaggagtttccaggtttct

SEQ ID NO: 297 - RNA, 20 nt, synthetic construct
  source 1..20: mol_type = other RNA
gaccacatggacgacgatga

SEQ ID NO: 298 - RNA, 20 nt, synthetic construct
  source 1..20: mol_type = other RNA
aacacagttccttactttgc

SEQ ID NO: 299 - RNA, 20 nt, synthetic construct
  source 1..20: mol_type = other RNA
gtatgtagaaacaagcaaat

SEQ ID NO: 300 - RNA, 20 nt, synthetic construct
  source 1..20: mol_type = other RNA
ccgaatttctcatgaattag

SEQ ID NO: 301 - RNA, 20 nt, synthetic construct
  source 1..20: mol_type = other RNA
tcatgaattagagagttcat

SEQ ID NO: 302 - RNA, 20 nt, synthetic construct
  source 1..20: mol_type = other RNA
gtttcatgcaaacaccgttg

SEQ ID NO: 303 - RNA, 20 nt, synthetic construct
  source 1..20: mol_type = other RNA
aacagtctatatcactatac

SEQ ID NO: 304 - RNA, 20 nt, synthetic construct
  source 1..20: mol_type = other RNA
tactgaaacaagcagaatta

SEQ ID NO: 305 - RNA, 20 nt, synthetic construct
  source 1..20: mol_type = other RNA
aatgcaaacggccactgcat

SEQ ID NO: 306 - RNA, 20 nt, synthetic construct
  source 1..20: mol_type = other RNA
ggtgatagcttggcttatgg

SEQ ID NO: 307 - RNA, 20 nt, synthetic construct
  source 1..20: mol_type = other RNA
aggtcaaagtctaggagttt

SEQ ID NO: 308 - RNA, 20 nt, synthetic construct
  source 1..20: mol_type = other RNA
tgataggtatctgaaattcc

SEQ ID NO: 309 - RNA, 20 nt, synthetic construct
  source 1..20: mol_type = other RNA
ttagcttagtctgttgtttc

SEQ ID NO: 310 - RNA, 20 nt, synthetic construct
  source 1..20: mol_type = other RNA
gcagttagtattcctgctta

SEQ ID NO: 311 - RNA, 20 nt, synthetic construct
  source 1..20: mol_type = other RNA
tctgcttgtgtactagcagt

SEQ ID NO: 312 - RNA, 20 nt, synthetic construct
  source 1..20: mol_type = other RNA
tggaagagtttcttgcttaa

SEQ ID NO: 313 - RNA, 20 nt, synthetic construct
  source 1..20: mol_type = other RNA
aaatatatgctaattataaa

SEQ ID NO: 314 - RNA, 20 nt, synthetic construct
  source 1..20: mol_type = other RNA
gtaaatttgcttgtttctac

SEQ ID NO: 315 - RNA, 20 nt, synthetic construct
  source 1..20: mol_type = other RNA
gagaaattcggaatttcaga

SEQ ID NO: 316 - RNA, 20 nt, synthetic construct
  source 1..20: mol_type = other RNA
tcaagtctgtgtgtttccag

SEQ ID NO: 317 - RNA, 20 nt, synthetic construct
  source 1..20: mol_type = other RNA
ctgacttgactcatggctgc

SEQ ID NO: 318 - RNA, 20 nt, synthetic construct
  source 1..20: mol_type = other RNA
taaatttgcttgtttctaca

SEQ ID NO: 319 - RNA, 20 nt, synthetic construct
  source 1..20: mol_type = other RNA
gcaacagggatgacatcgag

SEQ ID NO: 320 - RNA, 20 nt, synthetic construct
  source 1..20: mol_type = other RNA
aagagtttcttgcttaaagt

SEQ ID NO: 321 - RNA, 20 nt, synthetic construct
  source 1..20: mol_type = other RNA
acacagcattctgtggcgca

SEQ ID NO: 322 - RNA, 20 nt, synthetic construct
  source 1..20: mol_type = other RNA
aagaacagaagcaaagtgca

SEQ ID NO: 323 - RNA, 20 nt, synthetic construct
  source 1..20: mol_type = other RNA
aatattacctctctttctct

SEQ ID NO: 324 - RNA, 20 nt, synthetic construct
  source 1..20: mol_type = other RNA
ccgaatggtgagattcgtca

SEQ ID NO: 325 - RNA, 20 nt, synthetic construct
  source 1..20: mol_type = other RNA
cagactaagctaagagccca

SEQ ID NO: 326 - RNA, 20 nt, synthetic construct
  source 1..20: mol_type = other RNA
agggaggaggcaatgccaaa

SEQ ID NO: 327 - RNA, 20 nt, synthetic construct
  source 1..20: mol_type = other RNA
gaatggtgagattcgtcaga

SEQ ID NO: 328 - RNA, 20 nt, synthetic construct
  source 1..20: mol_type = other RNA
ttaattctgcttgtttcagt

SEQ ID NO: 329 - RNA, 20 nt, synthetic construct
  source 1..20: mol_type = other RNA
tggctttggaacttgcttga

SEQ ID NO: 330 - RNA, 20 nt, synthetic construct
  source 1..20: mol_type = other RNA
cctcttctttagttgacctc

SEQ ID NO: 331 - RNA, 20 nt, synthetic construct
  source 1..20: mol_type = other RNA
gaaattcggaatttcagata

SEQ ID NO: 332 - RNA, 20 nt, synthetic construct
  source 1..20: mol_type = other RNA
caaagtaaggaactgtgttt

SEQ ID NO: 333 - RNA, 20 nt, synthetic construct
  source 1..20: mol_type = other RNA
caaatcactgccaatctcat

SEQ ID NO: 334 - RNA, 20 nt, synthetic construct
  source 1..20: mol_type = other RNA
aacggtgtttgcatgaaaca

SEQ ID NO: 335 - RNA, 20 nt, synthetic construct
  source 1..20: mol_type = other RNA
tgaaagtgtctgcttgtgta

SEQ ID NO: 336 - RNA, 20 nt, synthetic construct
  source 1..20: mol_type = other RNA
tctaggactagcttgtcctt

SEQ ID NO: 337 - RNA, 20 nt, synthetic construct
  source 1..20: mol_type = other RNA
actaaatgcaaagtaaggaa

SEQ ID NO: 338 - RNA, 20 nt, synthetic construct
  source 1..20: mol_type = other RNA
tggccgtttgcatttcttgt

SEQ ID NO: 339 - RNA, 20 nt, synthetic construct
  source 1..20: mol_type = other RNA
ggaaactcctagactttgac

SEQ ID NO: 340 - RNA, 20 nt, synthetic construct
  source 1..20: mol_type = other RNA
ttggaagagtttcttgctta

SEQ ID NO: 341 - RNA, 20 nt, synthetic construct
  source 1..20: mol_type = other RNA
cagtaaatttgcttgtttct

SEQ ID NO: 342 - RNA, 20 nt, synthetic construct
  source 1..20: mol_type = other RNA
ggtgtttgcatgaaacaaca

SEQ ID NO: 343 - RNA, 20 nt, synthetic construct
  source 1..20: mol_type = other RNA
aaacaacagactaagctaag

SEQ ID NO: 344 - RNA, 20 nt, synthetic construct
  source 1..20: mol_type = other RNA
agaaacctggaaactcctag

SEQ ID NO: 345 - RNA, 20 nt, synthetic construct
  source 1..20: mol_type = other RNA
tcatcgtcgtccatgtggtc

SEQ ID NO: 346 - RNA, 20 nt, synthetic construct
  source 1..20: mol_type = other RNA
gcaaagtaaggaactgtgtt

SEQ ID NO: 347 - RNA, 20 nt, synthetic construct
  source 1..20: mol_type = other RNA
atttgcttgtttctacatac

SEQ ID NO: 348 - RNA, 20 nt, synthetic construct
  source 1..20: mol_type = other RNA
ctaattcatgagaaattcgg

SEQ ID NO: 349 - RNA, 20 nt, synthetic construct
  source 1..20: mol_type = other RNA
atgaactctctaattcatga

SEQ ID NO: 350 - RNA, 20 nt, synthetic construct
  source 1..20: mol_type = other RNA
caacggtgtttgcatgaaac

SEQ ID NO: 351 - RNA, 20 nt, synthetic construct
  source 1..20: mol_type = other RNA
gtatagtgatatagactgtt

SEQ ID NO: 352 - RNA, 20 nt, synthetic construct
  source 1..20: mol_type = other RNA
taattctgcttgtttcagta

SEQ ID NO: 353 - RNA, 20 nt, synthetic construct
  source 1..20: mol_type = other RNA
atgcagtggccgtttgcatt

SEQ ID NO: 354 - RNA, 20 nt, synthetic construct
  source 1..20: mol_type = other RNA
ccataagccaagctatcacc

SEQ ID NO: 355 - RNA, 20 nt, synthetic construct
  source 1..20: mol_type = other RNA
aaactcctagactttgacct

SEQ ID NO: 356 - RNA, 20 nt, synthetic construct
  source 1..20: mol_type = other RNA
ggaatttcagatacctatca

SEQ ID NO: 357 - RNA, 20 nt, synthetic construct
  source 1..20: mol_type = other RNA
gaaacaacagactaagctaa

SEQ ID NO: 358 - DNA, 45 bp, Homo sapiens
  source 1..45: mol_type = other DNA
atatttaataactagtatgtcttgacaacagtctttagattaaaa

SEQ ID NO: 359 - DNA, 45 bp, Homo sapiens
  source 1..45: mol_type = other DNA
atcaatcagctgtgttatgccaaagaatagtaaggttttcttatt

SEQ ID NO: 360 - DNA, 45 bp, Homo sapiens
  source 1..45: mol_type = other DNA
ttttggaagtccatttgaggaatcactgaactatgaggtcagcct

SEQ ID NO: 361 - DNA, 45 bp, Homo sapiens
  source 1..45: mol_type = other DNA
aagactttgaaactcagcggaatcaatcagctgtgttatgccaaa

SEQ ID NO: 362 - DNA, 45 bp, Homo sapiens
  source 1..45: mol_type = other DNA
aaagaatagtaaggttttcttatttatgattatttaaaatggaaa

SEQ ID NO: 363 - DNA, 45 bp, Homo sapiens
  source 1..45: mol_type = other DNA
tactataaattataaagggatatatatatatggatatatatatgt

SEQ ID NO: 364 - DNA, 45 bp, Homo sapiens
  source 1..45: mol_type = other DNA
taagttccagtaggtgttcaaatgttaaatgttaaacataggtca

SEQ ID NO: 365 - DNA, 45 bp, Homo sapiens
  source 1..45: mol_type = other DNA
ctggctatcatattcttggctcagaacaatatttcccattactat

SEQ ID NO: 366 - DNA, 45 bp, Homo sapiens
  source 1..45: mol_type = other DNA
atttaaaatggaaatgtgagaatgtggcaagatgaccgtcacatt

SEQ ID NO: 367 - DNA, 45 bp, Homo sapiens
  source 1..45: mol_type = other DNA
taatttgcaagcactccaggcaaatatattagatgtttgaaaaca

SEQ ID NO: 368 - DNA, 45 bp, Homo sapiens
  source 1..45: mol_type = other DNA
aaaaaaaaaaagaattaaagcattcatacagtttagtgattttgt

SEQ ID NO: 369 - DNA, 45 bp, Homo sapiens
  source 1..45: mol_type = other DNA
gggtttaaagtcaccgttggaactacactgtgtgatcttataaac

SEQ ID NO: 370 - DNA, 45 bp, Homo sapiens
  source 1..45: mol_type = other DNA
ctagtaaaaagacactttgagttagtccaggcttaatgtgcaata

SEQ ID NO: 371 - DNA, 45 bp, Homo sapiens
  source 1..45: mol_type = other DNA
attttaaagcaagacctggtcagtatattactctacattgtccca

SEQ ID NO: 372 - DNA, 45 bp, Homo sapiens
  source 1..45: mol_type = other DNA
attactgtcgcactttttggtatcctgagtttccatagggaacta

SEQ ID NO: 373 - DNA, 45 bp, Homo sapiens
  source 1..45: mol_type = other DNA
atgtttcaaagctggtctgccattctagatgagcctccttgctta

SEQ ID NO: 374 - DNA, 45 bp, Homo sapiens
  source 1..45: mol_type = other DNA
ttgatacgggtactttgtgtcaatatctaatcgtctccactactt

SEQ ID NO: 375 - DNA, 45 bp, Homo sapiens
  source 1..45: mol_type = other DNA
attttcatggaaacatttaacagaaagaactagtaaaaagacact

SEQ ID NO: 376 - DNA, 45 bp, Homo sapiens
  source 1..45: mol_type = other DNA
tttttcagcatttttgtctctattattattattgtttttttccca

SEQ ID NO: 377 - DNA, 45 bp, Homo sapiens
  source 1..45: mol_type = other DNA
actggacagaacgatttcgagatttactactgcctccatctagtc

SEQ ID NO: 378 - DNA, 45 bp, Homo sapiens
  source 1..45: mol_type = other DNA
tgtttcaggagggctgctgaagtatcaaactaatttagataccca

SEQ ID NO: 379 - DNA, 45 bp, Homo sapiens
  source 1..45: mol_type = other DNA
gaatgtggcaagatgaccgtcacattacatgtttaatctggaaac

SEQ ID NO: 380 - DNA, 45 bp, Homo sapiens
  source 1..45: mol_type = other DNA
ttgcataacaagttttggcaagagaacagacctgactatgtcaac

SEQ ID NO: 381 - DNA, 45 bp, Homo sapiens
  source 1..45: mol_type = other DNA
atgtgtgtcagtagggactgaatataagatttatctcctttgcca

SEQ ID NO: 382 - DNA, 45 bp, Homo sapiens
  source 1..45: mol_type = other DNA
tttggtatcctgagtttccatagggaactattgggtttaaagtca

SEQ ID NO: 383 - DNA, 45 bp, Homo sapiens
  source 1..45: mol_type = other DNA
aacttaagtttagaatccagaatattactgtcgcactttttggta

SEQ ID NO: 384 - DNA, 45 bp, Homo sapiens
  source 1..45: mol_type = other DNA
ttctctggggatcattcttataatacaagccttaaatcactaatt

SEQ ID NO: 385 - DNA, 45 bp, Homo sapiens
  source 1..45: mol_type = other DNA
atgtttcaggagggctgctgaagtatcaaactaatttagataccc

SEQ ID NO: 386 - DNA, 45 bp, Homo sapiens
  source 1..45: mol_type = other DNA
ctaactatgatatcttctggtatactcataacctcttctttgtct

SEQ ID NO: 387 - DNA, 45 bp, Homo sapiens
  source 1..45: mol_type = other DNA
atttgtgaagatttgtatggaagagcccagattccaagctaattt

SEQ ID NO: 388 - DNA, 45 bp, Homo sapiens
  source 1..45: mol_type = other DNA
tttcagctgaaaacttttgccatgaagcaggactctaaagaagga

SEQ ID NO: 389 - DNA, 45 bp, Homo sapiens
  source 1..45: mol_type = other DNA
tattattattgtttttttcccaggtagggtttgtcttaggctgta

SEQ ID NO: 390 - DNA, 45 bp, Homo sapiens
  source 1..45: mol_type = other DNA
aagaatagtaaggttttcttatttatgattatttaaaatggaaat

SEQ ID NO: 391 - DNA, 45 bp, Homo sapiens
  source 1..45: mol_type = other DNA
atgcaataagagtttctaactatgatatcttctggtatactcata

SEQ ID NO: 392 - DNA, 45 bp, Homo sapiens
  source 1..45: mol_type = other DNA
tatgcaataagagtttctaactatgatatcttctggtatactcat

SEQ ID NO: 393 - DNA, 45 bp, Homo sapiens
  source 1..45: mol_type = other DNA
atcattcttataatacaagccttaaatcactaattttagtagcaa

SEQ ID NO: 394 - DNA, 45 bp, Homo sapiens
  source 1..45: mol_type = other DNA
ttaccttttaagtaagaggaaatatgatattcttattcaaactta

SEQ ID NO: 395 - DNA, 45 bp, Homo sapiens
  source 1..45: mol_type = other DNA
atttctctggggatcattcttataatacaagccttaaatcactaa

SEQ ID NO: 396 - DNA, 45 bp, Homo sapiens
  source 1..45: mol_type = other DNA
atcacagcctctacatatgcaataagagtttctaactatgatatc

SEQ ID NO: 397 - DNA, 45 bp, Homo sapiens
  source 1..45: mol_type = other DNA
ttacataaaacataacatgatagatgctaaggcctttttttgcta

SEQ ID NO: 398 - DNA, 45 bp, Homo sapiens
  source 1..45: mol_type = other DNA
ggtcaaagaaaattttaaagcaagacctggtcagtatattactct

SEQ ID NO: 399 - DNA, 45 bp, Homo sapiens
  source 1..45: mol_type = other DNA
attctagatgagcctccttgcttatttaagttccagtaggtgttc

SEQ ID NO: 400 - DNA, 45 bp, Homo sapiens
  source 1..45: mol_type = other DNA
gtccctttcatcttcatttaaatctatcactattgcatggtaatg

SEQ ID NO: 401 - DNA, 45 bp, Homo sapiens
  source 1..45: mol_type = other DNA
tagattaaaaagaattttcatggaaacatttaacagaaagaacta

SEQ ID NO: 402 - DNA, 45 bp, Homo sapiens
  source 1..45: mol_type = other DNA
tgagtaaccagaacaactcatatgggacaagatttgaatacaata

SEQ ID NO: 403 - DNA, 45 bp, Homo sapiens
  source 1..45: mol_type = other DNA
cgaaattctgcccttcattcaatctagaaattatcccaagctgta

SEQ ID NO: 404 - DNA, 45 bp, Homo sapiens
  source 1..45: mol_type = other DNA
ctgaatgatatttacttgtagatatttcttcctttccttgaaatt

SEQ ID NO: 405 - DNA, 45 bp, Homo sapiens
  source 1..45: mol_type = other DNA
agactatatgctaactgaaccttcctcaaatgttgttatagtatc

SEQ ID NO: 406 - DNA, 45 bp, Homo sapiens
  source 1..45: mol_type = other DNA
tatgggacaagatttgaatacaataaagagtctttcagctgaaaa

SEQ ID NO: 407 - DNA, 45 bp, Homo sapiens
  source 1..45: mol_type = other DNA
atcgtctccactacttatgctaatacctctatttgatatctgaag

SEQ ID NO: 408 - RNA, 20 nt, synthetic construct
  source 1..20: mol_type = other RNA
tatgtcttgacaacagtctt

SEQ ID NO: 409 - RNA, 20 nt, synthetic construct
  source 1..20: mol_type = other RNA
tatgccaaagaatagtaagg

SEQ ID NO: 410 - RNA, 20 nt, synthetic construct
  source 1..20: mol_type = other RNA
tgaggaatcactgaactatg

SEQ ID NO: 411 - RNA, 20 nt, synthetic construct
  source 1..20: mol_type = other RNA
agcggaatcaatcagctgtg

SEQ ID NO: 412 - RNA, 20 nt, synthetic construct
  source 1..20: mol_type = other RNA
tttcttatttatgattattt

SEQ ID NO: 413 - RNA, 20 nt, synthetic construct
  source 1..20: mol_type = other RNA
agggatatatatatatggat

SEQ ID NO: 414 - RNA, 20 nt, synthetic construct
  source 1..20: mol_type = other RNA
gttcaaatgttaaatgttaa

SEQ ID NO: 415 - RNA, 20 nt, synthetic construct
  source 1..20: mol_type = other RNA
ttggctcagaacaatatttc

SEQ ID NO: 416 - RNA, 20 nt, synthetic construct
  source 1..20: mol_type = other RNA
gtgagaatgtggcaagatga

SEQ ID NO: 417 - RNA, 20 nt, synthetic construct
  source 1..20: mol_type = other RNA
ccaggcaaatatattagatg

SEQ ID NO: 418 - RNA, 20 nt, synthetic construct
  source 1..20: mol_type = other RNA
taaagcattcatacagttta

SEQ ID NO: 419 - RNA, 20 nt, synthetic construct
  source 1..20: mol_type = other RNA
gttggaactacactgtgtga

SEQ ID NO: 420 - RNA, 20 nt, synthetic construct
  source 1..20: mol_type = other RNA
tttgagttagtccaggctta

SEQ ID NO: 421 - RNA, 20 nt, synthetic construct
  source 1..20: mol_type = other RNA
ctggtcagtatattactcta

SEQ ID NO: 422 - RNA, 20 nt, synthetic construct
  source 1..20: mol_type = other RNA
tttggtatcctgagtttcca

SEQ ID NO: 423 - RNA, 20 nt, synthetic construct
  source 1..20: mol_type = other RNA
tctgccattctagatgagcc

SEQ ID NO: 424 - RNA, 20 nt, synthetic construct
  source 1..20: mol_type = other RNA
tgtgtcaatatctaatcgtc

SEQ ID NO: 425 - RNA, 20 nt, synthetic construct
  source 1..20: mol_type = other RNA
tttaacagaaagaactagta

SEQ ID NO: 426 - RNA, 20 nt, synthetic construct
  source 1..20: mol_type = other RNA
gtctctattattattattgt

SEQ ID NO: 427 - RNA, 20 nt, synthetic construct
  source 1..20: mol_type = other RNA
ttcgagatttactactgcct

SEQ ID NO: 428 - RNA, 20 nt, synthetic construct
  source 1..20: mol_type = other RNA
gctgaagtatcaaactaatt

SEQ ID NO: 429 - RNA, 20 nt, synthetic construct
  source 1..20: mol_type = other RNA
accgtcacattacatgttta

SEQ ID NO: 430 - RNA, 20 nt, synthetic construct
  source 1..20: mol_type = other RNA
tggcaagagaacagacctga

SEQ ID NO: 431 - RNA, 20 nt, synthetic construct
  source 1..20: mol_type = other RNA
gactgaatataagatttatc

SEQ ID NO: 432 - RNA, 20 nt, synthetic construct
  source 1..20: mol_type = other RNA
ttccatagggaactattggg

SEQ ID NO: 433 - RNA, 20 nt, synthetic construct
  source 1..20: mol_type = other RNA
tccagaatattactgtcgca

SEQ ID NO: 434 - RNA, 20 nt, synthetic construct
  source 1..20: mol_type = other RNA
tcttataatacaagccttaa

SEQ ID NO: 435 - RNA, 20 nt, synthetic construct
  source 1..20: mol_type = other RNA
tgctgaagtatcaaactaat

SEQ ID NO: 436 - RNA, 20 nt, synthetic construct
  source 1..20: mol_type = other RNA
tctggtatactcataacctc

SEQ ID NO: 437 - RNA, 20 nt, synthetic construct
  source 1..20: mol_type = other RNA
tatggaagagcccagattcc

SEQ ID NO: 438 - RNA, 20 nt, synthetic construct
  source 1..20: mol_type = other RNA
tttgccatgaagcaggactc

SEQ ID NO: 439 - RNA, 20 nt, synthetic construct
  source 1..20: mol_type = other RNA
tttcccaggtagggtttgtc

SEQ ID NO: 440 - RNA, 20 nt, synthetic construct
  source 1..20: mol_type = other RNA
ttcttatttatgattattta

SEQ ID NO: 441 - RNA, 20 nt, synthetic construct
  source 1..20: mol_type = other RNA
ctaactatgatatcttctgg

SEQ ID NO: 442 - RNA, 20 nt, synthetic construct
  source 1..20: mol_type = other RNA
tctaactatgatatcttctg

SEQ ID NO: 443 - RNA, 20 nt, synthetic construct
  source 1..20: mol_type = other RNA
caagccttaaatcactaatt

SEQ ID NO: 444 - RNA, 20 nt, synthetic construct
  source 1..20: mol_type = other RNA
gaggaaatatgatattctta

SEQ ID NO: 445 - RNA, 20 nt, synthetic construct
  source 1..20: mol_type = other RNA
attcttataatacaagcctt

SEQ ID NO: 446 - RNA, 20 nt, synthetic construct
  source 1..20: mol_type = other RNA
tatgcaataagagtttctaa

SEQ ID NO: 447 - RNA, 20 nt, synthetic construct
  source 1..20: mol_type = other RNA
catgatagatgctaaggcct

SEQ ID NO: 448 - RNA, 20 nt, synthetic construct
  source 1..20: mol_type = other RNA
taaagcaagacctggtcagt

SEQ ID NO: 449 - RNA, 20 nt, synthetic construct
  source 1..20: mol_type = other RNA
ccttgcttatttaagttcca

SEQ ID NO: 450 - RNA, 20 nt, synthetic construct
  source 1..20: mol_type = other RNA
atttaaatctatcactattg

SEQ ID NO: 451 - RNA, 20 nt, synthetic construct
  source 1..20: mol_type = other RNA
tttcatggaaacatttaaca

SEQ ID NO: 452 - RNA, 20 nt, synthetic construct
  source 1..20: mol_type = other RNA
actcatatgggacaagattt

SEQ ID NO: 453 - RNA, 20 nt, synthetic construct
  source 1..20: mol_type = other RNA
cattcaatctagaaattatc

SEQ ID NO: 454 - RNA, 20 nt, synthetic construct
  source 1..20: mol_type = other RNA
ttgtagatatttcttccttt

SEQ ID NO: 455 - RNA, 20 nt, synthetic construct
  source 1..20: mol_type = other RNA
tgaaccttcctcaaatgttg

SEQ ID NO: 456 - RNA, 20 nt, synthetic construct
  source 1..20: mol_type = other RNA
gaatacaataaagagtcttt

SEQ ID NO: 457 - RNA, 20 nt, synthetic construct
  source 1..20: mol_type = other RNA
tatgctaatacctctatttg

SEQ ID NO: 458 - RNA, 20 nt, synthetic construct
  source 1..20: mol_type = other RNA
aagactgttgtcaagacata

SEQ ID NO: 459 - RNA, 20 nt, synthetic construct
  source 1..20: mol_type = other RNA
ccttactattctttggcata

SEQ ID NO: 460 - RNA, 20 nt, synthetic construct
  source 1..20: mol_type = other RNA
catagttcagtgattcctca

SEQ ID NO: 461 - RNA, 20 nt, synthetic construct
  source 1..20: mol_type = other RNA
cacagctgattgattccgct

SEQ ID NO: 462 - RNA, 20 nt, synthetic construct
  source 1..20: mol_type = other RNA
aaataatcataaataagaaa

SEQ ID NO: 463 - RNA, 20 nt, synthetic construct
  source 1..20: mol_type = other RNA
atccatatatatatatccct

SEQ ID NO: 464 - RNA, 20 nt, synthetic construct
  source 1..20: mol_type = other RNA
ttaacatttaacatttgaac

SEQ ID NO: 465 - RNA, 20 nt, synthetic construct
  source 1..20: mol_type = other RNA
gaaatattgttctgagccaa

SEQ ID NO: 466 - RNA, 20 nt, synthetic construct
  source 1..20: mol_type = other RNA
tcatcttgccacattctcac

SEQ ID NO: 467 - RNA, 20 nt, synthetic construct
  source 1..20: mol_type = other RNA
catctaatatatttgcctgg

SEQ ID NO: 468 - RNA, 20 nt, synthetic construct
  source 1..20: mol_type = other RNA
taaactgtatgaatgcttta

SEQ ID NO: 469 - RNA, 20 nt, synthetic construct
  source 1..20: mol_type = other RNA
tcacacagtgtagttccaac

SEQ ID NO: 470 - RNA, 20 nt, synthetic construct
  source 1..20: mol_type = other RNA
taagcctggactaactcaaa

SEQ ID NO: 471 - RNA, 20 nt, synthetic construct
  source 1..20: mol_type = other RNA
tagagtaatatactgaccag

SEQ ID NO: 472 - RNA, 20 nt, synthetic construct
  source 1..20: mol_type = other RNA
tggaaactcaggataccaaa

SEQ ID NO: 473 - RNA, 20 nt, synthetic construct
  source 1..20: mol_type = other RNA
ggctcatctagaatggcaga

SEQ ID NO: 474 - RNA, 20 nt, synthetic construct
  source 1..20: mol_type = other RNA
gacgattagatattgacaca

SEQ ID NO: 475 - RNA, 20 nt, synthetic construct
  source 1..20: mol_type = other RNA
tactagttctttctgttaaa

SEQ ID NO: 476 - RNA, 20 nt, synthetic construct
  source 1..20: mol_type = other RNA
acaataataataatagagac

SEQ ID NO: 477 - RNA, 20 nt, synthetic construct
  source 1..20: mol_type = other RNA
aggcagtagtaaatctcgaa

SEQ ID NO: 478 - RNA, 20 nt, synthetic construct
  source 1..20: mol_type = other RNA
aattagtttgatacttcagc

SEQ ID NO: 479 - RNA, 20 nt, synthetic construct
  source 1..20: mol_type = other RNA
taaacatgtaatgtgacggt

SEQ ID NO: 480 - RNA, 20 nt, synthetic construct
  source 1..20: mol_type = other RNA
tcaggtctgttctcttgcca

SEQ ID NO: 481 - RNA, 20 nt, synthetic construct
  source 1..20: mol_type = other RNA
gataaatcttatattcagtc

SEQ ID NO: 482 - RNA, 20 nt, synthetic construct
  source 1..20: mol_type = other RNA
cccaatagttccctatggaa

SEQ ID NO: 483 - RNA, 20 nt, synthetic construct
  source 1..20: mol_type = other RNA
tgcgacagtaatattctgga

SEQ ID NO: 484 - RNA, 20 nt, synthetic construct
  source 1..20: mol_type = other RNA
ttaaggcttgtattataaga

SEQ ID NO: 485 - RNA, 20 nt, synthetic construct
  source 1..20: mol_type = other RNA
attagtttgatacttcagca

SEQ ID NO: 486 - RNA, 20 nt, synthetic construct
  source 1..20: mol_type = other RNA
gaggttatgagtataccaga

SEQ ID NO: 487 - RNA, 20 nt, synthetic construct
  source 1..20: mol_type = other RNA
ggaatctgggctcttccata

SEQ ID NO: 488 - RNA, 20 nt, synthetic construct
  source 1..20: mol_type = other RNA
gagtcctgcttcatggcaaa

SEQ ID NO: 489 - RNA, 20 nt, synthetic construct
  source 1..20: mol_type = other RNA
gacaaaccctacctgggaaa

SEQ ID NO: 490 - RNA, 20 nt, synthetic construct
  source 1..20: mol_type = other RNA
taaataatcataaataagaa

SEQ ID NO: 491 - RNA, 20 nt, synthetic construct
  source 1..20: mol_type = other RNA
ccagaagatatcatagttag

SEQ ID NO: 492 - RNA, 20 nt, synthetic construct
  source 1..20: mol_type = other RNA
cagaagatatcatagttaga

SEQ ID NO: 493 - RNA, 20 nt, synthetic construct
  source 1..20: mol_type = other RNA
aattagtgatttaaggcttg

SEQ ID NO: 494 - RNA, 20 nt, synthetic construct
  source 1..20: mol_type = other RNA
taagaatatcatatttcctc

SEQ ID NO: 495 - RNA, 20 nt, synthetic construct
  source 1..20: mol_type = other RNA
aaggcttgtattataagaat

SEQ ID NO: 496 - RNA, 20 nt, synthetic construct
  source 1..20: mol_type = other RNA
ttagaaactcttattgcata

SEQ ID NO: 497 - RNA, 20 nt, synthetic construct
  source 1..20: mol_type = other RNA
aggccttagcatctatcatg

SEQ ID NO: 498 - RNA, 20 nt, synthetic construct
  source 1..20: mol_type = other RNA
actgaccaggtcttgcttta

SEQ ID NO: 499 - RNA, 20 nt, synthetic construct
  source 1..20: mol_type = other RNA
tggaacttaaataagcaagg

SEQ ID NO: 500 - RNA, 20 nt, synthetic construct
  source 1..20: mol_type = other RNA
caatagtgatagatttaaat

SEQ ID NO: 501 - RNA, 20 nt, synthetic construct
  source 1..20: mol_type = other RNA
tgttaaatgtttccatgaaa

SEQ ID NO: 502 - RNA, 20 nt, synthetic construct
  source 1..20: mol_type = other RNA
aaatcttgtcccatatgagt

SEQ ID NO: 503 - RNA, 20 nt, synthetic construct
  source 1..20: mol_type = other RNA
gataatttctagattgaatg

SEQ ID NO: 504 - RNA, 20 nt, synthetic construct
  source 1..20: mol_type = other RNA
aaaggaagaaatatctacaa

SEQ ID NO: 505 - RNA, 20 nt, synthetic construct
  source 1..20: mol_type = other RNA
caacatttgaggaaggttca

SEQ ID NO: 506 - RNA, 20 nt, synthetic construct
  source 1..20: mol_type = other RNA
aaagactctttattgtattc

SEQ ID NO: 507 - RNA, 20 nt, synthetic construct
  source 1..20: mol_type = other RNA
caaatagaggtattagcata

SEQ ID NO: 508 - DNA, 45 bp, Mus musculus
  source 1..45: mol_type = other DNA
catactaaaacatttagcatcaaataaccacctgtatatttagtt

SEQ ID NO: 509 - DNA, 45 bp, Mus musculus
  source 1..45: mol_type = other DNA
gtttatcaacttaatttggtatataattattaatgctatcaaata

SEQ ID NO: 510 - DNA, 45 bp, Mus musculus
  source 1..45: mol_type = other DNA
atgattacaataaatcctgttattatttggatttcatgtgattca

SEQ ID NO: 511 - DNA, 45 bp, Mus musculus
  source 1..45: mol_type = other DNA
ttttgtatatcacatgattacaataaatcctgttattatttggat

SEQ ID NO: 512 - DNA, 45 bp, Mus musculus
  source 1..45: mol_type = other DNA
aagacttagaaactcagctgaatcaaacagctgtgctatgccaaa

SEQ ID NO: 513 - DNA, 45 bp, Mus musculus
  source 1..45: mol_type = other DNA
agatttaagtcccctgttcatgtgatccaatcagtttatcaccta

SEQ ID NO: 514 - RNA, 20 nt, synthetic construct
  source 1..20: mol_type = other RNA
ggaaactctgcactgaggag

SEQ ID NO: 515 - DNA, 45 bp, Mus musculus
  source 1..45: mol_type = other DNA
aaagaataccaagggtttgctatttatgattatttaaaatgagaa

SEQ ID NO: 516 - DNA, 45 bp, Mus musculus
  source 1..45: mol_type = other DNA
aagcatactaaaacatttagcatcaaataaccacctgtatattta

SEQ ID NO: 517 - DNA, 45 bp, Mus musculus
  source 1..45: mol_type = other DNA
atgcttggtaatcttttgtatatcacatgattacaataaatcctg

SEQ ID NO: 518 - DNA, 45 bp, Mus musculus
  source 1..45: mol_type = other DNA
atttggaagcccatttgaggagtcactgaactatgaagttagtct

SEQ ID NO: 519 - DNA, 45 bp, Mus musculus
  source 1..45: mol_type = other DNA
attatttttgcactgtttgatatcacggtgagttactagatttaa

SEQ ID NO: 520 - DNA, 45 bp, Mus musculus
  source 1..45: mol_type = other DNA
atcataacttatttttatgcctatgtgaattcatcacatttcaaa

SEQ ID NO: 521 - DNA, 45 bp, Mus musculus
  source 1..45: mol_type = other DNA
tgtggagacataaactatgacatcatagcaatgctgtctagatta

SEQ ID NO: 522 - DNA, 45 bp, Mus musculus
  source 1..45: mol_type = other DNA
taagaaagaagatattcatacagtttatcaacttaatttggtata

SEQ ID NO: 523 - DNA, 45 bp, Mus musculus
  source 1..45: mol_type = other DNA
agtttatcaacttaatttggtatataattattaatgctatcaaat

SEQ ID NO: 524 - DNA, 45 bp, Mus musculus
  source 1..45: mol_type = other DNA
actttaaagcaagacctggccagtatattattctccattgcccca

SEQ ID NO: 525 - DNA, 45 bp, Mus musculus
  source 1..45: mol_type = other DNA
ttatacccatgtggatgtagtatctacttatgtttgcatcttatc

SEQ ID NO: 526 - DNA, 45 bp, Mus musculus
  source 1..45: mol_type = other DNA
agaagcacaagacctctctcctttgactcactttgatttcagata

SEQ ID NO: 527 - DNA, 45 bp, Mus musculus
  source 1..45: mol_type = other DNA
attatttaatagaactgtttgatcaccgatttgttcaatttagtc

SEQ ID NO: 528 - RNA, 20 nt, synthetic construct
  source 1..20: mol_type = other RNA
agaaacgaccagcgtggcaa

SEQ ID NO: 529 - DNA, 45 bp, Mus musculus
  source 1..45: mol_type = other DNA
gtgcaatactgattccttaccaatcataacttatttttatgccta

SEQ ID NO: 530 - DNA, 45 bp, Mus musculus
  source 1..45: mol_type = other DNA
agctttatcacacatatagaagacagcttcatttctttttagatt

SEQ ID NO: 531 - DNA, 45 bp, Mus musculus
  source 1..45: mol_type = other DNA
aagaataccaagggtttgctatttatgattatttaaaatgagaat

SEQ ID NO: 532 - DNA, 45 bp, Mus musculus
  source 1..45: mol_type = other DNA
gagccaaaggggccctgaagaatatctgatgtggtgattcacatt

SEQ ID NO: 533 - DNA, 45 bp, Mus musculus
  source 1..45: mol_type = other DNA
gtttatcacctattttttaatattcaggaatcagaaggaaaattt

SEQ ID NO: 534 - DNA, 45 bp, Mus musculus
  source 1..45: mol_type = other DNA
tttcctatccctcctatgggcaatgtgcaatgctcatgtagactg

SEQ ID NO: 535 - DNA, 45 bp, Mus musculus
  source 1..45: mol_type = other DNA
tgagattctgcccttcattcactctagaaattaccctaagttata

SEQ ID NO: 536 - DNA, 45 bp, Mus musculus
  source 1..45: mol_type = other DNA
tttatctgggtgtgcagagacatccaatcattccagtggtttgca

SEQ ID NO: 537 - DNA, 45 bp, Mus musculus
  source 1..45: mol_type = other DNA
ttatttttatgcctatgtgaattcatcacatttcaaaaattcagg

SEQ ID NO: 538 - DNA, 45 bp, Mus musculus
  source 1..45: mol_type = other DNA
tgcaatactgattccttaccaatcataacttatttttatgcctat

SEQ ID NO: 539 - DNA, 45 bp, Mus musculus
  source 1..45: mol_type = other DNA
aactccagtgcatgttcaaggatctatatgttaaacatggtatat

SEQ ID NO: 540 - DNA, 45 bp, Mus musculus
  source 1..45: mol_type = other DNA
cctctttaaattgttttcggttgaacacattcaagattgaacaaa

SEQ ID NO: 541 - DNA, 45 bp, Mus musculus
  source 1..45: mol_type = other DNA
ttgtatatcacatgattacaataaatcctgttattatttggattt

SEQ ID NO: 542 - DNA, 45 bp, Mus musculus
  source 1..45: mol_type = other DNA
gatttcatgtgattcagtactaacagacaagcatactaaaacatt

SEQ ID NO: 543 - DNA, 45 bp, Mus musculus
  source 1..45: mol_type = other DNA
acttgtgaagatttgcctggaagaacccaagttccaagctcattt

SEQ ID NO: 544 - DNA, 45 bp, Mus musculus
  source 1..45: mol_type = other DNA
agatattcatacagtttatcaacttaatttggtatataattatta

SEQ ID NO: 545 - DNA, 45 bp, Mus musculus
  source 1..45: mol_type = other DNA
tactttatctgggtgtgcagagacatccaatcattccagtggttt

SEQ ID NO: 546 - DNA, 45 bp, Mus musculus
  source 1..45: mol_type = other DNA
ccattttctaggttgtgcataagtggagtagctttcatttggata

SEQ ID NO: 547 - DNA, 45 bp, Mus musculus
  source 1..45: mol_type = other DNA
gaaaatttactaccttaagaaagaagatattcatacagtttatca

SEQ ID NO: 548 - DNA, 45 bp, Mus musculus
  source 1..45: mol_type = other DNA
cactctactggatgactggaaaccatacaagttaagaagactgta

SEQ ID NO: 549 - DNA, 45 bp, Mus musculus
  source 1..45: mol_type = other DNA
ctaattatgatatcttctggtatactcacaacctcttctttgtct

SEQ ID NO: 550 - DNA, 45 bp, Mus musculus
  source 1..45: mol_type = other DNA
atttaatagaactgtttgatcaccgatttgttcaatttagtccca

SEQ ID NO: 551 - DNA, 45 bp, Mus musculus
  source 1..45: mol_type = other DNA
ctttttagattctatggtggtggttactgttgtataaatcttaaa

SEQ ID NO: 552 - DNA, 45 bp, Mus musculus
  source 1..45: mol_type = other DNA
tctacatgctgctgctgttgcatgtttcaggtggtttgttgaagt

SEQ ID NO: 553 - DNA, 45 bp, Mus musculus
  source 1..45: mol_type = other DNA
tggtatataattattaatgctatcaaataggacacattgctgaaa

SEQ ID NO: 554 - DNA, 45 bp, Mus musculus
  source 1..45: mol_type = other DNA
gatattcatacagtttatcaacttaatttggtatataattattaa

SEQ ID NO: 555 - DNA, 45 bp, Mus musculus
  source 1..45: mol_type = other DNA
cacattgctgaaattagtaccttaacttatgcttggtaatctttt

SEQ ID NO: 556 - DNA, 45 bp, Mus musculus
  source 1..45: mol_type = other DNA
gtatttattcaaatttcagtttagaatcactaatattatttttgc

SEQ ID NO: 557 - DNA, 45 bp, Mus musculus
  source 1..45: mol_type = other DNA
acttccaagatgaaccatgccttcttactgtgtcctactgaaacc

SEQ ID NO: 558 - RNA, 20 nt, synthetic construct
  source 1..20: mol_type = other RNA
agcatcaaataaccacctgt

SEQ ID NO: 559 - RNA, 20 nt, synthetic construct
  source 1..20: mol_type = other RNA
ttggtatataattattaatg

SEQ ID NO: 560 - RNA, 20 nt, synthetic construct
  source 1..20: mol_type = other RNA
cctgttattatttggatttc

SEQ ID NO: 561 - RNA, 20 nt, synthetic construct
  source 1..20: mol_type = other RNA
gattacaataaatcctgtta

SEQ ID NO: 562 - RNA, 20 nt, synthetic construct
  source 1..20: mol_type = other RNA
agctgaatcaaacagctgtg

SEQ ID NO: 563 - RNA, 20 nt, synthetic construct
  source 1..20: mol_type = other RNA
gttcatgtgatccaatcagt

SEQ ID NO: 564 - RNA, 20 nt, synthetic construct
  source 1..20: mol_type = other RNA
tcagtcatcaatcatccctc

SEQ ID NO: 565 - RNA, 20 nt, synthetic construct
  source 1..20: mol_type = other RNA
tttgctatttatgattattt

SEQ ID NO: 566 - RNA, 20 nt, synthetic construct
  source 1..20: mol_type = other RNA
tttagcatcaaataaccacc

SEQ ID NO: 567 - RNA, 20 nt, synthetic construct
  source 1..20: mol_type = other RNA
ttgtatatcacatgattaca

SEQ ID NO: 568 - RNA, 20 nt, synthetic construct
  source 1..20: mol_type = other RNA
tgaggagtcactgaactatg

SEQ ID NO: 569 - RNA, 20 nt, synthetic construct
  source 1..20: mol_type = other RNA
tttgatatcacggtgagtta

SEQ ID NO: 570 - RNA, 20 nt, synthetic construct
  source 1..20: mol_type = other RNA
tatgcctatgtgaattcatc

SEQ ID NO: 571 - RNA, 20 nt, synthetic construct
  source 1..20: mol_type = other RNA
tatgacatcatagcaatgct

SEQ ID NO: 572 - RNA, 20 nt, synthetic construct
  source 1..20: mol_type = other RNA
tcatacagtttatcaactta

SEQ ID NO: 573 - RNA, 20 nt, synthetic construct
  source 1..20: mol_type = other RNA
tttggtatataattattaat

SEQ ID NO: 574 - RNA, 20 nt, synthetic construct
  source 1..20: mol_type = other RNA
ctggccagtatattattctc

SEQ ID NO: 575 - RNA, 20 nt, synthetic construct
  source 1..20: mol_type = other RNA
tgtagtatctacttatgttt

SEQ ID NO: 576 - RNA, 20 nt, synthetic construct
  source 1..20: mol_type = other RNA
ctctcctttgactcactttg

SEQ ID NO: 577 - RNA, 20 nt, synthetic construct
  source 1..20: mol_type = other RNA
tgtttgatcaccgatttgtt

SEQ ID NO: 578 - RNA, 20 nt, synthetic construct
  source 1..20: mol_type = other RNA
tacgcaataagagtttctaa

SEQ ID NO: 579 - RNA, 20 nt, synthetic construct
  source 1..20: mol_type = other RNA
cttaccaatcataacttatt

SEQ ID NO: 580 - RNA, 20 nt, synthetic construct
  source 1..20: mol_type = other RNA
atagaagacagcttcatttc

SEQ ID NO: 581 - RNA, 20 nt, synthetic construct
  source 1..20: mol_type = other RNA
ttgctatttatgattattta

SEQ ID NO: 582 - RNA, 20 nt, synthetic construct
  source 1..20: mol_type = other RNA
tgaagaatatctgatgtggt

SEQ ID NO: 583 - RNA, 20 nt, synthetic construct
  source 1..20: mol_type = other RNA
tttaatattcaggaatcaga

SEQ ID NO: 584 - RNA, 20 nt, synthetic construct
  source 1..20: mol_type = other RNA
atgggcaatgtgcaatgctc

SEQ ID NO: 585 - RNA, 20 nt, synthetic construct
  source 1..20: mol_type = other RNA
cattcactctagaaattacc

SEQ ID NO: 586 - RNA, 20 nt, synthetic construct
  source 1..20: mol_type = other RNA
agagacatccaatcattcca

SEQ ID NO: 587 - RNA, 20 nt, synthetic construct
  source 1..20: mol_type = other RNA
tgtgaattcatcacatttca

SEQ ID NO: 588 - RNA, 20 nt, synthetic construct
  source 1..20: mol_type = other RNA
ttaccaatcataacttattt

SEQ ID NO: 589 - RNA, 20 nt, synthetic construct
  source 1..20: mol_type = other RNA
tcaaggatctatatgttaaa

SEQ ID NO: 590 - RNA, 20 nt, synthetic construct
  source 1..20: mol_type = other RNA
ttcggttgaacacattcaag

SEQ ID NO: 591 - RNA, 20 nt, synthetic construct
  source 1..20: mol_type = other RNA
ttacaataaatcctgttatt

SEQ ID NO: 592 - RNA, 20 nt, synthetic construct
  source 1..20: mol_type = other RNA
agtactaacagacaagcata

SEQ ID NO: 593 - RNA, 20 nt, synthetic construct
  source 1..20: mol_type = other RNA
cctggaagaacccaagttcc

SEQ ID NO: 594 - RNA, 20 nt, synthetic construct
  source 1..20: mol_type = other RNA
ttatcaacttaatttggtat

SEQ ID NO: 595 - RNA, 20 nt, synthetic construct
  source 1..20: mol_type = other RNA
tgcagagacatccaatcatt

SEQ ID NO: 596 - RNA, 20 nt, synthetic construct
  source 1..20: mol_type = other RNA
tgcataagtggagtagcttt

SEQ ID NO: 597 - RNA, 20 nt, synthetic construct
  source 1..20: mol_type = other RNA
taagaaagaagatattcata

SEQ ID NO: 598 - RNA, 20 nt, synthetic construct
  source 1..20: mol_type = other RNA
ctggaaaccatacaagttaa

SEQ ID NO: 599 - RNA, 20 nt, synthetic construct
  source 1..20: mol_type = other RNA
tctggtatactcacaacctc

SEQ ID NO: 600 - RNA, 20 nt, synthetic construct
  source 1..20: mol_type = other RNA
ttgatcaccgatttgttcaa

SEQ ID NO: 601 - RNA, 20 nt, synthetic construct
  source 1..20: mol_type = other RNA
ggtggtggttactgttgtat

SEQ ID NO: 602 - RNA, 20 nt, synthetic construct
  source 1..20: mol_type = other RNA
tgttgcatgtttcaggtggt

SEQ ID NO: 603 - RNA, 20 nt, synthetic construct
  source 1..20: mol_type = other RNA
aatgctatcaaataggacac

SEQ ID NO: 604 - RNA, 20 nt, synthetic construct
  source 1..20: mol_type = other RNA
tatcaacttaatttggtata

SEQ ID NO: 605 - RNA, 20 nt, synthetic construct
  source 1..20: mol_type = other RNA
agtaccttaacttatgcttg

SEQ ID NO: 606 - RNA, 20 nt, synthetic construct
  source 1..20: mol_type = other RNA
tcagtttagaatcactaata

SEQ ID NO: 607 - RNA, 20 nt, synthetic construct
  source 1..20: mol_type = other RNA
catgccttcttactgtgtcc

SEQ ID NO: 608 - RNA, 20 nt, synthetic construct
  source 1..20: mol_type = other RNA
acaggtggttatttgatgct

SEQ ID NO: 609 - RNA, 20 nt, synthetic construct
  source 1..20: mol_type = other RNA
cattaataattatataccaa

SEQ ID NO: 610 - RNA, 20 nt, synthetic construct
  source 1..20: mol_type = other RNA
gaaatccaaataataacagg

SEQ ID NO: 611 - RNA, 20 nt, synthetic construct
  source 1..20: mol_type = other RNA
taacaggatttattgtaatc

SEQ ID NO: 612 - RNA, 20 nt, synthetic construct
  source 1..20: mol_type = other RNA
cacagctgtttgattcagct

SEQ ID NO: 613 - RNA, 20 nt, synthetic construct
  source 1..20: mol_type = other RNA
actgattggatcacatgaac

SEQ ID NO: 614 - RNA, 20 nt, synthetic construct
  source 1..20: mol_type = other RNA
gagggatgattgatgactga

SEQ ID NO: 615 - RNA, 20 nt, synthetic construct
  source 1..20: mol_type = other RNA
aaataatcataaatagcaaa

SEQ ID NO: 616 - RNA, 20 nt, synthetic construct
  source 1..20: mol_type = other RNA
ggtggttatttgatgctaaa

SEQ ID NO: 617 - RNA, 20 nt, synthetic construct
  source 1..20: mol_type = other RNA
tgtaatcatgtgatatacaa

SEQ ID NO: 618 - RNA, 20 nt, synthetic construct
  source 1..20: mol_type = other RNA
catagttcagtgactcctca

SEQ ID NO: 619 - RNA, 20 nt, synthetic construct
  source 1..20: mol_type = other RNA
taactcaccgtgatatcaaa

SEQ ID NO: 620 - RNA, 20 nt, synthetic construct
  source 1..20: mol_type = other RNA
gatgaattcacataggcata

SEQ ID NO: 621 - RNA, 20 nt, synthetic construct
  source 1..20: mol_type = other RNA
agcattgctatgatgtcata

SEQ ID NO: 622 - RNA, 20 nt, synthetic construct
  source 1..20: mol_type = other RNA
taagttgataaactgtatga

SEQ ID NO: 623 - RNA, 20 nt, synthetic construct
  source 1..20: mol_type = other RNA
attaataattatataccaaa

SEQ ID NO: 624 - RNA, 20 nt, synthetic construct
  source 1..20: mol_type = other RNA
gagaataatatactggccag

SEQ ID NO: 625 - RNA, 20 nt, synthetic construct
  source 1..20: mol_type = other RNA
aaacataagtagatactaca

SEQ ID NO: 626 - RNA, 20 nt, synthetic construct
  source 1..20: mol_type = other RNA
caaagtgagtcaaaggagag

SEQ ID NO: 627 - RNA, 20 nt, synthetic construct
  source 1..20: mol_type = other RNA
aacaaatcggtgatcaaaca

SEQ ID NO: 628 - RNA, 20 nt, synthetic construct
  source 1..20: mol_type = other RNA
ttagaaactcttattgcgta

SEQ ID NO: 629 - RNA, 20 nt, synthetic construct
  source 1..20: mol_type = other RNA
aataagttatgattggtaag

SEQ ID NO: 630 - RNA, 20 nt, synthetic construct
  source 1..20: mol_type = other RNA
gaaatgaagctgtcttctat

SEQ ID NO: 631 - RNA, 20 nt, synthetic construct
  source 1..20: mol_type = other RNA
taaataatcataaatagcaa

SEQ ID NO: 632 - RNA, 20 nt, synthetic construct
  source 1..20: mol_type = other RNA
accacatcagatattcttca

SEQ ID NO: 633 - RNA, 20 nt, synthetic construct
  source 1..20: mol_type = other RNA
tctgattcctgaatattaaa

SEQ ID NO: 634 - RNA, 20 nt, synthetic construct
  source 1..20: mol_type = other RNA
gagcattgcacattgcccat

SEQ ID NO: 635 - RNA, 20 nt, synthetic construct
  source 1..20: mol_type = other RNA
ggtaatttctagagtgaatg

SEQ ID NO: 636 - RNA, 20 nt, synthetic construct
  source 1..20: mol_type = other RNA
tggaatgattggatgtctct

SEQ ID NO: 637 - RNA, 20 nt, synthetic construct
  source 1..20: mol_type = other RNA
tgaaatgtgatgaattcaca

SEQ ID NO: 638 - RNA, 20 nt, synthetic construct
  source 1..20: mol_type = other RNA
aaataagttatgattggtaa

SEQ ID NO: 639 - RNA, 20 nt, synthetic construct
  source 1..20: mol_type = other RNA
tttaacatatagatccttga

SEQ ID NO: 640 - RNA, 20 nt, synthetic construct
  source 1..20: mol_type = other RNA
cttgaatgtgttcaaccgaa

SEQ ID NO: 641 - RNA, 20 nt, synthetic construct
  source 1..20: mol_type = other RNA
aataacaggatttattgtaa

SEQ ID NO: 642 - RNA, 20 nt, synthetic construct
  source 1..20: mol_type = other RNA
tatgcttgtctgttagtact

SEQ ID NO: 643 - RNA, 20 nt, synthetic construct
  source 1..20: mol_type = other RNA
ggaacttgggttcttccagg

SEQ ID NO: 644 - RNA, 20 nt, synthetic construct
  source 1..20: mol_type = other RNA
ataccaaattaagttgataa

SEQ ID NO: 645 - RNA, 20 nt, synthetic construct
  source 1..20: mol_type = other RNA
aatgattggatgtctctgca

SEQ ID NO: 646 - RNA, 20 nt, synthetic construct
  source 1..20: mol_type = other RNA
aaagctactccacttatgca

SEQ ID NO: 647 - RNA, 20 nt, synthetic construct
  source 1..20: mol_type = other RNA
tatgaatatcttctttctta

SEQ ID NO: 648 - RNA, 20 nt, synthetic construct
  source 1..20: mol_type = other RNA
ttaacttgtatggtttccag

SEQ ID NO: 649 - RNA, 20 nt, synthetic construct
  source 1..20: mol_type = other RNA
gaggttgtgagtataccaga

SEQ ID NO: 650 - RNA, 20 nt, synthetic construct
  source 1..20: mol_type = other RNA
ttgaacaaatcggtgatcaa

SEQ ID NO: 651 - RNA, 20 nt, synthetic construct
  source 1..20: mol_type = other RNA
atacaacagtaaccaccacc

SEQ ID NO: 652 - RNA, 20 nt, synthetic construct
  source 1..20: mol_type = other RNA
accacctgaaacatgcaaca

SEQ ID NO: 653 - RNA, 20 nt, synthetic construct
  source 1..20: mol_type = other RNA
gtgtcctatttgatagcatt

SEQ ID NO: 654 - RNA, 20 nt, synthetic construct
  source 1..20: mol_type = other RNA
tataccaaattaagttgata

SEQ ID NO: 655 - RNA, 20 nt, synthetic construct
  source 1..20: mol_type = other RNA
caagcataagttaaggtact

SEQ ID NO: 656 - RNA, 20 nt, synthetic construct
  source 1..20: mol_type = other RNA
tattagtgattctaaactga

SEQ ID NO: 657 - RNA, 20 nt, synthetic construct
  source 1..20: mol_type = other RNA
ggacacagtaagaaggcatg

SEQ ID NO: 658 - DNA, 45 bp, Homo sapiens
  source 1..45: mol_type = other DNA
ggcaccaactccatgtttggtgtttgtctgtgtttgtttttcaag

SEQ ID NO: 659 - DNA, 45 bp, Homo sapiens
  source 1..45: mol_type = other DNA
aattgctgcttggattttgaaatttactgtaactgtcagtgtaca

SEQ ID NO: 660 - DNA, 45 bp, Homo sapiens
  source 1..45: mol_type = other DNA
ttatgcccggcgccgggcaccaactccatgtttggtgtttgtctg

SEQ ID NO: 661 - DNA, 45 bp, Homo sapiens
  source 1..45: mol_type = other DNA
tgtttttcaagaaatgattcaaattgctgcttggattttgaaatt

SEQ ID NO: 662 - DNA, 45 bp, Homo sapiens
  source 1..45: mol_type = other DNA
ctgagcacagacgagagcacctttgatgacatcgtccattcgttt

SEQ ID NO: 663 - DNA, 45 bp, Homo sapiens
  source 1..45: mol_type = other DNA
gtccgtggcggcaacgtgggcatcaactcctttggcttcgggggc

SEQ ID NO: 664 - DNA, 45 bp, Homo sapiens
  source 1..45: mol_type = other DNA
tacgcacggtcgcttcctggaaattggcaaattcgacctttctca

SEQ ID NO: 665 - DNA, 45 bp, Homo sapiens
  source 1..45: mol_type = other DNA
accatcctgaacgccggcaccaatacagatggcttcaaggagcaa

SEQ ID NO: 666 - DNA, 45 bp, Homo sapiens
  source 1..45: mol_type = other DNA
tctgtgtttgtttttcaagaaatgattcaaattgctgcttggatt

SEQ ID NO: 667 - DNA, 45 bp, Homo sapiens
  source 1..45: mol_type = other DNA
gtttttcaagaaatgattcaaattgctgcttggattttgaaattt

SEQ ID NO: 668 - DNA, 45 bp, Homo sapiens
  source 1..45: mol_type = other DNA
gggtctgcgcttggtctttctgtgcttggatttgcatatttattg

SEQ ID NO: 669 - DNA, 45 bp, Homo sapiens
  source 1..45: mol_type = other DNA
tctcagaaccacccgctcggcatggctatcttcctgaagaacgtg

SEQ ID NO: 670 - DNA, 45 bp, Homo sapiens
  source 1..45: mol_type = other DNA
tcctacggggcctgcgtggcctttgaaatgtgctcccagctgcag

SEQ ID NO: 671 - DNA, 45 bp, Homo sapiens
  source 1..45: mol_type = other DNA
ctgcgcttggtctttctgtgcttggatttgcatatttattgcatt

SEQ ID NO: 672 - DNA, 45 bp, Homo sapiens
  source 1..45: mol_type = other DNA
attgctgcttggattttgaaatttactgtaactgtcagtgtacac

SEQ ID NO: 673 - DNA, 45 bp, Homo sapiens
  source 1..45: mol_type = other DNA
cttggtctttctgtgcttggatttgcatatttattgcattgctgg

SEQ ID NO: 674 - DNA, 45 bp, Homo sapiens
  source 1..45: mol_type = other DNA
atcgacgccaaccccaatgccttgttcccacctgtggagttccca

SEQ ID NO: 675 - DNA, 45 bp, Homo sapiens
  source 1..45: mol_type = other DNA
gacgagagcacctttgatgacatcgtccattcgtttgtgagcctg

SEQ ID NO: 676 - DNA, 45 bp, Homo sapiens
  source 1..45: mol_type = other DNA
gtcgcttcctggaaattggcaaattcgacctttctcagaaccacc

SEQ ID NO: 677 - DNA, 45 bp, Homo sapiens
  source 1..45: mol_type = other DNA
aacctctcccaggtatgcgacgggaaagtatccgtccacgtcatc

SEQ ID NO: 678 - DNA, 45 bp, Homo sapiens
  source 1..45: mol_type = other DNA
aggtgactcggggaggctgctgtggaaggataactgggtgagctt

SEQ ID NO: 679 - DNA, 45 bp, Homo sapiens
  source 1..45: mol_type = other DNA
cgtggcaatgcgggacagagcaactacggctttgccaattccgcc

SEQ ID NO: 680 - DNA, 45 bp, Homo sapiens
  source 1..45: mol_type = other DNA
gcgggacagagcaactacggctttgccaattccgccatggagcgt

SEQ ID NO: 681 - RNA, 20 nt, synthetic construct
  source 1..20: mol_type = other RNA
tcagggtgtcccatgttgga

SEQ ID NO: 682 - DNA, 45 bp, Homo sapiens
  source 1..45: mol_type = other DNA
ttctgtgcttggatttgcatatttattgcattgctggtagagacc

SEQ ID NO: 683 - DNA, 45 bp, Homo sapiens
  source 1..45: mol_type = other DNA
cagagcaactacggctttgccaattccgccatggagcgtatctgt

SEQ ID NO: 684 - DNA, 45 bp, Homo sapiens
  source 1..45: mol_type = other DNA
acagacgagagcacctttgatgacatcgtccattcgtttgtgagc

SEQ ID NO: 685 - DNA, 45 bp, Homo sapiens
  source 1..45: mol_type = other DNA
tttttcaagaaatgattcaaattgctgcttggattttgaaattta

SEQ ID NO: 686 - DNA, 45 bp, Homo sapiens
  source 1..45: mol_type = other DNA
gtcttcaacctggccgtggtcttgagagatggcttgctggagaac

SEQ ID NO: 687 - DNA, 45 bp, Homo sapiens
  source 1..45: mol_type = other DNA
ctgactgccatccagataggcctcatagacctgctgagctgcatg

SEQ ID NO: 688 - DNA, 45 bp, Homo sapiens
  source 1..45: mol_type = other DNA
atggcccaagggaagcacattggcaaagtcgtcgtgcaggtgctt

SEQ ID NO: 689 - DNA, 45 bp, Homo sapiens
  source 1..45: mol_type = other DNA
tcgtttgtgagcctgactgccatccagataggcctcatagacctg

SEQ ID NO: 690 - DNA, 45 bp, Homo sapiens
  source 1..45: mol_type = other DNA
tcagctgcggctgctgctggaagtcacctatgaagccatcgtgga

SEQ ID NO: 691 - DNA, 45 bp, Homo sapiens
  source 1..45: mol_type = other DNA
acgcacggtcgcttcctggaaattggcaaattcgacctttctcag

SEQ ID NO: 692 - DNA, 45 bp, Homo sapiens
  source 1..45: mol_type = other DNA
tggcacacgggcgggaagggcgttgacctggtcttgaactccttg

SEQ ID NO: 693 - DNA, 45 bp, Homo sapiens
  source 1..45: mol_type = other DNA
gacctcccaggacagcctgctaggtatggagttctcgggccgaga

SEQ ID NO: 694 - DNA, 45 bp, Homo sapiens
  source 1..45: mol_type = other DNA
caccaactccatgtttggtgtttgtctgtgtttgtttttcaagaa

SEQ ID NO: 695 - DNA, 45 bp, Homo sapiens
  source 1..45: mol_type = other DNA
ttggcaaattcgacctttctcagaaccacccgctcggcatggcta

SEQ ID NO: 696 - DNA, 45 bp, Homo sapiens
  source 1..45: mol_type = other DNA
gagcagtacacacccaaggccaagtaccatggcaacgtgatgcta

SEQ ID NO: 697 - DNA, 45 bp, Homo sapiens
  source 1..45: mol_type = other DNA
acacacccaaggccaagtaccatggcaacgtgatgctactgcgcg

SEQ ID NO: 698 - DNA, 45 bp, Homo sapiens
  source 1..45: mol_type = other DNA
tggtctttctgtgcttggatttgcatatttattgcattgctggta

SEQ ID NO: 699 - DNA, 45 bp, Homo sapiens
  source 1..45: mol_type = other DNA
gtctgtgtttgtttttcaagaaatgattcaaattgctgcttggat

SEQ ID NO: 700 - DNA, 45 bp, Homo sapiens
  source 1..45: mol_type = other DNA
gaagctgccagagtcggagaacttgcaggagttctgggacaacct

SEQ ID NO: 701 - DNA, 45 bp, Homo sapiens
  source 1..45: mol_type = other DNA
gtgccttcgaggtgtcagagaacggcaacctggtagtgagtggga

SEQ ID NO: 702 - DNA, 45 bp, Homo sapiens
  source 1..45: mol_type = other DNA
ggctgctgctggaagtcacctatgaagccatcgtggacggaggca

SEQ ID NO: 703 - DNA, 45 bp, Homo sapiens
  source 1..45: mol_type = other DNA
ttggattttgaaatttactgtaactgtcagtgtacacgtctggac

SEQ ID NO: 704 - DNA, 45 bp, Homo sapiens
  source 1..45: mol_type = other DNA
cgtcgagcagctgcctgtggtgtttgaggatgtggtgctgcacca

SEQ ID NO: 705 - RNA, 20 nt, synthetic construct
  source 1..20: mol_type = other RNA
tttggtgtttgtctgtgttt

SEQ ID NO: 706 - RNA, 20 nt, synthetic construct
  source 1..20: mol_type = other RNA
tttgaaatttactgtaactg

SEQ ID NO: 707 - RNA, 20 nt, synthetic construct
  source 1..20: mol_type = other RNA
ggcaccaactccatgtttgg

SEQ ID NO: 708 - RNA, 20 nt, synthetic construct
  source 1..20: mol_type = other RNA
gattcaaattgctgcttgga

SEQ ID NO: 709 - RNA, 20 nt, synthetic construct
  source 1..20: mol_type = other RNA
agcacctttgatgacatcgt

SEQ ID NO: 710 - RNA, 20 nt, synthetic construct
  source 1..20: mol_type = other RNA
gtgggcatcaactcctttgg

SEQ ID NO: 711 - RNA, 20 nt, synthetic construct
  source 1..20: mol_type = other RNA
cctggaaattggcaaattcg

SEQ ID NO: 712 - RNA, 20 nt, synthetic construct
  source 1..20: mol_type = other RNA
ggcaccaatacagatggctt

SEQ ID NO: 713 - RNA, 20 nt, synthetic construct
  source 1..20: mol_type = other RNA
caagaaatgattcaaattgc

SEQ ID NO: 714 - RNA, 20 nt, synthetic construct
  source 1..20: mol_type = other RNA
attcaaattgctgcttggat

SEQ ID NO: 715 - RNA, 20 nt, synthetic construct
  source 1..20: mol_type = other RNA
ctttctgtgcttggatttgc

SEQ ID NO: 716 - RNA, 20 nt, synthetic construct
  source 1..20: mol_type = other RNA
ctcggcatggctatcttcct

SEQ ID NO: 717 - RNA, 20 nt, synthetic construct
  source 1..20: mol_type = other RNA
gtggcctttgaaatgtgctc

SEQ ID NO: 718 - RNA, 20 nt, synthetic construct
  source 1..20: mol_type = other RNA
ctgtgcttggatttgcatat

SEQ ID NO: 719 - RNA, 20 nt, synthetic construct
  source 1..20: mol_type = other RNA
ttgaaatttactgtaactgt

SEQ ID NO: 720 - RNA, 20 nt, synthetic construct
  source 1..20: mol_type = other RNA
cttggatttgcatatttatt

SEQ ID NO: 721 - RNA, 20 nt, synthetic construct
  source 1..20: mol_type = other RNA
aatgccttgttcccacctgt

SEQ ID NO: 722 - RNA, 20 nt, synthetic construct
  source 1..20: mol_type = other RNA
gatgacatcgtccattcgtt

SEQ ID NO: 723 - RNA, 20 nt, synthetic construct
  source 1..20: mol_type = other RNA
ttggcaaattcgacctttct

SEQ ID NO: 724 - RNA, 20 nt, synthetic construct
  source 1..20: mol_type = other RNA
tgcgacgggaaagtatccgt

SEQ ID NO: 725 - RNA, 20 nt, synthetic construct
  source 1..20: mol_type = other RNA
gctgctgtggaaggataact

SEQ ID NO: 726 - RNA, 20 nt, synthetic construct
  source 1..20: mol_type = other RNA
cagagcaactacggctttgc

SEQ ID NO: 727 - RNA, 20 nt, synthetic construct
  source 1..20: mol_type = other RNA
tacggctttgccaattccgc

SEQ ID NO: 728 - RNA, 20 nt, synthetic construct
  source 1..20: mol_type = other RNA
attggcaaattcgacctttc

SEQ ID NO: 729 - RNA, 20 nt, synthetic construct
  source 1..20: mol_type = other RNA
tgcatatttattgcattgct

SEQ ID NO: 730 - RNA, 20 nt, synthetic construct
  source 1..20: mol_type = other RNA
tttgccaattccgccatgga

SEQ ID NO: 731 - RNA, 20 nt, synthetic construct
  source 1..20: mol_type = other RNA
tttgatgacatcgtccattc

SEQ ID NO: 732 - RNA, 20 nt, synthetic construct
  source 1..20: mol_type = other RNA
ttcaaattgctgcttggatt

SEQ ID NO: 733 - RNA, 20 nt, synthetic construct
  source 1..20: mol_type = other RNA
gtggtcttgagagatggctt

SEQ ID NO: 734 - RNA, 20 nt, synthetic construct
  source 1..20: mol_type = other RNA
ataggcctcatagacctgct

SEQ ID NO: 735 - RNA, 20 nt, synthetic construct
  source 1..20: mol_type = other RNA
cacattggcaaagtcgtcgt

SEQ ID NO: 736 - RNA, 20 nt, synthetic construct
  source 1..20: mol_type = other RNA
actgccatccagataggcct

SEQ ID NO: 737 - RNA, 20 nt, synthetic construct
  source 1..20: mol_type = other RNA
gctggaagtcacctatgaag

SEQ ID NO: 738 - RNA, 20 nt, synthetic construct
  source 1..20: mol_type = other RNA
ctggaaattggcaaattcga

SEQ ID NO: 739 - RNA, 20 nt, synthetic construct
  source 1..20: mol_type = other RNA
aagggcgttgacctggtctt

SEQ ID NO: 740 - RNA, 20 nt, synthetic construct
  source 1..20: mol_type = other RNA
cctgctaggtatggagttct

SEQ ID NO: 741 - RNA, 20 nt, synthetic construct
  source 1..20: mol_type = other RNA
tggtgtttgtctgtgtttgt

SEQ ID NO: 742 - RNA, 20 nt, synthetic construct
  source 1..20: mol_type = other RNA
tttctcagaaccacccgctc

SEQ ID NO: 743 - RNA, 20 nt, synthetic construct
  source 1..20: mol_type = other RNA
aagaaatgattcaaattgct

SEQ ID NO: 744 - RNA, 20 nt, synthetic construct
  source 1..20: mol_type = other RNA
tccgccgagtacaatgtcaa

SEQ ID NO: 745 - RNA, 20 nt, synthetic construct
  source 1..20: mol_type = other RNA
aaggccaagtaccatggcaa

SEQ ID NO: 746 - RNA, 20 nt, synthetic construct
  source 1..20: mol_type = other RNA
agtaccatggcaacgtgatg

SEQ ID NO: 747 - RNA, 20 nt, synthetic construct
  source 1..20: mol_type = other RNA
tggatttgcatatttattgc

SEQ ID NO: 748 - RNA, 20 nt, synthetic construct
  source 1..20: mol_type = other RNA
tcaagaaatgattcaaattg

SEQ ID NO: 749 - RNA, 20 nt, synthetic construct
  source 1..20: mol_type = other RNA
ggagaacttgcaggagttct

SEQ ID NO: 750 - RNA, 20 nt, synthetic construct
  source 1..20: mol_type = other RNA
cagagaacggcaacctggta

SEQ ID NO: 751 - RNA, 20 nt, synthetic construct
  source 1..20: mol_type = other RNA
agaaatgattcaaattgctg

SEQ ID NO: 752 - RNA, 20 nt, synthetic construct
  source 1..20: mol_type = other RNA
tcacctatgaagccatcgtg

SEQ ID NO: 753 - RNA, 20 nt, synthetic construct
  source 1..20: mol_type = other RNA
tactgtaactgtcagtgtac

SEQ ID NO: 754 - RNA, 20 nt, synthetic construct
  source 1..20: mol_type = other RNA
tgtggtgtttgaggatgtgg

SEQ ID NO: 755 - RNA, 20 nt, synthetic construct
  source 1..20: mol_type = other RNA
aaacacagacaaacaccaaa

SEQ ID NO: 756 - RNA, 20 nt, synthetic construct
  source 1..20: mol_type = other RNA
cagttacagtaaatttcaaa

SEQ ID NO: 757 - RNA, 20 nt, synthetic construct
  source 1..20: mol_type = other RNA
ccaaacatggagttggtgcc

SEQ ID NO: 758 - RNA, 20 nt, synthetic construct
  source 1..20: mol_type = other RNA
tccaagcagcaatttgaatc

SEQ ID NO: 759 - RNA, 20 nt, synthetic construct
  source 1..20: mol_type = other RNA
acgatgtcatcaaaggtgct

SEQ ID NO: 760 - RNA, 20 nt, synthetic construct
  source 1..20: mol_type = other RNA
ccaaaggagttgatgcccac

SEQ ID NO: 761 - RNA, 20 nt, synthetic construct
  source 1..20: mol_type = other RNA
cgaatttgccaatttccagg

SEQ ID NO: 762 - RNA, 20 nt, synthetic construct
  source 1..20: mol_type = other RNA
aagccatctgtattggtgcc

SEQ ID NO: 763 - RNA, 20 nt, synthetic construct
  source 1..20: mol_type = other RNA
gcaatttgaatcatttcttg

SEQ ID NO: 764 - RNA, 20 nt, synthetic construct
  source 1..20: mol_type = other RNA
atccaagcagcaatttgaat

SEQ ID NO: 765 - RNA, 20 nt, synthetic construct
  source 1..20: mol_type = other RNA
gcaaatccaagcacagaaag

SEQ ID NO: 766 - RNA, 20 nt, synthetic construct
  source 1..20: mol_type = other RNA
aggaagatagccatgccgag

SEQ ID NO: 767 - RNA, 20 nt, synthetic construct
  source 1..20: mol_type = other RNA
gagcacatttcaaaggccac

SEQ ID NO: 768 - RNA, 20 nt, synthetic construct
  source 1..20: mol_type = other RNA
atatgcaaatccaagcacag

SEQ ID NO: 769 - RNA, 20 nt, synthetic construct
  source 1..20: mol_type = other RNA
acagttacagtaaatttcaa

SEQ ID NO: 770 - RNA, 20 nt, synthetic construct
  source 1..20: mol_type = other RNA
aataaatatgcaaatccaag

SEQ ID NO: 771 - RNA, 20 nt, synthetic construct
  source 1..20: mol_type = other RNA
acaggtgggaacaaggcatt

SEQ ID NO: 772 - RNA, 20 nt, synthetic construct
  source 1..20: mol_type = other RNA
aacgaatggacgatgtcatc

SEQ ID NO: 773 - RNA, 20 nt, synthetic construct
  source 1..20: mol_type = other RNA
agaaaggtcgaatttgccaa

SEQ ID NO: 774 - RNA, 20 nt, synthetic construct
  source 1..20: mol_type = other RNA
acggatactttcccgtcgca

SEQ ID NO: 775 - RNA, 20 nt, synthetic construct
  source 1..20: mol_type = other RNA
agttatccttccacagcagc

SEQ ID NO: 776 - RNA, 20 nt, synthetic construct
  source 1..20: mol_type = other RNA
gcaaagccgtagttgctctg

SEQ ID NO: 777 - RNA, 20 nt, synthetic construct
  source 1..20: mol_type = other RNA
gcggaattggcaaagccgta

SEQ ID NO: 778 - RNA, 20 nt, synthetic construct
  source 1..20: mol_type = other RNA
gaaaggtcgaatttgccaat

SEQ ID NO: 779 - RNA, 20 nt, synthetic construct
  source 1..20: mol_type = other RNA
agcaatgcaataaatatgca

SEQ ID NO: 780 - RNA, 20 nt, synthetic construct
  source 1..20: mol_type = other RNA
tccatggcggaattggcaaa

SEQ ID NO: 781 - RNA, 20 nt, synthetic construct
  source 1..20: mol_type = other RNA
gaatggacgatgtcatcaaa

SEQ ID NO: 782 - RNA, 20 nt, synthetic construct
  source 1..20: mol_type = other RNA
aatccaagcagcaatttgaa

SEQ ID NO: 783 - RNA, 20 nt, synthetic construct
  source 1..20: mol_type = other RNA
aagccatctctcaagaccac

SEQ ID NO: 784 - RNA, 20 nt, synthetic construct
  source 1..20: mol_type = other RNA
agcaggtctatgaggcctat

SEQ ID NO: 785 - RNA, 20 nt, synthetic construct
  source 1..20: mol_type = other RNA
acgacgactttgccaatgtg

SEQ ID NO: 786 - RNA, 20 nt, synthetic construct
  source 1..20: mol_type = other RNA
aggcctatctggatggcagt

SEQ ID NO: 787 - RNA, 20 nt, synthetic construct
  source 1..20: mol_type = other RNA
cttcataggtgacttccagc

SEQ ID NO: 788 - RNA, 20 nt, synthetic construct
  source 1..20: mol_type = other RNA
tcgaatttgccaatttccag

SEQ ID NO: 789 - RNA, 20 nt, synthetic construct
  source 1..20: mol_type = other RNA
aagaccaggtcaacgccctt

SEQ ID NO: 790 - RNA, 20 nt, synthetic construct
  source 1..20: mol_type = other RNA
agaactccatacctagcagg

SEQ ID NO: 791 - RNA, 20 nt, synthetic construct
  source 1..20: mol_type = other RNA
acaaacacagacaaacacca

SEQ ID NO: 792 - RNA, 20 nt, synthetic construct
  source 1..20: mol_type = other RNA
gagcgggtggttctgagaaa

SEQ ID NO: 793 - RNA, 20 nt, synthetic construct
  source 1..20: mol_type = other RNA
agcaatttgaatcatttctt

SEQ ID NO: 794 - RNA, 20 nt, synthetic construct
  source 1..20: mol_type = other RNA
ttgacattgtactcggcgga

SEQ ID NO: 795 - RNA, 20 nt, synthetic construct
  source 1..20: mol_type = other RNA
ttgccatggtacttggcctt

SEQ ID NO: 796 - RNA, 20 nt, synthetic construct
  source 1..20: mol_type = other RNA
catcacgttgccatggtact

SEQ ID NO: 797 - RNA, 20 nt, synthetic construct
  source 1..20: mol_type = other RNA
gcaataaatatgcaaatcca

SEQ ID NO: 798 - RNA, 20 nt, synthetic construct
  source 1..20: mol_type = other RNA
caatttgaatcatttcttga

SEQ ID NO: 799 - RNA, 20 nt, synthetic construct
  source 1..20: mol_type = other RNA
agaactcctgcaagttctcc

SEQ ID NO: 800 - RNA, 20 nt, synthetic construct
  source 1..20: mol_type = other RNA
taccaggttgccgttctctg

SEQ ID NO: 801 - RNA, 20 nt, synthetic construct
  source 1..20: mol_type = other RNA
cagcaatttgaatcatttct

SEQ ID NO: 802 - RNA, 20 nt, synthetic construct
  source 1..20: mol_type = other RNA
cacgatggcttcataggtga

SEQ ID NO: 803 - RNA, 20 nt, synthetic construct
  source 1..20: mol_type = other RNA
gtacactgacagttacagta

SEQ ID NO: 804 - RNA, 20 nt, synthetic construct
  source 1..20: mol_type = other RNA
ccacatcctcaaacaccaca

SEQ ID NO: 805 - DNA, 45 bp, Mus musculus
  source 1..45: mol_type = other DNA
ggctgctgttggaagtcagctatgaagcaattgtggatggaggta

SEQ ID NO: 806 - DNA, 45 bp, Mus musculus
  source 1..45: mol_type = other DNA
cctagaagagacccctgtggtatttgagaatgtgtcgtttcatca

SEQ ID NO: 807 - DNA, 45 bp, Mus musculus
  source 1..45: mol_type = other DNA
atttgccacgctggtcgtttctccattaaattctcatatttattg

SEQ ID NO: 808 - DNA, 45 bp, Mus musculus
  source 1..45: mol_type = other DNA
tgccacgctggtcgtttctccattaaattctcatatttattgcat

SEQ ID NO: 809 - DNA, 45 bp, Mus musculus
  source 1..45: mol_type = other DNA
atctctggacctcaggctgcagtgaatgaatttgtggagcagcta

SEQ ID NO: 810 - DNA, 45 bp, Mus musculus
  source 1..45: mol_type = other DNA
tctaacaaccaccctctgggcatggctatcttcttgaagaacgtc

SEQ ID NO: 811 - DNA, 45 bp, Mus musculus
  source 1..45: mol_type = other DNA
tgaggccttatgcttcttcataaagcagtttcttgatgtggaaca

SEQ ID NO: 812 - DNA, 45 bp, Mus musculus
  source 1..45: mol_type = other DNA
gttcgtggtggcaacgtgggcatcaactcatttggcttcggaggc

SEQ ID NO: 813 - DNA, 45 bp, Mus musculus
  source 1..45: mol_type = other DNA
gccccagcggtagagaatagcatcatcctctacttgtagtcaact

SEQ ID NO: 814 - DNA, 45 bp, Mus musculus
  source 1..45: mol_type = other DNA
gctgaggccttatgcttcttcataaagcagtttcttgatgtggaa

SEQ ID NO: 815 - DNA, 45 bp, Mus musculus
  source 1..45: mol_type = other DNA
ttctaccgtgtggcatttgccacgctggtcgtttctccattaaat

SEQ ID NO: 816 - DNA, 45 bp, Mus musculus
  source 1..45: mol_type = other DNA
cacacttagaaaaattcttacagcactcgtcatgagttgtccatt

SEQ ID NO: 817 - DNA, 45 bp, Mus musculus
  source 1..45: mol_type = other DNA
tagtacaagcagaatttgtccacctttaagttgccctgagggcat

SEQ ID NO: 818 - DNA, 45 bp, Mus musculus
  source 1..45: mol_type = other DNA
gactgaagcatctggttggctctagtcacacttagaaaaattctt

SEQ ID NO: 819 - DNA, 45 bp, Mus musculus
  source 1..45: mol_type = other DNA
tgtgctgccaggggctcagcccaccctgatttctgccatctccaa

SEQ ID NO: 820 - DNA, 45 bp, Mus musculus
  source 1..45: mol_type = other DNA
gacgggaaggtgtctgtgcacatcattgagggtgaccaccgcaca

SEQ ID NO: 821 - DNA, 45 bp, Mus musculus
  source 1..45: mol_type = other DNA
agaccccagagctcttccaggatgtcaacaagcccaaatacaatg

SEQ ID NO: 822 - DNA, 45 bp, Mus musculus
  source 1..45: mol_type = other DNA
gtggcatttgccacgctggtcgtttctccattaaattctcatatt

SEQ ID NO: 823 - DNA, 45 bp, Mus musculus
  source 1..45: mol_type = other DNA
caaactaactacggcttcgccaactctaccatggagcgtatatgt

SEQ ID NO: 824 - DNA, 45 bp, Mus musculus
  source 1..45: mol_type = other DNA
gtgtgcggtgcttggctcagcatggtcgcttcttagagattggca

SEQ ID NO: 825 - DNA, 45 bp, Mus musculus
  source 1..45: mol_type = other DNA
cttggaaaagttcgttttcacaccgcacatggaggctgagtgcct

SEQ ID NO: 826 - DNA, 45 bp, Mus musculus
  source 1..45: mol_type = other DNA
gggtccaggagagactgtcacaaggccgcagcattcgatcacatc

SEQ ID NO: 827 - DNA, 45 bp, Mus musculus
  source 1..45: mol_type = other DNA
aggccttgccactgtagtacaagcagaatttgtccacctttaagt

SEQ ID NO: 828 - DNA, 45 bp, Mus musculus
  source 1..45: mol_type = other DNA
aacctctcccaggtgtgtgacgggaaggtgtctgtgcacatcatt

SEQ ID NO: 829 - DNA, 45 bp, Mus musculus
  source 1..45: mol_type = other DNA
cagcatggtcgcttcttagagattggcaaatttgatctttctaac

SEQ ID NO: 830 - DNA, 45 bp, Mus musculus
  source 1..45: mol_type = other DNA
tagggtacaaaggtgttcagaggatcccagtctgagtgattagcc

SEQ ID NO: 831 - DNA, 45 bp, Mus musculus
  source 1..45: mol_type = other DNA
gctggtcgtttctccattaaattctcatatttattgcattgctgg

SEQ ID NO: 832 - DNA, 45 bp, Mus musculus
  source 1..45: mol_type = other DNA
tcatttggagcctgtgtagccttcgagatgtgctcccagctgcag

SEQ ID NO: 833 - DNA, 45 bp, Mus musculus
  source 1..45: mol_type = other DNA
cttcatggacacaatgctgcaggtatccattctgggttctagcca

SEQ ID NO: 834 - DNA, 45 bp, Mus musculus
  source 1..45: mol_type = other DNA
ttttctgcagctgtcagtgtgaagaagtgtctggactgtgtcatt

SEQ ID NO: 835 - DNA, 45 bp, Mus musculus
  source 1..45: mol_type = other DNA
agcatggtcgcttcttagagattggcaaatttgatctttctaaca

SEQ ID NO: 836 - DNA, 45 bp, Mus musculus
  source 1..45: mol_type = other DNA
ttgagcacagatgagcgcacctttgatgacatcgtgcatgccttt

SEQ ID NO: 837 - DNA, 45 bp, Mus musculus
  source 1..45: mol_type = other DNA
gaggctgtgaagccgttgggagtgaaagtgtcagatctgctgttg

SEQ ID NO: 838 - DNA, 45 bp, Mus musculus
  source 1..45: mol_type = other DNA
tcgcttcttagagattggcaaatttgatctttctaacaaccaccc

SEQ ID NO: 839 - DNA, 45 bp, Mus musculus
  source 1..45: mol_type = other DNA
aggccttatgcttcttcataaagcagtttcttgatgtggaacaca

SEQ ID NO: 840 - DNA, 45 bp, Mus musculus
  source 1..45: mol_type = other DNA
cctggccacctcagtcctgctatcatctgacttcctctgggatgt

SEQ ID NO: 841 - DNA, 45 bp, Mus musculus
  source 1..45: mol_type = other DNA
tccagtgagactccttggagggtgtgccattctgtcagtataagg

SEQ ID NO: 842 - DNA, 45 bp, Mus musculus
  source 1..45: mol_type = other DNA
ggcaataaaaccttgaggttccttcctgttctttgtgattgccta

SEQ ID NO: 843 - DNA, 45 bp, Mus musculus
  source 1..45: mol_type = other DNA
gtttctccattaaattctcatatttattgcattgctgggaaagac

SEQ ID NO: 844 - DNA, 45 bp, Mus musculus
  source 1..45: mol_type = other DNA
aggccacccttgaaggtgaacaaggcaagctgctctggaaagata

SEQ ID NO: 845 - DNA, 45 bp, Mus musculus
  source 1..45: mol_type = other DNA
ctgctagaggcctcccatgcctttgaggtgtctgacactggcaat

SEQ ID NO: 846 - DNA, 45 bp, Mus musculus
  source 1..45: mol_type = other DNA
ctggctggaggccttgccactgtagtacaagcagaatttgtccac

SEQ ID NO: 847 - DNA, 45 bp, Mus musculus
  source 1..45: mol_type = other DNA
atcaatgtcaaccctaacgccttgttcccacctgtggagttcccg

SEQ ID NO: 848 - DNA, 45 bp, Mus musculus
  source 1..45: mol_type = other DNA
ttcccttgatgaagagggatcataaagataacttggagttctttc

SEQ ID NO: 849 - DNA, 45 bp, Mus musculus
  source 1..45: mol_type = other DNA
ctagaagagacccctgtggtatttgagaatgtgtcgtttcatcag

SEQ ID NO: 850 - DNA, 45 bp, Mus musculus
  source 1..45: mol_type = other DNA
ctggcccgcacatcttctgccgagtacaatgtcaacaacctggtg

SEQ ID NO: 851 - DNA, 45 bp, Mus musculus
  source 1..45: mol_type = other DNA
agtgtctctacctagttagagtttactgactcaagggagcagatt

SEQ ID NO: 852 - DNA, 45 bp, Mus musculus
  source 1..45: mol_type = other DNA
cgtggccctggtgtcctcctcagtgcagagtttccagtgtgacca

SEQ ID NO: 853 - DNA, 45 bp, Mus musculus
  source 1..45: mol_type = other DNA
tctaccgtgtggcatttgccacgctggtcgtttctccattaaatt

SEQ ID NO: 854 - DNA, 45 bp, Mus musculus
  source 1..45: mol_type = other DNA
attggctccaccaaatccaacatgggacaccctgagcctgcctct

SEQ ID NO: 855 - RNA, 20 nt, synthetic construct
  source 1..20: mol_type = other RNA
tcagctatgaagcaattgtg

SEQ ID NO: 856 - RNA, 20 nt, synthetic construct
  source 1..20: mol_type = other RNA
tgtggtatttgagaatgtgt

SEQ ID NO: 857 - RNA, 20 nt, synthetic construct
  source 1..20: mol_type = other RNA
cgtttctccattaaattctc

SEQ ID NO: 858 - RNA, 20 nt, synthetic construct
  source 1..20: mol_type = other RNA
ttctccattaaattctcata

SEQ ID NO: 859 - RNA, 20 nt, synthetic construct
  source 1..20: mol_type = other RNA
gctgcagtgaatgaatttgt

SEQ ID NO: 860 - RNA, 20 nt, synthetic construct
  source 1..20: mol_type = other RNA
ctgggcatggctatcttctt

SEQ ID NO: 861 - RNA, 20 nt, synthetic construct
  source 1..20: mol_type = other RNA
cttcataaagcagtttcttg

SEQ ID NO: 862 - RNA, 20 nt, synthetic construct
  source 1..20: mol_type = other RNA
gtgggcatcaactcatttgg

SEQ ID NO: 863 - RNA, 20 nt, synthetic construct
  source 1..20: mol_type = other RNA
aatagcatcatcctctactt

SEQ ID NO: 864 - RNA, 20 nt, synthetic construct
  source 1..20: mol_type = other RNA
ttcttcataaagcagtttct

SEQ ID NO: 865 - RNA, 20 nt, synthetic construct
  source 1..20: mol_type = other RNA
tttgccacgctggtcgtttc

SEQ ID NO: 866 - RNA, 20 nt, synthetic construct
  source 1..20: mol_type = other RNA
tcttacagcactcgtcatga

SEQ ID NO: 867 - RNA, 20 nt, synthetic construct
  source 1..20: mol_type = other RNA
ttgtccacctttaagttgcc

SEQ ID NO: 868 - RNA, 20 nt, synthetic construct
  source 1..20: mol_type = other RNA
ttggctctagtcacacttag

SEQ ID NO: 869 - RNA, 20 nt, synthetic construct
  source 1..20: mol_type = other RNA
tcagcccaccctgatttctg

SEQ ID NO: 870 - RNA, 20 nt, synthetic construct
  source 1..20: mol_type = other RNA
gtgcacatcattgagggtga

SEQ ID NO: 871 - RNA, 20 nt, synthetic construct
  source 1..20: mol_type = other RNA
tccaggatgtcaacaagccc

SEQ ID NO: 872 - RNA, 20 nt, synthetic construct
  source 1..20: mol_type = other RNA
ctggtcgtttctccattaaa

SEQ ID NO: 873 - RNA, 20 nt, synthetic construct
  source 1..20: mol_type = other RNA
ttcgccaactctaccatgga

SEQ ID NO: 874 - RNA, 20 nt, synthetic construct
  source 1..20: mol_type = other RNA
ctcagcatggtcgcttctta

SEQ ID NO: 875 - RNA, 20 nt, synthetic construct
  source 1..20: mol_type = other RNA
tttcacaccgcacatggagg

SEQ ID NO: 876 - RNA, 20 nt, synthetic construct
  source 1..20: mol_type = other RNA
tgtcacaaggccgcagcatt

SEQ ID NO: 877 - RNA, 20 nt, synthetic construct
  source 1..20: mol_type = other RNA
agtacaagcagaatttgtcc

SEQ ID NO: 878 - RNA, 20 nt, synthetic construct
  source 1..20: mol_type = other RNA
tgtgacgggaaggtgtctgt

SEQ ID NO: 879 - RNA, 20 nt, synthetic construct
  source 1..20: mol_type = other RNA
ttagagattggcaaatttga

SEQ ID NO: 880 - RNA, 20 nt, synthetic construct
  source 1..20: mol_type = other RNA
ttcagaggatcccagtctga

SEQ ID NO: 881 - RNA, 20 nt, synthetic construct
  source 1..20: mol_type = other RNA
attaaattctcatatttatt

SEQ ID NO: 882 - RNA, 20 nt, synthetic construct
  source 1..20: mol_type = other RNA
gtagccttcgagatgtgctc

SEQ ID NO: 883 - RNA, 20 nt, synthetic construct
  source 1..20: mol_type = other RNA
gctgcaggtatccattctgg

SEQ ID NO: 884 - RNA, 20 nt, synthetic construct
  source 1..20: mol_type = other RNA
agtgtgaagaagtgtctgga

SEQ ID NO: 885 - RNA, 20 nt, synthetic construct
  source 1..20: mol_type = other RNA
tagagattggcaaatttgat

SEQ ID NO: 886 - RNA, 20 nt, synthetic construct
  source 1..20: mol_type = other RNA
cgcacctttgatgacatcgt

SEQ ID NO: 887 - RNA, 20 nt, synthetic construct
  source 1..20: mol_type = other RNA
ttgggagtgaaagtgtcaga

SEQ ID NO: 888 - RNA, 20 nt, synthetic construct
  source 1..20: mol_type = other RNA
tggcaaatttgatctttcta

SEQ ID NO: 889 - RNA, 20 nt, synthetic construct
  source 1..20: mol_type = other RNA
tcataaagcagtttcttgat

SEQ ID NO: 890 - RNA, 20 nt, synthetic construct
  source 1..20: mol_type = other RNA
cctgctatcatctgacttcc

SEQ ID NO: 891 - RNA, 20 nt, synthetic construct
  source 1..20: mol_type = other RNA
tggagggtgtgccattctgt

SEQ ID NO: 892 - RNA, 20 nt, synthetic construct
  source 1..20: mol_type = other RNA
aggttccttcctgttctttg

SEQ ID NO: 893 - RNA, 20 nt, synthetic construct
  source 1..20: mol_type = other RNA
tctcatatttattgcattgc

SEQ ID NO: 894 - RNA, 20 nt, synthetic construct
  source 1..20: mol_type = other RNA
gtgaacaaggcaagctgctc

SEQ ID NO: 895 - RNA, 20 nt, synthetic construct
  source 1..20: mol_type = other RNA
catgcctttgaggtgtctga

SEQ ID NO: 896 - RNA, 20 nt, synthetic construct
  source 1..20: mol_type = other RNA
gccactgtagtacaagcaga

SEQ ID NO: 897 - RNA, 20 nt, synthetic construct
  source 1..20: mol_type = other RNA
aacgccttgttcccacctgt

SEQ ID NO: 898 - RNA, 20 nt, synthetic construct
  source 1..20: mol_type = other RNA
gggatcataaagataacttg

SEQ ID NO: 899 - RNA, 20 nt, synthetic construct
  source 1..20: mol_type = other RNA
gtggtatttgagaatgtgtc

SEQ ID NO: 900 - RNA, 20 nt, synthetic construct
  source 1..20: mol_type = other RNA
tctgccgagtacaatgtcaa

SEQ ID NO: 901 - RNA, 20 nt, synthetic construct
  source 1..20: mol_type = other RNA
ttagagtttactgactcaag

SEQ ID NO: 902 - RNA, 20 nt, synthetic construct
  source 1..20: mol_type = other RNA
ctcctcagtgcagagtttcc

SEQ ID NO: 903 - RNA, 20 nt, synthetic construct
  source 1..20: mol_type = other RNA
ttgccacgctggtcgtttct

SEQ ID NO: 904 - RNA, 20 nt, synthetic construct
  source 1..20: mol_type = other RNA
tccaacatgggacaccctga

SEQ ID NO: 905 - RNA, 20 nt, synthetic construct
  source 1..20: mol_type = other RNA
cacaattgcttcatagctga

SEQ ID NO: 906 - RNA, 20 nt, synthetic construct
  source 1..20: mol_type = other RNA
acacattctcaaataccaca

SEQ ID NO: 907 - RNA, 20 nt, synthetic construct
  source 1..20: mol_type = other RNA
gagaatttaatggagaaacg

SEQ ID NO: 908 - RNA, 20 nt, synthetic construct
  source 1..20: mol_type = other RNA
tatgagaatttaatggagaa

SEQ ID NO: 909 - RNA, 20 nt, synthetic construct
  source 1..20: mol_type = other RNA
acaaattcattcactgcagc

SEQ ID NO: 910 - RNA, 20 nt, synthetic construct
  source 1..20: mol_type = other RNA
aagaagatagccatgcccag

SEQ ID NO: 911 - RNA, 20 nt, synthetic construct
  source 1..20: mol_type = other RNA
caagaaactgctttatgaag

SEQ ID NO: 912 - RNA, 20 nt, synthetic construct
  source 1..20: mol_type = other RNA
ccaaatgagttgatgcccac

SEQ ID NO: 913 - RNA, 20 nt, synthetic construct
  source 1..20: mol_type = other RNA
aagtagaggatgatgctatt

SEQ ID NO: 914 - RNA, 20 nt, synthetic construct
  source 1..20: mol_type = other RNA
agaaactgctttatgaagaa

SEQ ID NO: 915 - RNA, 20 nt, synthetic construct
  source 1..20: mol_type = other RNA
gaaacgaccagcgtggcaaa

SEQ ID NO: 916 - RNA, 20 nt, synthetic construct
  source 1..20: mol_type = other RNA
tcatgacgagtgctgtaaga

SEQ ID NO: 917 - RNA, 20 nt, synthetic construct
  source 1..20: mol_type = other RNA
ggcaacttaaaggtggacaa

SEQ ID NO: 918 - RNA, 20 nt, synthetic construct
  source 1..20: mol_type = other RNA
ctaagtgtgactagagccaa

SEQ ID NO: 919 - RNA, 20 nt, synthetic construct
  source 1..20: mol_type = other RNA
cagaaatcagggtgggctga

SEQ ID NO: 920 - RNA, 20 nt, synthetic construct
  source 1..20: mol_type = other RNA
tcaccctcaatgatgtgcac

SEQ ID NO: 921 - RNA, 20 nt, synthetic construct
  source 1..20: mol_type = other RNA
gggcttgttgacatcctgga

SEQ ID NO: 922 - RNA, 20 nt, synthetic construct
  source 1..20: mol_type = other RNA
tttaatggagaaacgaccag

SEQ ID NO: 923 - RNA, 20 nt, synthetic construct
  source 1..20: mol_type = other RNA
tccatggtagagttggcgaa

SEQ ID NO: 924 - RNA, 20 nt, synthetic construct
  source 1..20: mol_type = other RNA
taagaagcgaccatgctgag

SEQ ID NO: 925 - RNA, 20 nt, synthetic construct
  source 1..20: mol_type = other RNA
cctccatgtgcggtgtgaaa

SEQ ID NO: 926 - RNA, 20 nt, synthetic construct
  source 1..20: mol_type = other RNA
aatgctgcggccttgtgaca

SEQ ID NO: 927 - RNA, 20 nt, synthetic construct
  source 1..20: mol_type = other RNA
ggacaaattctgcttgtact

SEQ ID NO: 928 - RNA, 20 nt, synthetic construct
  source 1..20: mol_type = other RNA
acagacaccttcccgtcaca

SEQ ID NO: 929 - RNA, 20 nt, synthetic construct
  source 1..20: mol_type = other RNA
tcaaatttgccaatctctaa

SEQ ID NO: 930 - RNA, 20 nt, synthetic construct
  source 1..20: mol_type = other RNA
tcagactgggatcctctgaa

SEQ ID NO: 931 - RNA, 20 nt, synthetic construct
  source 1..20: mol_type = other RNA
aataaatatgagaatttaat

SEQ ID NO: 932 - RNA, 20 nt, synthetic construct
  source 1..20: mol_type = other RNA
gagcacatctcgaaggctac

SEQ ID NO: 933 - RNA, 20 nt, synthetic construct
  source 1..20: mol_type = other RNA
ccagaatggatacctgcagc

SEQ ID NO: 934 - RNA, 20 nt, synthetic construct
  source 1..20: mol_type = other RNA
tccagacacttcttcacact

SEQ ID NO: 935 - RNA, 20 nt, synthetic construct
  source 1..20: mol_type = other RNA
atcaaatttgccaatctcta

SEQ ID NO: 936 - RNA, 20 nt, synthetic construct
  source 1..20: mol_type = other RNA
acgatgtcatcaaaggtgcg

SEQ ID NO: 937 - RNA, 20 nt, synthetic construct
  source 1..20: mol_type = other RNA
tctgacactttcactcccaa

SEQ ID NO: 938 - RNA, 20 nt, synthetic construct
  source 1..20: mol_type = other RNA
tagaaagatcaaatttgcca

SEQ ID NO: 939 - RNA, 20 nt, synthetic construct
  source 1..20: mol_type = other RNA
atcaagaaactgctttatga

SEQ ID NO: 940 - RNA, 20 nt, synthetic construct
  source 1..20: mol_type = other RNA
ggaagtcagatgatagcagg

SEQ ID NO: 941 - RNA, 20 nt, synthetic construct
  source 1..20: mol_type = other RNA
acagaatggcacaccctcca

SEQ ID NO: 942 - RNA, 20 nt, synthetic construct
  source 1..20: mol_type = other RNA
caaagaacaggaaggaacct

SEQ ID NO: 943 - RNA, 20 nt, synthetic construct
  source 1..20: mol_type = other RNA
gcaatgcaataaatatgaga

SEQ ID NO: 944 - RNA, 20 nt, synthetic construct
  source 1..20: mol_type = other RNA
gagcagcttgccttgttcac

SEQ ID NO: 945 - RNA, 20 nt, synthetic construct
  source 1..20: mol_type = other RNA
tcagacacctcaaaggcatg

SEQ ID NO: 946 - RNA, 20 nt, synthetic construct
  source 1..20: mol_type = other RNA
tctgcttgtactacagtggc

SEQ ID NO: 947 - RNA, 20 nt, synthetic construct
  source 1..20: mol_type = other RNA
acaggtgggaacaaggcgtt

SEQ ID NO: 948 - RNA, 20 nt, synthetic construct
  source 1..20: mol_type = other RNA
caagttatctttatgatccc

SEQ ID NO: 949 - RNA, 20 nt, synthetic construct
  source 1..20: mol_type = other RNA
gacacattctcaaataccac

SEQ ID NO: 950 - RNA, 20 nt, synthetic construct
  source 1..20: mol_type = other RNA
ttgacattgtactcggcaga

SEQ ID NO: 951 - RNA, 20 nt, synthetic construct
  source 1..20: mol_type = other RNA
cttgagtcagtaaactctaa

SEQ ID NO: 952 - DNA, 16 bp, synthetic construct
  source 1..16: mol_type = other DNA
gtgtgggcgcttgtgc

SEQ ID NO: 953 - DNA, 16 bp, synthetic construct
  source 1..16: mol_type = other DNA
agtttccggagcgtga

(19) United States
(12) Patent Application Publication
(10) Pub. No.: US 2025/0034575 A1
(43) Pub. Date: Jan. 30, 2025

(54) OLIGONUCLEOTIDES FOR DGAT2, FASN, OPN, AND NOX4 MODULATIONS

(71) Applicant: UNIVERSITY OF MASSACHUSETTS, Boston, MA (US)

(72) Inventors: Michael Czech, Westborough, MA (US); Batuhan Orbay Yenilmez, Westborough, MA (US); Anastasia Khvorova, Westborough, MA (US); Jonathan Kenneth Watts, Worcester, MA (US)

(21) Appl. No.: 18/666,191

(22) Filed: May 16, 2024

Related U.S. Application Data
(60) Provisional application No. 63/467,039, filed on May 17, 2023.

(51) Int. Cl. C12N 15/113

(52) U.S. Cl. CPC C12N 15/1137; C12N 2310/11; C12N 2310/14; C12N 2310/315; C12N 2310/321; C12N 2310/351

(57) ABSTRACT

This disclosure relates to the combination of DGAT2 targeting sequences with FASN, OPN, and/or NOX4 targeting sequences, and methods for treating and preventing non-alcoholic fatty liver disease (NAFLD), non-alcoholic steatohepatitis (NASH), and lipodystrophy syndromes.

### CROSS REFERENCE TO RELATED APPLICATION

This application claims the benefit of U.S. Provisional Patent Application Ser. No. 63/467,039, filed May 17, 2023. The entire content of the above-referenced patent application is incorporated by reference in its entirety herein.

### STATEMENT OF FEDERALLY SPONSORED RESEARCH

This invention was made with government support under Grant No. DK103047 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML file, created on Sep. 24, 2024, is named 753325_UM9-299_ST26.xml and is 913,056 bytes in size.

### FIELD OF THE DISCLOSURE

This disclosure relates to the combination of DGAT2 targeting sequences with FASN, OPN, and/or NOX4 targeting sequences, and methods for treating and preventing non-alcoholic fatty liver disease (NAFLD), non-alcoholic steatohepatitis (NASH), and lipodystrophy syndromes.

### BACKGROUND

One of the many severe complications of type 2 diabetes is triglyceride accumulation in the liver (denoted non-alcoholic fatty liver disease, NAFLD), which can then lead to severe inflammation and fibrosis of the liver (denoted non-alcoholic steatohepatitis, NASH). At least some of the features of NASH appear to be driven by fat accumulation since human mutations in genes that cause NAFLD also lead to the development of NASH. The severity of NASH is underscored by the fact that it is rapidly becoming the leading cause of liver transplant. The development of NAFLD is associated with human obesity and well as lipodystrophies, and the severity of both NAFLD and NASH is often most severe in type 2 diabetes, which now affects 10% of the U.S. population. There is no approved therapeutic for NASH.

The mechanisms in the liver that lead to NAFLD are multiple, and can include fatty acids entering liver from adipose tissues, from lipoproteins in the circulation, or from the synthesis of fatty acids in the liver from carbohydrate and other substrates (denoted de novo lipogenesis). Fatty acids in the liver are esterified into triglyceride (which accumulates and leads to NAFLD) through a synthesis pathway that joins fatty acids with glycerol. The last step in this triglyceride synthetic pathway in liver is catalyzed largely by the enzyme diacylglycerol O-acyltransferase 2 (DGAT2), which joins the third fatty acid onto diacylglycerol to make triglyceride.

Other proteins or precursors that promote or are associated with NAFLD and NASH progression include for example, without limitations: fatty acid synthase (FASN), osteopontin (OPN), and nicotinamide adenine dinucleotide phosphate oxidase (NOX4). FASN is an enzyme in the de novo lipogenesis (DNL) pathway that converts the metabolites of dietary sugars, acetyl-coenzyme A (CoA) and malonyl-CoA, into palmitate, a saturated fatty acid at the final committed step of the DNL pathway. Hepatic DNL is increased in patients with metabolic syndrome and NAFLD. OPN is involved in the pathogenesis of NAFL associated with obesity and is a reliable biomarker for NASH/fibrosis in human NAFLD. Nicotinamide adenine dinucleotide phosphate hydrogen oxidases 4 (NOX4) contributes in the liver, through the generation of reactive oxygen species (ROS), to hepatic fibrosis by acting through multiple pathways, including hepatic stellate cell activation, proliferation, survival, and migration of hepatic stellate cells. Inhibition of DGAT2 and FASN, OPN, and/or NOX4 may represent a useful therapeutic approach for the treatment of liver diseases, such as NAFLD and NASH.

### SUMMARY

In one aspect, the disclosures provide a combination of oligonucleotides comprising: a first oligonucleotide comprising a 5′ end, a 3′ end, and a sequence substantially complementary to a Diacylglycerol O-Acyltransferase 2 (DGAT2) nucleic acid sequence; and a second oligonucleotide comprising a 5′ end, a 3′ end, and a sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence.

In certain embodiments, the first oligonucleotide comprises a sequence substantially complementary to a DGAT2 nucleic acid sequence of any one of SEQ ID NOs: 1-5. In certain embodiments, the first oligonucleotide comprises a sequence substantially complementary to a DGAT2 nucleic acid sequence of SEQ ID NO: 1. In certain embodiments, the first oligonucleotide comprises a sequence substantially complementary to a DGAT2 nucleic acid sequence of SEQ ID NO: 2. In certain embodiments, the first oligonucleotide comprises a sequence substantially complementary to a DGAT2 nucleic acid sequence of SEQ ID NO: 3. In certain embodiments, the first oligonucleotide comprises a sequence substantially complementary to a DGAT2 nucleic acid sequence of SEQ ID NO: 4. In certain embodiments, the first oligonucleotide comprises a sequence substantially complementary to a DGAT2 nucleic acid sequence of SEQ ID NO: 5.

In certain embodiments, the first oligonucleotide comprises a sequence substantially complementary to a DGAT2 nucleic acid sequence of any one of SEQ ID NOs: 6-10. In certain embodiments, the first oligonucleotide comprises a sequence substantially complementary to a DGAT2 nucleic acid sequence of SEQ ID NO: 6. In certain embodiments, the first oligonucleotide comprises a sequence substantially complementary to a DGAT2 nucleic acid sequence of SEQ ID NO: 7. In certain embodiments, the first oligonucleotide comprises a sequence substantially complementary to a DGAT2 nucleic acid sequence of SEQ ID NO: 8. In certain embodiments, the first oligonucleotide comprises a sequence substantially complementary to a DGAT2 nucleic acid sequence of SEQ ID NO: 9. In certain embodiments, the first oligonucleotide comprises a sequence substantially complementary to a DGAT2 nucleic acid sequence of SEQ ID NO: 10.

In certain embodiments, the first oligonucleotide comprises complementarity to at least 10, 11, 12, or 13 contiguous nucleotides of the DGAT2 nucleic acid sequence of any one of SEQ ID NOs: 1-10.

In certain embodiments, the first oligonucleotide comprises no more than 3 mismatches with the DGAT2 nucleic acid sequence of any one of SEQ ID NOs: 1-10.

In certain embodiments, the first oligonucleotide comprises a sequence fully complementary to the DGAT2 nucleic acid sequence of any one of SEQ ID NOs: 1-10.

In certain embodiments, the second oligonucleotide comprises a sequence substantially complementary to a FASN nucleic acid sequence of any one of Table 5. In certain embodiments, the second oligonucleotide comprises a sequence substantially complementary to a FASN nucleic acid sequence of SEQ ID NO: 64. In certain embodiments, the second oligonucleotide comprises a sequence substantially complementary to a FASN nucleic acid sequence of SEQ ID NO: 65. In certain embodiments, the second oligonucleotide comprises a sequence substantially complementary to a FASN nucleic acid sequence of SEQ ID NO: 66. In certain embodiments, the second oligonucleotide comprises a sequence substantially complementary to a FASN nucleic acid sequence of SEQ ID NO: 67.

In certain embodiments, the second oligonucleotide comprises complementarity to at least 10, 11, 12, or 13 contiguous nucleotides of the FASN nucleic acid sequence of any one of Table 5.

In certain embodiments, the second oligonucleotide comprises no more than 3 mismatches with the FASN nucleic acid sequence of any one of Table 5.

In certain embodiments, the second oligonucleotide comprises a sequence fully complementary to the FASN nucleic acid sequence of any one of Table 5.

In certain embodiments, the second oligonucleotide comprises a sequence substantially complementary to a OPN nucleic acid sequence of any one of Table 8. In certain embodiments, the second oligonucleotide comprises a sequence substantially complementary to a OPN nucleic acid sequence of SEQ ID NO: 68. In certain embodiments, the second oligonucleotide comprises a sequence substantially complementary to a OPN nucleic acid sequence of SEQ ID NO: 69. In certain embodiments, the second oligonucleotide comprises a sequence substantially complementary to a OPN nucleic acid sequence of SEQ ID NO: 70. In certain embodiments, the second oligonucleotide comprises a sequence substantially complementary to a OPN nucleic acid sequence of SEQ ID NO: 71.

In certain embodiments, the second oligonucleotide comprises complementarity to at least 10, 11, 12, or 13 contiguous nucleotides of the OPN nucleic acid sequence of any one of Table 8.

In certain embodiments, the second oligonucleotide comprises no more than 3 mismatches with the OPN nucleic acid sequence of any one of Table 8.

In certain embodiments, the second oligonucleotide comprises a sequence fully complementary to the OPN nucleic acid sequence of any one of Table 8.

In certain embodiments, the second oligonucleotide comprises a sequence substantially complementary to a NOX4 nucleic acid sequence of any one of Table 10. In certain embodiments, the second oligonucleotide comprises a sequence substantially complementary to a NOX4 nucleic acid sequence of SEQ ID NO: 72. In certain embodiments, the second oligonucleotide comprises a sequence substantially complementary to a NOX4 nucleic acid sequence of SEQ ID NO: 73.

In certain embodiments, the second oligonucleotide comprises complementarity to at least 10, 11, 12, or 13 contiguous nucleotides of the NOX4 nucleic acid sequence of any one of Table 10.

In certain embodiments, the second oligonucleotide comprises no more than 3 mismatches with the NOX4 nucleic acid sequence of any one of Table 10.

In certain embodiments, the second oligonucleotide comprises a sequence fully complementary to the NOX4 nucleic acid sequence of any one of Table 10.

In certain embodiments, each of the first and the second oligonucleotides is independently an antisense oligonucleotide (ASO) or a double stranded RNA (dsRNA) moiety, and the dsRNA moiety comprises a sense strand and an antisense strand, each strand comprising separately a 3′ end and a 5′ end.

In certain embodiments, the antisense strand comprises about 15 nucleotides to about 25 nucleotides in length.

In certain embodiments, the sense strand comprises about 15 nucleotides to about 25 nucleotides in length.

In certain embodiments, the antisense strand is 20 nucleotides in length.

In certain embodiments, the antisense strand is 21 nucleotides in length.

In certain embodiments, the antisense strand is 22 nucleotides in length.

In certain embodiments, the sense strand is 15 nucleotides in length.

In certain embodiments, the sense strand is 16 nucleotides in length.

In certain embodiments, the sense strand is 18 nucleotides in length.

In certain embodiments, the sense strand is 20 nucleotides in length or 21 nucleotides in length.

In certain embodiments, the combination comprises a double-stranded region of 15 base pairs to 20 base pairs.

In certain embodiments, the combination comprises a double-stranded region of 15 base pairs.

In certain embodiments, the combination comprises a double-stranded region of 16 base pairs.

In certain embodiments, the combination comprises a double-stranded region of 18 base pairs.

In certain embodiments, the combination comprises a double-stranded region of 20 base pairs or 21 base pairs.

In certain embodiments, the dsRNA moiety comprises a blunt-end.

In certain embodiments, the dsRNA moiety comprises at least one single stranded nucleotide overhang.

In certain embodiments, the dsRNA moiety comprises an about 2-nucleotide to about 5-nucleotide single stranded nucleotide overhang.

In certain embodiments, the dsRNA moiety comprises 2-nucleotide single stranded nucleotide overhang.

In certain embodiments, the dsRNA moiety comprises 5-nucleotide single stranded nucleotide overhang.

In certain embodiments, the dsRNA moiety comprises naturally occurring nucleotides.

In certain embodiments, the dsRNA moiety comprises at least one modified nucleotide.

In certain embodiments, the at least one modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the dsRNA moiety comprises at least one modified internucleotide linkage.

In certain embodiments, the at least one modified internucleotide linkage comprises a phosphorothioate internucleotide linkage.

In certain embodiments, the combination comprises 4-16 phosphorothioate internucleotide linkages.

In certain embodiments, the combination comprises 4-13 phosphorothioate internucleotide linkages.

In certain embodiments, the dsRNA comprises 8 or 13 phosphorothioate internucleotide linkages.

In certain embodiments, the dsRNA moiety comprises at least one modified internucleotide linkage of Formula I:

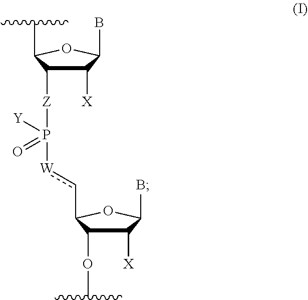

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
R is a protecting group; and
[symbol] is an optional double bond.

In certain embodiments, the dsRNA moiety comprises at least 80% chemically modified nucleotides.

In certain embodiments, the dsRNA moiety is fully chemically modified.

In certain embodiments, the dsRNA moiety comprises at least 70% 2′-O-methyl nucleotide modifications.

In certain embodiments, the antisense strand comprises at least 70% 2′-O-methyl nucleotide modifications.

In certain embodiments, the antisense strand comprises about 70% to about 90% 2′-O-methyl nucleotide modifications.

In certain embodiments, the sense strand comprises at least 65% 2′-O-methyl nucleotide modifications.

In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the sense strand comprises about 15 nucleotides to about 25 nucleotides in length, and the one or more nucleotide mismatches are present at positions 2, 6, and 12 from the 5′ end of the sense strand.

In certain embodiments, the sense strand comprises about 15 nucleotides to about 25 nucleotides in length, and the one or more nucleotide mismatches consist of nucleotide mismatches present at positions 2, 6, and 12 from the 5′ end of the sense strand.

In certain embodiments, the antisense strand comprises a 5′ phosphate, a 5′-alkyl phosphonate, a 5′ alkylene phosphonate, or a 5′ alkenyl phosphonate.

In certain embodiments, the antisense strand comprises a 5′ vinyl phosphonate.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 80% 2′-O-methyl modifications;
(3) the nucleotides at positions 2, 4, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 80% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, 14, 16, and 20 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7 and 9-11 from the 3′ end of the sense strand are not 2′-methoxy-ribonucleotides; and
(8) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the first oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7, 10, and 11 from the 3′ end of the sense strand are not 2′-methoxy-ribonucleotides; and
(8) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, and:
(1) the antisense strand comprises a sequence substantially complementary to a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In certain embodiments, the nucleotide at position 20 from the 5′ end of the antisense strand is not a 2′-methoxy-ribonucleotide.

In certain embodiments, the functional moiety is linked to the 5′ end and/or 3′ end of the antisense strand.

In certain embodiments, the functional moiety is linked to the 5′ end and/or 3′ end of the sense strand.

In certain embodiments, the functional moiety is linked to the 3′ end of the sense strand.

In certain embodiments, the functional moiety comprises an N-acetylgalactosamine (GalNAc) moiety.

In certain embodiments, the functional moiety comprises a hydrophobic moiety.

In certain embodiments, the hydrophobic moiety is selected from the group consisting of fatty acids, steroids, secosteroids, lipids, gangliosides, nucleoside analogs, endocannabinoids, vitamins, and a mixture thereof.

In certain embodiments, the steroid is selected from the group consisting of cholesterol and lithocholic acid (LCA).

In certain embodiments, the fatty acid is selected from the group consisting of eicosapentaenoic acid (EPA), docosahexaenoic acid (DHA) and docosanoic acid (DCA).

In certain embodiments, the functional moiety is linked to the antisense strand and/or the sense strand by a linker.

In certain embodiments, the linker is a cleavable linker.

In certain embodiments, the cleavable linker comprises a phosphodiester linkage, a disulfide linkage, an acid-labile linkage, a photocleavable linkage, or a dTdT dinucleotide with phosphodiester internucleotide linkages.

In certain embodiments, the acid-labile linkage comprises a β-thiopropionate linkage or a carboxydimethylmaleic anhydride (CDM) linkage.

In certain embodiments, the linker comprises a divalent or trivalent linker.

In certain embodiments, the divalent or trivalent linker is selected from the group consisting of:

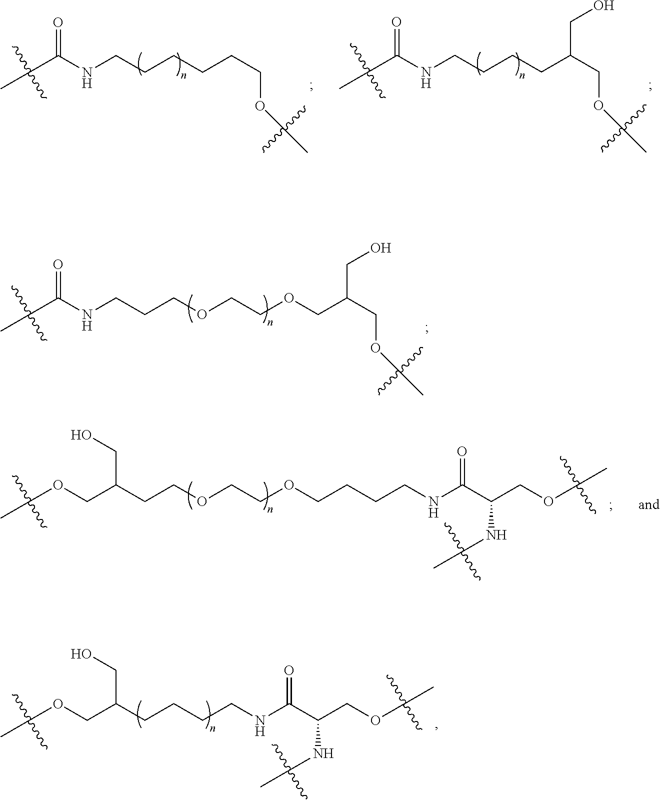

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

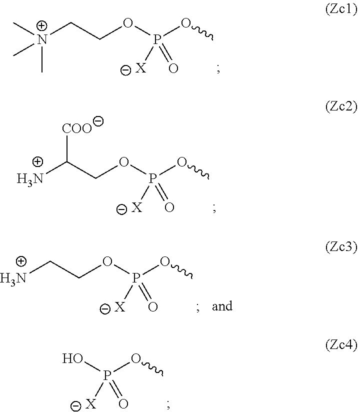

wherein X is O, S or BH3.

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the first oligonucleotide inhibits expression of the DGAT2 nucleic acid sequence by at least about 50%.

In certain embodiments, the second oligonucleotide inhibits expression of one of the FASN, OPN, and NOX4 nucleic acid sequences by at least about 50%.

In another aspect, the disclosures provide a pharmaceutical composition for inhibiting the expression of a Diacylglycerol O-Acyltransferase 2 (DGAT2) gene and a Fatty Acid Synthase (FASN) gene, an Osteopontin (OPN) gene, or a Nicotinamide Adenine Dinucleotide Phosphate Oxidase (NOX4) gene in an organism. In such aspect, the pharmaceutical composition comprises the combination of any one of claims 1-80 and a pharmaceutically acceptable carrier.

In certain embodiments, the first oligonucleotide inhibits the expression of a DGAT2 gene by at least about 50%.

In certain embodiments, the second oligonucleotide inhibits the expression of one of a FASN gene, an OPN gene, and a NOX4 gene by at least about 80%.

In another aspect, the disclosures provides a method of treating or managing a disease associated with DGAT2 and FASN, OPN, or NOX4, the method comprising administering to a patient in need of such treatment a therapeutically effective amount of a combination of oligonucleotides comprising: a first oligonucleotide comprising a 5′ end, a 3′ end, and a sequence substantially complementary to a Diacylglycerol O-Acyltransferase 2 (DGAT2) nucleic acid sequence; and a second oligonucleotide comprising a 5′ end, a 3′ end, and a sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence. The first oligonucleotide inhibits DGAT2 gene expression by at least about 50% for four weeks post administration, and the second oligonucleotide inhibits a FASN gene, an OPN gene, or a NOX4 gene expression by at least about 50% for four weeks post administration.

In certain embodiments, the disease is non-alcoholic fatty liber disease (NAFLD), non-alcoholic steatohepatitis (NASH), lipodystrophy, or a combination thereof

In certain embodiments, the combination is administered by intracerebroventricular (ICV) injection, intrastriatal (IS) injection, intravenous (IV) injection, subcutaneous (SQ) injection, or a combination thereof.

In certain embodiments, the first and second oligonucleotides are administered concurrently or simultaneously.

In certain embodiments, the combination is administered to the liver.

In another aspect, the disclosures provide a method for delivering a combination of oligonucleotides to the liver of a patient, the method comprising administering the combination of oligonucleotides to the patient.

In another aspect, the disclosures provide a vector comprising: a first regulatory sequence operably linked to a first nucleotide sequence that encodes a first oligonucleotide substantially complementary to a Diacylglycerol O-Acyltransferase 2 (DGAT2) nucleic acid sequence; and a second regulatory sequence operably linked to a second nucleotide sequence that encodes a second oligonucleotide substantially complementary to a (FASN) nucleic acid sequence, an Osteopontin (OPN) nucleic acid sequence, or a Adenine Dinucleotide Phosphate Oxidase (NOX4) nucleic acid sequence.

In certain embodiments, the first oligonucleotide inhibits expression of the DGAT2 nucleic acid sequence by at least 30%, at least 50%, or at least 80%, and the second oligonucleotide inhibits expression of the FASN nucleic acid sequence, OPN nucleic acid sequence, and NOX4 nucleic acid sequence by at least 30%, at least 50%, or at least 80%.

In another aspect, the disclosures provide a cell comprising a vector.

In another aspect, the disclosures provide a recombinant adeno-associated virus (rAAV) comprising the vector and an AAV capsid.

In another aspect, the disclosures provide a branched compound comprising: a first oligonucleotide comprising a 5′ end, a 3′ end, and a sequence substantially complementary to a Diacylglycerol O-Acyltransferase 2 (DGAT2) nucleic acid sequence; and a second oligonucleotide comprising a 5′ end, a 3′ end, and a sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence. The first and second oligonucleotide are linked together by a linker.

In certain embodiments, the linker has a structure L1:

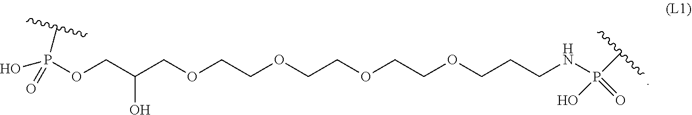 [SMILES: *P(=O)(O)NCCCOCCOCCOCCOCC(O)COP(*)(=O)O]

In certain embodiments, the linker has a structure L2:

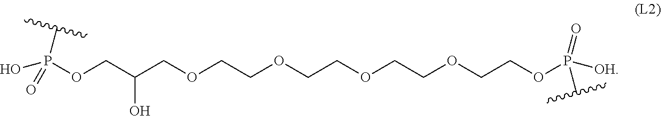 [SMILES: *P(=O)(O)OCCOCCOCCOCCOCC(O)COP(*)(=O)O]

In certain embodiments, the at least one of the first and second oligonucleotides is a double stranded RNA (dsRNA) moiety comprising a sense strand and an antisense strand, and the antisense strand comprises a 5′ terminal group R selected from the group consisting of:

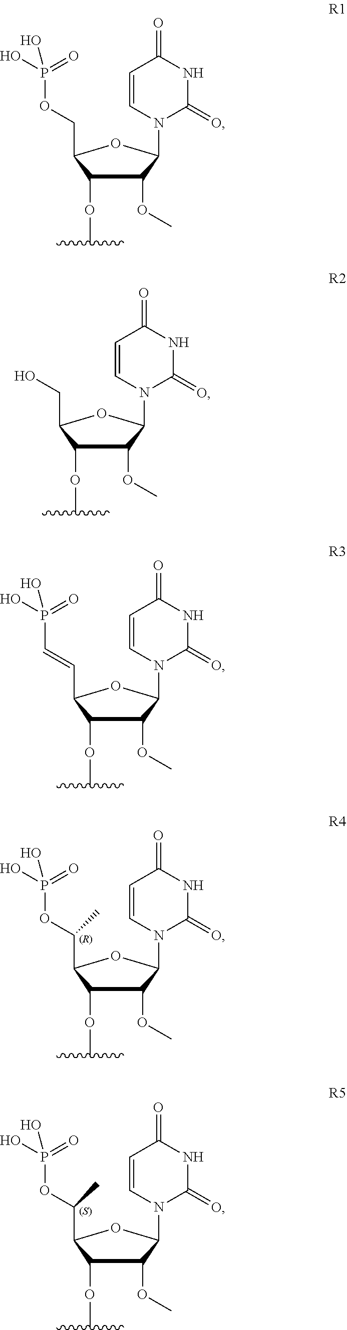

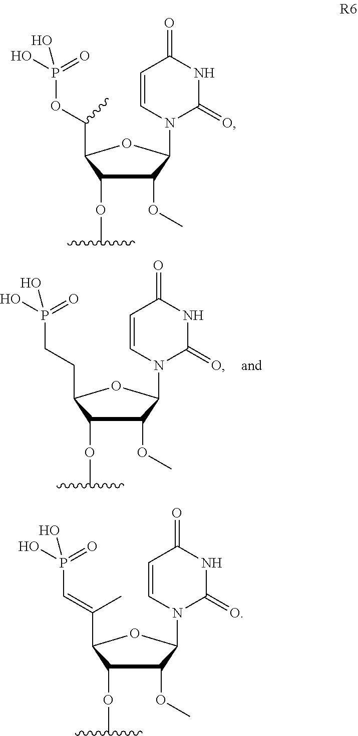

In certain embodiments, at least one of the first and second oligonucleotides is an antisense oligonucleotide (ASO).

In certain embodiments, the first oligonucleotide is a double stranded RNA (dsRNA) comprising a sense strand and an antisense strand, and the second oligonucleotide is an antisense oligonucleotide (ASO). In certain embodiments, the ASO is a gapmer.

In one aspect, the disclosures provide an oligonucleotide comprising a 5′ end, a 3′ end, and a sequence substantially complementary to a Fatty Acid Synthase (FASN) nucleic acid sequence.

In certain embodiments, the second oligonucleotide comprises a sequence substantially complementary to a FASN nucleic acid sequence of any one of Table 5. In certain embodiments, the second oligonucleotide comprises a sequence substantially complementary to a FASN nucleic acid sequence of SEQ ID NO: 64. In certain embodiments, the second oligonucleotide comprises a sequence substantially complementary to a FASN nucleic acid sequence of SEQ ID NO: 65. In certain embodiments, the second oligonucleotide comprises a sequence substantially complementary to a FASN nucleic acid sequence of SEQ ID NO: 66. In certain embodiments, the second oligonucleotide comprises a sequence substantially complementary to a FASN nucleic acid sequence of SEQ ID NO: 67.

In one aspect, the disclosures provide an oligonucleotide comprising a 5′ end, a 3′ end, and a sequence substantially complementary to an Osteopontin (OPN) nucleic acid sequence.

In certain embodiments, the second oligonucleotide comprises a sequence substantially complementary to a OPN nucleic acid sequence of any one of Table 8. In certain embodiments, the second oligonucleotide comprises a sequence substantially complementary to a OPN nucleic acid sequence of SEQ ID NO: 68. In certain embodiments, the second oligonucleotide comprises a sequence substantially complementary to a OPN nucleic acid sequence of SEQ ID NO: 69. In certain embodiments, the second oligonucleotide comprises a sequence substantially complementary to a OPN nucleic acid sequence of SEQ ID NO: 70. In certain embodiments, the second oligonucleotide comprises a sequence substantially complementary to a OPN nucleic acid sequence of SEQ ID NO: 71.

In one aspect, the disclosures provide an oligonucleotide comprising a 5′ end, a 3′ end, and a sequence substantially complementary to a Nicotinamide Adenine Dinucleotide Phosphate Oxidase (NOX4) nucleic acid sequence.

In certain embodiments, the second oligonucleotide comprises a sequence substantially complementary to a NOX4 nucleic acid sequence of any one of Table 10. In certain embodiments, the second oligonucleotide comprises a sequence substantially complementary to a NOX4 nucleic acid sequence of SEQ ID NO: 72. In certain embodiments, the second oligonucleotide comprises a sequence substantially complementary to a NOX4 nucleic acid sequence of SEQ ID NO: 73.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other features and advantages of the present disclosure will be more fully understood from the following detailed description of illustrative embodiments taken in conjunction with the accompanying drawings.

FIG. 1A-FIG. 1G depict the FASN screening of siRNA compounds. FIG. 1A shows a schematic representation of an oligonucleotide. FIG. 1B shows the in vitro and in vivo screening of compounds in mouse. FIG. 1C shows FASN human screening of siRNA compounds. FIG. 1D (FASN_NM_004104_2314_as (SEQ ID NO: 56), FASN_NM_004104_2314_s (SEQ ID NO: 57), FASN_NM_004104_5428_AS (SEQ ID NO: 58), and FASN_NM_004104_5428_S (SEQ ID NO: 59)) and FIG. 1E (FASN_NM_004104_8323_as (SEQ ID NO: 60), FASN_NM_004104_8323_s (SEQ ID NO: 61), FASN_NM_004104_8324_as (SEQ ID NO: 62), FASN_NM_004104_8324_s (SEQ ID NO: 63)) show the 8-point dose response from the human FASN screening of siRNA compounds. FIG. 1F shows the mouse FASN screening of ASO compounds in vivo. FIG. 1G (F_12 (SEQ ID NO: 952) and F_18 (SEQ ID NO: 953)) shows the in vitro silencing of FASN with ASOs in Human Huh7 cells.

FIG. 2A-FIG. 2E depict the FASN inhibition that blunts the activation and collagen production in human stellate cells. LX2 human stellate cell line was plated in 6 well plates and serum-starved overnight. After 4-hour pre-treatment with 1 nM or 100 nM FASN inhibitor (TVB-3664), stellate cell activation was initiated with addition of recombinant human Tgf-β (5 ng/mL final concentration). FIG. 2A shows the cell morphology difference upon FASN inhibition. FIG. 2B shows the protein levels of collagen type-1 and phosphorylated Smad3. FIG. 2C shows fibrotic gene expression changes. FIG. 2D shows the oxidative stress and mitochondrial function related gene expression changes. (ns: Not significant, *: p<0.05, **: p<0.005, ***: p<0.0005, ****: p<0.00005). FIG. 2E shows the gel analysis of untreated control, cholesterol conjugated non-targeting control, and cholesterol conjugated Fasn-2314, and the protein level of Smad3.

FIG. 3A-FIG. 3F depict stellate cell-specific deletion of Fasn, in combination with Dgat2 silencing, ameliorating liver stiffness and plasma markers of NASH in CDAHFD fed mice. FIG. 3A schematics of the assay and an oligonucleotide. 10-week-old male Fasn fl/fl mice were injected with corresponding AAVs to obtain hepatocyte specific KO (AAV8-TBG-Cre) and/or stellate cell specific KO (AAV9-Lrat-Cre) via IV injection. Additionally, for the indicated groups, sdDgat2 (10 mg/kg) was injected via single subcutaneous injection. After one week of injections, mice were put on CDAHFD for 7 weeks. FIG. 3B shows the liver stiffness measurements by shear-wave elastography. FIG. 3C shows the plasma ALT activity. FIG. 3D shows the plasma total bilirubin level. FIG. 3E shows the plasma TNF alpha level. FIG. 3F shows the plasma MCP1 protein level. (ns: Not significant, *: p<0.05, **: p<0.005, ***: p<0.0005, ****: p<0.00005).

FIG. 4A-FIG. 4F depict stellate cell-specific deletion of Fasn, in combination with Dgat2 silencing, preventing NASH in CDAHFD fed mice. FIG. 4A shows mRNA levels of Dgat2 and Fasn. FIG. 4B shows the total liver triglyceride measurement. FIG. 4C shows the immunohistochemical analysis of type 1 collagen. FIG. 4D shows the quantification of type 1 collagen positive area in IHC. FIG. 4E shows the total collagen measurement via OH-Pro assay. FIG. 4F shows liver weights. (ns: Not significant, *: p<0.05, **: p<0.005, ***: p<0.0005, ****: p<0.00005).

FIG. 5A-FIG. 5B depict the effect of Dgat2-Fasn combination in MASH development in human liver organoids. FIG. 5A shows mRNA levels of DGAT2 mRNA, FASN mRNA, COL1A1 mRNA, and COL3A1 mRNA. FIG. 5B shows Collagen1 protein and DAPI measured by immunofluorescence in human liver organoids (ns: Not significant, *: p<0.05, **: p<0.005, ***: p<0.0005, ****: p<0.00005).

FIG. 6A-FIG. 6F depict the effect of Dgat2-Fasn combination in NASH development in CDAHFD mouse model. FIG. 6A shows a schematic of the assay. FIG. 6B shows liver stiffness measurements by shear-wave elastography. FIG. 6C shows the liver to body ratio. FIG. 6D shows the liver triglyceride content. FIG. 6E shows the Dgat2/Fasn silencing. FIG. 6F shows the collagen content in liver sections (ns: Not significant, *: p<0.05, **: p<0.005, ***: p<0.0005, ****: p<0.00005).

FIG. 7A-FIG. 7C depict OPN screening of siRNA compounds. FIG. 7A shows a schematic representation of an oligonucleotide. FIG. 7B shows the mouse OPN screening of siRNA compounds. FIG. 7C shows the human OPN screening of siRNA compounds.

FIG. 8A-FIG. 8C depict NOX4 screening of siRNA compounds. FIG. 8A shows a schematic representation of an oligonucleotide. FIG. 8B shows the mouse NOX4 screening of siRNA compounds. FIG. 8C shows the human NOX4 screening of siRNA compounds.

FIG. 9A-FIG. 9C depict DGAT2 and FASN silencing using siRNA-siRNA and siRNA-ASO compounds. FIG. 9A shows schematics of siRNA-siRNA and siRNA-ASO compounds. FIG. 9B shows a schematic of an assay and results of a siRNA-siRNA KD efficiency whole liver study. FIG. 9C shows a schematic of an assay and results of a siRNA-siRNA biodistribution study.

FIG. 10A-10C depict DGAT2 and MALAT1 silencing using a siRNA-ASO compound. FIG. 10A shows schematics of the compound and assay. FIG. 10B shows the validation of separation of hepatocytes and non-hepatocytes. FIG. 10C shows DGAT2 and MALAT1 silencing in hepatocytes and non-hepatocytes.

FIG. 11 shows an FASN-DGAT2 siASO in vivo screen.

### DETAILED DESCRIPTION

The combination of DGAT2 targeting sequences with FASN, OPN, and/or NOX4 targeting sequences, and methods for treating and preventing non-alcoholic fatty liver disease (NAFLD), non-alcoholic steatohepatitis (NASH), and lipodystrophy syndromes are provided.

Unless otherwise specified, nomenclature used in connection with cell and tissue culture, molecular biology, immunology, microbiology, genetics, and protein and nucleic acid chemistry and hybridization described herein are those well-known and commonly used in the art. Unless otherwise specified, the methods and techniques provided herein are performed according to conventional methods well-known in the art and as described in various general and more specific references that are cited and discussed throughout the present specification. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21, or 22 nucleotides or nucleotide analogs). The term “short” siRNA refers to a siRNA comprising about 21 nucleotides (or nucleotide analogs), for example, 19, 20, 21, or 22 nucleotides. The term “long” siRNA refers to a siRNA comprising about 24-25 nucleotides, for example, 23, 24, 25, or 26 nucleotides. Short siRNAs may, in some instances, include fewer than 19 nucleotides, e.g., 16, 17, or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide that may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified nucleotides (e.g., alkylated, e.g., N6-methyl adenosine, or as otherwise known in the art); and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotide. For example, the 2′ OH-group may be replaced by a group selected from H, OR, R, F, Cl, Br, I, SH, SR, NH2, NHR, NR2, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is ““sequence sufficiently complementary to a target mRNA sequence to direct target-specific RNA interference (RNAi”)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or ““silencing”” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target“polynucleotide sequence,” e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g., promoter or enhancer elements) of a target gene. In other embodiments, the target polynucleotide sequence is a target mRNA encoded by a target gene.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene.

As described herein, the term “DGAT2” refers to the gene encoding for the enzyme, Diacylglycerol O-Acyltransferase 2. DGAT2 catalyzes the reaction that covalently joins diacylglycerol to long chain fatty acyl-CoAs in the final step of triglyceride synthesis. The DGAT2 gene is located on chromosome 11, is made up of 9 exons and is mainly expressed in the liver and white adipose tissue. The DGAT2 protein is 388 amino acids in length and has a molecular mass of approximately 43,831 Da.

As described herein, the term “FASN” refers to the gene encoding for the enzyme, Fatty Acid Synthase. FASN is a multifunctional enzyme that catalyzes the de novo biosynthesis of long-chain saturated fatty acids starting from acetyl-CoA and malonyl-CoA in the presence of NADPH. This multifunctional protein contains 7 catalytic activities and a site for the binding of the prosthetic group 4′-phosphopantetheine of the acyl carrier protein (ACP) domain. The FASN gene is located on chromosome 17 and is mainly expressed in brain, lung, liver, and mammary gland. The FASN protein is 2511 amino acids in length and has a molecular mass of approximately 273,427 Da.

As described herein, the term “OPN” refers to the gene encoding for the enzyme, Osteopontin. OPN acts as a cytokine involved in enhancing production of interferon-gamma and interleukin-12 and reducing production of interleukin-10 and is essential in the pathway that leads to type I immunity. The OPN gene is located on chromosome 4 and is mainly expressed in bones. The OPN protein is 314 amino acids in length and has a molecular mass of approximately 35,423 Da.

As described herein, the term “NOX4” refers to the gene encoding for the enzyme, Nicotinamide Adenine Dinucleotide Phosphate Hydrogen Oxidases 4 (NOX4). NOX4 is a constitutive NADPH oxidase which generates superoxide intracellularly upon formation of a complex with CYBA/p22phox. The NOX4 gene is located on chromosome 11 and is strongly expressed in kidney and to a lower extent in heart, adipocytes, hepatoma, endothelial cells, skeletal muscle, brain, several brain tumor cell lines and airway epithelial cells. The NOX4 protein is 578 amino acids in length and has a molecular mass of approximately 66,932 Da.

The phrase “examining the function of a gene in a cell or organism” refers to examining or studying the expression, activity, function or phenotype arising therefrom.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine, and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and p is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the terms “linking moiety” or “linking portion” refer to a domain, portion or region of the RNA-silencing agent which covalently joins or links the mRNA.

As used herein, the terms “miRNA recruiting moiety” or miRNA targeting moiety” or “miRNA recruiting portion” refer to a domain, portion or region of the dual functional oligonucleotide having sufficient size and sufficient complementarity to a portion or region of a miRNA chosen or targeted for recruitment to the target mRNA.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23, or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog, such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of an RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from short hairpin RNA (shRNA) precursors. Both RNAi and translational repression are mediated by RNA-induced silencing complex (RISC). Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

Various aspects of the disclosure are described in further detail in the following subsections.

### I. Target Sequences

In certain exemplary embodiments, oligonucleotide silencing agents of the disclosure are capable of targeting a DGAT2 nucleic acid sequence of any one of SEQ ID NOs: 1-10, as recited in Table 1. In certain exemplary embodiments, oligonucleotide silencing agents of the disclosure comprise double stranded RNA (dsRNA) molecules comprising a sense strand and an antisense strand, wherein the antisense strand comprises a sequence substantially complementary to a DGAT2 nucleic acid sequence of SEQ ID NO: 1-10, as recited in Table 1. Exemplary antisense and sense strands are recited in Tables 2-4.

In certain exemplary embodiments, oligonucleotide silencing agents of the disclosure are capable of targeting a FASN nucleic acid sequence of any one of Table 5. In certain exemplary embodiments, oligonucleotide silencing agents of the disclosure comprise double stranded RNA (dsRNA) molecules comprising a sense strand and an antisense strand, wherein the antisense strand comprises a sequence substantially complementary to a FASN nucleic acid sequence of Table 5. Exemplary antisense and sense strands are recited in Table 7.

In certain exemplary embodiments, oligonucleotide silencing agents of the disclosure are capable of targeting a OPN nucleic acid sequence of any one of Table 8. In certain exemplary embodiments, oligonucleotide silencing agents of the disclosure comprise double stranded RNA (dsRNA) molecules comprising a sense strand and an antisense strand, wherein the antisense strand comprises a sequence substantially complementary to a OPN nucleic acid sequence of Table 8.

In certain exemplary embodiments, oligonucleotide silencing agents of the disclosure are capable of targeting a NOX4 nucleic acid sequence of any one of Table 10. In certain exemplary embodiments, oligonucleotide silencing agents of the disclosure comprise double stranded RNA (dsRNA) molecules comprising a sense strand and an antisense strand, wherein the antisense strand comprises a sequence substantially complementary to a NOX4 nucleic acid sequence of Table 10.

The genomic sequence for each target sequence can be found in, for example, the publicly available database maintained by the NCBI.

### II. siRNA Design

In some embodiments, siRNAs are designed as follows. First, a portion of the target gene (e.g., a DGAT2 gene, a FASN gene, an OPN gene, and a NOX4 gene), e.g., one or more of the target sequences set forth in Tables 1, 5, 7, and 9 is selected. Cleavage of mRNA at these sites should eliminate translation of corresponding protein. Antisense strands were designed based on the target sequence and sense strands were designed to be complementary to the antisense strand. Hybridization of the antisense and sense strands forms the siRNA duplex. The antisense strand includes about 19 to about 25 nucleotides, e.g., 19, 20, 21, 22, 23, 24, or 25 nucleotides. In other embodiments, the antisense strand includes 20, 21, 22 or 23 nucleotides. The sense strand includes about 14 to about 25 nucleotides, e.g., 14, 15, 16, 17, 18, 19, 20, 21, 22, 23, 24, or 25 nucleotides. In other embodiments, the sense strand is 15 nucleotides. In other embodiments, the sense strand is 18 nucleotides. In other embodiments, the sense strand is 20 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant disclosure, provided that they retain the ability to mediate RNAi. Longer RNAi agents have been demonstrated to elicit an interferon or PKR response in certain mammalian cells, which may be undesirable. In certain embodiments, the RNAi agents of this disclosure do not elicit a PKR response (i.e., are of a sufficiently short length). However, longer RNAi agents may be useful, for example, in cell types incapable of generating a PKR response or in situations where the PKR response has been down-regulated or dampened by alternative means.

The sense strand sequence can be designed such that the target sequence is essentially in the middle of the strand. Moving the target sequence to an off-center position can, in some instances, reduce efficiency of cleavage by the siRNA. Such compositions, i.e., less efficient compositions, may be desirable for use if off-silencing of the wild-type mRNA is detected.

The antisense strand can be the same length as the sense strand and includes complementary nucleotides. In one embodiment, the strands are fully complementary, i.e., the strands are blunt-ended when aligned or annealed. In another embodiment, the strands align or anneal such that 1-, 2-, 3-, 4-, 5-, 6-, 7-, or 8-nucleotide overhangs are generated, i.e., the 3′ end of the sense strand extends 1, 2, 3, 4, 5, 6, 7, or 8 nucleotides further than the 5′ end of the antisense strand and/or the 3′ end of the antisense strand extends 1, 2, 3, 4, 5, 6, 7, or 8 nucleotides further than the 5′ end of the sense strand. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced, as described in detail in U.S. Pat. Nos. 7,459,547, 7,772,203 and 7,732,593, entitled “Methods and Compositions for Controlling Efficacy of RNA Silencing” (filed Jun. 2, 2003) and U.S. Pat. Nos. 8,309,704, 7,750,144, 8,304,530, 8,329,892 and 8,309,705, entitled “Methods and Compositions for Enhancing the Efficacy and Specificity of RNAi” (filed Jun. 2, 2003), the contents of which are incorporated in their entirety by this reference. In one embodiment of these aspects of the disclosure, the base-pair strength is less due to fewer G:C base pairs between the 5′ end of the first or antisense strand and the 3′ end of the second or sense strand than between the 3′ end of the first or antisense strand and the 5′ end of the second or sense strand. In another embodiment, the base pair strength is less due to at least one mismatched base pair between the 5′ end of the first or antisense strand and the 3′ end of the second or sense strand. In certain exemplary embodiments, the mismatched base pair is selected from the group consisting of G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair comprising a rare nucleotide, e.g., inosine (I). In certain exemplary embodiments, the base pair is selected from the group consisting of an I:A, I:U and I:C. In yet another embodiment, the base pair strength is less due to at least one base pair comprising a modified nucleotide. In certain exemplary embodiments, the modified nucleotide is selected from the group consisting of 2-amino-G, 2-amino-A, 2,6-diamino-G, and 2,6-diamino-A.

The design of siRNAs suitable for targeting the DGAT2, FASN, OPN, and NOX4 target sequences set forth in Tables 1, 5, 7, and 9 is described in detail below. siRNAs can be designed according to the above exemplary teachings for any other target sequences found in a DGAT2 gene, a FASN gene, an OPN gene, and a NOX4 gene. Moreover, the technology is applicable to targeting any other target sequences, e.g., non-disease-causing target sequences.

To validate the effectiveness by which siRNAs destroy mRNAs (e.g., DGAT2 mRNA, FASN mRNA, OPN mRNA, NOX4 mRNA), the siRNA can be incubated with cDNA (e.g., DGAT2 cDNA, FASN cDNA, OPN cDNA, and NOX4 cDNA) in a Drosophila-based in vitro mRNA expression system. Radiolabeled with 32P, newly synthesized mRNAs (e.g., DGAT2mRNA, FASNmRNA, OPNmRNA, and NOX4mRNA) are detected autoradiographically on an agarose gel. The presence of cleaved mRNA indicates mRNA nuclease activity. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence. Sites of siRNA-mRNA complementation are selected which result in optimal mRNA specificity and maximal mRNA cleavage.

### III. RNAi Agents

The present disclosure includes RNAi molecules, such as siRNA molecules designed, as described above. The siRNA molecules of the disclosure can be chemically synthesized, or can be transcribed in vitro from a DNA template, or in vivo from e.g., shRNA, or by using recombinant human DICER enzyme, to cleave in vitro transcribed dsRNA templates into pools of 20-, 21- or 23-bp duplex RNA mediating RNAi. The siRNA molecules can be designed using any method known in the art.

In one aspect, instead of the RNAi agent being an interfering ribonucleic acid, the RNAi agent can encode an interfering ribonucleic acid, as described above. In other words, the RNAi agent can be a transcriptional template of the interfering ribonucleic acid. Thus, RNAi agents of the present disclosure can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides (Brummelkamp et al., 2002; Lee et al., 2002, Supra; Miyagishi et al., 2002; Paddison et al., 2002, supra; Paul et al., 2002, supra; Sui et al., 2002 supra; Yu et al., 2002, supra. More information about shRNA design and use can be found on the internet at the following addresses: katandin.cshl.org:9331/RNAi/docs/BseRI-BamHI_Strategy.pdf and katandin.cshl.org:9331/RNAi/docs/Web_version_of_PCR_strategyl.pdf).

Expression constructs of the present disclosure include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems, such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. (Tuschl, T., 2002, Supra).

Synthetic siRNAs can be delivered into cells by methods known in the art, including cationic liposome transfection and electroporation. To obtain longer term suppression of the target genes (e.g., DGAT2 genes, FASN genes, OPN genes, and NOX4 genes) and to facilitate delivery under certain circumstances, one or more siRNAs can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, T., 2002, supra) capable of expressing functional double-stranded siRNAs; (Bagella et al., 1998; Lee et al., 2002, supra; Miyagishi et al., 2002, supra; Paul et al., 2002, supra; Yu et al., 2002, supra; Sui et al., 2002, supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998; Lee et al., 2002, supra; Miyagishi et al., 2002, supra; Paul et al., 2002, supra; Yu et al., 2002), supra; Sui et al., 2002, supra). Constructs containing siRNA sequence under the control of T7 promoter also make functional siRNAs when co-transfected into the cells with a vector expressing T7 RNA polymerase (Jacque et al., 2002, supra). A single construct may contain multiple sequences coding for siRNAs, such as multiple regions of the gene encoding DGAT2, FASN, OPN, and NOX4 targeting the same gene or multiple genes, and can be driven, for example, by separate PolIII promoter sites.

Animal cells express a range of noncoding RNAs of approximately 22 nucleotides termed micro-RNA (miRNAs), which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with sequence complementary to the target mRNA, a vector construct that expresses the engineered precursor can be used to produce siRNAs to initiate RNAi against specific mRNA targets in mammalian cells (Zeng et al., 2002, supra). When expressed by DNA vectors containing polymerase III promoters, micro-RNA designed hairpins can silence gene expression (McManus et al., 2002, supra). MicroRNAs targeting polymorphisms may also be useful for blocking translation of mutant proteins in the absence of siRNA-mediated gene-silencing. Such applications may be useful in situations, for example, where a designed siRNA caused off-target silencing of wild type protein.

Viral-mediated delivery mechanisms can also be used to induce specific silencing of targeted genes through expression of siRNA, for example, by generating recombinant adenoviruses harboring siRNA under RNA Pol II promoter transcription control (Xia et al., 2002, supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., 2002). In adult mice, efficient delivery of siRNA can be accomplished by “high-pressure” delivery technique, a rapid injection (within 5 seconds) of a large volume of siRNA containing solution into animal via the tail vein (Liu et al., 1999, supra; McCaffrey et al., 2002, supra; Lewis et al., 2002.) Nanoparticles and liposomes can also be used to deliver siRNA into animals. In certain exemplary embodiments, recombinant adeno-associated viruses (rAAVs) and their associated vectors can be used to deliver one or more siRNAs into cells, e.g., neural cells (e.g., brain cells) (US Patent Applications 2014/0296486, 2010/0186103, 2008/0269149, 2006/0078542 and 2005/0220766).

The nucleic acid compositions of the disclosure include both unmodified siRNAs and modified siRNAs, such as crosslinked siRNA derivatives or derivatives having non-nucleotide moieties linked, for example, to their 3′ or 5′ ends. Modifying siRNA derivatives in this way may improve cellular uptake or enhance cellular targeting activities of the resulting siRNA derivative, as compared to the corresponding siRNA, and are useful for tracing the siRNA derivative in the cell, or improving the stability of the siRNA derivative compared to the corresponding siRNA.

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA will be targeted by the siRNA generated from the engineered RNA precursor and will be depleted from the cell or organism leading to a decrease in the concentration of the protein encoded by that mRNA in the cell or organism. The RNA precursors are typically nucleic acid molecules that individually encode either one strand of a dsRNA or encode the entire nucleotide sequence of an RNA hairpin loop structure.

The nucleic acid compositions of the disclosure can be unconjugated or can be conjugated to another moiety, such as a nanoparticle, to enhance a property of the compositions, e.g., a pharmacokinetic parameter such as absorption, efficacy, bioavailability and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present disclosure can also be labeled using any method known in the art. For instance, the nucleic acid compositions can be labeled with a fluorophore, e.g., Cy3, fluorescein, or rhodamine. The labeling can be carried out using a kit, e.g., the SILENCERTM siRNA labeling kit (Ambion). Additionally, the siRNA can be radiolabeled, e.g., using 3H, 32P or another appropriate isotope.

Moreover, because RNAi is believed to progress via at least one single-stranded RNA intermediate, the skilled artisan will appreciate that ss-siRNAs (e.g., the antisense strand of a ds-siRNA) can also be designed (e.g., for chemical synthesis), generated (e.g., enzymatically generated), or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, such as about 200-500, for example, about 250, 300, 350, 400, or 450 nucleotides in length) acting as effectors of RNAi. (Brondani et al., Proc Natl Acad Sci USA. 2001 Dec. 4; 98(25):14428-33. Epub 2001 Nov. 27.)

### IV. Anti-RNA Silencing Agents

In certain embodiment, the present disclosure provides anti-RNA silencing agents (e.g., anti-DGAT2 RNA silencing agents, anti-FASN RNA silencing agents, anti-OPN RNA silencing agents, and anti-NOX4 RNA silencing agents) such as siRNA and antisense oligonucleotides, methods of making said RNA silencing agents, and methods (e.g., research and/or therapeutic methods) for using said improved RNA silencing agents (or portions thereof) for RNA silencing of proteins (e.g., DGAT2 proteins, FASN proteins, OPN proteins, and NOX4 proteins). The RNA silencing agents comprise an antisense strand (or portions thereof), wherein the antisense strand has sufficient complementary to a target mRNA (e.g., DGAT2 mRNA, FASN mRNA, OPN mRNA, and NOX4 mRNA) to mediate an RNA-mediated silencing mechanism (e.g., RNAi).

In certain embodiments, siRNA compounds are provided having one or any combination of the following properties: (1) fully chemically-stabilized (i.e., no unmodified 2′-OH residues); (2) asymmetry; (3) 11-21 base pair duplexes; (4) at least 50% 2′-methoxy modifications, such as 70%-100% 2′-methoxy modifications, although an alternating pattern of chemically-modified nucleotides (e.g., 2′-fluoro and 2′-methoxy modifications), are also contemplated; and (5) single-stranded, fully phosphorothioated tails of 2-8 bases. In certain embodiments, the number of phosphorothioate modifications is varied from 4 to 16 total. In certain embodiments, the number of phosphorothioate modifications is varied from 8 to 13 total.

In certain embodiments, the siRNA compounds described herein can be conjugated to a variety of targeting agents, including, but not limited to, cholesterol, docosahexaenoic acid (DHA), phenyltropanes, cortisol, vitamin A, vitamin D, N-acetylgalactosamine (GalNac), and gangliosides.

Certain compounds of the disclosure having the structural properties described above, herein may be referred to as “hsiRNA-ASP” (hydrophobically-modified, small interfering RNA, featuring an advanced stabilization pattern). In addition, this hsiRNA-ASP pattern showed a dramatically improved distribution through several tissues, including, but not limited to, the liver, placenta, kidney, and spleen, making them accessible for therapeutic intervention.

The compounds of the disclosure can be described in the following aspects and embodiments.

In a first aspect, provided herein is a double stranded RNA (dsRNA) comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 80% 2′-O-methyl modifications;
(3) the nucleotides at positions 2, 4, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 80% 2′-O-methyl modifications; and
(7) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, 16, and 20 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7 and 9-11 from the 3′ end of the sense strand are not 2′-methoxy-ribonucleotides; and
(8) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein:
(1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7, 10, and 11 from the 3′ end of the sense strand are not 2′-methoxy-ribonucleotides; and
(8) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein: (1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10; (2) the antisense strand comprises at least 50% 2′-O-methyl modifications; (3) the nucleotides at positions 2, 4, 5, 6, 8, 10, 12, 14, and 16 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises at least 65% 2′-O-methyl modifications; (7) the nucleotides at positions 3, 7, 9, 11, and 13 from the 3′ end of the sense strand are not 2′-methoxy-ribonucleotides; and (8) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein: (1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10; (2) the antisense strand comprises at least 85% 2′-O-methyl modifications; (3) the nucleotides at positions 2, 14, and 20 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises 100% 2′-O-methyl modifications; and (7) the nucleotides at positions 1-2 from the 5′ end of the sense strand are connected to each other via phosphorothioate internucleotide linkages.

In another aspect, provided herein is a dsRNA comprising an antisense strand and a sense strand, each strand comprising separately a 5′ end and a 3′ end, wherein: (1) the antisense strand comprises a sequence substantially complementary to a DGAT2 nucleic acid sequence of any one of SEQ ID NOs: 1-10, a FASN nucleic acid sequence of any one of Table 5, a OPN nucleic acid sequence of any one of Table 8, or a NOX4 nucleic acid sequence of any one of Table 10; (2) the antisense strand comprises at least 75% 2′-O-methyl modifications; (3) the nucleotides at positions 2, 6, 14, 16, and 20 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises at least 75% 2′-O-methyl modifications; (7) the nucleotides at positions 7, 9, 10, and 11 from the 3′ end of the sense strand are not 2′-methoxy-ribonucleotides; and (8) the nucleotides at positions 1-2 from the 5′ end of the sense strand are connected to each other via phosphorothioate internucleotide linkages.

In any of the above aspects of the disclosure, the antisense strand may comprise a length of 20 or 21 nucleotides.

In any of the above aspects of the disclosure, the sense strand may comprise a length of 16, 18, 19, 20, or 21 nucleotides.

In certain embodiments of the dsRNA, the nucleotide at position 20 from the 5′ end of the antisense strand is not a 2′-methoxy-ribonucleotide.

a) Design of Anti-DGAT2, Anti-FASN, Anti-OPN, and Anti-NOX4 siRNA Molecules

An siRNA molecule of the application is a duplex made of a sense strand and complementary antisense strand, the antisense strand having sufficient complementary to a DGAT2 mRNA, FASN mRNA, OPN mRNA, or NOX4 mRNA to mediate RNAi. In certain embodiments, the siRNA molecule has a length from about 10-50 or more nucleotides, i.e., each strand comprises 10-50 nucleotides (or nucleotide analogs). In other embodiments, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In certain embodiments, the strands are aligned such that there are at least 1, 2, 3, 4, 5, 6, 7, 8, 9, or 10 bases at the end of the strands, which do not align (i.e., for which no complementary bases occur in the opposing strand), such that an overhang of 1, 2, 3, 4, 5, 6, 7, 8, 9, or 10 residues occurs at one or both ends of the duplex when strands are annealed.

Usually, siRNAs can be designed by using any method known in the art, for instance, by using the following protocol:

1. The siRNA should be specific for a target sequence, e.g., a target sequence set forth in the Examples. The first strand should be complementary to the target sequence, and the other strand is substantially complementary to the first strand. (See Examples for exemplary sense and antisense strands.) Exemplary target sequences are selected from any region of the target gene that leads to potent gene silencing. Regions of the target gene include, but are not limited to, the 5′ untranslated region (5′-UTR) of a target gene, the 3′ untranslated region (3′-UTR) of a target gene, an exon of a target gene, or an intron of a target gene. Cleavage of mRNA at these sites should eliminate translation of corresponding DGAT2, FASN, OPN, and NOX4 proteins. Target sequences from other regions of the DGAT2, FASN, OPN, NOX4, and MALAT1 genes are also suitable for targeting. A sense strand is designed based on the target sequence.

2. The sense strand of the siRNA is designed based on the sequence of the selected target site. In certain embodiments, the sense strand includes about 15 to about 25 nucleotides, e.g., 15, 16, 17, 18, 19, 20, 21, 22, 23, 24, or 25 nucleotides. In certain embodiments, the sense strand includes 15, 16, 17, 18, 19, or 20 nucleotides. In certain embodiments, the sense strand is 15 nucleotides in length. In certain embodiments, the sense strand is 18 nucleotides in length. In certain embodiments, the sense strand is 20 nucleotides in length. The skilled artisan will appreciate, however, that siRNAs having a length of less than 15 nucleotides or greater than 25 nucleotides can also function to mediate RNAi. Accordingly, siRNAs of such length are also within the scope of the instant disclosure, provided that they retain the ability to mediate RNAi. Longer RNA silencing agents have been demonstrated to elicit an interferon or Protein Kinase R (PKR) response in certain mammalian cells which may be undesirable. In certain embodiments, the RNA silencing agents of the disclosure do not elicit a PKR response (i.e., are of a sufficiently short length). However, longer RNA silencing agents may be useful, for example, in cell types incapable of generating a PKR response or in situations where the PKR response has been down-regulated or dampened by alternative means.

The siRNA molecules of the disclosure have sufficient complementarity with the target sequence such that the siRNA can mediate RNAi. In general, siRNA containing nucleotide sequences sufficiently complementary to a target sequence portion of the target gene to effect RISC-mediated cleavage of the target gene are contemplated. Accordingly, in a certain embodiment, the antisense strand of the siRNA is designed to have a sequence sufficiently complementary to a portion of the target. For example, the antisense strand may have 100% complementarity to the target site. However, 100% complementarity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99%, or even 100% complementarity, between the antisense strand and the target RNA sequence is contemplated. The present application has the advantage of being able to tolerate certain sequence variations to enhance efficiency and specificity of RNAi. In one embodiment, the antisense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and the percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 7 (e.g., 2, 3, 4, 5, 6, or 7), or 1 to 4, e.g., 2, 3, or 4 nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet your criteria for evaluation.

Further general information about the design and use of siRNA may be found in “The siRNA User Guide,” available at The Max-Plank-Institut fur Biophysikalische Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(#of A+T bases)+4(#of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant DGAT2 mRNA, FASN mRNA, OPN mRNA, and NOX4 mRNA), the siRNA may be incubated with target cDNA (e.g., DGAT2 cDNA, FASN cDNA, OPN cDNA, and NOX4 cDNA) in a Drosophila-based in vitro mRNA expression system. Radiolabeled with 32P, newly synthesized target mRNAs (e.g., DGAT2 mRNA, FASN mRNA, OPN mRNA, and NOX4 mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

Anti-DGAT2 mRNA siRNAs, anti-FASN mRNA siRNAs, anti-OPN mRNA siRNAs, and anti-NOX4 mRNA siRNAs may be designed to target any of the target sequences described supra. Said siRNAs comprise an antisense strand, which is sufficiently complementary with the target sequence to mediate silencing of the target sequence. In certain embodiments, the RNA silencing agent is a siRNA.

In certain embodiments, the siRNA comprises a sense and an antisense strand comprising a sequence set forth in Tables 2-4.

Sites of siRNA-mRNA complementation are selected, which result in optimal mRNA specificity and maximal mRNA cleavage.

b) siRNA-Like Molecules

siRNA-like molecules of the disclosure have a sequence (i.e., have a strand having a sequence) that is “sufficiently complementary” to a target sequence of an DGAT2 mRNA, FASN mRNA, OPN mRNA, and NOX4 mRNA to direct gene silencing either by RNAi or translational repression. siRNA-like molecules are designed in the same way as siRNA molecules, but the degree of sequence identity between the sense strand and target RNA approximates that observed between a miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further embodiment, the “bulge” is centered at nucleotide positions 12 and 13 from the 5′ end of the miRNA molecule.

c) Short Hairpin RNA (shRNA) Molecules

In certain featured embodiments, the instant disclosure provides shRNAs capable of mediating RNA silencing of a target sequence with enhanced selectivity (e.g., a DGAT2 target sequence, an FASN target sequence, an OPN target sequence, and a NOX4 target sequence). In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

miRNAs are noncoding RNAs of approximately 22 nucleotides, which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the present application are artificial constructs based on these naturally occurring pre-miRNAs, which are engineered to deliver desired RNA silencing agents (e.g., siRNAs of the disclosure). By substituting the stem sequences of the pre-miRNA with sequence complementary to the target mRNA, a shRNA is formed. The shRNA is processed by the entire gene silencing pathway of the cell, thereby efficiently mediating RNAi.

The requisite elements of a shRNA molecule include a first portion and a second portion having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In shRNAs (or engineered precursor RNAs) of the instant disclosure, one portion of the duplex stem is a nucleic acid sequence that is complementary (or anti-sense) to the target sequence (e.g., the DGAT2 target sequence, the FASN target sequence, the OPN target sequence, and the NOX4 target sequence). In certain embodiments, one strand of the stem portion of the shRNA is sufficiently complementary (e.g., antisense) to a target RNA (e.g., mRNA) sequence to mediate degradation or cleavage of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are about 15 to about 50 nucleotides in length. In certain embodiments, the two stem portions are about 18 or 19 to about 21, 22, 23, 24, 25, 30, 35, 37, 38, 39, or 40 or more nucleotides in length. In certain embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to and including the entire mRNA). In fact, a stem portion can include much larger sections complementary to the target mRNA (up to and including the entire mRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop in the shRNAs or engineered RNA precursors can be 2, 3, 4, 5, 6, 7, 8, 9, or more, e.g., 15 or 20, or more nucleotides in length.

The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop portion in the shRNA can be about 2 to about 20 nucleotides in length, i.e., about 2, 3, 4, 5, 6, 7, 8, 9, or more, e.g., 15 or 20, or more nucleotides in length. In certain embodiments, a loop consists of or comprises a “tetraloop” sequence. Exemplary tetraloop sequences include, but are not limited to, the sequences GNRA, where N is any nucleotide and R is a purine nucleotide, GGGG, and UUUU.

In certain embodiments, shRNAs of the present application include the sequences of a desired siRNA molecule described supra. In other embodiments, the sequence of the antisense portion of a shRNA can be designed essentially as described above or generally by selecting an 18, 19, 20, 21 nucleotide, or longer, sequence from within the target RNA (e.g., DGAT2 mRNA, FASN mRNA, OPN mRNA, and NOX4 mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include, in the duplex stem, the 21-22 or so nucleotide sequences of the siRNA or siRNA-like duplex desired to be produced in vivo. Thus, the stem portion of the engineered RNA precursor includes at least 18 or 19 nucleotide pairs corresponding to the sequence of an exonic portion of the gene whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding mRNA for translational repression or destruction by RNAi in vivo and in vitro.

In certain embodiments, shRNAs of the disclosure include miRNA sequences, optionally end-modified miRNA sequences to enhance entry into RISC. The miRNA sequence can be similar or identical to that of any naturally occurring miRNA (see e.g., The miRNA Registry; Griffiths-Jones S, Nuc. Acids Res., 2004). Over one thousand natural miRNAs have been identified to date and together they are thought to comprise about 1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g., MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, Mus musculus, and Rattus norvegicus as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al., Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex, but only one strand is taken up by the RISC complex to direct gene silencing. Certain miRNAs, e.g., plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between a miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In certain embodiments, the miRNA sequence is that of a naturally-occurring miRNA sequence, the aberrant expression or activity of which is correlated with a miRNA disorder.

### d) Dual Functional Oligonucleotide Tethers

In other embodiments, the RNA silencing agents of the present disclosure include dual functional oligonucleotide tethers useful for the intercellular recruitment of a miRNA. Animal cells express a range of miRNAs, noncoding RNAs of approximately 22 nucleotides, which can regulate gene expression at the post transcriptional or translational level. By binding a miRNA bound to RISC and recruiting it to a target mRNA, a dual functional oligonucleotide tether can repress the expression of genes involved e.g., in the arteriosclerotic process. The use of oligonucleotide tethers offers several advantages over existing techniques to repress the expression of a particular gene. First, the methods described herein allow an endogenous molecule (often present in abundance), a miRNA, to mediate RNA silencing. Accordingly, the methods described herein obviate the need to introduce foreign molecules (e.g., siRNAs) to mediate RNA silencing. Second, the RNA-silencing agents and the linking moiety (e.g., oligonucleotides such as the 2′-O-methyl oligonucleotide), can be made stable and resistant to nuclease activity. As a result, the tethers of the present disclosure can be designed for direct delivery, obviating the need for indirect delivery (e.g., viral) of a precursor molecule or plasmid designed to make the desired agent within the cell. Third, tethers and their respective moieties, can be designed to conform to specific mRNA sites and specific miRNAs. The designs can be cell and gene product specific. Fourth, the methods disclosed herein leave the mRNA intact, allowing one skilled in the art to block protein synthesis in short pulses using the cell's own machinery. As a result, these methods of RNA silencing are highly regulatable.

The dual functional oligonucleotide tethers (“tethers”) of the disclosure are designed such that they recruit miRNAs (e.g., endogenous cellular miRNAs) to a target mRNA so as to induce the modulation of a gene of interest. In certain embodiments, the tethers have the formula T-L-μ, wherein T is an mRNA targeting moiety, L is a linking moiety, and D is a miRNA recruiting moiety. Any one or more moieties may be double stranded. In certain embodiments, each moiety is single stranded.

Moieties within the tethers can be arranged or linked (in the 5′ to 3′ direction) as depicted in the formula T-L-μ (i.e., the 3′ end of the targeting moiety linked to the 5′ end of the linking moiety and the 3′ end of the linking moiety linked to the 5′ end of the miRNA recruiting moiety). Alternatively, the moieties can be arranged or linked in the tether as follows: μ-T-L (i.e., the 3′ end of the miRNA recruiting moiety linked to the 5′ end of the linking moiety and the 3′ end of the linking moiety linked to the 5′ end of the targeting moiety).

The mRNA targeting moiety, T, as described above, is capable of capturing a specific target mRNA. According to the disclosure, expression of the target mRNA is undesirable, and, thus, translational repression of the mRNA is desired. The mRNA targeting moiety should be of sufficient size to effectively bind the target mRNA. The length of the targeting moiety will vary greatly, depending, in part, on the length of the target mRNA and the degree of complementarity between the target mRNA and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a certain embodiment, the targeting moiety is about 15 to about 25 nucleotides in length.

The miRNA recruiting moiety, Q, as described above, is capable of associating with a miRNA. According to the present application, the miRNA may be any miRNA capable of repressing the target mRNA. Mammals are reported to have over 250 endogenous miRNAs (Lagos-Quintana et al. (2002) Current Biol. 12:735-739; Lagos-Quintana et al. (2001) Science 294:858-862; and Lim et al. (2003) Science 299:1540). In various embodiments, the miRNA may be any art-recognized miRNA.

The linking moiety, L, is any agent capable of linking the targeting moieties such that the activity of the targeting moieties is maintained. Linking moieties can be oligonucleotide moieties comprising a sufficient number of nucleotides, such that the targeting agents can sufficiently interact with their respective targets. Linking moieties have little or no sequence homology with cellular mRNA or miRNA sequences. Exemplary linking moieties include one or more 2′-O-methylnucleotides, e.g., 2′-O-methyladenosine, 2′-O-methylthymidine, 2′-O-methylguanosine or 2′-O-methyluridine.

### e) Gene Silencing Oligonucleotides

In certain exemplary embodiments, gene expression (i.e., a DGAT2 gene expression, a FASN gene expression, an OPN gene expression, and a NOX4 gene expression) can be modulated using oligonucleotide-based compounds comprising two or more single stranded antisense oligonucleotides that are linked through their 5′-ends that allow the presence of two or more accessible 3′-ends to effectively inhibit or decrease gene expression (i.e., a DGAT2 gene expression, a FASN gene expression, a OPN gene expression, and a NOX4 gene expression). Such linked oligonucleotides are also known as Gene Silencing Oligonucleotides (GSOs). (See, e.g., U.S. Pat. No. 8,431,544 assigned to Idera Pharmaceuticals, Inc., incorporated herein by reference in its entirety for all purposes.)

The linkage at the 5′ ends of the GSOs is independent of the other oligonucleotide linkages and may be directly via 5′, 3′ or 2′hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

GSOs can comprise two identical or different sequences conjugated at their 5′-5′ ends via a phosphodiester, phosphorothioate or non-nucleoside linker. Such compounds may comprise 15 to 27 nucleotides that are complementary to specific portions of mRNA targets of interest for antisense down regulation of a gene product. GSOs that comprise identical sequences can bind to a specific mRNA via Watson-Crick hydrogen bonding interactions and inhibit protein expression. GSOs that comprise different sequences are able to bind to two or more different regions of one or more mRNA target and inhibit protein expression. Such compounds are comprised of heteronucleotide sequences complementary to target mRNA and form stable duplex structures through Watson-Crick hydrogen bonding. Under certain conditions, GSOs containing two free 3′-ends (5′-5′-attached antisense) can be more potent inhibitors of gene expression than those containing a single free 3′-end or no free 3′-end.

In some embodiments, the non-nucleotide linker is glycerol or a glycerol homolog of the formula HO—(CH2)o-CH(OH)—(CH2)p-OH, wherein o and p independently are integers from 1 to about 6, from 1 to about 4 or from 1 to about 3. In some other embodiments, the non-nucleotide linker is a derivative of 1,3-diamino-2-hydroxypropane. Some such derivatives have the formula HO—(CH2)m-C(O)NH—CH2-CH(OH)—CH2-NHC(O)—(CH2)m-OH, wherein m is an integer from 0 to about 10, from 0 to about 6, from 2 to about 6 or from 2 to about 4.

Some non-nucleotide linkers permit attachment of more than two GSO components. For example, the non-nucleotide linker glycerol has three hydroxyl groups to which GSO components may be covalently attached. Some oligonucleotide-based compounds of the disclosure, therefore, comprise two or more oligonucleotides linked to a nucleotide or a non-nucleotide linker. Such oligonucleotides according to the disclosure are referred to as being “branched.”

In certain embodiments, GSOs are at least 14 nucleotides in length. In certain exemplary embodiments, GSOs are 15 to 40 nucleotides long or 20 to 30 nucleotides in length. Thus, the component oligonucleotides of GSOs can independently be 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

These oligonucleotides can be prepared by the art recognized methods, such as phosphoramidate or H-phosphonate chemistry, which can be carried out manually or by an automated synthesizer. These oligonucleotides may also be modified in a number of ways without compromising their ability to hybridize to mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate hydroxyl, acetamidate, carboxymethyl ester, or a combination of these and other internucleotide linkages between the 5′ end of one nucleotide and the 3′ end of another nucleotide, in which the 5′ nucleotide phosphodiester linkage has been replaced with any number of chemical groups.

### f) Antisense Oligonucleotide (ASO)

In certain embodiments, the oligonucleotide with a sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence, is an antisense oligonucleotide (ASO).

In certain embodiments, the ASO comprises a sequence substantially complementary to a FASN nucleic acid sequence of any one of Table 5.

In certain embodiments, the ASO comprises a sequence substantially complementary to a OPN nucleic acid sequence of any one of Table 8.

In certain embodiments, the ASO comprises a sequence substantially complementary to a NOX4 nucleic acid sequence of any one of Table 10.

As used herein, the term “antisense oligonucleotide” or “antisense compound” refers to an oligonucleotide molecule which is capable of binding to RNA inside cells by Watson-Crick base pairing. Depending on the sequence and chemistry of the antisense oligonucleotide, this interaction can lead to silencing of a target gene (i.e. reducing the level of expression of mature mRNA and/or protein from that gene) or activation of a target gene (i.e. increasing the level of expression of mature mRNA and/or protein from that gene). The antisense oligonucleotides of the present disclosure are focused on activating gene expression, which can be done utilizing different mechanisms. Some antisense oligonucleotides are designed to recruit RNase H to cleave their target RNAs. RNase H is a family of non-sequence-specific endonuclease enzymes that catalyze the cleavage of RNA in an RNA/DNA substrate via a hydrolytic mechanism. In certain embodiments, the antisense oligonucleotides of the disclosure trigger RNase H-mediated cleavage of a pre-mRNA target, which can be compatible with activation of overall target gene expression. Other antisense oligonucleotides, called steric blockers, are designed not to elicit cleavage of their targets but to block interactions with cellular factors. For example, these cellular factors could modulate splicing, block interactions of noncoding RNAs or of RNA-binding proteins, stabilize mRNA to prolong its half-life, or increase the efficiency of translation of an mRNA.

As used herein, the term “heteroduplex oligonucleotide” or “HDO” refers to an antisense oligonucleotide-based compound that comprises an antisense oligonucleotide as described herein and a complementary oligonucleotide, annealed to said antisense oligonucleotide, thereby producing a duplex (the HDO). The HDO complementary oligonucleotide may comprise any of the chemical modifications employed in the ASO. HDOs are described in further detail in Nishina et al. (Nature Communications volume 6, Article number: 7969. 2015), WO2014192310A1, and WO2014203518A1 each of which is incorporated herein by reference.

Antisense oligonucleotides designed to recruit RNase H are often designed as “gapmers.” The term “gapmer” means a chimeric antisense oligonucleotide in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.” “Chimeric antisense oligonucleotide” means an antisense oligonucleotide that has at least two chemically distinct regions.

The term “antisense activity” means any detectable or measurable activity attributable to the hybridization of an antisense compound to its target nucleic acid. In some embodiments, antisense activity is an increase in the amount or expression of a target nucleic acid or protein encoded by such target nucleic acid.

The term “antisense inhibition” means reduction of target nucleic acid levels in the presence of an antisense oligonucleotide having a sequence that is sufficiently complementary to a target nucleic acid compared to target nucleic acid levels in the absence of the antisense compound. A target nucleic acid can be any nucleic acid.

The term “target-recognition sequence” refers to the portion of an antisense compound that recognizes a target nucleic acid. The target-recognition sequence has a nucleobase sequence that permits hybridization to a corresponding region or segment of a target nucleic acid.

The term “conserved region” refers to a portion, or portions, of a nucleic acid sequence that is conserved, i.e. a portion, or portions of the nucleic acid sequence having a similar or identical sequence across species. A conserved region can be computationally identified, e.g., using any sequence alignment software available in the art.

As used herein, a “region of complementarity” refers to a portion of the antisense oligonucleotide that is complementary to the target. For example, but in no way limiting, an 18-nucleotide long antisense oligonucleotide can comprise a contiguous 12-nucleotide portion that is complementary to the target transcript. In certain embodiments, the antisense oligonucleotide is complementary to the target transcript over the full length of the antisense oligonucleotide.

In some embodiments, an antisense compound of the present disclosure is an antisense oligonucleotide. Chimeric antisense oligonucleotides typically contain at least one region modified so as to confer increased resistance to nuclease degradation, increased cellular uptake, increased binding affinity for the target nucleic acid, and/or increased activity. A second region of a chimeric antisense compound can optionally serve as a substrate for the cellular endonuclease RNase H, which cleaves the RNA strand of an RNA:DNA duplex. In some embodiments, an antisense compound of the present disclosure is a chimeric antisense oligonucleotide having a gapmer motif. In a gapmer, an internal region having a plurality of nucleotides that supports RNase H cleavage is positioned between external regions having a plurality of nucleotides that are chemically distinct from the nucleosides of the internal region.

In some embodiments, the present disclosure provides an antisense oligonucleotide having a target-recognition sequence that is sufficiently complementary to a target transcript or portion thereof, to direct cleavage of the target transcript by RNase H. The target-recognition sequence of the antisense oligonucleotide can be the full length of the antisense oligonucleotide, or a portion thereof. In some embodiments, the antisense oligonucleotide comprises a gapmer motif.

In the case of an antisense compound having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃ (i.e., OMe), among others), and bicyclic sugar modified nucleosides (such bicyclic sugar modified nucleosides can include those having a 4′-(CH₂)ₙ—O-2′ bridge, where n=1 or n=2). In some embodiments, the wing segments of the gapmer contain one or more tricyclo-DNA (tcDNA) modifications. In some embodiments, each distinct region comprises uniform sugar moieties. In some embodiments, each wing segment comprises a mixture of different nucleotide modifications. For example, in one embodiment, a LNA modification and a 2′-MOE modification could be used in combination for one antisense compound. In one embodiment, a LNA modification and a 2′-O-Methyl modification could be used in combination for one antisense compound. In one embodiment, a LNA modification and a 2′-deoxy modification could be used in combination for one antisense compound. In one embodiment, a LNA modification and a tricyclo-DNA modification could be used in combination for one antisense compound. In one embodiment, a 2′-MOE modification and a tricyclo-DNA modification could be used in combination for one antisense compound.

The gapmer motif can be described using the formula “A-B—C”, where “A” represents the length of the 5′ wing region, “B” represents the length of the gap region, and “C” represents the length of the 3′ wing region. As such, in some embodiments, an antisense oligonucleotide of the present disclosure has the formula:

A-B—C.

As used herein, a gapmer described as “A-B—C” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment.

In some embodiments, the 5′ wing region represented by “A” comprises from about 0 to about 8 modified nucleotides, e.g., from about 1 to about 6 modified nucleotides. For example, the 5′ wing region represented by “A” can be 0, 1, 2, 3, 4, 5, 6, 7, or 8 nucleotides in length. In some embodiments, the 3′ wing region represented by “C” comprises about 0 to about 8 modified nucleotides, e.g., from about 1 to about 6 modified nucleotides. For example, the 3′ wing region represented by “C” can be 0, 1, 2, 3, 4, 5, 6, 7, or 8 nucleotides in length. In some embodiments, “A” and “C” are the same, in some embodiments, they are different.

In some embodiments, the gap region represented by “B” comprises from about 6 to about 18 DNA nucleotides and/or DNA-like nucleotides, e.g., from about 6 to about 12 DNA nucleotides and/or DNA-like nucleotides. For example, the gap region represented by “B” can be 6, 7, 8, 9, 10, 11, 12, 13, 14, 15, 16, 17, or 18 DNA nucleotides and/or DNA-like nucleotides in length. Thus, an antisense oligonucleotide of the present disclosure having a target-recognition sequence with the formula “A-B—C” include, but are not limited to the following gapmer formats, for example 1-10-1 (i.e., one nucleotide—ten nucleotides—one nucleotide), 1-10-1, 1-11-1, 1-12-1, 2-8-2, 2-9-2, 2-10-2, 2-11-2, 2-12-2, 3-6-3, 3-7-3, 3-8-3, 3-9-3, 3-10-3, 3-11-3, 3-12-3, 4-6-4, 4-7-4, 4-8-4, 4-9-4, 4-10-4, 4-11-4, 4-12-4, 5-6- 5, 5-7-5, 5-8-5, 5-9-5, 5-10-5, 5-11-5, 5-12-5, 6-6-6, 6-7-6, 6-8-6, 6-9-6, 6-10-6, 6-11-6, or 6- 12-6. The wings can also be of different lengths, such as 1-10-6, 3-9-5, 7-9-2, 4-10-5, or other asymmetric combinations of wing lengths flanking a central DNA gap. In certain embodiments, the gapmer of “A-B—C” is at least 12 nucleotides in length. In certain embodiments, “B” is at least 6 nucleotides in length. A person of skill in the art will be able to identify additional asymmetric combinations of wing lengths.

In certain embodiments, antisense compounds targeted to a target nucleic acid possess a 5-9-4 gapmer format. In some embodiments, the antisense compound is an antisense oligonucleotide having a target-recognition sequence with the 5-9-4 format that is sufficiently complementary to a target transcript, or a portion thereof, to direct cleavage of the target transcript by RNase H. In some embodiments, the target-recognition sequence has the formula “A-B—C”, wherein “A” comprises about 2 to 6 modified nucleotides, “B” comprises about 6 to 12 DNA nucleotides and/or DNA-like nucleotides, and “C” comprises about 2 to 6 modified nucleotides. In some embodiments, the target-recognition sequence has the formula “A-B—C”, wherein “A” comprises 5 modified nucleotides, “B” comprises 9 DNA nucleotides and/or DNA-like nucleotides, and “C” comprises 4 modified nucleotides. In some embodiments, the target-recognition sequence has the formula “A-B—C”, wherein “A” comprises 2 to 6 2′-O-(2-methoxyethyl) (MOE) modified nucleotides, “B” comprises 6 to 12 DNA nucleotides and/or DNA-like nucleotides, and “C” comprises 2 to 6 2′-O-(2-methoxyethyl) (MOE) modified nucleotides. In some embodiments, the target-recognition sequence has the formula “A-B—C”, wherein “A” comprises 5 2′-O-(2-methoxyethyl) (MOE) modified nucleotides, “B” comprises 9 DNA nucleotides and/or DNA-like nucleotides, and “C” comprises 4 2′-O-(2-methoxyethyl) (MOE) modified nucleotides.

In some embodiments, antisense compounds that target a target nucleic acid possess a “wingmer” motif. The wingmer motif can be described using the formula “X—Y” or “Y—X”, where “X” represents the length of the wing region, and “Y” represents the length of the gap region. As such, in some embodiments, an antisense oligonucleotide of the present disclosure has the formula:

X—Y, or

Y—X.

As used herein, a wingmer described as “X—Y” or “Y—X” has a configuration such that the gap segment is positioned immediately adjacent to the wing segment. Thus, no intervening nucleotides exist between the wing segment and the gap segment. Non-limiting examples of wingmer configurations of an antisense compound of the present disclosure include, e.g., 1-15, 1-17, 1-19, 2-15, 2-17, 2-19, 2-22, 3-13, 3-17, 3-20, 3-21, 3-22, 4-12, 4-14, 4-16, 4-18, 4-19, 4-21, 5-11, 5-13, 5-14, 5-15, 5-16, 5-18, or 5-20.

In some embodiments, antisense compounds targeted to a target nucleic acid possess a gap-widened motif. As used herein, “gap-widened” refers to an antisense compound having a gap segment of 12 or more contiguous DNA nucleotides and/or DNA-like nucleotides adjacent to a wing region. In the case of a gap-widened gapmer, the gapmer comprises a gap region having 12 or more contiguous DNA nucleotides and/or DNA-like nucleotides positioned between and immediately adjacent to the 5′ and 3′ wing segments. In the case of a gap-widened wingmer, the wingmer comprises a gap region having 12 or more contiguous DNA nucleotides and/or DNA-like nucleotides positioned immediately adjacent to the wing segment.

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidates, and phosphorothioates. Methods of preparation of phosphorous-containing and non-phosphorous-containing linkages are well known.

In certain embodiments, antisense compounds targeted to a target nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

Antisense compounds of the disclosure can optionally contain one or more nucleosides wherein the sugar group has been modified. Such sugar-modified nucleosides can impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substituent groups (including 5′ and 2′ substituent groups, bridging of ring atoms to form bicyclic nucleic acids (BNA), replacement of the ribosyl ring oxygen atom with S, N(R), or C(R¹)(R²) (R, R¹, R²=H, C₁-C₁₂ alkyl or a protecting group) and combinations thereof. Examples of chemically modified sugars include 2′-F-5′-methyl substituted nucleoside (see WO 2008/101157 for other disclosed 5′,2′-bis substituted nucleosides) or replacement of the ribosyl ring oxygen atom with S with further substitution at the 2′-position (see U.S. Patent Application US20050130923) or alternatively 5′-substitution of a BNA (see WO 2007/134181, wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F (i.e., 2′-fluoro), 2′-OCH₃ (i.e., 2′-O-methyl) and 2′-O(CH₂)₂OCH₃ (i.e., 2′-O-methoxyethyl) substituent groups. The substituent at the 2′ position can also be selected from allyl, amino, azido, thio, 0-allyl, 0-C1-C10 alkyl, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. 2′-modified nucleotides are useful in the present disclosure, for example, 2′-O-methyl RNA, 2′-O-methoxyethyl RNA, 2′-fluoro RNA, and others envisioned by one of ordinary skill in the art.

Examples of bicyclic nucleic acids (BNAs) include without limitation nucleosides comprising a bridge between the 4′ and the 2′ ribosyl ring atoms. A BNA comprising a bridge between the 4′ and 2′ ribosyl ring atoms can be referred to as a locked nucleic acid (LNA), and is often referred to as inaccessible RNA. As used herein, the term “locked nucleotide” or “locked nucleic acid (LNA)” comprises nucleotides in which the 2′ deoxy ribose sugar moiety is modified by introduction of a structure containing a heteroatom bridging from the 2′ to the 4′ carbon atoms. The term “non-locked nucleotide” comprises nucleotides that do not contain a bridging structure in the ribose sugar moiety. Thus, the term comprises DNA and RNA nucleotide monomers (phosphorylated adenosine, guanosine, uridine, cytidine, deoxyadenosine, deoxyguanosine, deoxythymidine, deoxycytidine) and derivatives thereof as well as other nucleotides having a 2′-deoxy-erythro-pentofuranosyl sugar moiety or a ribo-pentofuranosyl moiety. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)—O-2′ (LNA); 4′-(CH₂)₂—O-2′ (ENA); 4′-C(CH₃)₂—O-2′ (see PCT/US2008/068922); 4′-CH(CH₃)—O-2′ and 4′-CH(CH₂OCH₃)—O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see published U.S. Patent Application US2004-0171570, published Sep. 2, 2004); 4′-CH₂—N(R)—O-2′ (see U.S. Pat. No. 7,427,672, issued on Sep. 23, 2008); 4′-CH₂—C(CH₃)-2′ and 4′-CH₂—C(═CH₂)-2′ (see PCT/US2008/066154); and wherein R is, independently, H, C₁-C₁₂ alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In some embodiments, antisense compounds provided herein include one or more 2′, 4′-constrained nucleotides. For example, antisense compounds provided by the present disclosure include those having one or more constrained ethyl (cEt) or constrained methoxyethyl (cMOE) nucleotides. In some embodiments, antisense compounds provided herein are antisense oligonucleotides comprising one or more constrained ethyl (cEt) nucleotides. The terms “constrained ethyl” and “ethyl-constrained” are used interchangeably.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

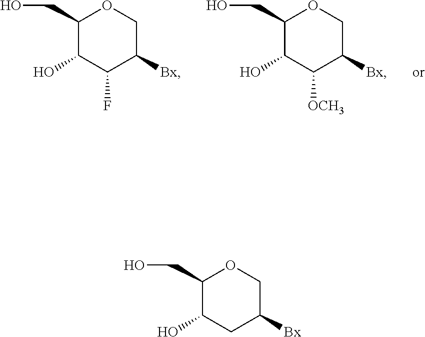

In certain embodiments, antisense oligonucleotides may comprise morpholino rings joined by phosphorodiamidate linkages. These may be referred to as PMO oligomers or phosphorodiamidate morpholino oligomers. In certain such embodiments, the backbone of these oligonucleotides may be uncharged. In other embodiments, one or more of the phosphorodiamidate linkages may comprise a charged moiety.

Many other bicyclo and tricyclo sugar surrogate ring systems are also known in the art that can be used to modify nucleosides for incorporation into antisense compounds (see for example review article: Leumann, J. C, Bioorganic & Medicinal Chemistry, 2002, 10, 841-854; Ito, K. R.; Obika, S., Recent Advances in Medicinal Chemistry of Antisense Oligonucleotides. In Comprehensive Medicinal Chemistry, 3rd edition, Elsevier: 2017). Such ring systems can undergo various additional substitutions to enhance activity.

Methods for the preparations of modified sugars are well known to those skilled in the art. In nucleotides having modified sugar moieties, the nucleobase moieties (natural, modified or a combination thereof) are maintained for hybridization with an appropriate nucleic acid target.

In certain embodiments, antisense compounds targeted to a target nucleic acid comprise one or more kinds of modified nucleotides. In one embodiment, antisense compounds targeted to a target nucleic acid comprise 2′-modified nucleotides. In one embodiment, antisense compounds targeted to a target nucleic acid comprise a 2′-O-methyl RNA, a 2′-O-methoxyethyl RNA, or a 2′-fluoro RNA. In one embodiment, antisense compounds targeted to a target nucleic acid comprise tricyclo-DNA (tcDNA). Tricyclo-DNA belongs to a class of constrained DNA analogs that display improved hybridizing capacities to complementary RNA, see, e.g., Ittig et al., Nucleic Acids Res. 32:346-353 (2004); Ittig et al., Prague, Academy of Sciences of the Czech Republic. 7:21-26 (Coll. Symp. Series, Hocec, M., 2005); Ivanova et al., Oligonucleotides 17:54-65 (2007); Renneberg et al., Nucleic Acids Res. 30:2751-2757 (2002); Renneberg et al., Chembiochem. 5:1114-1118 (2004); and Renneberg et al., JACS. 124:5993-6002 (2002). In one embodiment, antisense compounds targeted to a target nucleic acid comprise a locked nucleotide, an ethyl-constrained nucleotide, or an alpha-L-locked nucleic acid. Various alpha-L-locked nucleic acids are known by those of ordinary skill in the art, and are described in, e.g., Sorensen et al., J. Am. Chem. Soc. (2002) 124(10):2164-2176.

In certain embodiments, the antisense compounds targeting a target nucleic acid are fully chemically modified, i.e., every nucleotide is chemically modified. In certain embodiments, every nucleotide comprises a 2′-O-(2-methoxyethyl) (MOE) modification. In certain embodiments, every nucleotide comprises a tricyclo-DNA modification. In certain embodiments, the antisense compounds targeting a target nucleic acid comprise a mixture of tricyclo-DNA modifications and 2′-O-(2-methoxyethyl) (MOE) modifications, wherein every nucleotide of the antisense compounds is either tcDNA or MOE.

In certain embodiments, antisense compounds targeted to a target nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side.

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo such as 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a target nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side. In certain embodiments, antisense compounds targeted to a target nucleic acid comprise one or more modified nucleotides. In some embodiments, the modified nucleotide is 5-methylcytosine. In certain embodiments, each cytosine is a 5-methylcytosine. In some embodiments, the modified nucleotide is a 2′-O-(2-methoxyethyl) (MOE) modified nucleotide. In certain embodiments, the 2′-O— (MOE) modified nucleotides are arranged in a gapmer motif, e.g. a 5-9-4 gapmer format wherein 9 non-2′-O— (MOE) modified nucleotides are flanked by 4 or 5 2′-O— (MOE) modified nucleotides on one or both sides. In certain embodiments, antisense compounds targeted to a target nucleic acid comprise a steric blocking chemical modification format. In some embodiments of the steric blocking chemical modification format, every nucleotide of the antisense compound is a 2′-O-(2-methoxyethyl) (MOE) modified nucleotide. In some embodiments of the steric blocking chemical modification format, every nucleotide of the antisense compound is a tricyclo-DNA modified nucleotide. In some embodiments of the steric blocking chemical modification format, the antisense compound comprises at least one MOE modified nucleotide and at least one tricyclo-DNA modified nucleotide. Many different chemical modification patterns steric blocking antisense oligonucleotides are envisioned. For example, but in no way limiting, the steric blocking antisense oligonucleotide can comprise a mixture of different types of modifications, such as a mixture of 2′-O-(2-methoxyethyl) modifications, LNA modifications, tricyclo-DNA modifications, and DNA modifications where the DNA stretches are four nucleotides or less.

In some embodiments, an antisense compound of the present disclosure directs cleavage of a target transcript by RNase H. In such embodiments, the antisense compound can be referred to as an RNase H-dependent antisense compound. In some embodiments the antisense compound is an RNase H-dependent antisense oligonucleotide. In some embodiments, an antisense oligonucleotide of the present disclosure is an RNase H-dependent antisense oligonucleotide, and can be a single-stranded, chemically modified oligonucleotide that binds to a complementary sequence in the target transcript (e.g., a target transcript). An RNase H-dependent antisense oligonucleotide of the present disclosure reduces expression of a target gene by RNase H-mediated cleavage of the target transcript, and by inhibition of translation by steric blockade of ribosomes. In some embodiments, an antisense compound of the present disclosure is capable of mediating cleavage of at least 50%, at least 55%, at least 60%, at least 65%, at least 70%, at least 75%, at least 80%, at least 85%, at least 90%, at least 95% or more of target transcripts by RNase-H. In one embodiment, the antisense compound is capable of mediating cleavage of at least 80% of target transcripts by RNase-H. In one embodiment, the antisense compound is capable of mediating cleavage of at least 90% of target transcripts by RNase-H.

In certain embodiments, an antisense compound that targets a target transcript is from about 6 to about 24 subunits in length. In other embodiments, the antisense compound that targets a target transcript is from about 8 to about 80 subunits in length. For example, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments, the antisense compounds are less than 40 linked subunits in length. In some embodiments, the antisense compounds are from about 10 to about 30 linked subunits in length. In some embodiments, the antisense compounds are from about 12 to about 25 linked subunits in length. In some embodiments, the antisense compounds are from about 15 to about 20 linked subunits in length. In some embodiments, the antisense compound is an antisense oligonucleotide that targets a target transcript, and the linked subunits are linked nucleotides.

In certain embodiments antisense compounds targeted to a target transcript can be shortened or truncated. For example, a single subunit can be deleted from the 5′ end (5′ truncation), or alternatively from the 3′ end (3′ truncation). A shortened or truncated antisense compound targeted to a target transcript can have two subunits deleted from the 5′ end, or alternatively can have two subunits deleted from the 3′ end, of the antisense compound. Alternatively, the deleted nucleosides can be dispersed throughout the antisense compound, for example, in an antisense compound having one nucleoside deleted from the 5′ end and one nucleoside deleted from the 3′ end.

When a single additional subunit is present in a lengthened antisense compound, the additional subunit can be located at the 5′ or 3′ end of the antisense compound. When two or more additional subunits are present, the added subunits can be adjacent to each other, for example, in an antisense compound having two subunits added to the 5′ end (5′ addition), or alternatively to the 3′ end (3′ addition), of the antisense compound. Alternatively, the added subunits can be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

In certain embodiments, the antisense oligonucleotide comprises the formula:

A-B—C, wherein:
A comprises from about 0 to about 18 modified nucleotides;
B comprises from about 0 to about 4 deoxyribonucleic acid (DNA) nucleotides and/or DNA-like nucleotides; and
C comprises from about 0 to about 18 modified nucleotides;
and the overall length of the antisense oligonucleotide is about 10 to about 30 nucleotides.

Antisense oligonucleotides that contain 4 or fewer DNA and/or DNA-like nucleotides in “B” should not recruit RNase H and direct cleavage of a target. In these instances, the antisense oligonucleotide is not a gapmer format, but can rather act as a steric blocker.

### V. Modified Anti-DGAT2, Anti-FASN, Anti-OPN, and Anti-NOX4 Silencing Agents

In certain aspects of the disclosure, an RNA silencing agent (or any portion thereof) of the present application, as described supra, may be modified, such that the activity of the agent is further improved. For example, the RNA silencing agents described in Section II supra, may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the present application may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of the RNA silencing agent for a non-target mRNA (e.g., wild-type mRNA), without appreciably affecting the specificity of the RNA silencing agent for a target mRNA (e.g., gain-of-function mutant mRNA).

In certain embodiments, the RNA silencing agents of the present application are modified by the introduction of at least one universal nucleotide in the antisense strand thereof. Universal nucleotides comprise base portions that are capable of base pairing indiscriminately with any of the four conventional nucleotide bases (e.g., A, G, C, U). A universal nucleotide is contemplated because it has relatively minor effect on the stability of the RNA duplex, or the duplex formed by the guide strand of the RNA silencing agent and the target mRNA. Exemplary universal nucleotides include those having an inosine base portion or an inosine analog base portion selected from the group consisting of deoxyinosine (e.g., 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In certain embodiments, the universal nucleotide is an inosine residue or a naturally occurring analog thereof.

In certain embodiments, the RNA silencing agents of the disclosure are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In certain embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the disclosure may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the present application or an siRNA produced from a shRNA) into RISC in favor of the sense strand, such that the antisense strand preferentially guides cleavage or translational repression of a target mRNA, and thus increasing or improving the efficiency of target cleavage and silencing. In certain embodiments, the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the present application may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the disclosure may be enhanced such that there is at least one mismatched base pair between the 5′ end of the first or antisense strand and the 3′ end of the sense strand portion. In certain embodiments, the mismatched base pair is selected from the group consisting of G:A, C:A, C:U, G:G, A:A, C:C and U:U. In another embodiment, the asymmetry of an RNA silencing agent of the disclosure may be enhanced such that there is at least one wobble base pair, e.g., G:U, between the 5′ end of the first or antisense strand and the 3′ end of the sense strand portion. In another embodiment, the asymmetry of an RNA silencing agent of the disclosure may be enhanced such that there is at least one base pair comprising a rare nucleotide, e.g., inosine (I). In certain embodiments, the base pair is selected from the group consisting of an I:A, I:U and I:C. In yet another embodiment, the asymmetry of an RNA silencing agent of the disclosure may be enhanced such that there is at least one base pair comprising a modified nucleotide. In certain embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present application can be modified to improve stability in serum or in growth medium for cell cultures. In order to enhance the stability, the 3′-residues may be stabilized against degradation, e.g., they may be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a one aspect, the present application features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25, or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95%, or more of the internal nucleotides. In yet another embodiment, the sense strand and/or antisense strand is modified by the substitution of all of the internal nucleotides.

In one aspect, the present application features RNA silencing agents that are at least 80% chemically modified. In certain embodiments, the RNA silencing agents may be fully chemically modified, i.e., 100% of the nucleotides are chemically modified. In another aspect, the present application features RNA silencing agents comprising 2′-OH ribose groups that are at least 80% chemically modified. In certain embodiments, the RNA silencing agents comprise 2′-OH ribose groups that are about 80%, 85%, 90%, 95%, or 100% chemically modified.

In certain embodiments, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Moreover, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphodiester group connecting to adjacent ribonucleotides is replaced by a modified group, e.g., of phosphorothioate group. In exemplary sugar-modified ribonucleotides, the 2′ OH-group is replaced by a group selected from H, OR, R, halo, SH, SR, NH2, NHR, NR2 or ON, wherein R is C₁-C₆ alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agent moieties of the instant disclosure. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

In a certain embodiment, the RNA silencing agent of the present application comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the present application comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone, which is highly resistant to nuclease digestion and imparts improved binding specificity to the molecule (Nielsen, et al., Science, (2001), 254: 1497-1500).

Also contemplated are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the present application includes RNA silencing agents having two complementary strands of nucleic acid, wherein the two strands are crosslinked. The present application also includes RNA silencing agents which are conjugated or unconjugated (e.g., at its 3′ terminus) to another moiety (e.g., a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a O with an S; (c) replacement of the U with a C₅ amino linker; (d) replacement of an A with a G (sequence changes can be located on the sense strand and not the antisense strand in certain embodiments); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a O with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Heavily Modified RNA Silencing Agents

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a dsRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in U.S. Patent Publication No. 2020/0087663A1 and U.S. Ser. No. 16/999,759 (filed Aug. 21, 2020), each of which is incorporated herein by reference.

### Internucleotide Linkage Modifications

In certain embodiments, at least one internucleotide linkage, intersubunit linkage, or nucleotide backbone is modified in the RNA silencing agent. In certain embodiments, all of the internucleotide linkages in the RNA silencing agent are modified. In certain embodiments, the modified internucleotide linkage comprises a phosphorothioate internucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate internucleotide linkages. In certain embodiments, the RNA silencing agent comprises 4-16 phosphorothioate internucleotide linkages. In certain embodiments, the RNA silencing agent comprises 8-13 phosphorothioate internucleotide linkages. In certain embodiments, the RNA silencing agent is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate internucleotide linkages. In certain embodiments, the nucleotides at positions 1 and 2 from the 3′ end of sense strand are connected to adjacent ribonucleotides via phosphorothioate internucleotide linkages. In certain embodiments, the nucleotides at positions 1 and 2 from the 5′ end of antisense strand are connected to adjacent ribonucleotides via phosphorothioate internucleotide linkages. In certain embodiments, the nucleotides at positions 1-2 to 1-8 from the 3′ end of antisense strand are connected to adjacent ribonucleotides via phosphorothioate internucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate internucleotide linkages. In certain embodiments, the nucleotides at positions 1-2 to 1-7 from the 3′ end of antisense strand are connected to adjacent ribonucleotides via phosphorothioate internucleotide linkages.

In one aspect, the disclosure provides a modified oligonucleotide, said oligonucleotide having a 5′ end, a 3′ end, that is complementary to a target, wherein the oligonucleotide comprises a sense and antisense strand, and at least one modified intersubunit linkage of Formula (I):

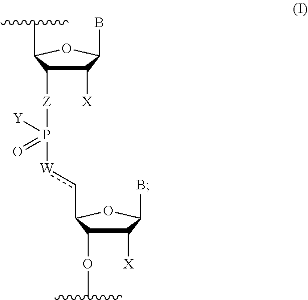

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
R is a protecting group; and
[symbol] is an optional double bond.

In an embodiment of Formula (I), when W is CH, [symbol] is a double bond.

In an embodiment of Formula (I), when W selected from the group consisting of O, OCH₂, OCH, CH₂, [symbol] is a single bond.

In an embodiment of Formula (I), when Y is O⁻, either Z or W is not O.

In an embodiment of Formula (I), Z is CH₂ and W is CH₂. In another embodiment, the modified intersubunit linkage of Formula (I) is a modified intersubunit linkage of Formula (II):

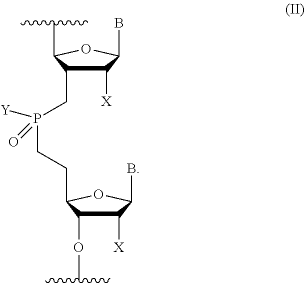

In an embodiment of Formula (I), Z is CH₂ and W is O. In another embodiment, wherein the modified intersubunit linkage of Formula (I) is a modified intersubunit linkage of Formula (III):

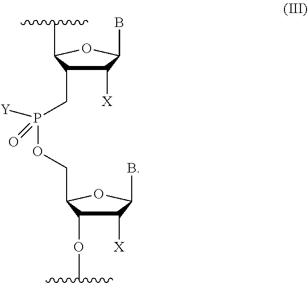

In an embodiment of Formula (I), Z is O and W is CH₂. In another embodiment, the modified intersubunit linkage of Formula (I) is a modified intersubunit linkage of Formula (IV):

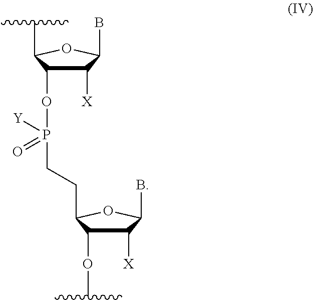

In an embodiment of Formula (I), Z is O and W is CH. In another embodiment, the modified intersubunit linkage of Formula (I) is a modified intersubunit linkage of Formula V:

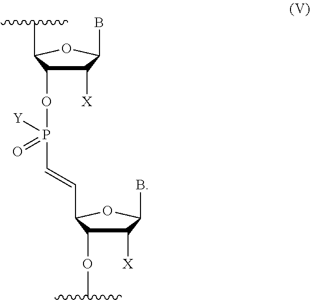

In an embodiment of Formula (I), Z is O and W is OCH₂. In another embodiment, the modified intersubunit linkage of Formula (I) is a modified intersubunit linkage of Formula VI:

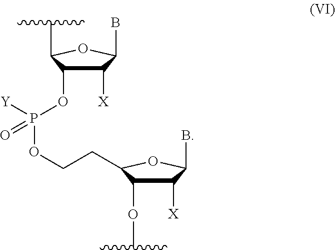

In an embodiment of Formula (I), Z is CH₂ and W is CH. In another embodiment, the modified intersubunit linkage of Formula (I) is a modified intersubunit linkage of Formula VII:

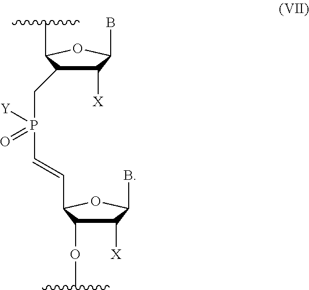

In an embodiment of Formula (I), the base pairing moiety B is selected from the group consisting of adenine, guanine, cytosine, and uracil.

In an embodiment, the modified oligonucleotide is incorporated into siRNA, said modified siRNA having a 5′ end, a 3′ end, that is complementary to a target, wherein the siRNA comprises a sense and antisense strand, and at least one modified intersubunit linkage of any one or more of Formula (I), Formula (II), Formula (III), Formula (IV), Formula (V), Formula (VI), or Formula (VII).

In an embodiment, the modified oligonucleotide is incorporated into siRNA, said modified siRNA having a 5′ end, a 3′ end, that is complementary to a target and comprises a sense and antisense strand, wherein the siRNA comprises at least one modified intersubunit linkage is of Formula VIII:

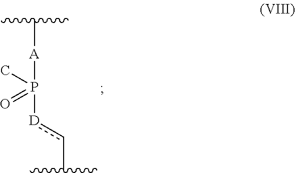

wherein:
D is selected from the group consisting of O, OCH₂, OCH, CH2, and CH;
C is selected from the group consisting of O⁻, OH, OR¹, NH⁻, NH₂, S⁻, and SH;
A is selected from the group consisting of O and CH₂;
R¹ is a protecting group;
[symbol] is an optional double bond; and
the intersubunit is bridging two optionally modified nucleosides.

In an embodiment, when C is O, either A or D is not O.

In an embodiment, D is CH₂. In another embodiment, the modified intersubunit linkage of Formula VIII is a modified intersubunit linkage of Formula (IX):

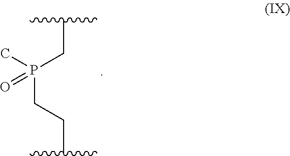 [SMILES: *CCP(C)(=O)C*]

In an embodiment, D is O. In another embodiment, the modified intersubunit linkage of Formula VIII is a modified intersubunit linkage of Formula (X):

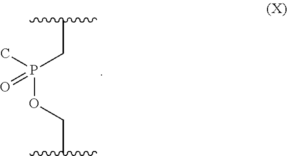 [SMILES: *COP(C)(=O)C*]

In an embodiment, D is CH₂. In another embodiment, the modified intersubunit linkage of Formula (VIII) is a modified intersubunit linkage of Formula (XI):

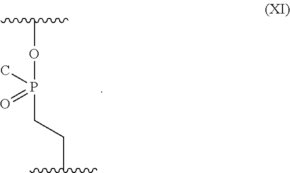 [SMILES: *CCP(C)(=O)O*]

In an embodiment, D is CH. In another embodiment, the modified intersubunit linkage of Formula VIII is a modified intersubunit linkage of Formula (XII):

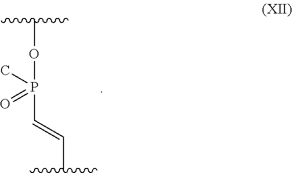 [SMILES: */C=C/P(C)(=O)O*]

In another embodiment, the modified intersubunit linkage of Formula (VII) is a modified intersubunit linkage of Formula (XIV):

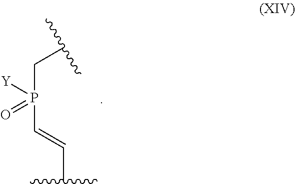

In an embodiment, D is OCH₂. In another embodiment, the modified intersubunit linkage of Formula (VII) is a modified intersubunit linkage of Formula (XIII):

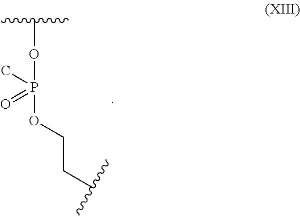 [SMILES: *CCOP(C)(=O)O*]

In another embodiment, the modified intersubunit linkage of Formula (VII) is a modified intersubunit linkage of Formula (XXa):

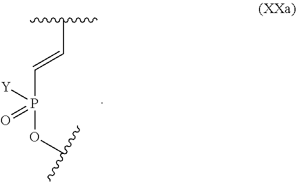

In an embodiment of the modified siRNA linkage, each optionally modified nucleoside is independently, at each occurrence, selected from the group consisting of adenosine, guanosine, cytidine, and uridine.

In certain exemplary embodiments of Formula (I), W is O. In another embodiment, W is CH2. In yet another embodiment, W is CH.

In certain exemplary embodiments of Formula (I), X is OH. In another embodiment, X is OCH3. In yet another embodiment, X is halo.

In a certain embodiment of Formula (I), the modified siRNA does not comprise a 2′-fluoro substituent.

In an embodiment of Formula (I), Y is O—. In another embodiment, Y is OH. In yet another embodiment, Y is OR. In still another embodiment, Y is NH—. In an embodiment, Y is NH2. In another embodiment, Y is S—. In yet another embodiment, Y is SH.

In an embodiment of Formula (I), Z is O. In another embodiment, Z is CH2.

In an embodiment, the modified intersubunit linkage is inserted on position 1-2 of the antisense strand. In another embodiment, the modified intersubunit linkage is inserted on position 6-7 of the antisense strand. In yet another embodiment, the modified intersubunit linkage is inserted on position 10-11 of the antisense strand. In still another embodiment, the modified intersubunit linkage is inserted on position 19-20 of the antisense strand. In an embodiment, the modified intersubunit linkage is inserted on positions 5-6 and 18-19 of the antisense strand.

In an exemplary embodiment of the modified siRNA linkage of Formula (VIII), C is O—. In another embodiment, C is OH. In yet another embodiment, C is OR1. In still another embodiment, C is NH—. In an embodiment, C is NH2. In another embodiment, C is S—In yet another embodiment, C is SH.

In an exemplary embodiment of the modified siRNA linkage of Formula (VIII), A is O. In another embodiment, A is CH2. In yet another embodiment, C is OR1. In still another embodiment, C is NH—. In an embodiment, C is NH2. In another embodiment, C is S—. In yet another embodiment, C is SH.

In a certain embodiment of the modified siRNA linkage of Formula (VIII), the optionally modified nucleoside is adenosine. In another embodiment of the modified siRNA linkage of Formula (VIII), the optionally modified nucleoside is guanosine. In another embodiment of the modified siRNA linkage of Formula (VIII), the optionally modified nucleoside is cytidine. In another embodiment of the modified siRNA linkage of Formula (VIII), the optionally modified nucleoside is uridine.

In an embodiment of the modified siRNA linkage, wherein the linkage is inserted on position 1-2 of the antisense strand. In another embodiment, the linkage is inserted on position 6-7 of the antisense strand. In yet another embodiment, the linkage is inserted on position 10-11 of the antisense strand. In still another embodiment, the linkage is inserted on position 19-20 of the antisense strand. In an embodiment, the linkage is inserted on positions 5-6 and 18-19 of the antisense strand.

In certain embodiments of Formula (I), the base pairing moiety B is adenine. In certain embodiments of Formula (I), the base pairing moiety B is guanine. In certain embodiments of Formula (I), the base pairing moiety B is cytosine. In certain embodiments of Formula (I), the base pairing moiety B is uracil.

In an embodiment of Formula (I), W is O. In an embodiment of Formula (I), W is CH2. In an embodiment of Formula (I), W is CH.

In an embodiment of Formula (I), X is OH. In an embodiment of Formula (I), X is OCH3. In an embodiment of Formula (I), X is halo.

In an exemplary embodiment of Formula (I), the modified oligonucleotide does not comprise a 2′-fluoro substituent.

In an embodiment of Formula (I), Y is O—. In an embodiment of Formula (I), Y is OH. In an embodiment of Formula (I), Y is OR. In an embodiment of Formula (I), Y is NH—. In an embodiment of Formula (I), Y is NH2. In an embodiment of Formula (I), Y is S—. In an embodiment of Formula (I), Y is SH.

In an embodiment of Formula (I), Z is O. In an embodiment of Formula (I), Z is CH2.

In an embodiment of the Formula (I), the linkage is inserted on position 1-2 of the antisense strand. In another embodiment of Formula (I), the linkage is inserted on position 6-7 of the antisense strand. In yet another embodiment of Formula (I), the linkage is inserted on position 10-11 of the antisense strand. In still another embodiment of Formula (I), the linkage is inserted on position 19-20 of the antisense strand. In an embodiment of Formula (I), the linkage is inserted on positions 5-6 and 18-19 of the antisense strand.

Modified intersubunit linkages are further described in WO 2020/198509 and U.S. Ser. No. 63/000,328 (filed Mar. 26, 2020), each of which is incorporated herein by reference.

### 4) Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., neuronal cells). Thus, the disclosure includes RNA silencing agents which are conjugated or unconjugated (e.g., at its 5′ and/or 3′ terminus) to another moiety (e.g., a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and lithocholic acid (LCA). In a certain embodiment, the fatty acid selected from the group consisting of eicosapentaenoic acid (EPA), docosahexaenoic acid (DHA) and docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the Y end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g., acridines and substituted acridines), cross-linkers (e.g., psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycosides, artificial endonucleases (e.g., EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP. In certain embodiments, the ligand is GalNAc or a derivative thereof.

In certain embodiments, the GalNAc is represented by the formula below:

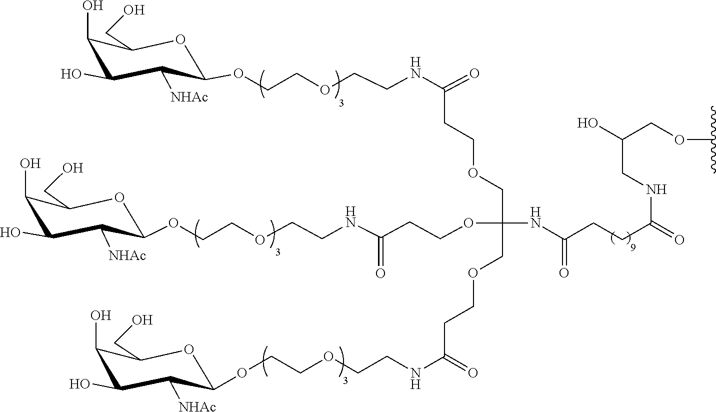

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNF□), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid-based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid-based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low-density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker is a cleavable linker. In certain embodiments, the cleavable linker comprises a phosphodiester linkage, a disulfide linkage, an acid-labile linkage, or a photocleavable linkage.

In certain embodiments, the cleavable linker comprises a dTdT dinucleotide with phosphodiester internucleotide linkages.

In certain embodiments, the acid-labile linkage comprises a β-thiopropionate linkage or a carboxydimethylmaleic anhydride (CDM) linkage.

In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

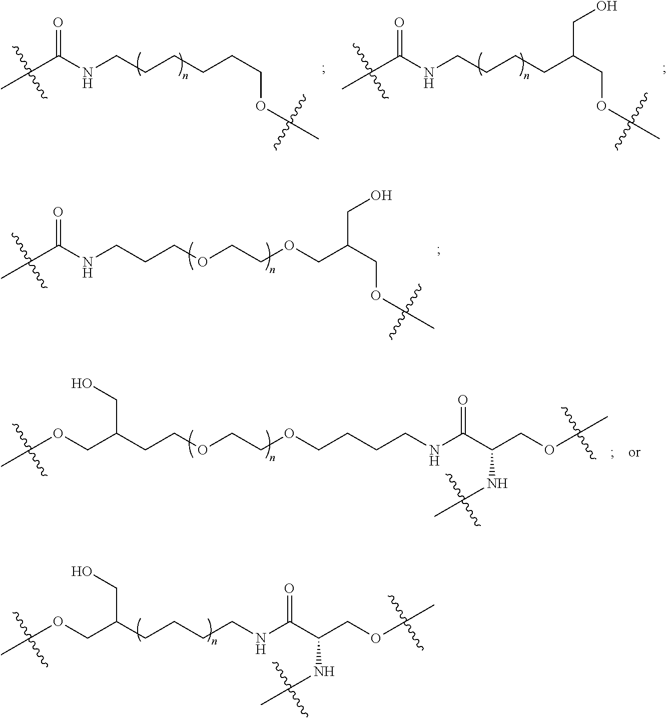

wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

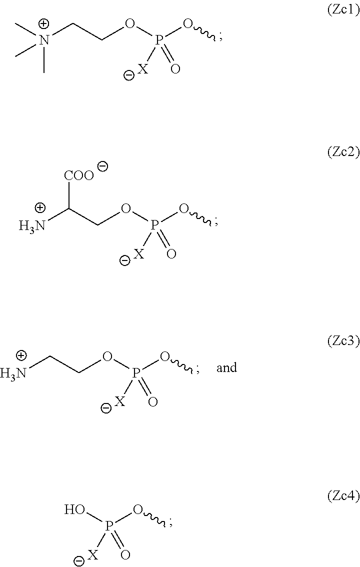

wherein X is O, S or BH₃.

The various functional moieties of the disclosure and means to conjugate them to RNA silencing agents are described in further detail in WO2017/030973A1 and WO2018/031933A2, incorporated herein by reference.

### Methods of Introducing Nucleic Acids, Vectors and Host Cells

RNA silencing agents of the disclosure may be directly introduced into the cell (e.g., a neural cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, inhibit annealing of single strands, stabilize the single strands, or other-wise increase inhibition of the target gene.

RNA may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the RNA may be introduced.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95%, or 99% as compared to a cell not treated according to the present disclosure. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of an RNAi agent of the disclosure (e.g., an siRNA targeting an DGAT2 target sequence, an siRNA targeting an FASN target sequence, an siRNA targeting an OPN target sequence, and an siRNA targeting an NOX4 target sequence,) is tested for its ability to specifically degrade target mRNA (e.g., DGAT2 mRNA and/or the production of DGAT2 protein, FASN mRNA and/or the production of FASN protein, OPN mRNA and/or the production of OPN protein, NOX4 mRNA and/or the production of NOX4 protein) in cells, such as cells in the liver or white adipose tissue. In certain embodiments, cells in the liver or white adipose tissue include, but are not limited to, hepatocytes, Kupffer cells, hepatic stellate cells, liver endothelial cells, and adipocytes. Also suitable for cell-based validation assays are other readily transfectable cells, for example, HeLa cells or COS cells. Cells are transfected with human cDNAs (e.g., human DGAT2 cDNA, human FASN cDNA, human OPN cDNA, and human NOX4 cDNA). Standard siRNA, modified siRNA or vectors able to produce siRNA from U-looped mRNA are co-transfected. Selective reduction in target mRNA (e.g., DGAT2 mRNA, FASN mRNA, OPN mRNA, NOX4 mRNA), and/or target protein (e.g., DGAT2 protein, FASN protein, OPN protein, and NOX4 protein) is measured. Reduction of target mRNA or protein can be compared to levels of target mRNA or protein in the absence of an RNAi agent or in the presence of an RNAi agent that does not target mRNA (e.g., DGAT2 mRNA, FASN mRNA, OPN mRNA, and NOX4 mRNA). Exogenously-introduced mRNA or protein (or endogenous mRNA or protein) can be assayed for comparison purposes.

### Recombinant Adeno-Associated Viruses and Vectors

In certain exemplary embodiments, recombinant adeno-associated viruses (rAAVs) and their associated vectors can be used to deliver one or more siRNAs into cells, e.g., liver cells (e.g., hepatocytes and Kupffer cells). AAV is able to infect many different cell types, although the infection efficiency varies based upon serotype, which is determined by the sequence of the capsid protein. Several native AAV serotypes have been identified, with serotypes 1-9 being the most commonly used for recombinant AAV. AAV-2 is the most well-studied and published serotype. The AAV-DJ system includes serotypes AAV-DJ and AAV-DJ/8. These serotypes were created through DNA shuffling of multiple AAV serotypes to produce AAV with hybrid capsids that have improved transduction efficiencies in vitro (AAV-DJ) and in vivo (AAV-DJ/8) in a variety of cells and tissues.

rAAVs may be delivered to a subject in compositions according to any appropriate methods known in the art. An rAAV can be suspended in a physiologically compatible carrier (i.e., in a composition), and may be administered to a subject, i.e., a host animal, such as a human, mouse, rat, cat, dog, sheep, rabbit, horse, cow, goat, pig, guinea pig, hamster, chicken, turkey, a non-human primate (e.g., Macaque) or the like. In certain embodiments, a host animal is a non-human host animal.

Delivery of one or more rAAVs to a mammalian subject may be performed, for example, by intramuscular injection or by administration into the bloodstream of the mammalian subject. Administration into the bloodstream may be by injection into a vein, an artery, or any other vascular conduit. In certain embodiments, one or more rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer virions into the vasculature of an isolated limb to potentially enhance transduction into muscle cells or tissue.

The compositions of the disclosure may comprise an rAAV alone, or in combination with one or more other viruses (e.g., a second rAAV encoding having one or more different transgenes). In certain embodiments, a composition comprises 1, 2, 3, 4, 5, 6, 7, 8, 9, 10 or more different rAAVs each having one or more different transgenes.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of one or more rAAVs is generally in the range of from about 1 mL to about 100 mL of solution containing from about 109 to 1016 genome copies. In some cases, a dosage between about 1011 to 1012 rAAV genome copies is appropriate. In certain embodiments, 1012 rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., about 1013 genome copies/mL or more). Methods for reducing aggregation of rAAVs are well known in the art and, include, for example, addition of surfactants, pH adjustment, salt concentration adjustment, etc. (See, e.g., Wright et al. (2005) Molecular Therapy 12:171-178, the contents of which are incorporated herein by reference.)

“Recombinant AAV (rAAV) vectors” comprise, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., siRNA) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are usually about 145 base pairs in length. In certain embodiments, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including mammalian AAV types described further herein.

### VI. Methods of Treatment

In one aspect, the present disclosure provides for both prophylactic and therapeutic methods of treating a subject at risk of (or susceptible to) developing diseases associated with the dysregulation of lipid metabolism by inhibiting DGAT2, FASN, OPN, and NOX4. Diseases associated with lipid metabolism dysregulation include; Nonalcoholic fatty liver disease (NAFLD), nonalcoholic steatohepatitis (NASH), dyslipidemia, lipodystrophy syndrome and metabolic syndrome (MetS) the latter of which is associated with an increased risk of developing atherosclerotic cardiovascular disease (CVD), stroke and type 2 diabetes. In general, treatment will result in a reduction in serum levels of at least one other hepatic enzyme besides DGAT2, (such as, e.g., Stearoyl-CoA desaturase-1 (SCD1) or Fatty acid synthase (FASN)), and/or a decrease in hepatic lipid accumulation.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the disclosure pertains to methods treating subjects therapeutically, i.e., alter onset of symptoms of the disease or disorder. In an exemplary embodiment, the modulatory method of the disclosure involves contacting a hepatocyte expressing DGAT2, FASN, OPN, and NOX4 with a therapeutic agent (e.g., a RNAi agent or vector or transgene encoding same) that is specific for a target sequence within the gene (e.g., DGAT2 target sequences of Table 1, FASN target sequences of Table 5, OPN target sequences of Table 8, and NOX4 target sequences of Table 10), such that sequence specific interference with the gene is achieved. These methods can be performed in vitro (e.g., by culturing the cell with the agent) or, alternatively, in vivo (e.g., by administering the agent to a subject).

### VII. Pharmaceutical Compositions and Methods of Administration

The disclosure pertains to uses of the above-described agents for prophylactic and/or therapeutic treatments as described infra. Accordingly, the modulators (e.g., RNAi agents) of the present disclosure can be incorporated into pharmaceutical compositions suitable for administration. Such compositions typically comprise the nucleic acid molecule or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, and subcutaneous.

The nucleic acid molecules of the disclosure can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow-release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the disclosure can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddison et al. (2002), supra; Paul (2002), supra; Sui (2002) supra; Yu et al. (2002), supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), Supra.

For example, compositions can include one or more species of a compound of the disclosure and a pharmaceutically acceptable carrier. The pharmaceutical compositions of the present disclosure may be administered by intravenous or subcutaneous injection. or.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### EXAMPLES

### Example 1. Material and Methods

### Human Stellate Cell Activation Studies

In vitro screening of LX2 human stellate cells was executed by plating 500,000 cells/well in 6-well plates overnight at 37° C., 5% CO2 in 0% FBS DMEM medium (11885-076, Gibco). Following a 4-hour pre-treatment with 1 nM or 100 nM Fasn inhibitor (TVB-3664-S8563, Selleck Chemicals), stellate cell activation was initiated by adding recombinant human Tgf-β (240-B-002, R & D Systems) for a 5 ng/mL final concentration. After 48 hours, protein and mRNA were isolated from the cells and further analysis was performed.

### AAV Constructs

AAV8-TBG-Cre and Control AAV constructs were produced by UMass Chan Viral Core, as previously described (see Sena-Esteves, M., and Gao, G. (2020). Introducing Genes into Mammalian Cells: Viral Vectors. Cold Spring Harb Protoc 2020, 095513. 10.1101/pdb.top095513, incorporated herein by reference). AAV9-Lrat-Cre was produced by Vector BioLabs.

### Animal Studies

Animal experiments were performed in accordance with animal care ethics approval and guidelines of University of Massachusetts Medical School Institutional Animal Care and Use Committee (protocol number: A-1600-19). For in vivo studies, ten-week-old, male, Fasn fl/fl mice were each injected with either AAV8-TBG-Cre (hepatocyte specific KO), AAV9-Lrat-Cre (stellate cell specific KO), or a combination of the two constructs via intravenous injection. Each mouse was injected with 3×1011 GC/mL. Specifically, hepatocyte-specific KO mice received 1×1011 AAV8-TBG-Cre and 2×1011 Control AAV, stellate cell-specific KO received 2×1011 AAV9-LRAT-Cre and 1×1011 Control AAV, and the multiple-KO groups were given both 1×1011 AAV8-TBG-Cre and 2×1011 AAV9-LRAT-Cre. Following administration of the AAV constructs, the mice were placed on a Chow diet for one week before transitioning to HFD for the remaining 7 weeks. On weeks 5 and 8, liver stiffness was measured via shear-wave elastography.

### Oligonucleotide Synthesis

Oligonucleotides were synthesized using modified (2′-F, 2′-O-Me) phosphoramidites with standard protecting groups. 5′-Vinyl Tetra phosphonate (pivaloyloxymethyl) 2′-O-Methyl Uridine 3′-CE phosphoramidite (VP) was used for the 5′-Vinyl-phosphonate coupling when needed. All amidites were purchased from (Chemgenes, Wilmington, MA). Phosphoramidite solid-phase synthesis was done on a MerMade12 (Biosearch Technologies, Novato, CA) using modified protocols. GalNAc conjugated oligonucleotides were grown on a 500 Å LCAA custom aminopropanediol-based trivalent GalNAc-CPG (Centernauchsnab, Minsk, Belarus). Phosphoramidites were prepared at 0.1 M in anhydrous acetonitrile (ACN), with added dry 15% dimethylformamide in the 2′-OMe-Uridine amidite. 5-(Benzylthio)-1H-tetrazole (BTT) was used as the activator at 0.25 M. Detritylations were performed using 3% trichloroacetic acid in dichloromethane. Capping reagents used were CAP A, 20% n-methylimidazole in ACN and CAP B, 20% acetic anhydride, 30% 2,6-lutidine in ACN (Synthesis reagents were purchased at AIC, Westborough, MA). Sulfurization was performed with 0.1 M solution of 3-[(dimethylaminomethylene)amino]-3H-1,2,4-dithiazole-5-thione (DDTT) in pyridine (Chemgenes, Wilmington, MA) for 3 min. Phosphoramidite coupling times were 4 min.

### Deprotection and Purification of Oligonucleotides

Conjugated oligonucleotides were cleaved and deprotected 28-30% ammonium hydroxide and 40% aq. methylamine (AMA) in a 1:1 ratio, for 2 h at room temperature. The VP containing oligonucleotides were cleaved and deprotected as previously described (see O'Shea, J., Theile, C. S., Das, R., Babu, I. R., Charisse, K., Manoharan, M., Maier, M. A., and Zlatev, I. (2018). An efficient deprotection method for 5′-[0,0-bis(pivaloyloxymethyl)]-(E)-vinylphosphonate containing oligonucleotides. Tetrahedron 74, 6182-6186. https://doi.org/10.1016/j.tet.2018.09.008, incorporated herein by reference). Briefly, CPG with VP-oligonucleotides was treated with a solution of 3% Diethylamine in 28-30% ammonium hydroxide at 35° C. for 20 h.

The solutions containing cleaved oligonucleotides were filtered to remove the CPG and dried under vacuum. The resulting pellets were re-suspended in 5% ACN in water. Purifications were performed on an Agilent 1290 Infinity II HPLC system. VP and GalNAc conjugated oligonucleotides were purified using a custom 20×150 mm column packed with Source 15Q anion exchange resin (Cytiva, Marlborough, MA); run conditions: eluent A, 10 mM sodium acetate in 20% ACN in water; eluent B, 1 M sodium perchlorate in 20% ACN in water; linear gradient, 10 to 35% B 20 min at 40° C. Flow was 40 mL/min and peaks were monitored at 260 nm. Fractions were analyzed by liquid chromatography mass spectrometry (LC-MS), pure fractions were dried under vacuum. Oligonucleotides were re-suspended in 5% ACN and desalted by size exclusion on a 50×250 mm custom column packed with Sephadex G-25 media (Cytiva, Marlborough, MA), and lyophilized.

### LC-MS Analysis of Oligonucleotides.

The identity of oligonucleotides was verified by LC-MS analysis on an Agilent 6530 accurate mass Q-TOF using the following conditions: buffer A: 100 mM 1,1,1,3,3,3-hexafluoroisopropanol (HFIP) and 9 mM triethylamine (TEA) in LC-MS grade water; buffer B:100 mM HFIP and 9 mM TEA in LC-MS grade methanol; column, Agilent AdvanceBio oligonucleotides C18; linear gradient 0-30% B 8 min (VP and GalNAc); temperature, 60° C.; flow rate, 0.5 ml/min. LC peaks were monitored at 260 nm. MS parameters: Source, electrospray ionization; ion polarity, negative mode; range, 100-3,200 m/z; scan rate, 2 spectra/s; capillary voltage, 4,000; fragmentor, 180 V.

Deprotection, purification and LC-MS reagents were purchased from Fisher Scientific, Sigma Aldrich and Oakwood Chemicals

### RNA Isolation and RT-Quantitative PCR

Frozen liver tissue punches (25-50 mg) were homogenized in TRIzol (15596018, Thermo Scientific) using the Qiagen TissueLyser II. Chloroform was added to the homogenate and centrifuged for 15 minutes at maximum speed. The clear upper layer was added to an equal volume of 100% isopropanol and incubated for 1 h at 4° C. After 10 min of centrifugation at maximum speed, the supernatant was discarded, and 70 to 75% ethanol was added to wash the pellet. After 5 min of centrifugation at maximum speed, the supernatant was discarded, and the pellet was briefly dried in the hood prior to resuspension in double-distilled water. RNA concentration was measured via the Thermo Scientific NanoDrop2000 spectrophotometer. Complementary DNA was then synthesized from 1 μg of total RNA using iScript cDNA Synthesis Kit (1708890, Bio-Rad) and Bio-Rad T100 thermocycler. Quantitative RT-PCR was performed using iQ SybrGreen Supermix (4368708, Applied Biosystems) on the Bio-Rad CFX96 C1000 Touch Thermal Cycler and analyzed.

### Immunoblotting

In the analysis of protein expression, cell lysates were homogenized in a RIPA buffer (25 mM Tris, pH 7.4 0.15 M NaCl 0.1% Tween 20) with 1:100 protease inhibitor (78442, Thermo Scientific). The lysates were denatured by boiling, separated on a 4 to 15% SDS/polyacrylamide gel electrophoresis gel (567-1094, Bio-Rad), and transferred to a nitrocellulose membrane (1704159, Bio-Rad). The membrane was blocked using TBS-T with 5% BSA for 1 hour at room temperature and incubated using p-SMAD3 (ab52903, Abcam), type I collagen (1310-30, Southern Biotech), and GAPDH (5174, Cell Signaling). The blot was further washed in TBS-T and incubated at room temperature with its corresponding secondary antibody for 30 minutes. Following another wash, the blot was incubated with ECL (32106, Thermo Scientific) and visualized with the ChemiDox XRS+ image-forming system.

### Plasma Measurements

Alanine transaminase levels were quantified using the Alanine Transaminase Colorimetric Activity Assay Kit® from Cayman Chemical. The instructions found in the user manual were followed utilizing plasma collected via retro orbital bleed prior to sacrifice. Absorbance was read by the Tecan safire2 microplate reader. Rest of the plasma measurements were done by UMass Chan Metabolic Core.

### Triglyceride Assay

Frozen liver punches (100-150 mg) were used to determine TG levels from tissue homogenates. Instructions found in the booklet of Cayman's Triglyceride Colorimetric Assay® (10010303) were followed to quantify glycerol fluorometric measurement resulting from the enzymatic hydrolysis of triglycerides via lipase.

### Histological Analysis

For the IHC, one lobe of the liver was fixed in 4% paraformaldehyde and embedded in paraffin. Sectioned slides were then stained type I collagen (Southern Biotech) at the UMass Medical School Morphology Core. Photos from the liver sections were taken with an Axiovert 35 Zeiss microscope (Zeiss) equipped with an Axiocam CCI camera at the indicated magnification.

### Collagen Assay

Total Collagen was measured from frozen liver punches (200-250 mg) using Quickzyme Total Collagen Assay® (QZBTOTCOL1). Instructions found in this Assay package were followed as it utilizes the presence of hydroxyproline to determine the amount of collagen present from tissue homogenates.

### Software & Statistics

All statistical analyses were performed using the GraphPad Prism 8 (GraphPad Software, Inc.). The data are presented as mean±SEM. For analysis of the statistical significance between four or more groups, two-way ANOVA and multiple comparison t tests were used. Ns is nonsignificant (p >0.05), *: p<0.05, **: p<0.005, and ***: p<0.0005.

### Example 2. Tgf-β Driven Activation of Human Stellate Cells In Vitro Promoted by Fasn

The Tgf-β driven activation of human stellate cells in vitro promoted by Fasn was assessed.

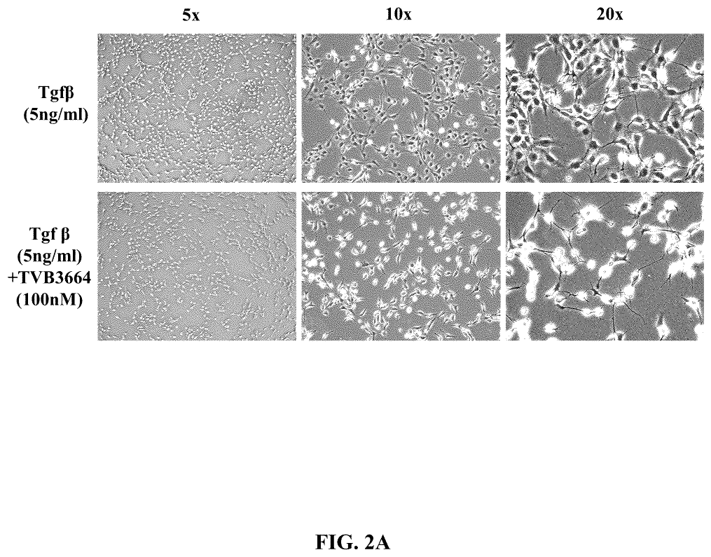

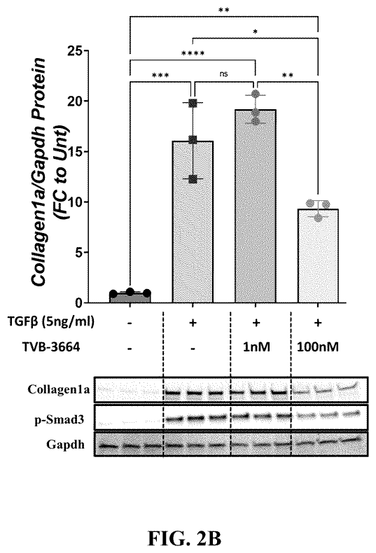

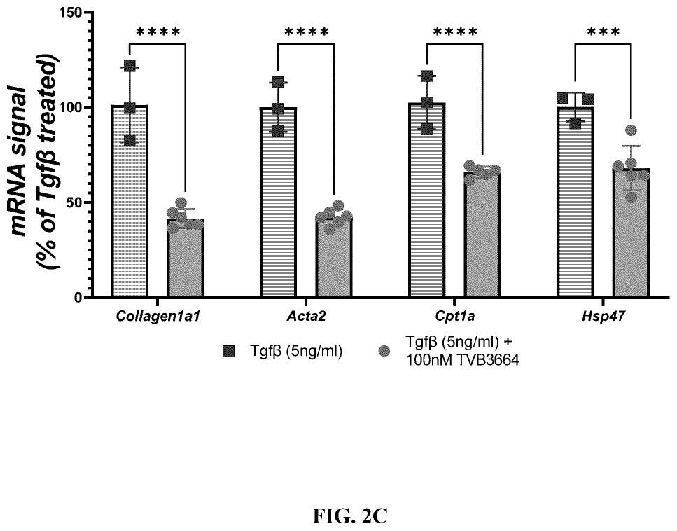

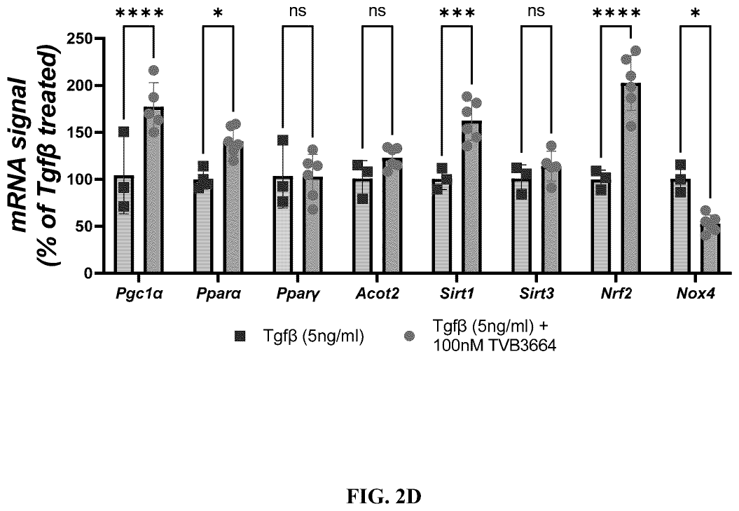

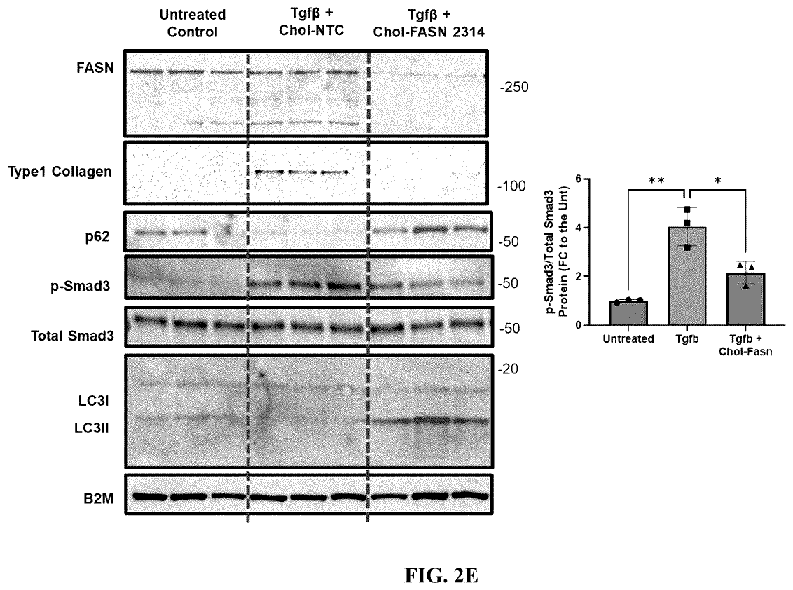

To investigate the role of palmitate production in stellate cell activation, LX2 human stellate cells were treated with a validated, potent and specific small molecule inhibitor of Fasn, TVB-3664, at a final concentration of either 1 nM or 100 nM for 4 hours. Cells were then subjected to 5 ng/mL Tgf-β, a strong stimulator of stellate cell activation. Forty-eight hours later, the morphology of activated stellate cells, with increased cell size and elongation of spindle-like structures, was easily observed in Tgf-β treated cells. In contrast, TVB-3664 severely inhibited this morphological change in LX-2 cells that were exposed to Tgf-β (FIG. 2A). Moreover, Fasn inhibition also blocked Tgf-β-induced type 1 collagen protein expression (FIG. 2B). Surprisingly, this reduction of collagen 1 protein in response to FASN inhibition correlates with reduced phosphorylated SMAD3 protein, a major driver of collagen production. Taken together, these results suggest that blocking palmitate synthesis attenuates upstream elements in TGF signaling that promote fibrosis, as supported by the reduction in mRNA levels of Col1a1 (FIG. 2B) and Acta2 (FIG. 2C) in response to TVB-3664.

Further analysis of these experiments showed that Fasn inhibition leads to a reduction in several genes that have been associated with NASH, including Cpt1a and Hsp47, consistent with a more quiescent stellate cell phenotype. (FIG. 2C). Strikingly, Fasn inhibition in Tgf-β activated stellate cells induced remarkable upregulation of Pgc-1α, Pparα, Sirt1 and Nrf2 (FIG. 2D), suggesting overall improvements in mitochondrial biogenesis as well as reduction in oxidative stress. Interestingly, Nrf2 has previously been reported to have an inhibitory role in profibrotic SMAD2/3 phosphorylation in stellate cells. These findings related to p-SMAD3 levels (FIG. 2B) and increased Nrf2 gene expression upon Fasn inhibition are consistent with a strong anti-fibrotic effect of TVB-3664. Upregulation of Nrf2 was also correlated with the reduction in expression of Nox4, a major enzyme in the ROS production pathway, supporting a lower oxidative stress state upon Fasn inhibition in activated stellate cells. (FIG. 2D)

Moreover, the protein level of Smad3 was also measured for untreated control, cholesterol conjugated non-targeting control, and cholesterol conjugated Fasn-2314. 1×106 of human Lx2 cells were plated in 10 cm2 plates and treated with 1.5 uM cholesterol conjugated Fasn-2314. 24 hrs post treatment Lx2 cells were activated with Tgfb (5 ng/ml) to induce fibrotic phenotype. 48 hrs post activation cells were harvested and processed for Western blotting analysis. The results show that: FASN 2314 achieved strong protein KD in human stellate cells (LX2s); FASN KD blunted the activation and collagen production in Tgfb treated stellate cells; FASN KD resulted in accumulation of p62 and LC3II indicating blunted autophagy which is an important energy source for stellate cells during activation; and FASN KD blunted phosphorylation of fibrotic pathway element, SMAD3, suggesting the important role of fatty acid synthesis in collagen production signaling pathway.

### Example 3. Effects of Single and Dual Hepatic Targeting of FASN and DGAT2 in Mice Placed on a NASH Inducing Diet

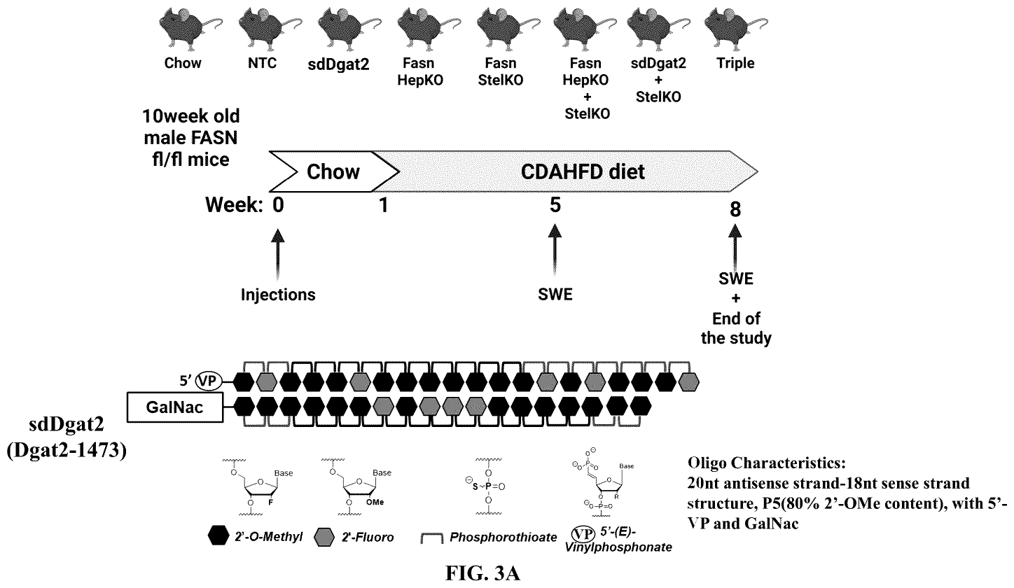

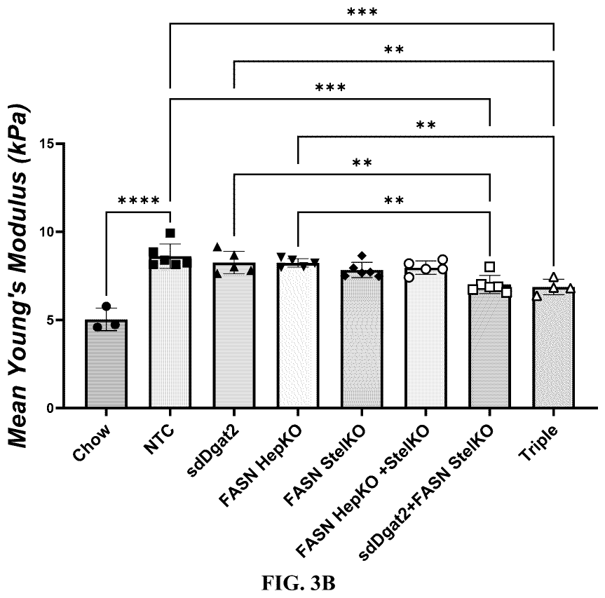

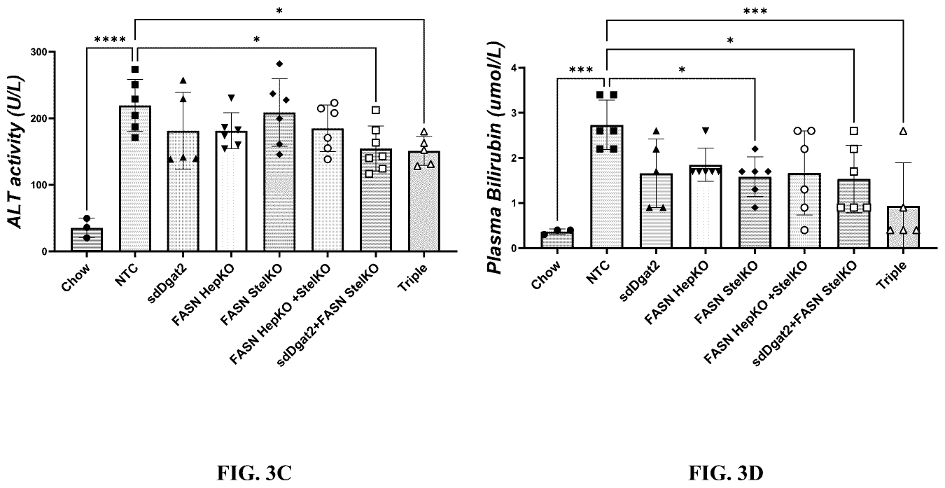

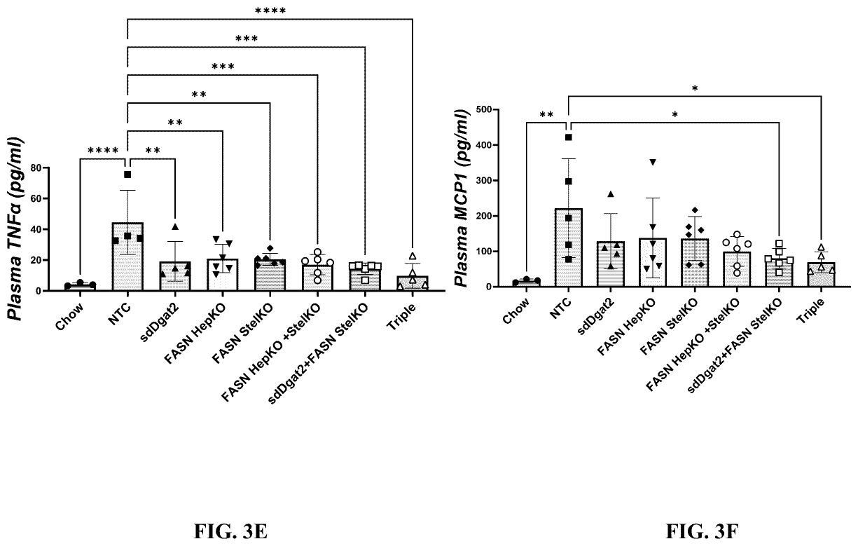

The effects of single and dual hepatic targeting of FASN and DGAT2 in mice placed on a NASH inducing diet were assessed (FIG. 3). Specifically, the effects of selective depletion of FASN in hepatocytes vs liver stellate cells in vivo were tested, and the effects of these selective FASNKO perturbations in combination with DGAT2 silencing in hepatocytes in vivo were tested as well. In order to induce such cell-selective FASNKO and DGAT2-depleted mice, Fasn fl/fl mice were injected with the following reagents: non-targeting control sdRNA plus control AAV (denoted NTC mice); hepatocyte selective sdDgat2-1473 (sdDgat2) compound for DGAT2 silencing); AAV8-TBG-Cre for hepatocyte selective Fasn KO (Fasn HepKO); AAV9-LRAT-Cre for stellate cell selective FASNKO (Fasn StelKO); both AAV8-TBG-Cre and AAV9-LRAT-Cre for hepatocyte plus stellate cell specific FASNKO (Fasn HepKO+StelKO). Combinations of these injections were also used to deplete both FASN and DGAT2 in selective cell types, as depicted in FIG. 3A. The sdDgat2 compound was injected once subcutaneously, while AAVs were injected once intravenously. One week following injections, mice were placed on a choline-deficient high fat diet (CDHFD) for 7 weeks (FIG. 3A).

Shear wave elastography (SWE) enabled quantitative measurements of liver stiffness, a biomarker for estimating inflammation and fibrosis, without sacrificing the mice (FIG. 3B). SWE was performed at week 5 as well as at the end of the study. At week 8, SWE measurements revealed that the combined depletions of FASN (in stellate cells or in stellate cells plus hepatocytes) plus DGAT2 in hepatocytes (sdDgat2+StelKO and Triple group) caused significantly less liver stiffness compared to NTC, sdDgat2, and Fasn HepKO groups (FIG. 3B). Furthermore, no stiffness difference was detected between the combined DGAT2 plus FASN depletion groups. Additionally, circulating indicators of liver damage and inflammation were measured, showing plasma ALT activity (FIG. 3C) and bilirubin (FIG. 3D) were significantly lower in mice that were depleted in both stellate cell FASN and DGAT2. All groups showed less plasma TNFα levels compared to the NTC group, suggesting depletions of either DGAT2 or FASN can attenuate inflammation through reducing lipotoxicity (FIG. 3E), and combined depletions of FASN plus DGAT2 significantly lowered plasma MCP1, while single depletions of FASN or DGAT2 did not (FIG. 3F).

### Example 4. Analysis of Livers from the Mice Described in Example 3

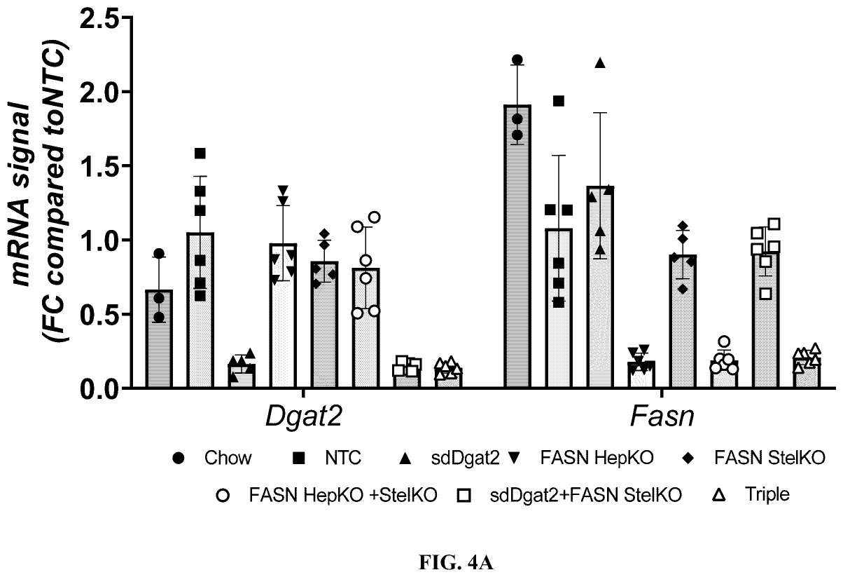

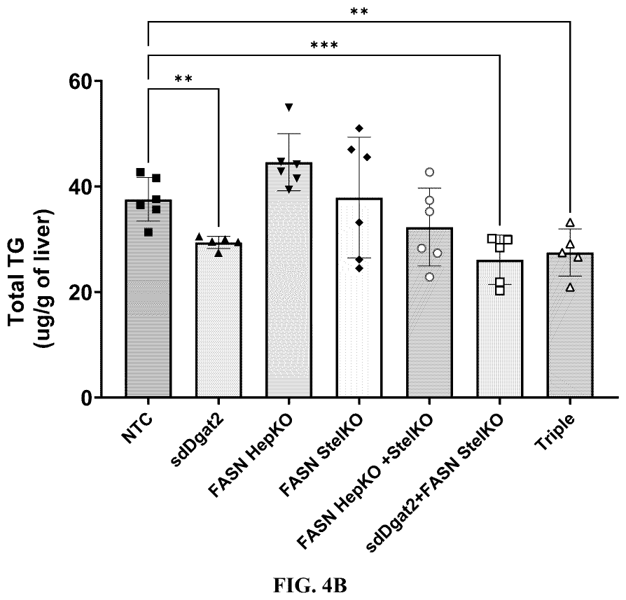

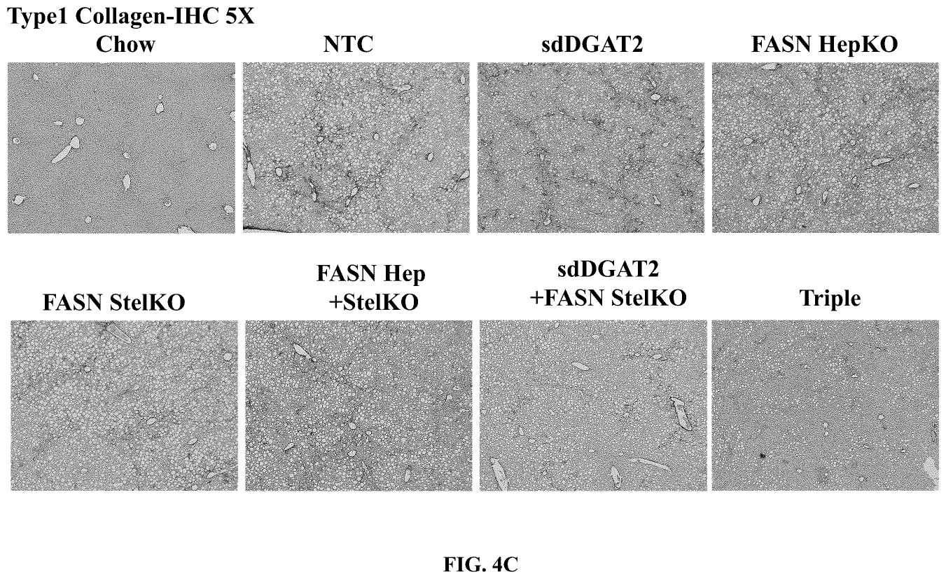

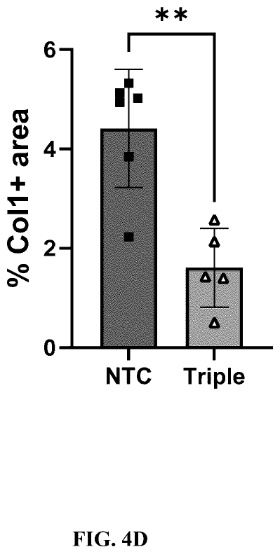

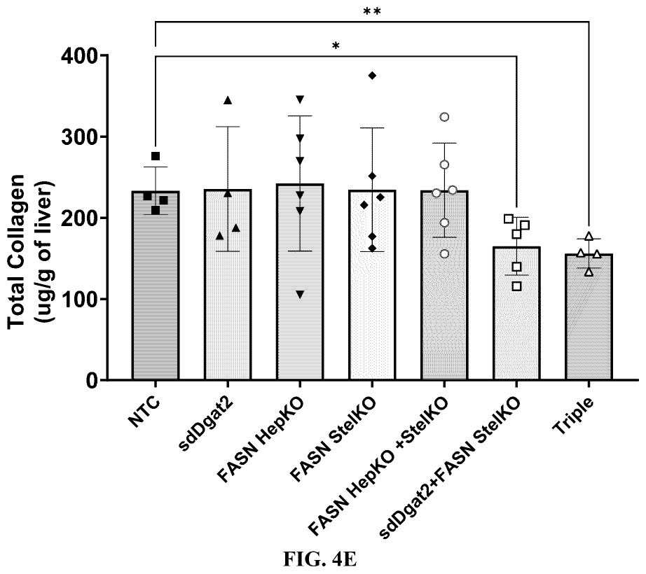

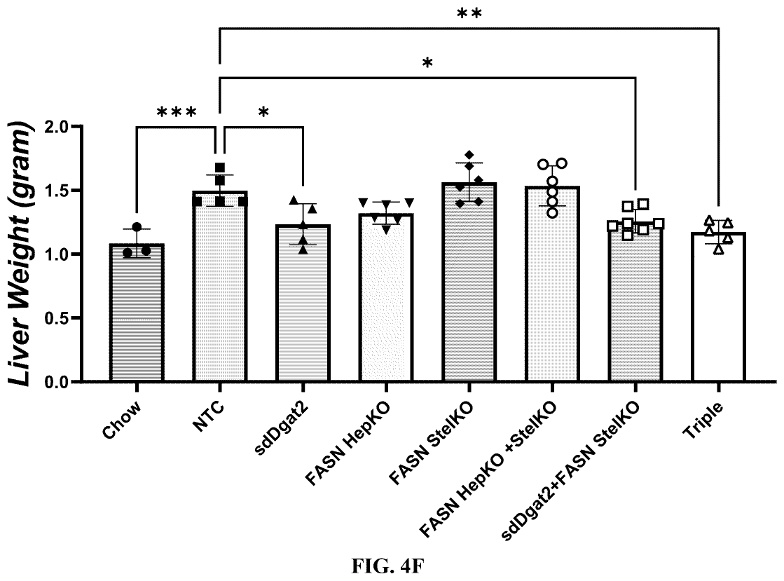

Analysis of livers from the mice described in Example 3 was performed. The analysis showed that Dgat2 mRNA was strongly silenced by the sdDgat2 compound under all conditions in which it was administered (FIG. 4A). Fasn mRNA levels were normal upon knockdown of Dgat2 under the conditions of these experiments. Marked Fasn mRNA depletion was detected in livers of the Fasn HepKO groups, but not in FASN StelKO groups, as stellate cells comprise only 5 to 8% of whole liver. Dgat2 mRNA was not affected in FASNKO groups in which sdDgat2 was not administered. It was confirmed in a separate study that the AAV9-LRAT-Cre construct efficiently and selectively depletes MCT1 in stellate cells when injected into Mct1 fl/fl mice. Furthermore, total liver triglycerides were significantly lowered in all sdDgat2 treated groups (FIG. 4B). Immunohistochemical analysis of liver sections with antibody against type 1 collagen revealed a striking prevention of fibrosis development in the Triple KO group (FIG. 4C). Type 1 collagen quantified in liver sections from all mice in the NTC and Triple KO groups showed significantly lower collagen signal in the sdDgat2 plus FASN StelKO group and the Triple group compared to the NTC. Total hydroxyproline (OH-Pro) content was also significantly lower in these same two combination KO groups. Taken together, these results demonstrate a remarkable efficacy in preventing NASH development by combining FASNKO in liver stellate cells with DGAT2 depletion in hepatocytes in the CDHFD mouse model.

### Example 5. Effects Dgat2-Fasn siRNA Combination on Fibrosis in Human Liver Organoid Model of MASH

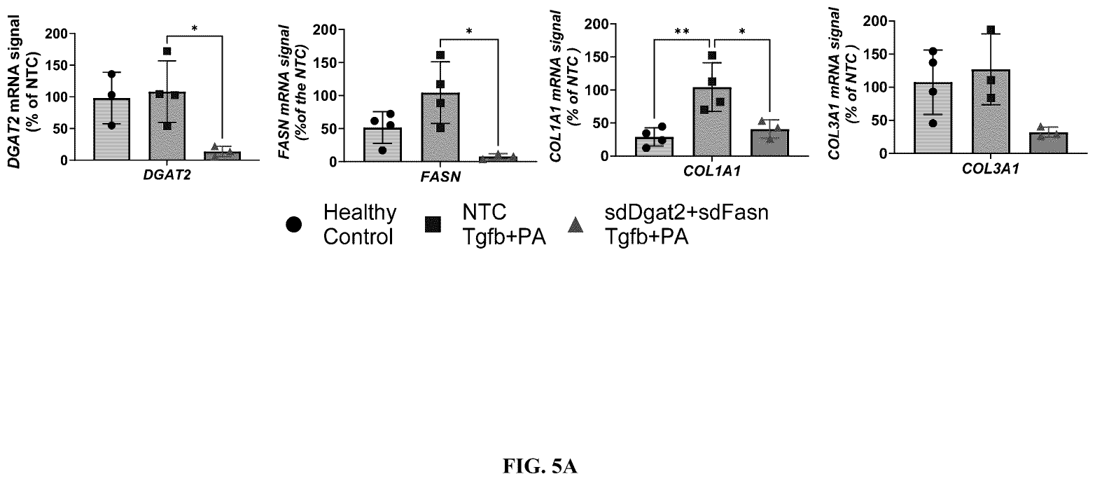

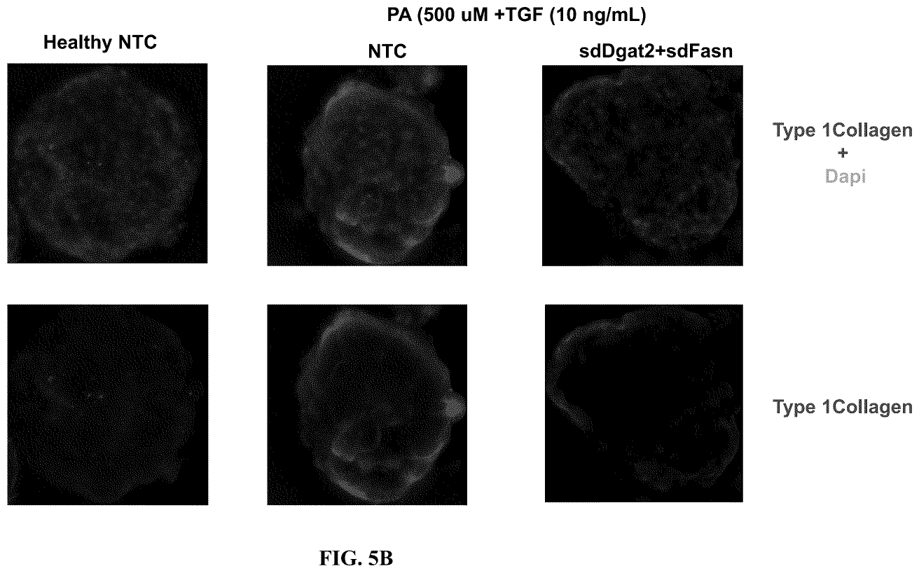

The effects Dgat2-Fasn siRNA combination on fibrosis in human liver organoid model of MASH were assessed (FIG. 5A-5B).

Dgat2-Fasn siRNA combination was found to blunt fibrosis in human liver organoid model of MASH. Human liver organoids (HLOs) were prepared as disclosed by Hess et al. (Hess et al, ingle-cell transcriptomics stratifies organoid models of metabolic dysfunction-associated steatotic liver disease. EMBO J. 2023 Dec. 11; 42(24):e113898. doi: 10.15252/embj.2023113898. Epub 2023 Nov. 14. PMID: 37962490; PMCID: PMC10711666). At the last day of differentiation, Tgf-b and palmitate was added in the media to induce MASH phenotype in HLOs. Concurrently, HLOs were treated either with cholesterol conjugated NTC or cholesterol conjugated Dgat2-1473+Fasn-2314 combination. Five days post treatment HLOs were harvested and mRNA levels of DGAT2, FASN, COL1A1, and COL3A1 were measured by qPCR (FIG. 5A) and Collagen1 protein was measured by immunofluorescence (FIG. 5B).

### Example 6. Effects of Dgat2-Fasn siRNA Combination on NASH Development in CDAHFD Mouse Model

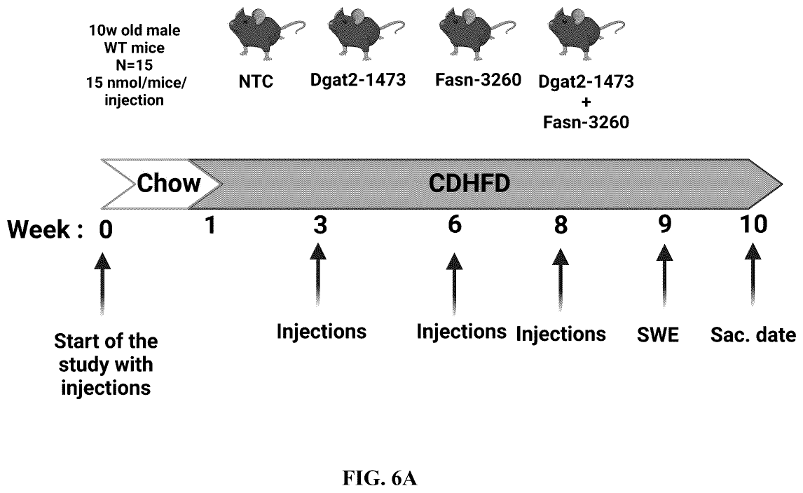

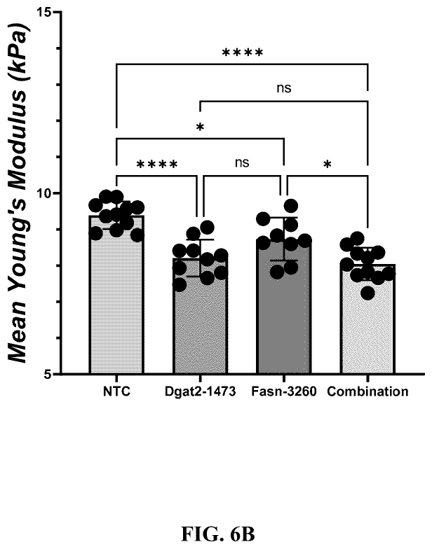

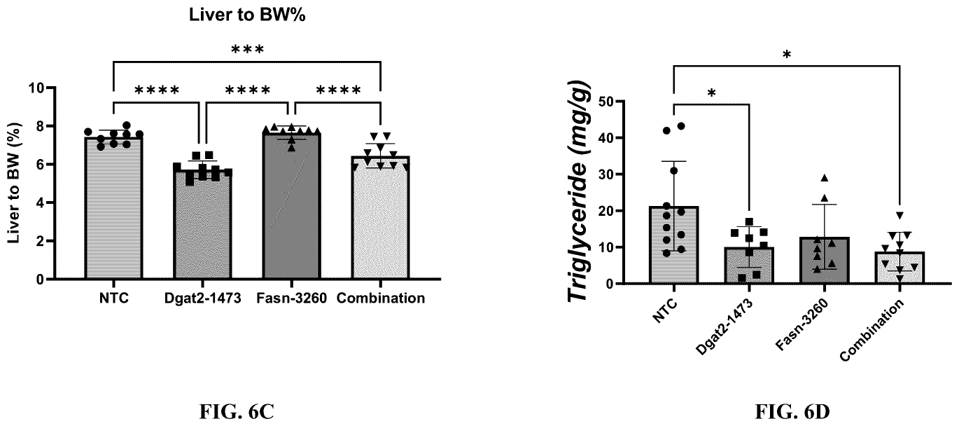

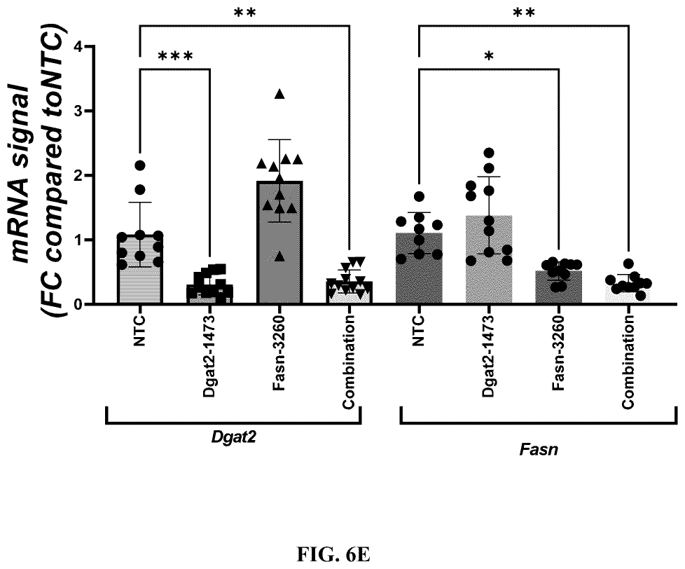

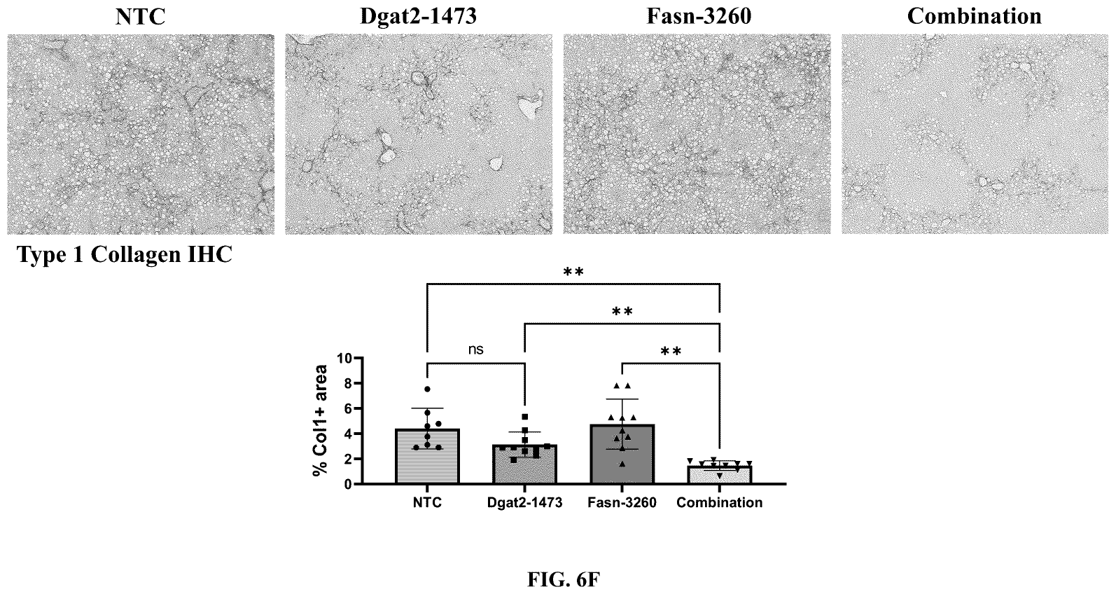

The effects of Dgat2-Fasn siRNA combination on NASH development in CDAHFD mouse model were assessed (FIG. 6A-6F).

Dgat2-Fasn siRNA combination was found to prevent NASH development in CDAHFD mouse model. Wild-type male C57BL6 Mice were injected with GalNac conjugated NTC, Dgat2-1473, Fasn-3260 or both Dgat2-1473 and Fasn-3260 together subcutaneously (10 mg/kg dose) (FIG. 6A). A week later from the injection mice were put on CDAHFD for 10 weeks to induce NASH. Liver stiffness was measured at 8 weeks of CDAHFD (FIG. 6B). Mice were sacrificed and tissues were harvested at 10th week. The liver to body ratio (FIG. 6C) and liver triglyceride content (FIG. 6D) were then measured. Liver stiffness and liver size were found to be lower in Dgat2-1473 only and combination groups. The Dgat2/Fasn silencing (FIG. 6E) and collagen content (FIG. 6F) in liver sections were also analyzed. Strong silencing of target genes was achieved with both Dgat2-1473 and Fasn-3260 in liver, and Dgat2-Fasn dual targeting resulted in lower type 1 collagen production.

### Example 7. OPN Screening of siRNA Compounds

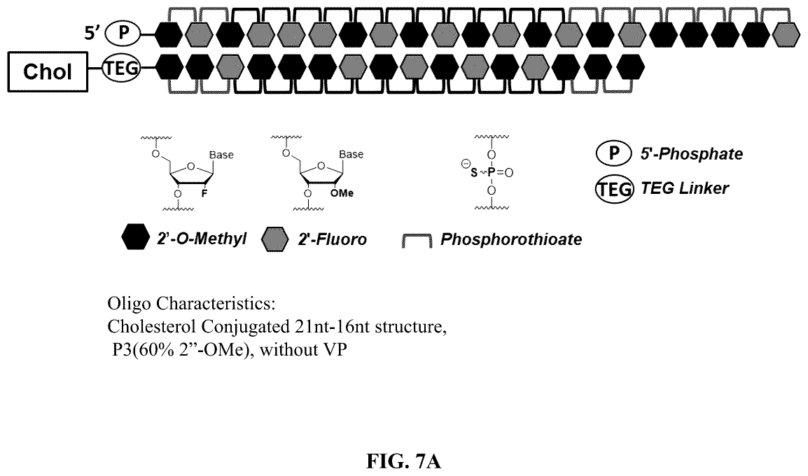

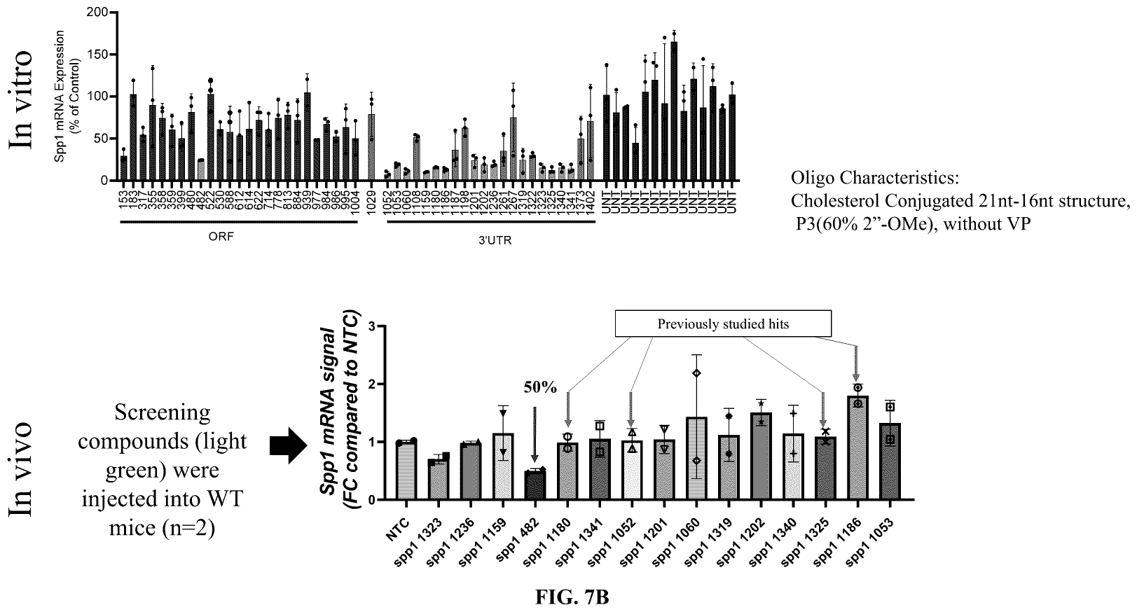

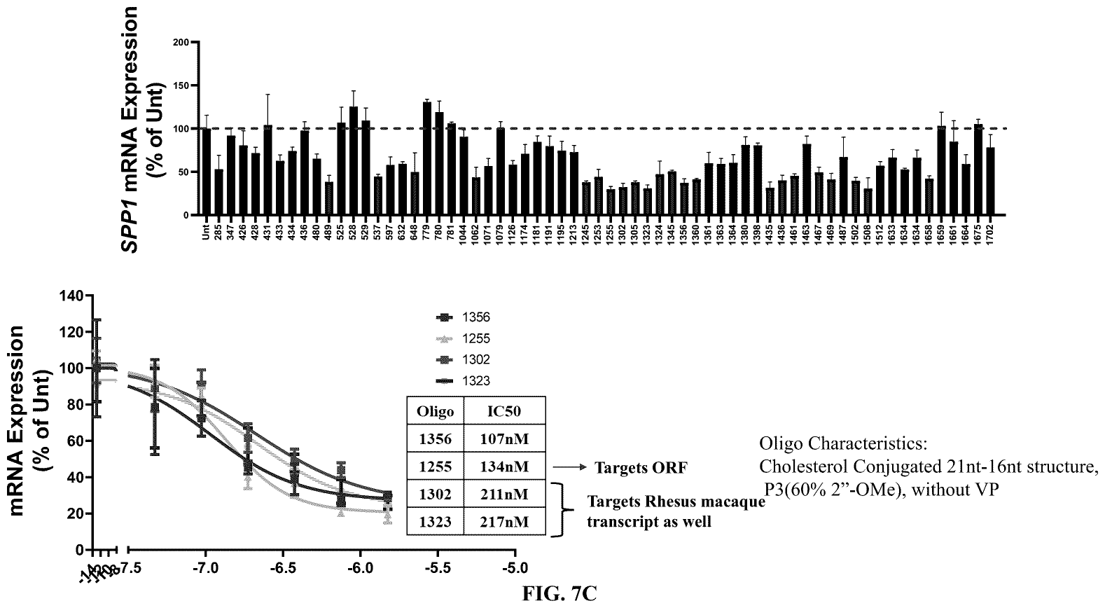

The OPN screening of siRNA compounds was assessed (FIG. 7A-7C).

The initial in vitro screening was carried out by plating 20K cells/well of HepG2 cells for human (FIG. 7C), FL83b cells for mouse (FIG. 7B), in a 96 well plate. The cells were treated with the candidate compounds in final concentration of 1.5 μM. Cells were treated for 72 hours at 37° C., 5% CO2 in 3% FBS DMEM medium. After 72 hours, mRNA levels were quantified using the QuantiGene 2.0 assay kit (Affymetrix, QS0011) as previously described by Coles et al, 2016 and Nikan et al, 2016. Briefly, cells were lysed in 300 μL diluted lysis mixture composed of one-part lysis mixture (Affymetrix, QG0503), 2 parts H2O, and 0.167 μg/μL proteinase K (ThermoScientific, E00491) for 1 hour at 55° C. Cell lysates were mixed thoroughly, and 20 μL/well of each lysate was added to the capture plate with 60 μL diluted lysis mixture without proteinase K. Probe sets for (OPN) Spp1 and B2m (Affymetrix; 80246, 80003) were used according to the manufacturer's recommended protocol. Datasets were normalized to B2m. The dose response investigation was done using serially diluted doses of the two candidate compounds (1.500, 0.750, 0.375, 0.188, 0.094, 0.047, 0.023, 0.012 μM), each condition in triplicate and QuantiGene 2.0 Assay was carried out. For mouse in vivo trials C57BL6 mice were injected with the hit siRNAs from in vitro screen (10 mg/kg) subcutaneously and sacrificed 1 week after injection. Livers were harvested and OPN (Spp1) mRNA level measurements were done via qPCR. (FIG. 7B)

### Example 8. NOX4 Screening of siRNA Compounds

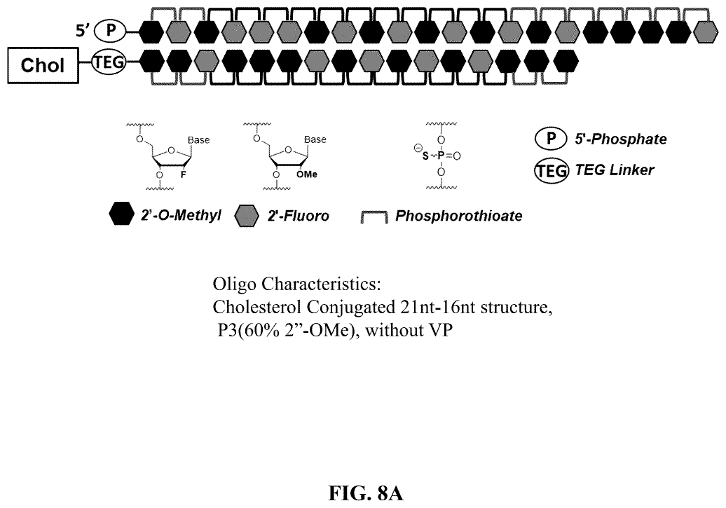

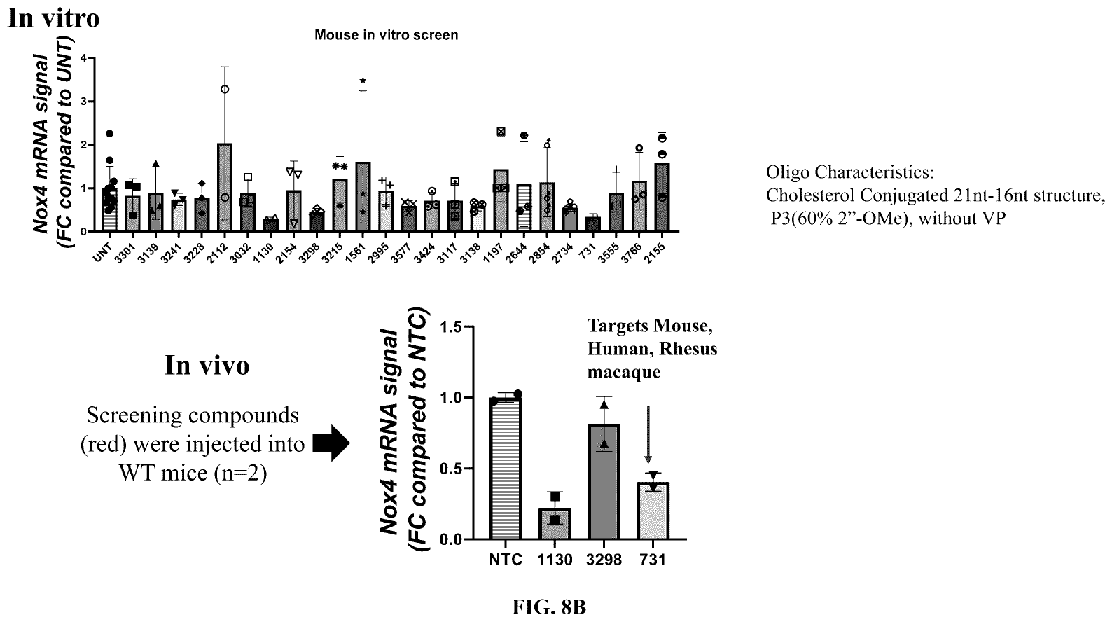

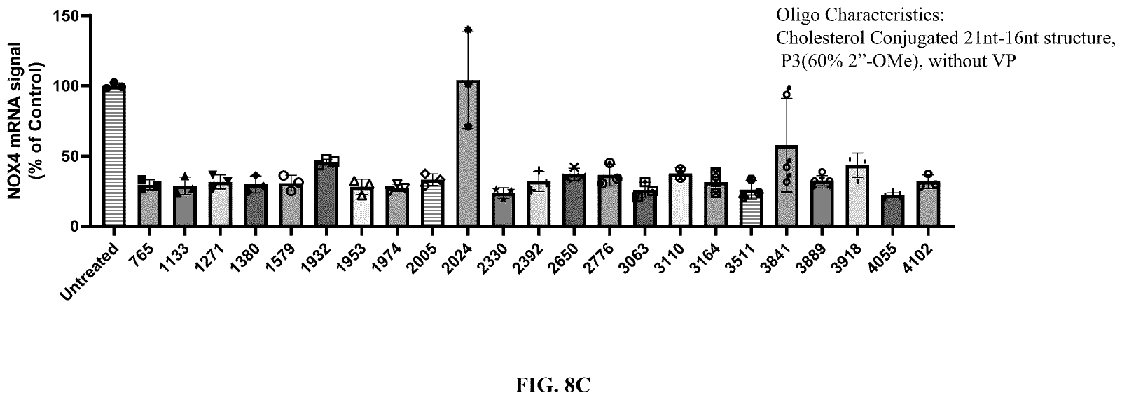

The NOX4 screening of siRNA compounds was assessed (FIG. 8A-8C).

The initial in vitro screening was carried out by plating 20K cells/well of Lx2 cells for human (FIG. 8C), 3T3 cells for mouse (FIG. 8B), in a 96 well plate. The cells were treated with the candidate compounds in final concentration of 1.5 μM. For human screening, Lx2 cells were activated with Tgfb (5 ng/ml) 10 hrs after the addition of siRNAs in the media. Cells were treated for 60 additional hours at 37° C., 5% CO2 in 3% FBS DMEM medium. For mouse screening 3T3 cell were treated for 70 hours at 37° C., 5% CO2 in 3% FBS DMEM medium After total of 70 hours, mRNA levels were quantified using the QuantiGene 2.0 assay kit (Affymetrix, QS0011) as previously described by Coles et al, 2016 and Nikan et al, 2016. Briefly, cells were lysed in 300 μL diluted lysis mixture composed of one-part lysis mixture (Affymetrix, QG0503), 2 parts H2O, and 0.167 μg/μL proteinase K (ThermoScientific, E00491) for 1 hour at 55° C. Cell lysates were mixed thoroughly, and 20 μL/well of each lysate was added to the capture plate with 60 μL diluted lysis mixture without proteinase K. Probe sets for Nox4 and B2m (Affymetrix; 80246, 80003) were used according to the manufacturer's recommended protocol. Datasets were normalized to B2m. The dose response investigation was done using serially diluted doses of the two candidate compounds (1.500, 0.750, 0.375, 0.188, 0.094, 0.047, 0.023, 0.012 μM), each condition in triplicate and QuantiGene 2.0 Assay was carried out. For mouse in vivo trials C57BL6 mice were injected with the hit siRNAs from in vitro screen (10 mg/kg) subcutaneously and sacrificed 1 week after injection. Livers were harvested and OPN (Spp1) mRNA level measurements were done via qPCR. (FIG. 8B)

### Example 9. DGAT2 and FASN Silencing Using siRNA-siRNA and siRNA-ASO Compounds

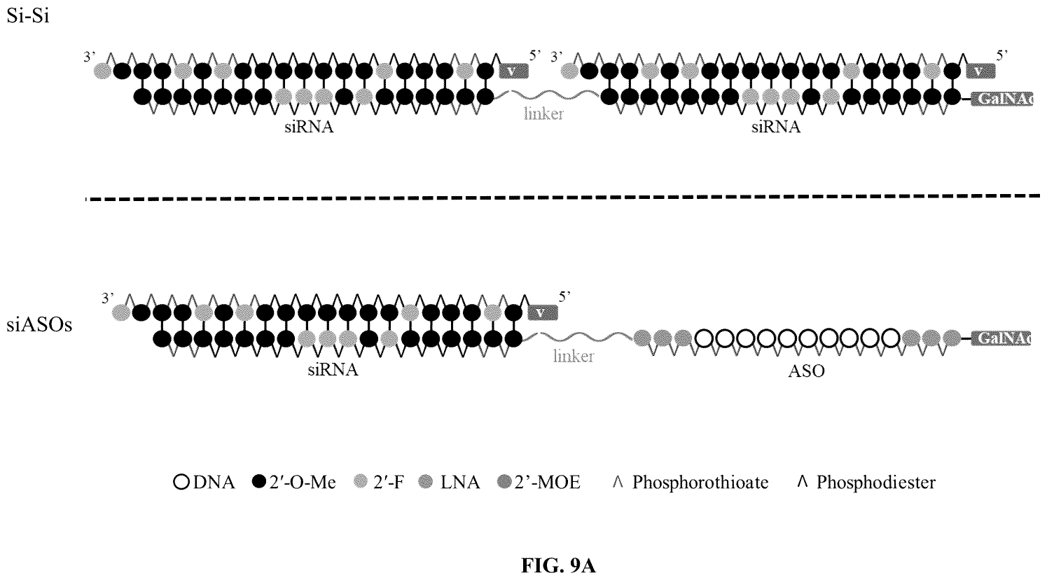

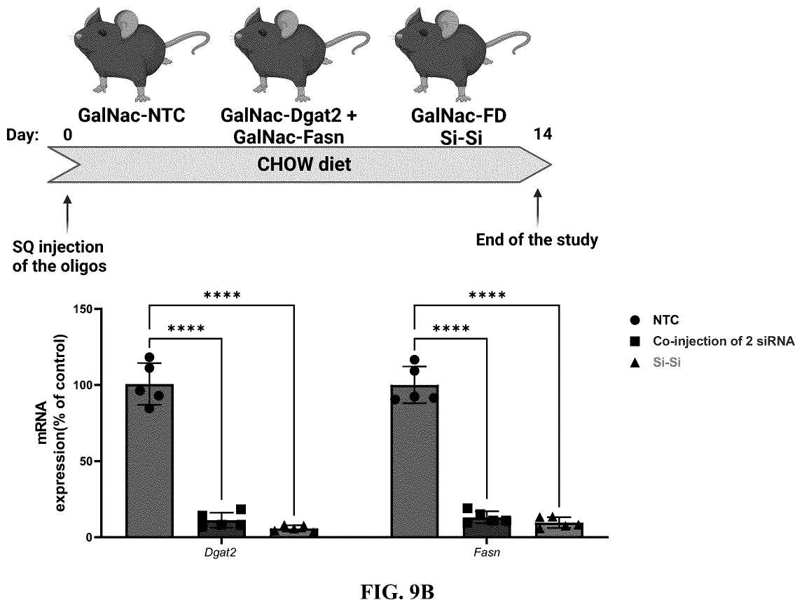

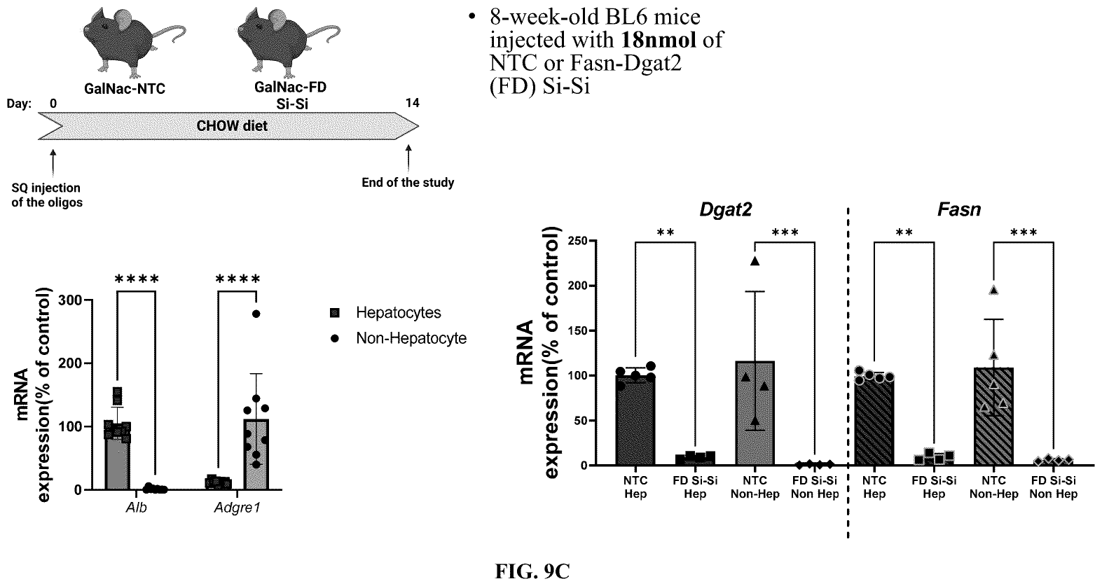

The DGAT2 and FASN silencing using siRNA-siRNA and siRNA-ASO compounds was assessed (FIG. 9A-9C).

8-week-old C57BL6 mice were subcutaneously injected with either GalNac conjugated NTC (20 nmols); co-injected with GalNac Dgat2 (10 nmol) and GalNac-Fasn, or injected with GalNac conjugated siRNA-siRNA (Si—Si) compounds targeting Dgat2 and Fasn (GalNac FD Si—Si) (10 nmols) 14 days post injection mice were sacrificed, livers were collected. Dgat2 and Fasn mRNA levels were measured by qPCR (FIG. 9B).

8-week-old C57BL6 mice were subcutaneously injected with either GalNac conjugated NTC (20 nmols) or injected with GalNac FD Si—Si (10 nmols) 14 days post injection mice were sacrificed (FIG. 9C). Livers were perfused with liberase to dissociate the hepatocyte cell population from non-hepatocyte population. RNA isolation of both fractions from each mouse were done via Trizol method. Alb mRNA (for hepatocyte marker) and Adgre1 (Kuppfer cell marker aka Non hepatocyte fraction marker in this case) were measured to ensure the separation of 2 fractions was successful. Later Dgat2 and Fasn mRNA levels were measured, and the data was normalized to the B2m mRNA levels.

### Example 10. DGAT2 and MALAT1 Silencing Using a siRNA-ASO Compound

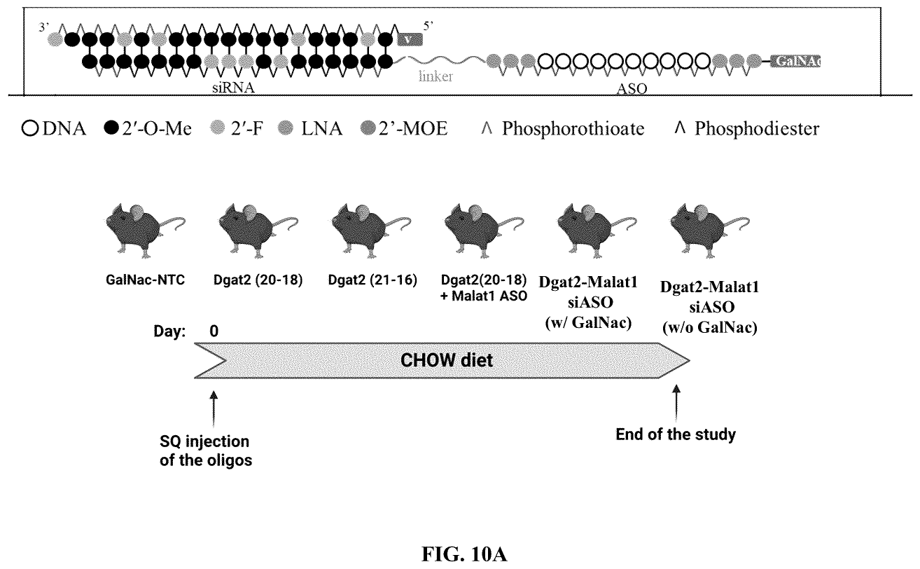

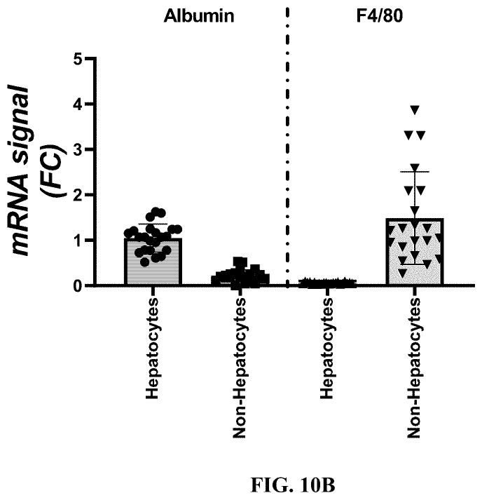

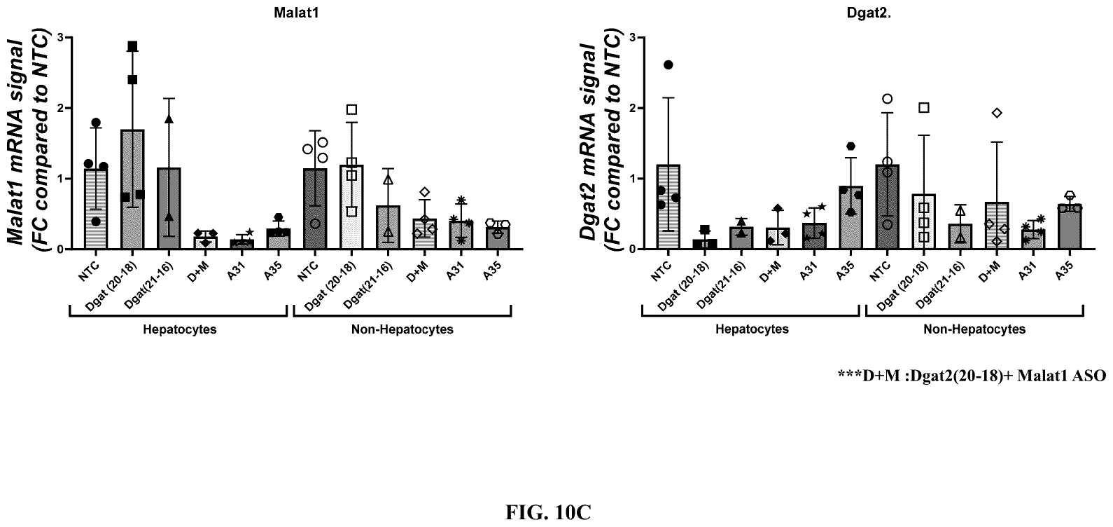

The DGAT2 and MALAT1 silencing using a siRNA-ASO compound was assessed (FIG. 10A-10B). Four male, C57BL6/J, and 8 weeks old mice were selected. Each mouse was injected with corresponding doses via subcutaneous injection. Mice were sacrificed at Day 7, and liver perfusion and digestion were performed in order to isolate hepatocyte and non-hepatocyte cell fractions. The total RNA was isolated via Trizol method. Alb mRNA (for hepatocyte marker) and Adgre1 (Kuppfer cell marker aka Non hepatocyte fraction marker in this case) were measured to ensure the separation of 2 fractions was successful. Later the silencing of Dgat2 and/or Malat1 as well as purity of hepatocyte vs non-hepatocyte fractions were examined via qPCR. The data was normalized to the B2m mRNA levels.

### Example 11. DGAT2 and FASN Silencing Using a siRNA-ASO Compound

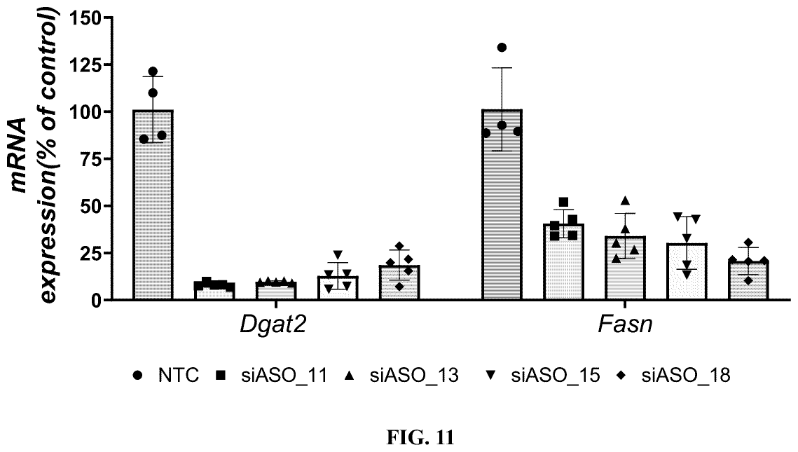

The DGAT2 and FASN silencing using a siRNA-ASO compound was assessed (FIG. 11)

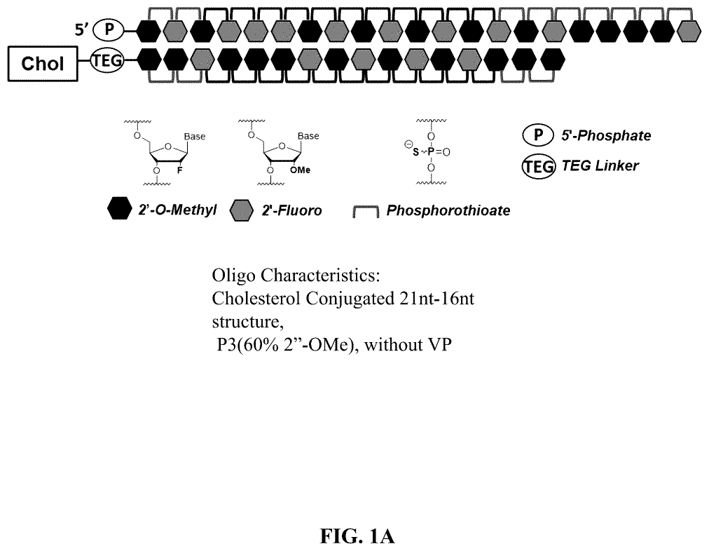

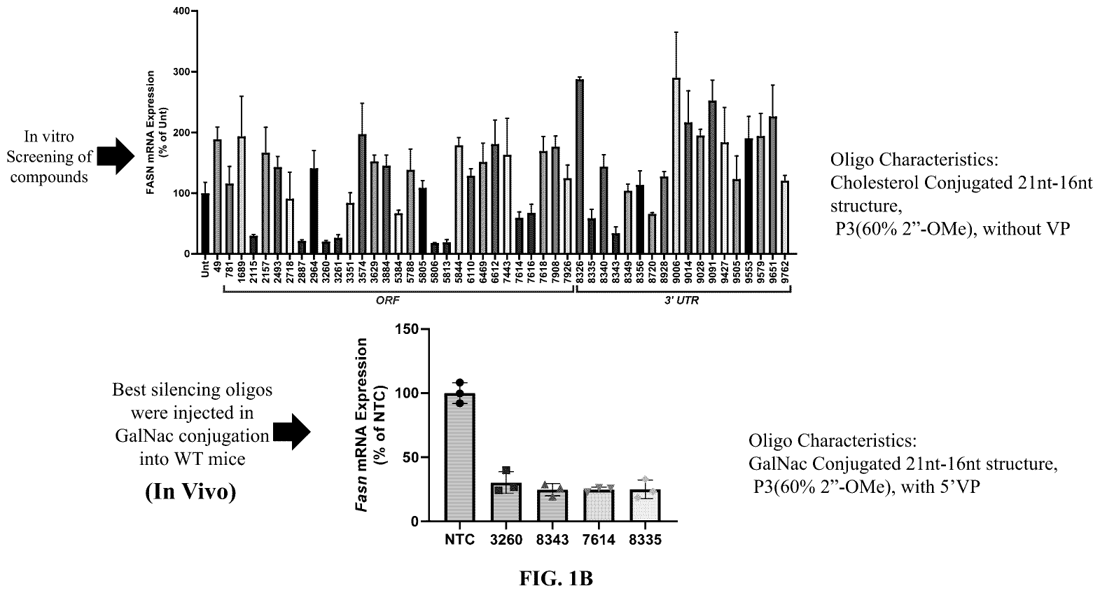

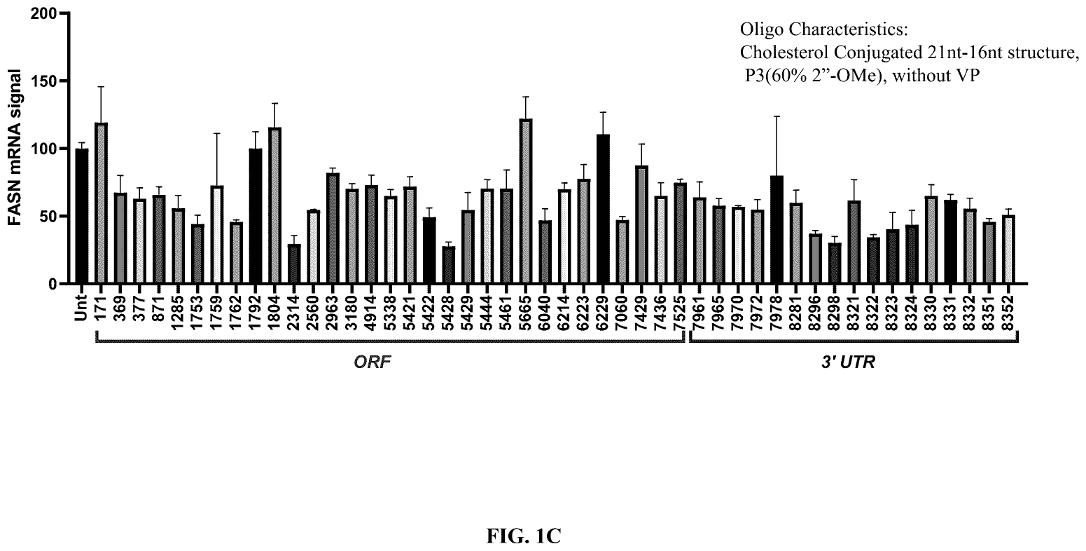

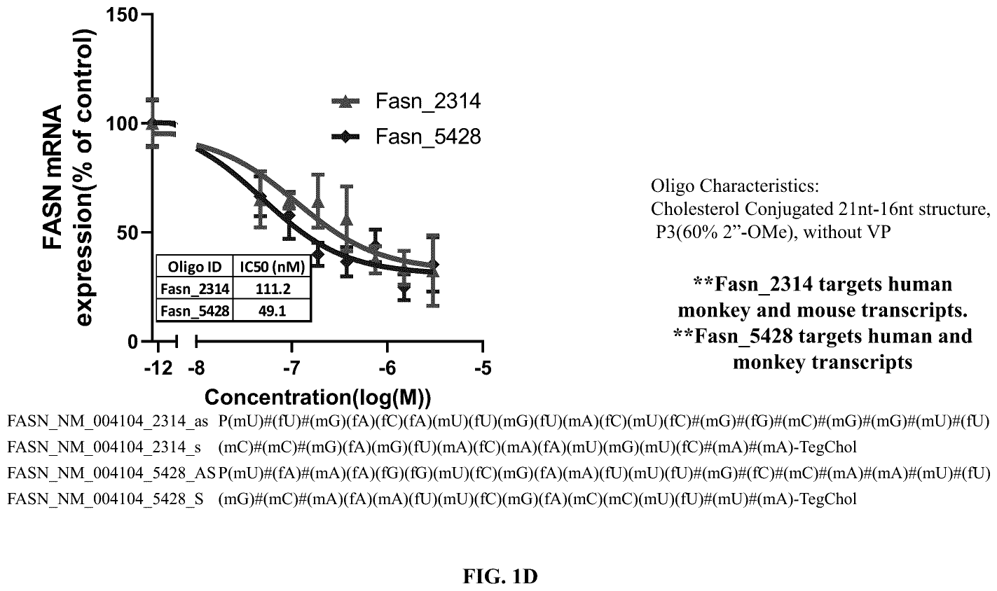

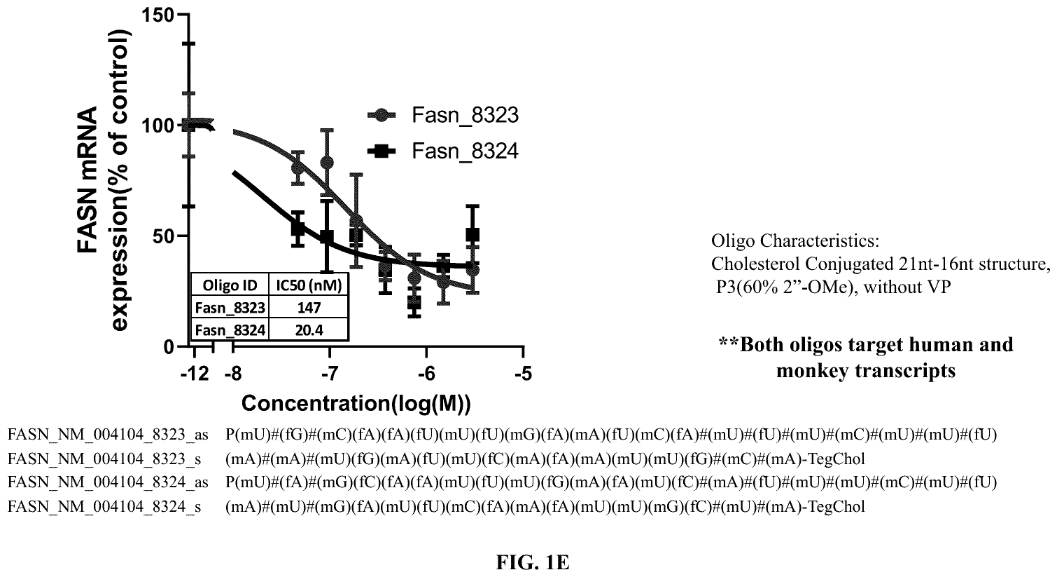

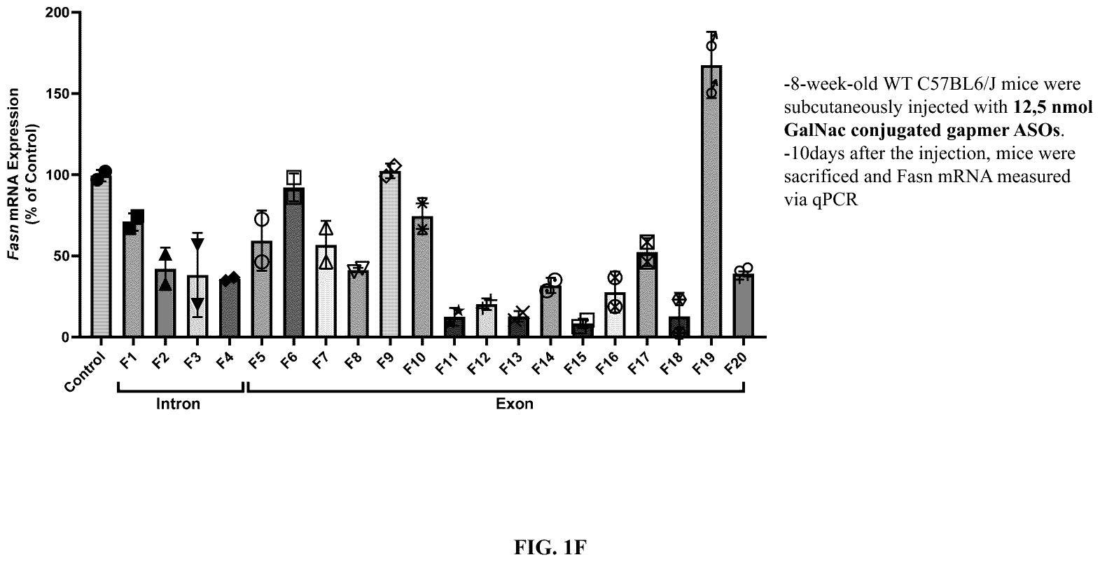

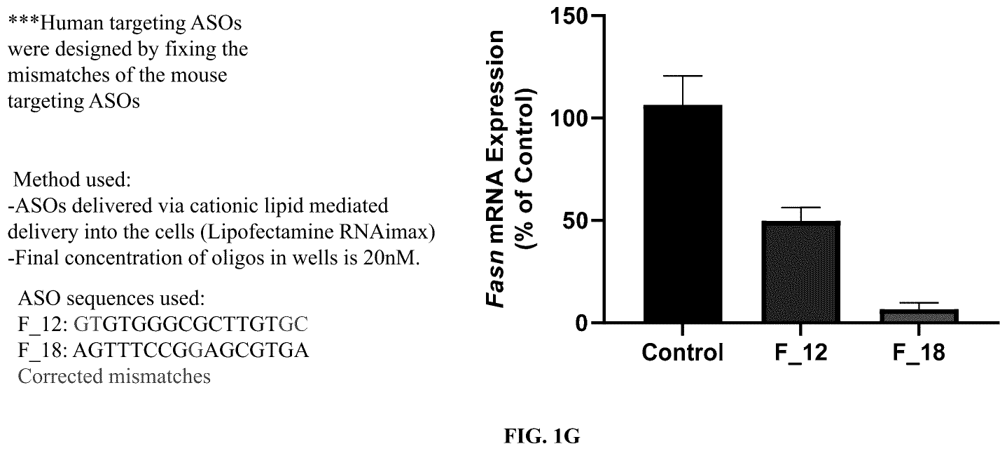

The DGAT2 and FASN silencing using a siRNA-ASO compound was assessed. ASO hits from initial in vivo screening (FIG. 1E) were linked to Dgat2 siRNA for this assessment. Five male, C57BL6/J, and 8 weeks old mice were selected. Each mouse were injected with 12 nmol of corresponding compound via subcutaneous injection. Mice were sacrificed at Day 7, and livers were harvested for RNA isolation via Trizol. Dgat2 and Fasn mRNA levels were measured via qPCR, and the data was normalized to the B2m mRNA level.

TABLE 1
DGAT2 gene target regions and antisense and sense strands targeting said DGAT2
gene target regions.

| | | | | Sense | Sense |
|---|---|---|---|---|---|
| Sequence | Target | Target Region | Antisense | Sequence | Sequence |
| ID | Region | (20 nucleotide) | Sequence | (Asymmetric) | (Blunt) |
| DGAT2_m14 | TGGAGGAACC | ATGGGTGTCT | UAUAACCCAC | GGGUGUCUGU | AUGGGUGU |
| 64_h1372 | AGCTGCAAAT | GTGGGTTATT | AGACACCCAU | GGGUUAUA | CUGUGGGU |
| | CACTTTTTTGC | (SEQ ID NO: 6) | (SEQ ID NO: 11) | (SEQ ID NO: 16) | UAUA (SEQ |
| | TCTGTAAATTT | | | | ID NO: 21) |
| | GGAAGTGTCA | | | | |
| | TGGGTGTCTGT | | | | |
| | GGGTTATTTA | | | | |
| | AAAGAAATTA | | | | |
| | TAACAATTTTG | | | | |
| | CTAAACCATT | | | | |
| | ACAATGTTAG | | | | |
| | GTCTTT (SEQ | | | | |
| | ID NO: 1) | | | | |
| DGAT2_m14 | TGGAGGAA | TGTGGGTTATT | UUUCUUUU | UGGGUUAU | UGUGGGUU |
| 73_h1381 | CCAGCTGC | TAAAAGAA | AAAUAACC | UUAAAAGA | AUUUAAAA |
| | AAATCACTT | (SEQ ID NO: 7) | CACA (SEQ | AA (SEQ ID | GAAA (SEQ |
| | TTTTGCTCT | | ID NO: 12) | NO: 17) | ID NO: 22) |
| | GTAAATTTG | | | | |
| | GAAGTGTC | | | | |
| | ATGGGTGT | | | | |
| | CTGTGGGTT | | | | |
| | ATTTAAAA | | | | |
| | GAAATTAT | | | | |
| | AACAATTTT | | | | |
| | GCTAAACC | | | | |
| | ATTACAAT | | | | |
| | GTTAGGTCT | | | | |
| | TT (SEQ ID | | | | |
| | NO: 2) | | | | |
| DGAT2_m14 | TGGAGGAA | GGGTTATTTAA | UAAUUUCU | GUUAUUUA | GGGUUAUU |
| 76_h1384 | CCAGCTGC | AAGAAATTA | UUUAAAUA | AAAGAAAU | UAAAAGAA |
| | AAATCACTT | (SEQ ID NO: 8) | ACCC (SEQ | UA (SEQ ID | AUUA (SEQ |
| | TTTTGCTCT | | ID NO: 13) | NO: 18) | ID NO: 23) |
| | GTAAATTTG | | | | |
| | GAAGTGTC | | | | |
| | ATGGGTGT | | | | |
| | CTGTGGGTT | | | | |
| | ATTTAAAA | | | | |
| | GAAATTAT | | | | |
| | AACAATTTT | | | | |
| | GCTAAACC | | | | |
| | ATTACAAT | | | | |
| | GTTAGGTCT | | | | |
| | TT (SEQ ID | | | | |
| | NO: 3) | | | | |
| DGAT2_m10 | AGGAGGGC | TGGGTCCAGA | UGGAACUU | GGUCCAGA | UGGGUCCA |
| 93_h1022 | TCCTGGGG | AGAAGTTCCA | CUUCUGGA | AGAAGUUC | GAAGAAGU |
| | CCGATGGG | (SEQ ID NO: 9) | CCCA (SEQ | CA (SEQ ID | UCCA (SEQ |
| | TCCAGAAG | | ID NO: 14) | NO: 19) | ID NO: 24) |
| | AAGTTCCA | | | | |
| | GAAATACA | | | | |
| | TTGGTTTCG | | | | |
| | CCCCATGC | | | | |
| | ATCTTCCAT | | | | |
| | GGTCG (SEQ | | | | |
| | ID NO: 4) | | | | |
| DGAT2_m10 | AGGAGGGC | GGGTCCAGAA | UUGGAACU | GUCCAGAA | GGGUCCAG |
| 94_h1023 | TCCTGGGG | GAAGTTCCAG | UCUUCUGG | GAAGUUCC | AAGAAGUU |
| | CCGATGGG | (SEQ ID | ACCC (SEQ | AA (SEQ ID | CCAA (SEQ |
| | TCCAGAAG | NO: 10) | ID NO: 15) | NO: 20) | ID NO: 25) |
| | AAGTTCCA | | | | |
| | GAAATACA | | | | |
| | TTGGTTTCG | | | | |
| | CCCCATGC | | | | |
| | ATCTTCCAT | | | | |
| | GGTCG (SEQ | | | | |
| | ID NO: 5) | | | | |

TABLE 2
Modified DGAT2 mRNA targeting antisense (as) strand
sequences with a 5′ phosphate and complementary cholesterol
conjugated sense (s) strand sequences.

| OLIGO ID | Modified Sequence |
|---|---|
| DGAT2_m146 | P(mU)#(fA)#(mU)(mA)(mA)(fC)(mC)(mC)(mA)(mC)(mA)(mG)(mA)#( |
| 4_h1372-as | fC)#(mA)#(fC)#(mC)#(mC)#(mA)#(fU) (SEQ ID NO: 26) |
| DGAT2_m147 | P(mU)#(fU)#(mU)(mC)(mU)(fU)(mU)(mU)(mA)(mA)(mA)(mU)(mA)# |
| 3_h1381-as | (fA)#(mC)#(fC)#(mC)#(mA)#(mC)#(fA) (SEQ ID NO: 27) |
| DGAT2_m147 | P(mU)#(fA)#(mA)(mU)(mU)(fU)(mC)(mU)(mU)(mU)(mU)(mA)(mA)# |
| 6_h1384-as | (fA)#(mU)#(fA)#(mA)#(mC)#(mC)#(fC) (SEQ ID NO: 28) |
| DGAT2_m109 | P(mU)#(fG)#(mG)(mA)(mA)(fC)(mU)(mU)(mC)(mU)(mU)(mC)(mU)# |
| 3_h1022-as | (fG)#(mG)#(fA)#(mC)#(mC)#(mC)#(fA) (SEQ ID NO: 29) |
| DGAT2_m109 | P(mU)#(fU)#(mG)(mG)(mA)(fA)(mC)(mU)(mU)(mC)(mU)(mU)(mC)# |
| 4_h1023-as | (fU)#(mG)#(fG)#(mA)#(mC)#(mC)#(fC) (SEQ ID NO: 30) |
| DGAT2_m146 | (mG)#(mG)#(mG)(mU)(mG)(mU)(mC)(fU)(fG)(fU)(mG)(fG)(mG)(mU) |
| 4_h1372-s (A) | (mU)(mA)#(mU)#(mA)-TegChol (SEQ ID NO: 31) |
| DGAT2_m147 | (mU)#(mG)#(mG)(mG)(mU)(mU)(mA)(fU)(fU)(fU)(mA)(fA)(mA)(mA) |
| 3_h1381-s (A) | (mG)(mA)#(mA)#(mA)-TegChol (SEQ ID NO: 32) |
| DGAT2_m147 | (mG)#(mU)#(mU)(mA)(mU)(mU)(mU)(fA)(fA)(fA)(mA)(fG)(mA) |
| 6_h1384-s (A) | (mA)(mA)(mU)#(mU)#(mA)-TegChol (SEQ ID NO: 33) |
| DGAT2_m109 | (mG)#(mG)#(mU)(mC)(mC)(mA)(mG)(fA)(fA)(fG)(mA)(fA)(mG)(mU) |
| 3_h1022-s (A) | (mU)(mC)#(mC)#(mA)-TegChol (SEQ ID NO: 34) |
| DGAT2_m109 | (mG)#(mU)#(mC)(mC)(mA)(mG)(mA)(fA)(fG)(fA)(mA)(fG)(mU) |
| 4_h1023-s (A) | (mU)(mC)(mC)#(mA)#(mA)-TegChol (SEQ ID NO: 35) |
| DGAT2_m146 | (mA)#(mU)#(mG)(mG)(mG)(mU)(mG)(mU)(mC)(fU)(fG)(fU)(mG) |
| 4_h1372-s (B) | (fG)(mG)(mU)(mU)(mA)#(mU)#(mA)-TegChol (SEQ ID NO: 36) |
| DGAT2_m147 | (mU)#(mG)#(mU)(mG)(mG)(mG)(mU)(mU)(mA)(fU)(fU)(fU)(mA) |
| 3_h1381-s (B) | (fA)(mA)(mA)(mG)(mA)#(mA)#(mA)-TegChol (SEQ ID NO: 37) |
| DGAT2_m147 | (mG)#(mG)#(mG)(mU)(mU)(mA)(mU)(mU)(mU)(fA)(fA)(fA)(mA) |
| 6_h1384-s (B) | (fG)(mA)(mA)(mA)(mU)#(mU)#(mA)-TegChol (SEQ ID NO: 38) |
| DGAT2_m109 | (mU)#(mG)#(mG)(mG)(mU)(mC)(mC)(mA)(mG)(fA)(fA)(fG)(mA)(fA) |
| 3_h1022-s (B) | (mG)(mU)(mU)(mC)#(mC)#(mA)-TegChol (SEQ ID NO: 39) |
| DGAT2_m109 | (mG)#(mG)#(mG)(mU)(mC)(mC)(mA)(mG)(mA)(fA)(fG)(fA)(mA)(fG) |
| 4_h1023-s (B) | (mU)(mU)(mC)(mC)#(mA)#(mA)-TegChol (SEQ ID NO: 40) |

“m” represents a 2′-O-methyl nucleotide modification; “f” represents a 2′-Fluoro nucleotide modification; “#” represents a phosphorothioate internucleotide linkage; “P” represents a 5′ phosphate; “TegChol” represents cholesterol with a TEG linker.

TABLE 3
Modified DGAT2 mRNA targeting antisense (as) strand sequences with a 5′-(E)-
vinylphosphonate (VP) and complementary cholesterol conjugated
sense (s) strand sequences.

| OLIGO ID | Modified Sequence |
|---|---|
| DGAT2_m146 | VP(mU)#(fA)#(mU)(mA)(mA)(fC)(mC)(mC)(mA)(mC)(mA)(mG)(mA) |
| 4_h1372-as | #(fC)#(mA)#(fC)#(mC)#(mC)#(mA)#(fU) (SEQ ID NO: 41) |
| DGAT2_m147 | VP(mU)#(fU)#(mU)(mC)(mU)(fU)(mU)(mU)(mA)(mA)(mA)(mU)(mA) |
| 3_h1381-as | #(fA)#(mC)#(fC)#(mC)#(mA)#(mC)#(fA) (SEQ ID NO: 42) |
| DGAT2_m147 | VP(mU)#(fA)#(mA)(mU)(mU)(fU)(mC)(mU)(mU)(mU)(mU)(mA)(mA) |
| 6_h1384-as | #(fA)#(mU)#(fA)#(mA)#(mC)#(mC)#(fC) (SEQ ID NO: 43) |
| DGAT2_m109 | VP(mU)#(fG)#(mG)(mA)(mA)(fC)(mU)(mU)(mC)(mU)(mU)(mC)(mU) |
| 3_h1022-as | #(fG)#(mG)#(fA)#(mC)#(mC)#(mC)#(fA) (SEQ ID NO: 44) |
| DGAT2_m109 | VP(mU)#(fU)#(mG)(mG)(mA)(fA)(mC)(mU)(mU)(mC)(mU)(mU)(mC) |
| 4_h1023-as | #(fU)#(mG)#(fG)#(mA)#(mC)#(mC)#(fC) (SEQ ID NO: 45) |
| DGAT2_m146 | (mG)#(mG)#(mG)(mU)(mG)(mU)(mC)(fU)(fG)(fU)(mG)(fG)(mG)(mU) |
| 4_h1372-s (A) | (mU)(mA)#(mU)#(mA)-TegChol (SEQ ID NO: 31) |
| DGAT2_m147 | (mU)#(mG)#(mG)(mG)(mU)(mU)(mA)(fU)(fU)(fU)(mA)(fA)(mA)(mA |
| 3_h1381-s (A) | )(mG)(mA)#(mA)#(mA)-TegChol (SEQ ID NO: 32) |
| DGAT2_m147 | (mG)#(mU)#(mU)(mA)(mU)(mU)(mU)(fA)(fA)(fA)(mA)(fG)(mA)(mA |
| 6_h1384-s (A) | )(mA)(mU)#(mU)#(mA)-TegChol (SEQ ID NO: 33) |
| DGAT2_m109 | (mG)#(mG)#(mU)(mC)(mC)(mA)(mG)(fA)(fA)(fG)(mA)(fA)(mG)(mU) |
| 3_h1022-s (A) | (mU)(mC)#(mC)#(mA)-TegChol (SEQ ID NO: 34) |
| DGAT2_m109 | (mG)#(mU)#(mC)(mC)(mA)(mG)(mA)(fA)(fG)(fA)(mA)(fG)(mU) |
| 4_h1023-s (A) | (mU)(mC)(mC)#(mA)#(mA)-TegChol (SEQ ID NO: 35) |
| DGAT2_m146 | (mA)#(mU)#(mG)(mG)(mG)(mU)(mG)(mU)(mC)(fU)(fG)(fU)(mG)(fG) |
| 4_h1372-s (B) | (mG)(mU)(mU)(mA)#(mU)#(mA)-TegChol (SEQ ID NO: 36) |
| DGAT2_m147 | (mU)#(mG)#(mU)(mG)(mG)(mG)(mU)(mU)(mA)(fU)(fU)(fU)(mA)(fA |
| 3_h1381-s (B) | )(mA)(mA)(mG)(mA)#(mA)#(mA)-TegChol (SEQ ID NO: 37) |
| DGAT2_m147 | (mG)#(mG)#(mG)(mU)(mU)(mA)(mU)(mU)(mU)(fA)(fA)(fA)(mA)(fG |
| 6_h1384-s (B) | )(mA)(mA)(mA)(mU)#(mU)#(mA)-TegChol (SEQ ID NO: 38) |
| DGAT2_m109 | (mU)#(mG)#(mG)(mG)(mU)(mC)(mC)(mA)(mG)(fA)(fA)(fG)(mA)(fA) |
| 3_h1022-s (B) | (mG)(mU)(mU)(mC)#(mC)#(mA)-TegChol (SEQ ID NO: 39) |
| DGAT2_m109 | (mG)#(mG)#(mG)(mU)(mC)(mC)(mA)(mG)(mA)(fA)(fG)(fA)(mA)(fG) |
| 4_h1023-s (B) | (mU)(mU)(mC)(mC)#(mA)#(mA)-TegChol (SEQ ID NO: 40) |

“m” represents a 2′-O-methyl nucleotide modification; “f” represents a 2′-Fluoro nucleotide modification; “#” represents a phosphorothioate internucleotide linkage; “VP” represents a 5′-(E)-vinylphosphonate; “TegChol” represents cholesterol with a TEG linker.

TABLE 4
Modified DGAT2 mRNA targeting antisense (as) strand sequences with
a 5′-(E)-vinylphosphonate (VP) and complementary GalNAc
conjugated sense (s) strand sequences.

| OLIGO ID | Modified Sequence |
|---|---|
| DGAT2_m146 | VP(mU)#(fA)#(mU)(mA)(mA)(fC)(mC)(mC)(mA)(mC)(mA)(mG)(mA) |
| 4_h1372-as | #(fC)#(mA)#(fC)#(mC)#(mC)#(mA)#(fU) (SEQ ID NO: 41) |
| DGAT2_m147 | VP(mU)#(fU)#(mU)(mC)(mU)(fU)(mU)(mU)(mA)(mA)(mA)(mU)(mA) |
| 3_h1381-as | #(fA)#(mC)#(fC)#(mC)#(mA)#(mC)#(fA) (SEQ ID NO: 42) |
| DGAT2_m147 | VP(mU)#(fA)#(mA)(mU)(mU)(fU)(mC)(mU)(mU)(mU)(mU)(mA)(mA) |
| 6_h1384-as | #(fA)#(mU)#(fA)#(mA)#(mC)#(mC)#(fC) (SEQ ID NO: 43) |
| DGAT2_m109 | VP(mU)#(fG)#(mG)(mA)(mA)(fC)(mU)(mU)(mC)(mU)(mU)(mC)(mU) |
| 3_h1022-as | #(fG)#(mG)#(fA)#(mC)#(mC)#(mC)#(fA) (SEQ ID NO: 44) |
| DGAT2_m109 | VP(mU)#(fU)#(mG)(mG)(mA)(fA)(mC)(mU)(mU)(mC)(mU)(mU)(mC) |
| 4_h1023-as | #(fU)#(mG)#(fG)#(mA)#(mC)#(mC)#(fC) (SEQ ID NO: 45) |
| DGAT2_m146 | (mG)#(mG)#(mG)(mU)(mG)(mU)(mC)(fU)(fG)(fU)(mG)(fG)(mG)(mU) |
| 4_h1372-s (A) | (mU)(mA)#(mU)#(mA)-GalNAc (SEQ ID NO: 46) |
| DGAT2_m147 | (mU)#(mG)#(mG)(mG)(mU)(mU)(mA)(fU)(fU)(fU)(mA)(fA)(mA)(mA |
| 3_h1381-s (A) | )(mG)(mA)#(mA)#(mA)-GalNAc (SEQ ID NO: 47) |
| DGAT2_m147 | (mG)#(mU)#(mU)(mA)(mU)(mU)(mU)(fA)(fA)(fA)(mA)(fG)(mA)(mA |
| 6_h1384-s (A) | )(mA)(mU)#(mU)#(mA)-GalNAc (SEQ ID NO: 48) |
| DGAT2_m109 | (mG)#(mG)#(mU)(mC)(mC)(mA)(mG)(fA)(fA)(fG)(mA)(fA)(mG)(mU) |
| 3_h1022-s (A) | (mU)(mC)#(mC)#(mA)-GalNAc (SEQ ID NO: 49) |
| DGAT2_m109 | (mG)#(mU)#(mC)(mC)(mA)(mG)(mA)(fA)(fG)(fA)(mA)(fG)(mU) |
| 4_h1023-s (A) | (mU)(mC)(mC)#(mA)#(mA)-GalNAc (SEQ ID NO: 50) |
| DGAT2_m146 | (mA)#(mU)#(mG)(mG)(mG)(mU)(mG)(mU)(mC)(fU)(fG)(fU)(mG)(fG) |
| 4_h1372-s (B) | (mG)(mU)(mU)(mA)#(mU)#(mA)-GalNAc (SEQ ID NO: 51) |
| DGAT2_m147 | (mU)#(mG)#(mU)(mG)(mG)(mG)(mU)(mU)(mA)(fU)(fU)(fU)(mA)(fA |
| 3_h1381-s (B) | )(mA)(mA)(mG)(mA)#(mA)#(mA)-GalNAc (SEQ ID NO: 52) |
| DGAT2_m147 | (mG)#(mG)#(mG)(mU)(mU)(mA)(mU)(mU)(mU)(fA)(fA)(fA)(mA)(fG |
| 6_h1384-s (B) | )(mA)(mA)(mA)(mU)#(mU)#(mA)-GalNAc (SEQ ID NO: 53) |
| DGAT2_m109 | (mU)#(mG)#(mG)(mG)(mU)(mC)(mC)(mA)(mG)(fA)(fA)(fG)(mA)(fA) |
| 3_h1022-s (B) | (mG)(mU)(mU)(mC)#(mC)#(mA)-GalNAc (SEQ ID NO: 54) |
| DGAT2_m109 | (mG)#(mG)#(mG)(mU)(mC)(mC)(mA)(mG)(mA)(fA)(fG)(fA)(mA)(fG) |
| 4_h1023-s (B) | (mU)(mU)(mC)(mC)#(mA)#(mA)-GalNAc (SEQ ID NO: 55) |

“m” represents a 2′-O-methyl nucleotide modification; “F” represents a 2′-Fluoro nucleotide modification; “#” represents a phosphorothioate internucleotide linkage; “VP” represents a 5′-(E)-vinylphosphonate; “GalNAc” represents a GalNAc conjugate.

TABLE 5
Sequences targeting human FASN gene target regions.

| Se- | | SEQ | | SEQ | | SEQ |
|---|---|---|---|---|---|---|
| quence | Target | ID | | ID | Reverse | ID |
| ID | Region | NO | Sequence | NO | Complement | NO |
| FASN_N | GGCACCAACT | 658 | UUUGGUGUU | 705 | AAACACAGA | 755 |
| M_00410 | CCATGTTTGG | | UGUCUGUGU | | CAAACACCA | |
| 4_8296 | TGTTTGTCTG | | UU | | AA | |
| | TGTTTGTTTT | | | | | |
| | TCAAG | | | | | |
| FASN_N | AATTGCTGCT | 659 | UUUGAAAUU | 706 | CAGUUACAG | 756 |
| M_00410 | TGGATTTTGA | | UACUGUAAC UG | | UAAAUUUCA | |
| 4_8351 | AATTTACTGT | | | | AA | |
| | AACTGTCAGT | | | | | |
| | GTACA | | | | | |
| FASN_N | TTATGCCCGG | 660 | GGCACCAAC | 707 | CCAAACAUG | 757 |
| M_00410 | CGCCGGGCAC | | UCCAUGUUU | | GAGUUGGUG | |
| 4_8281 | CAACTCCATG | | GG | | CC | |
| | TTTGGTGTTT | | | | | |
| | GTCTG | | | | | |
| FASN_N | TGTTTTTCAA | 661 | GAUUCAAAU | 708 | UCCAAGCAG | 758 |
| M_00410 | GAAATGATTC | | UGCUGCUUG | | CAAUUUGAA | |
| 4_8330 | AAATTGCTGC | | GA | | UC | |
| | TTGGATTTTG | | | | | |
| | AAATT | | | | | |
| FASN_N | CTGAGCACAG | 662 | AGCACCUUU | 709 | ACGAUGUCA | 759 |
| M_00410 | ACGAGAGCA | | GAUGACAUC | | UCAAAGGUG | |
| 4_1753 | CCTTTGATGA | | GU | | CU | |
| | CATCGTCCAT | | | | | |
| | TCGTTT | | | | | |
| FASN_N | GTCCGTGGCG | 663 | GUGGGCAUC | 710 | CCAAAGGAG | 760 |
| M_00410 | GCAACGTGGG | | AACUCCUUU | | UUGAUGCCC | |
| 4_1285 | CATCAACTCC | | GG | | AC | |
| | TTTGGCTTCG | | | | | |
| | GGGGC | | | | | |
| FASN_N | TACGCACGGT | 664 | CCUGGAAAU | 711 | CGAAUUUGC | 761 |
| M_00410 | CGCTTCCTGG | | UGGCAAAUU | | CAAUUUCCA | |
| 4_5421 | AAATTGGCAA | | CG | | GG | |
| | ATTCGACCTT | | | | | |
| | TCTCA | | | | | |
| FASN_N | ACCATCCTGA | 665 | GGCACCAAU | 712 | AAGCCAUCU | 762 |
| M_00410 | ACGCCGGCAC | | ACAGAUGGC | | GUAUUGGUG | |
| 4_871 | CAATACAGAT | | UU | | CC | |
| | GGCTTCAAGG | | | | | |
| | AGCAA | | | | | |
| FASN_N | TCTGTGTTTG | 666 | CAAGAAAUG | 713 | GCAAUUUGA | 763 |
| M_00410 | TTTTTCAAGA | | AUUCAAAUU | | AUCAUUUCU | |
| 4_8322 | AATGATTCAA | | GC | | UG | |
| | ATTGCTGCTT | | | | | |
| | GGATT | | | | | |
| FASN_N | GTTTTTCAAG | 667 | AUUCAAAUU | 714 | AUCCAAGCA | 764 |
| M_00410 | AAATGATTCA | | GCUGCUUGG | | GCAAUUUGA | |
| 4_8331 | AATTGCTGCT | | AU | | AU | |
| | TGGATTTTGA | | | | | |
| | AATTT | | | | | |
| FASN_N | GGGTCTGCGC | 668 | CUUUCUGUG | 715 | GCAAAUCCA | 765 |
| M_00410 | TTGGTCTTTC | | CUUGGAUUU | | AGCACAGAA | |
| 4_7961 | TGTGCTTGGA | | GC | | AG | |
| | TTTGCATATT | | | | | |
| | TATTG | | | | | |
| FASN_N | TCTCAGAACC | 669 | CUCGGCAUG | 716 | AGGAAGAUA | 766 |
| M_00410 | ACCCGCTCGG | | GCUAUCUUC | | GCCAUGCCG | |
| 4_5461 | CATGGCTATC | | CU | | AG | |
| | TTCCTGAAGA | | | | | |
| | ACGTG | | | | | |
| FASN_N | TCCTACGGGG | 670 | GUGGCCUUU | 717 | GAGCACAUU | 767 |
| M_00410 | CCTGCGTGGC | | GAAAUGUGC | | UCAAAGGCC | |
| 4_7060 | CTTTGAAATG | | UC | | AC | |
| | TGCTCCCAGC | | | | | |
| | TGCAG | | | | | |
| FASN_N | CTGCGCTTGG | 671 | CUGUGCUUG | 718 | AUAUGCAAA | 768 |
| M_00410 | TCTTTCTGTG | | GAUUUGCAU | | UCCAAGCAC | |
| 4_7965 | CTTGGATTTG | | AU | | AG | |
| | CATATTTATT | | | | | |
| | GCATT | | | | | |
| FASN_N | ATTGCTGCTT | 1672 | UUGAAAUUU | 719 | ACAGUUACA | 769 |
| M_00410 | GGATTTTGAA | | ACUGUAACU | | GUAAAUUUC | |
| 4_8352 | ATTTACTGTA | | GU | | AA | |
| | ACTGTCAGTG | | | | | |
| | TACAC | | | | | |
| FASN_N | CTTGGTCTTT | 673 | CUUGGAUUU | 720 | AAUAAAUAU | 770 |
| M_00410 | CTGTGCTTGG | | GCAUAUUUA | | GCAAAUCCA | |
| 4_7970 | ATTTGCATAT | | UU | | AG | |
| | TTATTGCATT | | | | | |
| | GCTGG | | | | | |
| FASN_N | ATCGACGCCA | 674 | AAUGCCUUG | 721 | ACAGGUGGG | 771 |
| M_00410 | ACCCCAATGC | | UUCCCACCUG | | AACAAGGCA | |
| 4_2560 | CTTGTTCCCA | | U | | UU | |
| | CCTGTGGAGT | | | | | |
| | TCCCA | | | | | |
| FASN_N | GACGAGAGC | 675 | GAUGACAUC | 722 | AACGAAUGG | 772 |
| M_00410 | ACCTTTGATG | | GUCCAUUCG | | ACGAUGUCA | |
| 4_1762 | ACATCGTCCA | | UU | | UC | |
| | TTCGTTTGTG | | | | | |
| | AGCCTG | | | | | |
| FASN_N | GTCGCTTCCT | 676 | UUGGCAAAU | 723 | AGAAAGGUC | 773 |
| M_00410 | GGAAATTGGC | | UCGACCUUU | | GAAUUUGCC | |
| 4_5429 | AAATTCGACC | | CU | | AA | |
| | TTTCTCAGAA | | | | | |
| | CCACC | | | | | |
| FASN_N | AACCTCTCCC | 677 | UGCGACGGG | 724 | ACGGAUACU | 774 |
| M_00410 | AGGTATGCGA | | AAAGUAUCC | | UUCCCGUCGC | |
| 4_7525 | CGGGAAAGT | | GU | | A | |
| | ATCCGTCCAC | | | | | |
| | GTCATC | | | | | |
| FASN_N | AGGTGACTCG | 678 | GCUGCUGUG | 725 | AGUUAUCCU | 775 |
| M_00410 | GGGAGGCTGC | | GAAGGAUAA | | UCCACAGCA | |
| 4_3180 | TGTGGAAGGA | | CU | | GC | |
| | TAACTGGGTG | | | | | |
| | AGCTT | | | | | |
| FASN_N | CGTGGCAATG | 679 | CAGAGCAAC | 726 | GCAAAGCCG | 776 |
| M_00410 | CGGGACAGA | | UACGGCUUU | | UAGUUGCUC | |
| 4_6214 | GCAACTACGG | | GC | | UG | |
| | CTTTGCCAAT | | | | | |
| | TCCGCC | | | | | |
| FASN_N | GCGGGACAG | 680 | UACGGCUUU | 727 | GCGGAAUUG | 777 |
| M_00410 | AGCAACTACG | | GCCAAUUCC | | GCAAAGCCG | |
| 4_6223 | GCTTTGCCAA | | GC | | UA | |
| | TTCCGCCATG | | | | | |
| | GAGCGT | | | | | |
| FASN_N | GGTCGCTTCC | | AUUGGCAAA | 728 | GAAAGGUCG | 778 |
| M_00410 | TGGAAATTGG | | UUCGACCUU | | AAUUUGCCA | |
| 4_5428 | CAAATTCGAC | | UC | | AU | |
| | CTTTCTCAGA | | | | | |
| | ACCAC | | | | | |
| | (SEQ ID | | | | | |
| | NO: 65) | | | | | |
| FASN_N | TTCTGTGCTT | 682 | UGCAUAUUU | 729 | AGCAAUGCA | 779 |
| M_00410 | GGATTTGCAT | | AUUGCAUUG | | AUAAAUAUG | |
| 4_7978 | ATTTATTGCA | | CU | | CA | |
| | TTGCTGGTAG | | | | | |
| | AGACC | | | | | |
| FASN_N | CAGAGCAACT | 683 | UUUGCCAAU | 730 | UCCAUGGCG | 780 |
| M_00410 | ACGGCTTTGC | | UCCGCCAUG | | GAAUUGGCA | |
| 4_6229 | CAATTCCGCC | | GA | | AA | |
| | ATGGAGCGTA | | | | | |
| | TCTGT | | | | | |
| FASN_N | ACAGACGAG | 684 | UUUGAUGAC | 731 | GAAUGGACG | 781 |
| M_00410 | AGCACCTTTG | | AUCGUCCAU | | AUGUCAUCA | |
| 4_1759 | ATGACATCGT | | UC | | AA | |
| | CCATTCGTTT | | | | | |
| | GTGAGC | | | | | |
| FASN_N | TTTTTCAAGA | 685 | UUCAAAUUG | 732 | AAUCCAAGC | 782 |
| M_00410 | AATGATTCAA | | CUGCUUGGA | | AGCAAUUUG | |
| 4_8332 | ATTGCTGCTT | | UU | | AA | |
| | GGATTTTGAA | | | | | |
| | ATTTA | | | | | |
| FASN_N | GTCTTCAACC | 686 | GUGGUCUUG | 733 | AAGCCAUCU | 783 |
| M_00410 | TGGCCGTGGT | | AGAGAUGGC | | CUCAAGACC | |
| 4_6040 | CTTGAGAGAT | | UU | | AC | |
| | GGCTTGCTGG | | | | | |
| | AGAAC | | | | | |
| FASN_N | CTGACTGCCA | 687 | AUAGGCCUC | 734 | AGCAGGUCU | 784 |
| M_00410 | TCCAGATAGG | | AUAGACCUG | | AUGAGGCCU | |
| 4_1804 | CCTCATAGAC | | CU | | AU | |
| | CTGCTGAGCT | | | | | |
| | GCATG | | | | | |
| FASN_N | ATGGCCCAAG | 688 | CACAUUGGC | 735 | ACGACGACU | 785 |
| M_00410 | GGAAGCACAT | | AAAGUCGUC | | UUGCCAAUG | |
| 4_5665 | TGGCAAAGTC | | GU | | UG | |
| | GTCGTGCAGG | | | | | |
| | TGCTT | | | | | |
| FASN_N | TCGTTTGTGA | 689 | ACUGCCAUCC | 736 | AGGCCUAUC | 786 |
| M_00410 | GCCTGACTGC | | AGAUAGGCC | | UGGAUGGCA | |
| 4_1792 | CATCCAGATA | | U | | GU | |
| | GGCCTCATAG | | | | | |
| | ACCTG | | | | | |
| FASN_N | TCAGCTGCGG | 690 | GCUGGAAGU | 737 | CUUCAUAGG | 787 |
| M_00410 | CTGCTGCTGG | | CACCUAUGA | | UGACUUCCA | |
| 4 369 | AAGTCACCTA | | AG | | GC | |
| | TGAAGCCATC | | | | | |
| | GTGGA | | | | | |
| FASN_N | ACGCACGGTC | 691 | CUGGAAAUU | 738 | UCGAAUUUG | 788 |
| M_00410 | GCTTCCTGGA | | GGCAAAUUC | | CCAAUUUCC | |
| 4_5422 | AATTGGCAAA | | GA | | AG | |
| | TTCGACCTTT | | | | | |
| | CTCAG | | | | | |
| FASN_N | TGGCACACGG | 692 | AAGGGCGUU | 739 | AAGACCAGG | 789 |
| M_00410 | GCGGGAAGG | | GACCUGGUC | | UCAACGCCCU | |
| 4_5338 | GCGTTGACCT | | UU | | U | |
| | GGTCTTGAAC | | | | | |
| | TCCTTG | | | | | |
| FASN_N | GACCTCCCAG | 693 | CCUGCUAGG | 740 | AGAACUCCA | 790 |
| M_00410 | GACAGCCTGC | | UAUGGAGUU | | UACCUAGCA | |
| 4_4914 | TAGGTATGGA | | CU | | GG | |
| | GTTCTCGGGC | | | | | |
| | CGAGA | | | | | |
| FASN_N | CACCAACTCC | 694 | UGGUGUUUG | 741 | ACAAACACA | 791 |
| M_00410 | ATGTTTGGTG | | UCUGUGUUU | | GACAAACAC | |
| 4_8298 | TTTGTCTGTG | | GU | | CA | |
| | TTTGTTTTTCA | | | | | |
| | AGAA | | | | | |
| FASN_N | TTGGCAAATT | 695 | UUUCUCAGA | 742 | GAGCGGGUG | 792 |
| M_00410 | CGACCTTTCT | | ACCACCCGCU | | GUUCUGAGA | |
| 4_5444 | CAGAACCACC | | C | | AA | |
| | CGCTCGGCAT | | | | | |
| | GGCTA | | | | | |
| FASN_N | CTGTGTTTGT | | AAGAAAUGA | 743 | AGCAAUUUG | 793 |
| M_00410 | TTTTCAAGAA | | UUCAAAUUG | | AAUCAUUUC | |
| 4_8323 | ATGATTCAAA | | CU | | UU | |
| | TTGCTGCTTG | | | | | |
| | GATTT | | | | | |
| | (SEQ ID | | | | | |
| | NO: 66) | | | | | |
| FASN_N | CTGGCACGCA | | UCCGCCGAG | 744 | UUGACAUUG | 794 |
| M_00410 | CGTCCTCCGC | | UACAAUGUC | | UACUCGGCG | |
| 4_2314 | CGAGTACAAT | | AA | | GA | |
| | GTCAACAACC | | | | | |
| | TGGTG | | | | | |
| | (SEQ ID | | | | | |
| | NO: 64) | | | | | |
| FASN_N | GAGCAGTACA | 696 | AAGGCCAAG | 745 | UUGCCAUGG | 795 |
| M_00410 | CACCCAAGGC | | UACCAUGGC | | UACUUGGCC | |
| 4_7429 | CAAGTACCAT | | AA | | UU | |
| | GGCAACGTGA | | | | | |
| | TGCTA | | | | | |
| FASN_N | ACACACCCAA | 697 | AGUACCAUG | 746 | CAUCACGUU | 796 |
| M_00410 | GGCCAAGTAC | | GCAACGUGA | | GCCAUGGUA | |
| 4_7436 | CATGGCAACG | | UG | | CU | |
| | TGATGCTACT | | | | | |
| | GCGCG | | | | | |
| FASN_N | TGGTCTTTCT | 698 | UGGAUUUGC | 747 | GCAAUAAAU | 797 |
| M_00410 | GTGCTTGGAT | | AUAUUUAUU | | AUGCAAAUC | |
| 4_7972 | TTGCATATTT | | GC | | CA | |
| | ATTGCATTGC | | | | | |
| | TGGTA | | | | | |
| FASN_N | GTCTGTGTTT | 699 | UCAAGAAAU | 748 | CAAUUUGAA | 798 |
| M_00410 | GTTTTTCAAG | | GAUUCAAAU | | UCAUUUCUU | |
| 4_8321 | AAATGATTCA | | UG | | GA | |
| | AATTGCTGCT | | | | | |
| | TGGAT | | | | | |
| FASN_N | GAAGCTGCCA | 700 | GGAGAACUU | 749 | AGAACUCCU | 799 |
| M_00410 | GAGTCGGAG | | GCAGGAGUU | | GCAAGUUCU | |
| 4_171 | AACTTGCAGG | | CU | | CC | |
| | AGTTCTGGGA | | | | | |
| | CAACCT | | | | | |
| FASN_N | GTGCCTTCGA | 701 | CAGAGAACG | 750 | UACCAGGUU | 800 |
| M_00410 | GGTGTCAGAG | | GCAACCUGG | | GCCGUUCUC | |
| 4_2963 | AACGGCAACC | | UA | | UG | |
| | TGGTAGTGAG | | | | | |
| | TGGGA | | | | | |
| FASN_N | TGTGTTTGTT | | AGAAAUGAU | 751 | CAGCAAUUU | 801 |
| M_00410 | TTTCAAGAAA | | UCAAAUUGC | | GAAUCAUUU | |
| 4_8324 | TGATTCAAAT | | UG | | CU | |
| | TGCTGCTTGG | | | | | |
| | ATTTT | | | | | |
| | (SEQ ID | | | | | |
| | NO: 67) | | | | | |
| FASN_N | GGCTGCTGCT | 702 | UCACCUAUG | 752 | CACGAUGGC | 802 |
| M_00410 | GGAAGTCACC | | AAGCCAUCG | | UUCAUAGGU | |
| 4_377 | TATGAAGCCA | | UG | | GA | |
| | TCGTGGACGG | | | | | |
| | AGGCA | | | | | |
| FASN_N | TTGGATTTTG | 703 | UACUGUAAC | 753 | GUACACUGA | 803 |
| M_00410 | AAATTTACTG | | UGUCAGUGU | | CAGUUACAG | |
| 4_8360 | TAACTGTCAG | | AC | | UA | |
| | TGTACACGTC | | | | | |
| | TGGAC | | | | | |
| FASN_N | CGTCGAGCAG | 704 | UGUGGUGUU | 754 | CCACAUCCUC | 804 |
| M_00410 | CTGCCTGTGG | | UGAGGAUGU | | AAACACCACA | |
| 4_2856 | TGTTTGAGGA | | GG | | | |
| | TGTGGTGCTG | | | | | |
| | CACCA | | | | | |

TABLE 6
Sequences targeting mouse FASN gene target regions.

| | | SEQ | | SEQ | | SEQ |
|---|---|---|---|---|---|---|
| Sequence | Target | ID | | ID | Reverse | ID |
| ID | Region | NO | Sequence | NO | complement | NO |
| Fasn_NM | GGCTGCTGTT | 805 | UCAGCUAUG | 855 | CACAAUUGC | 905 |
| 007988 | GGAAGTCAGC | | AAGCAAUUG | | UUCAUAGCU | |
| 781 | TATGAAGCAA | | UG | | GA | |
| | TTGTGGATGG | | | | | |
| | AGGTA | | | | | |
| Fasn_NM | CCTAGAAGAG | 806 | UGUGGUAUU | 856 | ACACAUUCU | 906 |
| _007988 | ACCCCTGTGG | | UGAGAAUGU | | CAAAUACCA | |
| 3260 | TATTTGAGAA | | GU | | CA | |
| | TGTGTCGTTT | | | | | |
| | CATCA | | | | | |
| Fasn_NM | ATTTGCCACG | 807 | CGUUUCUCC | 857 | GAGAAUUUA | 907 |
| _007988 | CTGGTCGTTT | | AUUAAAUUC | | AUGGAGAAA | |
| 8340 | CTCCATTAAA | | UC | | CG | |
| | TTCTCATATT | | | | | |
| | TATTG | | | | | |
| Fasn_NM | TGCCACGCTG | 808 | UUCUCCAUU | 858 | UAUGAGAAU | 908 |
| 007988 | GTCGTTTCTC | | AAAUUCUCA | | UUAAUGGAG | |
| 8343 | CATTAAATTC | | UA | | AA | |
| | TCATATTTAT | | | | | |
| | TGCAT | | | | | |
| Fasn_NM | ATCTCTGGAC | 809 | GCUGCAGUG | 859 | ACAAAUUCA | 909 |
| _007988 | CTCAGGCTGC | | AAUGAAUUU | | UUCACUGCA | |
| 2493 | AGTGAATGAA | | GU | | GC | |
| | TTTGTGGAGC | | | | | |
| | AGCTA | | | | | |
| Fasn_NM | TCTAACAACC | 810 | CUGGGCAUG | 860 | AAGAAGAUA | 910 |
| 007988 | ACCCTCTGGG | | GCUAUCUUC | | GCCAUGCCCA | |
| 5844 | CATGGCTATC | | UU | | G | |
| | TTCTTGAAGA | | | | | |
| | ACGTC | | | | | |
| Fasn_NM | TGAGGCCTTA | 811 | CUUCAUAAA | 861 | CAAGAAACU | 911 |
| 007988 | TGCTTCTTCA | | GCAGUUUCU | | GCUUUAUGA | |
| 7616 | TAAAGCAGTT | | UG | | AG | |
| | TCTTGATGTG | | | | | |
| | GAACA | | | | | |
| Fasn_NM | GTTCGTGGTG | 812 | GUGGGCAUC | 862 | CCAAAUGAG | 912 |
| _007988_ | GCAACGTGGG | | AACUCAUUU | | UUGAUGCCC | |
| 1689 | CATCAACTCA | | GG | | AC | |
| | TTTGGCTTCG | | | | | |
| | GAGGC | | | | | |
| Fasn_NM | GCCCCAGCGG | 813 | AAUAGCAUC | 863 | AAGUAGAGG | 913 |
| 007988 | TAGAGAATAG | | AUCCUCUAC | | AUGAUGCUA | |
| 9091 | CATCATCCTC | | UU | | UU | |
| | TACTTGTAGT | | | | | |
| | CAACT | | | | | |
| Fasn_NM | GCTGAGGCCT | 814 | UUCUUCAUA | 864 | AGAAACUGC | 914 |
| 007988 | TATGCTTCTT | | AAGCAGUUU | | UUUAUGAAG | |
| 7614 | CATAAAGCAG | | CU | | AA | |
| | TTTCTTGATG | | | | | |
| | TGGAA | | | | | |
| Fasn_NM | TTCTACCGTG | 815 | UUUGCCACG | 865 | GAAACGACC | 915 |
| 007988 | TGGCATTTGC | | CUGGUCGUU | | AGCGUGGCA | |
| 8326 | CACGCTGGTC | | UC | | AA | |
| | GTTTCTCCAT | | | | | |
| | TAAAT | | | | | |
| Fasn_NM | CACACTTAGA | 816 | UCUUACAGC | 866 | UCAUGACGA | 916 |
| 007988 | AAAATTCTTA | | ACUCGUCAU | | GUGCUGUAA | |
| 9579 | CAGCACTCGT | | GA | | GA | |
| | CATGAGTTGT | | | | | |
| | CCATT | | | | | |
| Fasn_NM | TAGTACAAGC | 817 | UUGUCCACC | 867 | GGCAACUUA | 917 |
| 007988 | AGAATTTGTC | | UUUAAGUUG | | AAGGUGGAC | |
| 9028 | CACCTTTAAG | | CC | | AA | |
| | TTGCCCTGAG | | | | | |
| | GGCAT | | | | | |
| Fasn_NM | GACTGAAGCA | 818 | UUGGCUCUA | 868 | CUAAGUGUG | 918 |
| _007988 | TCTGGTTGGC | | GUCACACUU | | ACUAGAGCC | |
| 9553 | TCTAGTCACA | | AG | | AA | |
| | CTTAGAAAAA | | | | | |
| | TTCTT | | | | | |
| Fasn_NM | TGTGCTGCCA | 819 | UCAGCCCACC | 869 | CAGAAAUCA | 919 |
| 007988 | GGGGCTCAGC | | CUGAUUUCU | | GGGUGGGCU | |
| 6110 | CCACCCTGAT | | G | | GA | |
| | TTCTGCCATC | | | | | |
| | TCCAA | | | | | |
| Fasn_NM | GACGGGAAG | 820 | GUGCACAUC | 870 | UCACCCUCAA | 920 |
| 007988 | GTGTCTGTGC | | AUUGAGGGU | | UGAUGUGCA | |
| 7926 | ACATCATTGA | | GA | | C | |
| | GGGTGACCAC | | | | | |
| | CGCACA | | | | | |
| Fasn_NM | AGACCCCAGA | 821 | UCCAGGAUG | 871 | GGGCUUGUU | 921 |
| 007988 | GCTCTTCCAG | | UCAACAAGC | | GACAUCCUG | |
| 6469 | GATGTCAACA | | CC | | GA | |
| | AGCCCAAATA | | | | | |
| | CAATG | | | | | |
| Fasn_NM | GTGGCATTTG | 822 | CUGGUCGUU | 872 | UUUAAUGGA | 922 |
| _007988 | CCACGCTGGT | | UCUCCAUUA | | GAAACGACC | |
| 8335 | CGTTTCTCCA | | AA | | AG | |
| | TTAAATTCTC | | | | | |
| | ATATT | | | | | |
| Fasn_NM | CAAACTAACT | 823 | UUCGCCAAC | 873 | UCCAUGGUA | 923 |
| 007988 | ACGGCTTCGC | | UCUACCAUG | | GAGUUGGCG | |
| 6612 | CAACTCTACC | | GA | | AA | |
| | ATGGAGCGTA | | | | | |
| | TATGT | | | | | |
| Fasn_NM | GTGTGCGGTG | 824 | CUCAGCAUG | 874 | UAAGAAGCG | 924 |
| 007988 | CTTGGCTCAG | | GUCGCUUCU | | ACCAUGCUG | |
| 5788 | CATGGTCGCT | | UA | | AG | |
| | TCTTAGAGAT | | | | | |
| | TGGCA | | | | | |
| Fasn_NM | CTTGGAAAAG | 825 | UUUCACACC | 875 | CCUCCAUGU | 925 |
| 007988 | TTCGTTTTCA | | GCACAUGGA | | GCGGUGUGA | |
| 3884 | CACCGCACAT | | GG | | AA | |
| | GGAGGCTGA | | | | | |
| | GTGCCT | | | | | |
| Fasn_NM | GGGTCCAGGA | 826 | UGUCACAAG | 876 | AAUGCUGCG | 926 |
| _007988 | GAGACTGTCA | | GCCGCAGCA | | GCCUUGUGA | |
| 9427 | CAAGGCCGCA | | UU | | CA | |
| | GCATTCGATC | | | | | |
| | ACATC | | | | | |
| Fasn_NM | AGGCCTTGCC | 827 | AGUACAAGC | 877 | GGACAAAUU | 927 |
| _007988 | ACTGTAGTAC | | AGAAUUUGU | | CUGCUUGUA | |
| 9014 | AAGCAGAATT | | CC | | CU | |
| | TGTCCACCTT | | | | | |
| | TAAGT | | | | | |
| Fasn_NM | AACCTCTCCC | 828 | UGUGACGGG | 878 | ACAGACACC | 928 |
| 007988 | AGGTGTGTGA | | AAGGUGUCU | | UUCCCGUCAC | |
| 7908 | CGGGAAGGT | | GU | | A | |
| | GTCTGTGCAC | | | | | |
| | ATCATT | | | | | |
| Fasn_NM | CAGCATGGTC | 829 | UUAGAGAUU | 879 | UCAAAUUUG | 929 |
| 007988 | GCTTCTTAGA | | GGCAAAUUU | | CCAAUCUCU | |
| 5805 | GATTGGCAAA | | GA | | AA | |
| | TTTGATCTTT | | | | | |
| | CTAAC | | | | | |
| Fasn_NM | TAGGGTACAA | 830 | UUCAGAGGA | 880 | UCAGACUGG | 930 |
| 007988_ | AGGTGTTCAG | | UCCCAGUCU | | GAUCCUCUG | |
| 9505 | AGGATCCCAG | | GA | | AA | |
| | TCTGAGTGAT | | | | | |
| | TAGCC | | | | | |
| Fasn_NM | GCTGGTCGTT | 831 | AUUAAAUUC | 881 | AAUAAAUAU | 931 |
| _007988 | TCTCCATTAA | | UCAUAUUUA | | GAGAAUUUA | |
| 8349 | ATTCTCATAT | | UU | | AU | |
| | TTATTGCATT | | | | | |
| | GCTGG | | | | | |
| Fasn_NM | TCATTTGGAG | 832 | GUAGCCUUC | 882 | GAGCACAUC | 932 |
| 007988 | CCTGTGTAGC | | GAGAUGUGC | | UCGAAGGCU | |
| 7443 | CTTCGAGATG | | UC | | AC | |
| | TGCTCCCAGC | | | | | |
| | TGCAG | | | | | |
| Fasn_NM | CTTCATGGAC | 833 | GCUGCAGGU | 883 | CCAGAAUGG | 933 |
| _007988 | ACAATGCTGC | | AUCCAUUCU | | AUACCUGCA | |
| 3629 | AGGTATCCAT | | GG | | GC | |
| | TCTGGGTTCT | | | | | |
| | AGCCA | | | | | |
| Fasn_NM | TTTTCTGCAG | 834 | AGUGUGAAG | 884 | UCCAGACAC | 934 |
| _007988 | CTGTCAGTGT | | AAGUGUCUG | | UUCUUCACA | |
| 8720 | GAAGAAGTGT | | GA | | CU | |
| | CTGGACTGTG | | | | | |
| | TCATT | | | | | |
| Fasn_NM | AGCATGGTCG | 835 | UAGAGAUUG | 885 | AUCAAAUUU | 935 |
| _007988 | CTTCTTAGAG | | GCAAAUUUG | | GCCAAUCUC | |
| 5806 | ATTGGCAAAT | | AU | | UA | |
| | TTGATCTTTC | | | | | |
| | TAACA | | | | | |
| Fasn_NM | TTGAGCACAG | 836 | CGCACCUUU | 886 | ACGAUGUCA | 936 |
| 007988 | ATGAGCGCAC | | GAUGACAUC | | UCAAAGGUG | |
| 2157 | CTTTGATGAC | | GU | | CG | |
| | ATCGTGCATG | | | | | |
| | CCTTT | | | | | |
| Fasn_NM | GAGGCTGTGA | 837 | UUGGGAGUG | 887 | UCUGACACU | 937 |
| _007988 | AGCCGTTGGG | | AAAGUGUCA | | UUCACUCCCA | |
| 2115 | AGTGAAAGTG | | GA | | A | |
| | TCAGATCTGC | | | | | |
| | TGTTG | | | | | |
| Fasn_NM | TCGCTTCTTA | 838 | UGGCAAAUU | 888 | UAGAAAGAU | 938 |
| 007988 | GAGATTGGCA | | UGAUCUUUC | | CAAAUUUGC | |
| 5813 | AATTTGATCT | | UA | | CA | |
| | TTCTAACAAC | | | | | |
| | CACCC | | | | | |
| Fasn_NM | AGGCCTTATG | 839 | UCAUAAAGC | 889 | AUCAAGAAA | 939 |
| 007988 | CTTCTTCATA | | AGUUUCUUG | | CUGCUUUAU | |
| 7618 | AAGCAGTTTC | | AU | | GA | |
| | TTGATGTGGA | | | | | |
| | ACACA | | | | | |
| Fasn_NM | CCTGGCCACC | 840 | CCUGCUAUC | 890 | GGAAGUCAG | 940 |
| 007988 | TCAGTCCTGC | | AUCUGACUU | | AUGAUAGCA | |
| 5384 | TATCATCTGA | | CC | | GG | |
| | CTTCCTCTGG | | | | | |
| | GATGT | | | | | |
| Fasn_NM | TCCAGTGAGA | 841 | UGGAGGGUG | 891 | ACAGAAUGG | 941 |
| 007988 | CTCCTTGGAG | | UGCCAUUCU | | CACACCCUCC | |
| 8928 | GGTGTGCCAT | | GU | | A | |
| | TCTGTCAGTA | | | | | |
| | TAAGG | | | | | |
| Fasn_NM | GGCAATAAA | 842 | AGGUUCCUU | 892 | CAAAGAACA | 942 |
| 007988 | ACCTTGAGGT | | CCUGUUCUU | | GGAAGGAAC | |
| 9762 | TCCTTCCTGT | | UG | | CU | |
| | TCTTTGTGAT | | | | | |
| | TGCCTA | | | | | |
| Fasn_NM | GTTTCTCCAT | 843 | UCUCAUAUU | 893 | GCAAUGCAA | 943 |
| 007988 | TAAATTCTCA | | UAUUGCAUU | | UAAAUAUGA | |
| 8356 | TATTTATTGC | | GC | | GA | |
| | ATTGCTGGGA | | | | | |
| | AAGAC | | | | | |
| Fasn_NM | AGGCCACCCT | 844 | GUGAACAAG | 894 | GAGCAGCUU | 944 |
| _007988 | TGAAGGTGAA | | GCAAGCUGC | | GCCUUGUUC | |
| 3574 | CAAGGCAAG | | UC | | AC | |
| | CTGCTCTGGA | | | | | |
| | AAGATA | | | | | |
| Fasn_NM | CTGCTAGAGG | 845 | CAUGCCUUU | 895 | UCAGACACC | 945 |
| 007988 | CCTCCCATGC | | GAGGUGUCU | | UCAAAGGCA | |
| 3351 | CTTTGAGGTG | | GA | | UG | |
| | TCTGACACTG | | | | | |
| | GCAAT | | | | | |
| Fasn_NM | CTGGCTGGAG | 846 | GCCACUGUA | 896 | UCUGCUUGU | 946 |
| 007988 | GCCTTGCCAC | | GUACAAGCA | | ACUACAGUG | |
| 9006 | TGTAGTACAA | | GA | | GC | |
| | GCAGAATTTG | | | | | |
| | TCCAC | | | | | |
| Fasn_NM | ATCAATGTCA | 847 | AACGCCUUG | 897 | ACAGGUGGG | 947 |
| 007988 | ACCCTAACGC | | UUCCCACCUG | | AACAAGGCG | |
| 2964 | CTTGTTCCCA | | U | | UU | |
| | CCTGTGGAGT | | | | | |
| | TCCCG | | | | | |
| Fasn_NM | TTCCCTTGAT | 848 | GGGAUCAUA | 898 | CAAGUUAUC | 948 |
| _007988 | GAAGAGGGA | | AAGAUAACU | | UUUAUGAUC | |
| 2887 | TCATAAAGAT | | UG | | CC | |
| | AACTTGGAGT | | | | | |
| | TCTTTC | | | | | |
| Fasn_NM | CTAGAAGAG | 849 | GUGGUAUUU | 899 | GACACAUUC | 949 |
| _007988_ | ACCCCTGTGG | | GAGAAUGUG | | UCAAAUACC | |
| 3261 | TATTTGAGAA | | UC | | AC | |
| | TGTGTCGTTT | | | | | |
| | CATCAG | | | | | |
| Fasn_NM | CTGGCCCGCA | 850 | UCUGCCGAG | 900 | UUGACAUUG | 950 |
| 007988 | CATCTTCTGC | | UACAAUGUC | | UACUCGGCA | |
| 2718 | CGAGTACAAT | | AA | | GA | |
| | GTCAACAACC | | | | | |
| | TGGTG | | | | | |
| Fasn_NM | AGTGTCTCTA | 851 | UUAGAGUUU | 901 | CUUGAGUCA | 951 |
| 007988 | CCTAGTTAGA | | ACUGACUCA | | GUAAACUCU | |
| 9651 | GTTTACTGAC | | AG | | AA | |
| | TCAAGGGAGC | | | | | |
| | AGATT | | | | | |
| Fasn_NM | CGTGGCCCTG | 852 | CUCCUCAGU | 902 | GGAAACUCU | 514 |
| _007988 | GTGTCCTCCT | | GCAGAGUUU | | GCACUGAGG | |
| 49 | CAGTGCAGAG | | CC | | AG | |
| | TTTCCAGTGT | | | | | |
| | GACCA | | | | | |
| Fasn_NM | TCTACCGTGT | 853 | UUGCCACGC | 903 | AGAAACGAC | 528 |
| 007988 | GGCATTTGCC | | UGGUCGUUU | | CAGCGUGGC | |
| 8327 | ACGCTGGTCG | | CU | | AA | |
| | TTTCTCCATT | | | | | |
| | AAATT | | | | | |
| Fasn_NM | ATTGGCTCCA | 854 | UCCAACAUG | 904 | UCAGGGUGU | 681 |
| 007988 | CCAAATCCAA | | GGACACCCU | | CCCAUGUUG | |
| 1506 | CATGGGACAC | | GA | | GA | |
| | CCTGAGCCTG | | | | | |
| | CCTCT | | | | | |

TABLE 7
Modified FASN mRNA targeting antisense (as) strand sequences.

| OLIGO ID | Modified Sequence |
|---|---|
| FASN_NM_ | P(mU)#(fU)#(mG)(fA)(fC)(fA)(mU)(fU)(mG)(fU)(mA)(fC)(mU)(fC) |
| 004104_2314_as | #(mG)#(fG)#(mC)#(mG)#(mG)#(mU)#(fU) (SEQ ID NO: 56) |
| FASN_NM_ | (mC)#(mC)#(mG)(fA)(mG)(fU)(mA)(fC)(mA)(fA)(mU)(mG)(mU) |
| 004104_2314_s | (fC)#(mA)#(mA)-TegChol (SEQ ID NO: 57) |
| FASN_NM_ | P(mU)#(fA)#(mA)(fA)(fG)(fG)(mU)(fC)(mG)(fA)(mA)(fU)(mU) |
| 004104_5428_AS | (fU)#(mG)#(fC)#(mC)#(mA)#(mA)#(mU)#(fU) (SEQ ID NO: 58) |
| FASN_NM_ | (mG)#(mC)#(mA)(fA)(mA)(fU)(mU)(fC)(mG)(fA)(mC)(mC)(mU) |
| 004104_5428_S | (fU)#(mU)#(mA)-TegChol (SEQ ID NO: 59) |
| FASN_NM_ | P(mU)#(fG)#(mC)(fA)(fA)(fU)(mU)(fU)(mG)(fA)(mA)(fU)(mC) |
| 004104_8323_as | (fA)#(mU)#(fU)#(mU)#(mC)#(mU)#(mU)#(fU) (SEQ ID NO: 60) |
| FASN_NM_ | (mA)#(mA)#(mU)(fG)(mA)(fU)(mU)(fC)(mA)(fA)(mA)(mU)(mU) |
| 004104_8323_S | (fG)#(mC)#(mA)-TegChol (SEQ ID NO: 61) |
| FASN_NM_ | P(mU)#(fA)#(mG)(fC)(fA)(fA)(mU)(fU)(mU)(fG)(mA)(fA)(mU) |
| 004104_8324_as | (fC)#(mA)#(fU)#(mU)#(mU)#(mC)#(mU)#(fU) (SEQ ID NO: 62) |
| FASN_NM_ | (mA)#(mU)#(mG)(fA)(mU)(fU)(mC)(fA)(mA)(fA)(mU)(mU)(mG) |
| 004104_8324_s | (fC)#(mU)#(mA)-TegChol (SEQ ID NO: 63) |

“m” represents a 2′-O-methyl nucleotide modification; “f” represents a 2′-Fluoro nucleotide modification; “#” represents a phosphorothioate internucleotide linkage; “VP” represents a 5′-(E)-vinylphosphonate; “GalNAc” represents a GalNAc conjugate.

TABLE 8
Sequences targeting human OPN gene target regions.

| Sequence | | SEQ ID | | SEQ ID | Reverse | SEQ ID |
|---|---|---|---|---|---|---|
| ID | Target Region | NO | Sequence | NO | complement | NO |
| SPP1_NM_ | ATAATTAGTTTA | 74 | UGUGGCUUC | 118 | GGAGUUUCCAU | 166 |
| 001251830_ | GTTTGTGGCTTC | | AUGGAAACU | | GAAGCCACA | |
| 1398 | ATGGAAACTCC | | CC | | | |
| | CTGTAAACTA | | | | | |
| SPP1_NM_ | GTGTATCTATTT | 75 | UCUGGAAAU | 119 | ACACAUUAGUU | 167 |
| 001251830_ | GAGTCTGGAAA | | AACUAAUGU | | AUUUCCAGA | |
| 1360 | TAACTAATGTGT | | GU | | | |
| | TTGATAATTA | | | | | |
| SPP1_NM_ | GAATGTGTATCT | | UGAGUCUGG | 120 | AUUAGUUAUUU | 168 |
| 001251830_ | ATTTGAGTCTGG | | AAAUAACUA | | CCAGACUCA | |
| 1356 | AAATAACTAAT | | AU | | | |
| | GTGTTTGATA | | | | | |
| | (SEQ ID NO: 68) | | | | | |
| SPP1_NM_ | TTGTGATTATCT | 76 | UGUGGUGUG | 121 | AAAGAUUUAUU | 169 |
| 001251830_ | TTTTGTGGTGTG | | AAUAAAUCU | | CACACCACA | |
| 1634 | AATAAATCTTTT | | UU | | | |
| | ATCTTGAAT | | | | | |
| SPP1_NM_ | CTATGTTCATTC | 77 | AGAAGAAAU | 122 | UGAUAGUUUGC | 170 |
| 001251830_ | TATAGAAGAAA | | GCAAACUAU | | AUUUCUUCU | |
| 1461 | TGCAAACTATCA | | CA | | | |
| | CTGTATTTTA | | | | | |
| SPP1_NM_ | TATCTATTTGAG | 78 | GGAAAUAAC | 123 | CAAACACAUUA | 171 |
| 001251830_ | TCTGGAAATAA | | UAAUGUGUU | | GUUAUUUCC | |
| 1363 | CTAATGTGTTTG | | UG | | | |
| | ATAATTAGTT | | | | | |
| SPP1_NM_ | AAGCGGAAAGC | 79 | GAUGAGAGC | 124 | GAAUGCUCAUU | 172 |
| 001251830_ | CAATGATGAGA | | AAUGAGCAU | | GCUCUCAUC | |
| 1044 | GCAATGAGCAT | | UC | | | |
| | TCCGATGTGATT | | | | | |
| SPP1_NM_ | TCATTCTATAGA | 80 | AAUGCAAAC | 125 | AUACAGUGAUA | 173 |
| 001251830_ | AGAAATGCAAA | | UAUCACUGU | | GUUUGCAUU | |
| 1467 | CTATCACTGTAT | | AU | | | |
| | TTTAATATTT | | | | | |
| SPP1_NM_ | GGAAGAAGATA | 81 | CCUGAAAUU | 126 | GAGAAAUACGA | 174 |
| 001251830_ | AACACCTGAAA | | UCGUAUUUC | | AAUUUCAGG | |
| 1181 | TTTCGTATTTCT | | UC | | | |
| | CATGAATTAGA | | | | | |
| SPP1_NM_ | GAATGTAATAA | 82 | UUGGUGGUG | 127 | UAAGCAAUUGA | 175 |
| 001251830_ | GAATTTGGTGGT | | UCAAUUGCU | | CACCACCAA | |
| 1675 | GTCAATTGCTTA | | UA | | | |
| | TTTGTTTTCC | | | | | |
| SPP1_NM_ | ATAAATCTTTTA | 83 | UGAAUGUAA | 128 | CCAAAUUCUUA | 176 |
| 001251830_ | TCTTGAATGTAA | | UAAGAAUUU | | UUACAUUCA | |
| 1659 | TAAGAATTTGGT | | GG | | | |
| | GGTGTCAAT | | | | | |
| SPP1_NM_ | AGAAAAAATAC | | UCUCACUUU | 129 | UGACUAAAUGC | 177 |
| 001251830_ | AATTTCTCACTT | | GCAUUUAGU | | AAAGUGAGA | |
| 1255 | TGCATTTAGTCA | | CA | | | |
| | AAAGAAAAAA | | | | | |
| | (SEQ ID NO: 69) | | | | | |
| SPP1_NM_ | ACAGCCATGAA | 84 | ACAGCCAUG | 130 | CAGCAUAUCUU | 178 |
| 001251830_ | TTTCACAGCCAT | | AAGAUAUGC | | CAUGGCUGU | |
| 1126 | GAAGATATGCT | | UG | | | |
| | GGTTGTAGACC | | | | | |
| SPP1_NM_ | TGTATCTATTTG | 85 | CUGGAAAUA | 131 | AACACAUUAGU | 179 |
| 001251830_ | AGTCTGGAAAT | | ACUAAUGUG | | UAUUUCCAG | |
| 1361 | AACTAATGTGTT | | UU | | | |
| | TGATAATTAG | | | | | |
| SPP1_NM_ | TAAACTAAAAG | 86 | AGGGUUAUG | 132 | AUGAACAUAGA | 180 |
| 001251830_ | CTTCAGGGTTAT | | UCUAUGUUC | | | |
| 1436 | GTCTATGTTCAT | | AU | | CAUAACCCU | |
| | TCTATAGAAG | | | | | |
| SPP1_NM_ | CTTTATAGCAAA | | AAAGAGAAC | 133 | AAGCAUUUCAU | 181 |
| 001251830_ | ATGAAAGAGAA | | AUGAAAUGC | | | |
| 1302 | CATGAAATGCTT | | UU | | GUUCUCUUU | |
| | CTTTCTCAGT | | | | | |
| | (SEQ ID NO: 70) | | | | | |
| SPP1_NM_ | GGAGAAAAAAT | 87 | UUUCUCACU | 134 | ACUAAAUGCAA | 182 |
| 001251830_ | ACAATTTCTCAC | | UUGCAUUUA | | AGUGAGAAA | |
| 1253 | TTTGCATTTAGT | | GU | | | |
| | CAAAAGAAAA | | | | | |
| SPP1_NM_ | GCTGCCTTGGGG | 88 | ACUGCAAUU | 135 | UUAAGCAGUCU | 183 |
| 001251830_ | GTCACTGCAATT | | AGACUGCUU | | AAUUGCAGU | |
| 347 | AGACTGCTTAAT | | AA | | | |
| | GAAGACATT | | | | | |
| SPP1_NM_ | TATAGCAAAAT | 89 | GAGAACAUG | 136 | AAGAAGCAUUU | 184 |
| 001251830_ | GAAAGAGAACA | | AAAUGCUUC | | CAUGUUCUC | |
| 1305 | TGAAATGCTTCT | | UU | | | |
| | TTCTCAGTTTA | | | | | |
| SPP1_NM_ | TTTAATATTTGT | 90 | UCUCUCAUG | 137 | AAAUUUCUAUU | 185 |
| 001251830_ | TATTCTCTCATG | | AAUAGAAAU | | CAUGAGAGA | |
| 1502 | AATAGAAATTT | | UU | | | |
| | ATGTAGAAGC | | | | | |
| SPP1_NM_ | AACATGAAATG | | UUUCUCAGU | 138 | UCAACCAAUAA | 186 |
| 001251830_ | CTTCTTTCTCAG | | UUAUUGGUU | | ACUGAGAAA | |
| 1323 | TTTATTGGTTGA | | GA | | | |
| | ATGTGTATCT | | | | | |
| | (SEQ ID NO: 71) | | | | | |
| SPP1_NM_ | ATCTATTTGAGT | 91 | GAAAUAACU | 139 | UCAAACACAUU | 187 |
| 001251830_ | CTGGAAATAAC | | AAUGUGUUU | | AGUUAUUUC | |
| 1364 | TAATGTGTTTGA | | GA | | | |
| | TAATTAGTTT | | | | | |
| SPP1_NM_ | ACCTGAAATTTC | 92 | UUUCUCAUG | 140 | ACUAUCUAAUU | 188 |
| 001251830_ | GTATTTCTCATG | | AAUUAGAUA | | CAUGAGAAA | |
| 1195 | AATTAGATAGT | | GU | | | |
| | GCATCTTCTG | | | | | |
| SPP1_NM_ | ATGTTCATTCTA | 93 | AAGAAAUGC | 141 | AGUGAUAGUUU | 189 |
| 001251830_ | TAGAAGAAATG | | AAACUAUCA | | GCAUUUCUU | |
| 1463 | CAAACTATCACT | | CU | | | |
| | GTATTTTAAT | | | | | |
| SPP1_NM_ | CGATGTGATTGA | 94 | UCAGGAACU | 142 | UGACUUUGGAA | 190 |
| 001251830_ | TAGTCAGGAAC | | UUCCAAAGU | | AGUUCCUGA | |
| 1079 | TTTCCAAAGTCA | | CA | | | |
| | GCCGTGAATT | | | | | |
| SPP1_NM_ | AAAGTAAGGAA | 95 | AUAAACACC | 143 | ACGAAAUUUCA | 191 |
| 001251830_ | GAAGATAAACA | | UGAAAUUUC | | GGUGUUUAU | |
| 1174 | CCTGAAATTTCG | | GU | | | |
| | TATTTCTCATG | | | | | |
| SPP1_NM_ | GTTTATTGGTTG | 96 | GUGUAUCUA | 144 | CCAGACUCAAA | 192 |
| 001251830_ | AATGTGTATCTA | | UUUGAGUCU | | UAGAUACAC | |
| 1345 | TTTGAGTCTGGA | | GG | | | |
| | AATAACTAA | | | | | |
| SPP1_NM_ | GACAGCCAGGA | 97 | AUUGACUCG | 145 | UCAGAGUCGUU | 193 |
| 001251830_ | CTCCATTGACTC | | AACGACUCU | | CGAGUCAAU | |
| 597 | GAACGACTCTG | | GA | | | |
| | ATGATGTAGAT | | | | | |
| SPP1_NM_ | GTTATTCTCTCA | 98 | AUAGAAAUU | 146 | GCUUCUACAUA | 194 |
| 001251830_ | TGAATAGAAAT | | UAUGUAGAA | | AAUUUCUAU | |
| 1512 | TTATGTAGAAGC | | GC | | | |
| | AAACAAAATA | | | | | |
| SPP1_NM_ | AATGCTGTGTCC | 99 | GAAGAAACC | 147 | UUAAAGUCAUU | 195 |
| 001251830_ | TCTGAAGAAAC | | AAUGACUUU | | GGUUUCUUC | |
| 480 | CAATGACTTTAA | | AA | | | |
| | ACAAGAGACC | | | | | |
| SPP1_NM_ | AGCTTGGACAA | 100 | GCACACAGA | 148 | UGGAAUUGAAC | 196 |
| 001251830_ | AAAGGCACACA | | GUUCAAUUC | | UCUGUGUGC | |
| 285 | GAGTTCAATTCC | | CA | | | |
| | AGTTGAACAGA | | | | | |
| SPP1_NM_ | ATTCTATAGAAG | 101 | UGCAAACUA | 149 | AAAUACAGUGA | 197 |
| 001251830_ | AAATGCAAACT | | UCACUGUAU | | UAGUUUGCA | |
| 1469 | ATCACTGTATTT | | UU | | | |
| | TAATATTTGT | | | | | |
| SPP1_NM_ | TGATGTAGATG | 102 | UGAUGAUUC | 150 | CAGACUGGUGA | 198 |
| 001251830_ | ACACTGATGATT | | UCACCAGUC | | GAAUCAUCA | |
| 632 | CTCACCAGTCTG | | UG | | | |
| | ATGAGTCTCA | | | | | |
| SPP1_NM_ | TCCAACGAAAG | 103 | GACCACAUG | 151 | UCCAUAUCAUC | 199 |
| 001251830_ | CCATGACCACAT | | GAUGAUAUG | | CAUGUGGUC | |
| 537 | GGATGATATGG | | GA | | | |
| | ATGATGAAGAT | | | | | |
| SPP1_NM_ | AATTAAAAGGA | 104 | AAAUACAAU | 152 | CAAAGUGAGAA | 200 |
| 001251830_ | GAAAAAATACA | | UUCUCACUU | | AUUGUAUUU | |
| 1245 | ATTTCTCACTTT | | UG | | | |
| | GCATTTAGTCA | | | | | |
| SPP1_NM_ | AAATAACTAAT | 105 | UUGAUAAUU | 153 | CAAACUAAACU | 201 |
| 001251830_ | GTGTTTGATAAT | | AGUUUAGUU | | AAUUAUCAA | |
| 1380 | TAGTTTAGTTTG | | UC | | | |
| | TGGCTTCATG | | | | | |
| SPP1_NM_ | AAATCTTTTATC | 106 | AAUGUAAUA | 154 | CACCAAAUUCU | 202 |
| 001251830_ | TTGAATGTAATA | | AGAAUUUGG | | UAUUACAUU | |
| 1661 | AGAATTTGGTG | | UG | | | |
| | GTGTCAATTG | | | | | |
| SPP1_NM_ | ATTGCTTATTTG | 107 | UCCCACGGU | 155 | AUUGCUGGACA | 203 |
| 001251830_ | TTTTCCCACGGT | | UGUCCAGCA | | ACCGUGGGA | |
| 1702 | TGTCCAGCAATT | | AU | | | |
| | AATAAAACA | | | | | |
| SPP1_NM_ | GAGCATTCCGAT | 108 | AUUGAUAGU | 156 | GAAAGUUCCUG | 204 |
| 001251830_ | GTGATTGATAGT | | CAGGAACUU | | ACUAUCAAU | |
| 1071 | CAGGAACTTTCC | | UC | | | |
| | AAAGTCAGC | | | | | |
| SPP1_NM_ | GTAAACTAAAA | 109 | CAGGGUUAU | 157 | UGAACAUAGAC | 205 |
| 001251830_ | GCTTCAGGGTTA | | GUCUAUGUU | | AUAACCCUG | |
| 1435 | TGTCTATGTTCA | | CA | | | |
| | TTCTATAGAA | | | | | |
| SPP1_NM_ | AAACTATCACTG | 110 | UUUAAUAUU | 158 | AGAGAAUAACA | 206 |
| 001251830_ | TATTTTAATATT | | UGUUAUUCU | | AAUAUUAAA | |
| 1487 | TGTTATTCTCTC | | CU | | | |
| | ATGAATAGA | | | | | |
| SPP1_NM_ | GAGAGCAATGA | 111 | UCCGAUGUG | 159 | UGACUAUCAAU | 207 |
| 001251830_ | GCATTCCGATGT | | AUUGAUAGU | | CACAUCGGA | |
| 1062 | GATTGATAGTCA | | CA | | | |
| | GGAACTTTCC | | | | | |
| SPP1_NM_ | GATGATTCTCAC | 112 | UCUGAUGAG | 160 | GAAUGGUGAGA | 208 |
| 001251830_ | CAGTCTGATGA | | UCUCACCAU | | CUCAUCAGA | |
| 648 | GTCTCACCATTC | | UC | | | |
| | TGATGAATCT | | | | | |
| SPP1_NM_ | AATAAATCTTTT | 113 | UUGAAUGUA | 161 | CAAAUUCUUAU | 209 |
| 001251830_ | ATCTTGAATGTA | | AUAAGAAUU | | UACAUUCAA | |
| 1658 | ATAAGAATTTG | | UC | | | |
| | GTGGTGTCAA | | | | | |
| SPP1_NM_ | TCCTCTGAAGAA | 114 | AAUGACUUU | 162 | GUCUCUUGUUU | 210 |
| 001251830_ | ACCAATGACTTT | | AAACAAGAG | | AAAGUCAUU | |
| 489 | AAACAAGAGAC | | AC | | | |
| | CCTTCCAAGT | | | | | |
| SPP1_NM_ | ATTTGTTATTCT | 115 | AUGAAUAGA | 163 | CUACAUAAAUU | 211 |
| 001251830_ | CTCATGAATAG | | AAUUUAUGU | | UCUAUUCAU | |
| 1508 | AAATTTATGTAG | | AG | | | |
| | AAGCAAACAA | | | | | |
| SPP1_NM_ | ACATGAAATGC | 116 | UUCUCAGUU | 164 | UUCAACCAAUA | 212 |
| 001251830_ | TTCTTTCTCAGT | | UAUUGGUUG | | AACUGAGAA | |
| 1324 | TTATTGGTTGAA | | AA | | | |
| | TGTGTATCTA | | | | | |
| SPP1_NM_ | TCTTTTATCTTG | 117 | GUAAUAAGA | 165 | CACCACCAAAU | 213 |
| 001251830_ | AATGTAATAAG | | AUUUGGUGG | | UCUUAUUAC | |
| 1664 | AATTTGGTGGTG | | UG | | | |
| | TCAATTGCTT | | | | | |

TABLE 9
Sequences targeting mouse OPN gene target regions.

| Sequence | Target | SEQ ID | | SEQ ID | Reverse | SEQ ID |
|---|---|---|---|---|---|---|
| ID | Region | NO | Sequence | NO | complement | NO |
| Spp1_NM_ | AGTGTTAGTG | 214 | UAAGCAGG | 262 | GCAGUUAGU | 310 |
| 001204201_ | AGGGTTAAGC | | AAUACUAA | | AUUCCUGCU | |
| 1108 | AGGAATACTA | | CUGC | | UA | |
| | ACTGCTCATT | | | | | |
| | TCTCA | | | | | |
| Spp1_NM_ | TCTGATGAGA | 215 | ACUGCUAG | 263 | UCUGCUUGU | 311 |
| 001204201_ | CCGTCACTGC | | UACACAAG | | GUACUAGCA | |
| 522 | TAGTACACAA | | CAGA | | GU | |
| | GCAGACACTT | | | | | |
| | TCACT | | | | | |
| Spp1_NM_ | AAGAAAAGG | 216 | UUAAGCAA | 264 | UGGAAGAGU | 312 |
| 001204201_ | ATGACTTTAA | | GAAACUCU | | UUCUUGCUU | |
| 358 | GCAAGAAACT | | UCCA | | AA | |
| | CTTCCAAGCA | | | | | |
| | ATTCCA | | | | | |
| Spp1_NM_ | CTATACCCTT | 217 | UUUAUAAU | 265 | AAAUAUAUG | 313 |
| 001204201_ | TTAGTTTTAT | | UAGCAUAU | | CUAAUUAUA | |
| 1402 | AATTAGCATA | | AUUU | | AA | |
| | TATTTTGTTG | | | | | |
| | TGATT | | | | | |
| Spp1_NM_ | ATGAATAAAA | 218 | GUAGAAAC | 266 | GUAAAUUUG | 314 |
| 001204201_ | TGTATGTAGA | | AAGCAAAU | | CUUGUUUCU | |
| 1323 | AACAAGCAA | | UUAC | | AC | |
| | ATTTACTGAA | | | | | |
| | ACAAGC | | | | | |
| Spp1_NM_ | GGAAGATGAT | 219 | UCUGAAAU | 267 | GAGAAAUUC | 315 |
| 001204201_ | AGGTATCTGA | | UCCGAAUU | | GGAAUUUCA | |
| 986 | AATTCCGAAT | | UCUC | | GA | |
| | TTCTCATGAA | | | | | |
| | TTAGA | | | | | |
| Spp1_NM_ | CTGGATGAAC | 220 | CUGGAAAC | 268 | UCAAGUCUG | 316 |
| 001204201_ | CAAGTCTGGA | | ACACAGAC | | UGUGUUUCC | |
| 813 | AACACACAG | | UUGA | | | |
| | ACTTGAGCAT | | | | AG | |
| | TCCAAA | | | | | |
| Spp1_NM_ | ACAACAACG | 221 | GCAGCCAU | 269 | CUGACUUGA | 317 |
| 001204201_ | GAAAGGGCA | | GAGUCAAG | | CUCAUGGCU | |
| 778 | GCCATGAGTC | | UCAG | | GC | |
| | AAGTCAGCTG | | | | | |
| | GATGAAC | | | | | |
| Spp1_NM_ | TATGAATAAA | 222 | UGUAGAAA | 270 | UAAAUUUGC | 318 |
| 001204201_ | ATGTATGTAG | | CAAGCAAA | | UUGUUUCUA | |
| 1322 | AAACAAGCA | | UUUA | | CA | |
| | AATTTACTGA | | | | | |
| | AACAAG | | | | | |
| Spp1_NM_ | GAGTCTAAGG | 223 | CUCGAUGU | 271 | GCAACAGGG | 319 |
| 001204201_ | AGTCCCTCGA | | CAUCCCUG | | AUGACAUCG | |
| 714 | TGTCATCCCT | | UUGC | | AG | |
| | GTTGCCCAGC | | | | | |
| | TTCTG | | | | | |
| Spp1_NM_ | CTGAAGAAA | 224 | ACUUUAAG | 272 | AAGAGUUUC | 320 |
| 001204201_ | AGGATGACTT | | CAAGAAAC | | UUGCUUAAA | |
| 355 | TAAGCAAGA | | UCUU | | GU | |
| | AACTCTTCCA | | | | | |
| | AGCAATT | | | | | |
| Spp1_NM_ | GAAGCAGAA | 225 | UGCGCCAC | 273 | ACACAGCAU | 321 |
| 001204201_ | TCTCCTTGCG | | AGAAUGCU | | UCUGUGGCG | |
| 317 | CCACAGAATG | | GUGU | | CA | |
| | CTGTGTCCTC | | | | | |
| | TGAAGA | | | | | |
| Spp1_NM_ | GTAACCAAAA | 226 | UGCACUUU | 274 | AAGAACAGA | 322 |
| 001204201_ | GCTTCTGCAC | | GCUUCUGU | | AGCAAAGUG | |
| 1236 | TTTGCTTCTG | | UCUU | | CA | |
| | TTCTTCCTGT | | | | | |
| | ACAAG | | | | | |
| Spp1_NM_ | TGGATATATG | 227 | AGAGAAAG | 275 | AAUAUUACC | 323 |
| 001204201_ | TATGTAGAGA | | AGAGGUAA | | UCUCUUUCU | |
| 1159 | AAGAGAGGT | | UAUU | | CU | |
| | AATATTTTGG | | | | | |
| | GCTCTT | | | | | |
| Spp1_NM_ | GGACTCGGAT | 228 | UGACGAAU | 276 | CCGAAUGGU | 324 |
| 001204201_ | GAATCTGACG | | CUCACCAU | | GAGAUUCGU | |
| 482 | AATCTCACCA | | UCGG | | CA | |
| | TTCGGATGAG | | | | | |
| | TCTGA | | | | | |
| Spp1_NM_ | AGAGAGGTA | 229 | UGGGCUCU | 277 | CAGACUAAG | 325 |
| 001204201_ | ATATTTTGGG | | UAGCUUAG | | CUAAGAGCC | |
| 1180 | CTCTTAGCTT | | UCUG | | CA | |
| | AGTCTGTTGT | | | | | |
| | TTCATG | | | | | |
| Spp1_NM_ | ATTTGCTTTT | 230 | UUUGGCAU | 278 | AGGGAGGAG | 326 |
| 001204201_ | GCCTGTTTGG | | UGCCUCCU | | GCAAUGCCA | |
| 183 | CATTGCCTCC | | CCCU | | AA | |
| | TCCCTCCCGG | | | | | |
| | TGAAA | | | | | |
| Spp1_NM_ | GTGGACTCGG | 231 | UCUGACGA | 279 | GAAUGGUGA | 327 |
| 001204201_ | ATGAATCTGA | | AUCUCACC | | GAUUCGUCA | |
| 480 | CGAATCTCAC | | AUUC | | GA | |
| | CATTCGGATG | | | | | |
| | AGTCT | | | | | |
| Spp1_NM_ | GAAACAAGC | 232 | ACUGAAAC | 280 | UUAAUUCUG | 328 |
| 001204201_ | AAATTTACTG | | AAGCAGAA | | CUUGUUUCA | |
| 1341 | AAACAAGCA | | UUAA | | GU | |
| | GAATTAAAAG | | | | | |
| | AGAAACT | | | | | |
| Spp1_NM_ | GGATGTGATC | 233 | UCAAGCAA | 281 | UGGCUUUGG | 329 |
| 001204201_ | GATAGTCAAG | | GUUCCAAA | | AACUUGCUU | |
| 884 | CAAGTTCCAA | | GCCA | | GA | |
| | AGCCAGCCTG | | | | | |
| | GAACA | | | | | |
| Spp1_NM_ | GAGAGTTCAT | 234 | GAGGUCAA | 282 | CCUCUUCUU | 330 |
| 001204201_ | CTTCTGAGGT | | CUAAAGAA | | UAGUUGACC | |
| 1029 | CAACTAAAGA | | GAGG | | UC | |
| | AGAGGCAAA | | | | | |
| | AACACA | | | | | |
| Spp1_NM_ | AAGGAAGAT | 235 | UAUCUGAA | 283 | GAAAUUCGG | 331 |
| 001204201_ | GATAGGTATC | | AUUCCGAA | | AAUUUCAGA | |
| 984 | TGAAATTCCG | | UUUC | | UA | |
| | AATTTCTCAT | | | | | |
| | GAATTA | | | | | |
| Spp1_NM_ | CTAAAGAAG | 236 | AAACACAG | 284 | CAAAGUAAG | 332 |
| 001204201_ | AGGCAAAAA | | UUCCUUAC | | GAACUGUGU | |
| 1052 | CACAGTTCCT | | UUUG | | UU | |
| | TACTTTGCAT | | | | | |
| | TTAGTAA | | | | | |
| Spp1_NM_ | GGACTAACTA | 237 | AUGAGAUU | 285 | CAAAUCACU | 333 |
| 001204201_ | CGACCATGAG | | GGCAGUGA | | GCCAAUCUC | |
| 153 | ATTGGCAGTG | | UUUG | | AU | |
| | ATTTGCTTTT | | | | | |
| | GCCTG | | | | | |
| Spp1_NM_ | CTTAGCTTAG | 238 | UGUUUCAU | 286 | AACGGUGUU | 334 |
| 001204201_ | TCTGTTGTTT | | GCAAACAC | | UGCAUGAAA | |
| 1201 | CATGCAAACA | | CGUU | | CA | |
| | CCGTTGTAAC | | | | | |
| | CAAAA | | | | | |
| Spp1_NM_ | GACCGTCACT | 239 | UACACAAG | 287 | UGAAAGUGU | 335 |
| 001204201_ | GCTAGTACAC | | CAGACACU | | CUGCUUGUG | |
| 530 | AAGCAGACA | | UUCA | | UA | |
| | CTTTCACTCC | | | | | |
| | AATCGT | | | | | |
| Spp1_NM_ | AAGTTTCACA | 240 | AAGGACAA | 288 | UCUAGGACU | 336 |
| 001204201_ | GCCACAAGG | | GCUAGUCC | | AGCUUGUCC | |
| 939 | ACAAGCTAGT | | UAGA | | UU | |
| | CCTAGACCCT | | | | | |
| | AAGAGT | | | | | |
| Spp1_NM_ | GAGGCAAAA | 241 | UUCCUUAC | 289 | ACUAAAUGC | 337 |
| 001204201_ | ACACAGTTCC | | UUUGCAUU | | AAAGUAAGG | |
| 1060 | TTACTTTGCA | | UAGU | | AA | |
| | TTTAGTAAAA | | | | | |
| | ACAAGA | | | | | |
| Spp1_NM_ | TTCTGTTCTTC | 242 | ACAAGAAA | 290 | UGGCCGUUU | 338 |
| 001204201_ | CTGTACAAGA | | UGCAAACG | | GCAUUUCUU | |
| 1261 | AATGCAAACG | | GCCA | | GU | |
| | GCCACTGCAT | | | | | |
| | TTTA | | | | | |
| Spp1_NM_ | GGCTTATGGA | 243 | GUCAAAGU | 291 | GGAAACUCC | 339 |
| 001204201_ | CTGAGGTCAA | | CUAGGAGU | | UAGACUUUG | |
| 614 | AGTCTAGGAG | | UUCC | | AC | |
| | TTTCCAGGTT | | | | | |
| | TCTGA | | | | | |
| Spp1_NM_ | AGAAAAGGA | 244 | UAAGCAAG | 292 | UUGGAAGAG | 340 |
| 001204201_ | TGACTTTAAG | | AAACUCUU | | UUUCUUGCU | |
| 359 | CAAGAAACTC | | CCAA | | UA | |
| | TTCCAAGCAA | | | | | |
| | TTCCAA | | | | | |
| Spp1_NM_ | GAATAAAATG | 245 | AGAAACAA | 293 | CAGUAAAUU | 341 |
| 001204201_ | TATGTAGAAA | | GCAAAUUU | | UGCUUGUUU | |
| 1325 | CAAGCAAATT | | ACUG | | CU | |
| | TACTGAAACA | | | | | |
| | AGCAG | | | | | |
| Spp1_NM_ | GCTCTTAGCT | 246 | UGUUGUUU | 294 | GGUGUUUGC | 342 |
| 001204201_ | TAGTCTGTTG | | CAUGCAAA | | AUGAAACAA | |
| 1198 | TTTCATGCAA | | CACC | | CA | |
| | ACACCGTTGT | | | | | |
| | AACCA | | | | | |
| Spp1_NM_ | GTAATATTTT | 247 | CUUAGCUU | 295 | AAACAACAG | 343 |
| 001204201_ | GGGCTCTTAG | | AGUCUGUU | | ACUAAGCUA | |
| 1186 | CTTAGTCTGT | | GUUU | | AG | |
| | TGTTTCATGC | | | | | |
| | AAACA | | | | | |
| Spp1_NM_ | GACTGAGGTC | 248 | CUAGGAGU | 296 | AGAAACCUG | 344 |
| 001204201_ | AAAGTCTAGG | | UUCCAGGU | | GAAACUCCU | |
| 622 | AGTTTCCAGG | | UUCU | | AG | |
| | TTTCTGATGA | | | | | |
| | ACAGT | | | | | |
| Spp1_NM_ | TCCAATGAAA | 249 | GACCACAU | 297 | UCAUCGUCG | 345 |
| 001204201_ | GCCATGACCA | | GGACGACG | | UCCAUGUGG | |
| 399 | CATGGACGAC | | AUGA | | UC | |
| | GATGATGACG | | | | | |
| | ATGAT | | | | | |
| Spp1_NM_ | TAAAGAAGA | 250 | AACACAGU | 298 | GCAAAGUAA | 346 |
| 001204201_ | GGCAAAAAC | | UCCUUACU | | GGAACUGUG | |
| 1053 | ACAGTTCCTT | | UUGC | | UU | |
| | ACTTTGCATT | | | | | |
| | TAGTAAA | | | | | |
| Spp1_NM_ | TTTTATGAAT | 251 | GUAUGUAG | 299 | AUUUGCUUG | 347 |
| 001204201_ | AAAATGTATG | | AAACAAGC | | UUUCUACAU | |
| 1319 | TAGAAACAA | | AAAU | | AC | |
| | GCAAATTTAC | | | | | |
| | TGAAAC | | | | | |
| Spp1_NM_ | TAGGTATCTG | 252 | CCGAAUUU | 300 | CUAAUUCAU | 348 |
| 001204201_ | AAATTCCGAA | | CUCAUGAA | | GAGAAAUUC | |
| 995 | TTTCTCATGA | | UUAG | | GG | |
| | ATTAGAGAGT | | | | | |
| | TCATC | | | | | |
| Spp1_NM_ | GAAATTCCGA | 253 | UCAUGAAU | 301 | AUGAACUCU | 349 |
| 001204201_ | ATTTCTCATG | | UAGAGAGU | | CUAAUUCAU | |
| 1004 | AATTAGAGAG | | UCAU | | GA | |
| | TTCATCTTCT | | | | | |
| | GAGGT | | | | | |
| Spp1_NM_ | TTAGCTTAGT | 254 | GUUUCAUG | 302 | CAACGGUGU | 350 |
| 001204201_ | CTGTTGTTTC | | CAAACACC | | UUGCAUGAA | |
| 1202 | ATGCAAACAC | | GUUG | | AC | |
| | CGTTGTAACC | | | | | |
| | AAAAG | | | | | |
| Spp1_NM_ | TAAAAGAGA | 255 | AACAGUCU | 303 | GUAUAGUGA | 351 |
| 001204201_ | AACTGTAACA | | AUAUCACU | | UAUAGACUG | |
| 1373 | GTCTATATCA | | AUAC | | UU | |
| | CTATACCCTT | | | | | |
| | TTAGTT | | | | | |
| Spp1_NM_ | AGAAACAAG | 256 | UACUGAAA | 304 | UAAUUCUGC | 352 |
| 001204201_ | CAAATTTACT | | CAAGCAGA | | UUGUUUCAG | |
| 1340 | GAAACAAGC | | AUUA | | UA | |
| | AGAATTAAAA | | | | | |
| | GAGAAAC | | | | | |
| Spp1_NM_ | TCTTCCTGTA | 257 | AAUGCAAA | 305 | AUGCAGUGG | 353 |
| 001204201_ | CAAGAAATGC | | CGGCCACU | | CCGUUUGCA | |
| 1267 | AAACGGCCAC | | GCAU | | UU | |
| | TGCATTTTAA | | | | | |
| | TGATT | | | | | |
| Spp1_NM_ | GTCCCCAACG | 258 | GGUGAUAG | 306 | CCAUAAGCC | 354 |
| 001204201_ | GCCGAGGTGA | | CUUGGCUU | | AAGCUAUCA | |
| 588 | TAGCTTGGCT | | AUGG | | CC | |
| | TATGGACTGA | | | | | |
| | GGTCA | | | | | |
| Spp1_NM_ | TTGGCTTATG | 259 | AGGUCAAA | 307 | AAACUCCUA | 355 |
| 001204201_ | GACTGAGGTC | | GUCUAGGA | | GACUUUGAC | |
| 612 | AAAGTCTAGG | | GUUU | | CU | |
| | AGTTTCCAGG | | | | | |
| | TTTCT | | | | | |
| Spp1_NM_ | TAAGAGTAAG | 260 | UGAUAGGU | 308 | GGAAUUUCA | 356 |
| 001204201_ | GAAGATGATA | | AUCUGAAA | | GAUACCUAU | |
| 977 | GGTATCTGAA | | UUCC | | CA | |
| | ATTCCGAATT | | | | | |
| | TCTCA | | | | | |
| Spp1_NM_ | TAATATTTTG | 261 | UUAGCUUA | 309 | GAAACAACA | 357 |
| 001204201_ | GGCTCTTAGC | | GUCUGUUG | | GACUAAGCU | |
| 1187 | TTAGTCTGTT | | UUUC | | AA | |
| | GTTTCATGCA | | | | | |
| | AACAC | | | | | |

TABLE 10
Sequences targeting human NOX4 gene target regions.

| Sequence | Target | SEQ ID | | SEQ ID | Reverse | SEQ ID |
|---|---|---|---|---|---|---|
| ID | Region | NO | Sequence | NO | complement | NO |
| NOX4_NM_ | ATATTTAAT | 358 | UAUGUCUU | 408 | AAGACUGUU | 458 |
| 016931_ | AACTAGTAT | | GACAACAG | | GUCAAGACA | |
| 3841 | GTCTTGACA | | UCUU | | UA | |
| | ACAGTCTTT | | | | | |
| | AGATTAAAA | | | | | |
| NOX4_NM_ | ATCAATCAG | 359 | UAUGCCAA | 409 | CCUUACUAU | 1459 |
| 016931_ | CTGTGTTAT | | AGAAUAGU | | UCUUUGGCA | |
| 1953 | GCCAAAGA | | AAGG | | UA | |
| | ATAGTAAGG | | | | | |
| | TTTTCTTATT | | | | | |
| NOX4_NM_ | TTTTGGAAG | 360 | UGAGGAAU | 410 | CAUAGUUCA | 1460 |
| 016931_ | TCCATTTGA | | CACUGAAC | | GUGAUUCCU | |
| 1380 | GGAATCACT | | UAUG | | CA | |
| | GAACTATGA | | | | | |
| | GGTCAGCCT | | | | | |
| NOX4_NM_ | AAGACTTTG | 361 | AGCGGAAU | 411 | CACAGCUGA | 461 |
| 016931_ | AAACTCAGC | | CAAUCAGC | | UUGAUUCCG | |
| 1932 | GGAATCAAT | | UGUG | | CU | |
| | CAGCTGTGT | | | | | |
| | TATGCCAAA | | | | | |
| NOX4_NM_ | AAAGAATA | 362 | UUUCUUAU | 412 | AAAUAAUCA | 462 |
| 016931_1 | GTAAGGTTT | | UUAUGAUU | | UAAAUAAGA | |
| 974 | TCTTATTTA | | AUUU | | AA | |
| | TGATTATTT | | | | | |
| | AAAATGGA | | | | | |
| | AA | | | | | |
| NOX4_ | TACTATAAA | 363 | AGGGAUAU | 413 | AUCCAUAUA | 463 |
| NM_016931_ | TTATAAAGG | | AUAUAUAU | | UAUAUAUCC | |
| 2650 | GATATATAT | | GGAU | | CU | |
| | ATATGGATA | | | | | |
| | TATATATGT | | | | | |
| NOX4_NM_ | TAAGTTCCA | 364 | GUUCAAAU | 414 | UUAACAUUU | 464 |
| 016931_ | GTAGGTGTT | | GUUAAAUG | | AACAUUUGA | |
| 4102 | CAAATGTTA | | UUAA | | AC | |
| | AATGTTAAA | | | | | |
| | CATAGGTCA | | | | | |
| NOX4_NM_ | CTGGCTATC | 365 | UUGGCUCA | 415 | GAAAUAUUG | 465 |
| 016931_ | ATATTCTTG | | GAACAAUA | | UUCUGAGCC | |
| 3164 | GCTCAGAAC | | UUUC | | AA | |
| | AATATTTCC | | | | | |
| | CATTACTAT | | | | | |
| NOX4_NM_ | ATTTAAAAT | 366 | GUGAGAAU | 416 | UCAUCUUGC | 466 |
| 016931_ | GGAAATGTG | | GUGGCAAG | | CACAUUCUC | |
| 2005 | AGAATGTGG | | AUGA | | AC | |
| | CAAGATGAC | | | | | |
| | CGTCACATT | | | | | |
| NOX4_NM_ | TAATTTGCA | 367 | CCAGGCAA | 417 | CAUCUAAUA | 467 |
| 016931_ | AGCACTCCA | | AUAUAUUA | | UAUUUGCCU | |
| 2330 | GGCAAATAT | | GAUG | | GG | |
| | ATTAGATGT | | | | | |
| | TTGAAAACA | | | | | |
| NOX4_NM_ | AAAAAAAA | 368 | UAAAGCAU | 418 | UAAACUGUA | 468 |
| 016931_ | AAAGAATTA | | UCAUACAG | | UGAAUGCUU | |
| 3511 | AAGCATTCA | | UUUA | | UA | |
| | TACAGTTTA | | | | | |
| | GTGATTTTG | | | | | |
| | T | | | | | |
| NOX4_NM_ | GGGTTTAAA | 369 | GUUGGAAC | 419 | UCACACAGU | 469 |
| 016931_ | GTCACCGTT | | UACACUGU | | GUAGUUCCA | |
| 3110 | GGAACTACA | | GUGA | | AC | |
| | CTGTGTGAT | | | | | |
| | CTTATAAAC | | | | | |
| NOX4_NM_ | CTAGTAAAA | 370 | UUUGAGUU | 420 | UAAGCCUGG | 470 |
| 016931_ | AGACACTTT | | AGUCCAGG | | ACUAACUCA | |
| 3918 | GAGTTAGTC | | CUUA | | AA | |
| | CAGGCTTAA | | | | | |
| | TGTGCAATA | | | | | |
| NOX4_NM_ | ATTTTAAAG | 371 | CUGGUCAG | 421 | UAGAGUAAU | 471 |
| 016931_ | CAAGACCTG | | UAUAUUAC | | AUACUGACC | |
| 1133 | GTCAGTATA | | UCUA | | AG | |
| | TTACTCTAC | | | | | |
| | ATTGTCCCA | | | | | |
| NOX4_NM_ | ATTACTGTC | 372 | UUUGGUAU | 422 | UGGAAACUC | 472 |
| 016931_ | GCACTTTTT | | CCUGAGUU | | AGGAUACCA | |
| 3063 | GGTATCCTG | | UCCA | | AA | |
| | AGTTTCCAT | | | | | |
| | AGGGAACTA | | | | | |
| NOX4_NM_ | ATGTTTCAA | 373 | UCUGCCAU | 423 | GGCUCAUCU | 473 |
| 016931_ | AGCTGGTCT | | UCUAGAUG | | AGAAUGGCA | |
| 4055 | GCCATTCTA | | AGCC | | GA | |
| | GATGAGCCT | | | | | |
| | CCTTGCTTA | | | | | |
| NOX4_NM_ | TTGATACGG | 374 | UGUGUCAA | 424 | GACGAUUAG | 474 |
| 016931_ | GTACTTTGT | | UAUCUAAU | | AUAUUGACA | |
| 2392 | GTCAATATC | | CGUC | | CA | |
| | TAATCGTCT | | | | | |
| | CCACTACTT | | | | | |
| NOX4_NM_ | ATTTTCATG | 375 | UUUAACAG | 425 | UACUAGUUC | 475 |
| 016931_ | GAAACATTT | | AAAGAACU | | UUUCUGUUA | |
| 3889 | AACAGAAA | | AGUA | | AA | |
| | GAACTAGTA | | | | | |
| | AAAAGACA | | | | | |
| | CT | | | | | |
| NOX4_NM_ | TTTTTCAGC | 376 | GUCUCUAU | 426 | ACAAUAAUA | 476 |
| 016931_ | ATTTTTGTC | | UAUUAUUA | | AUAAUAGAG | |
| 2776 | TCTATTATT | | UUGU | | AC | |
| | ATTATTGTT | | | | | |
| | TTTTTCCCA | | | | | |
| NOX4_NM_ | ACTGGACAG | 377 | UUCGAGAU | 427 | AGGCAGUAG | 477 |
| 016931_ | AACGATTTC | | UUACUACU | | UAAAUCUCG | |
| 1271 | GAGATTTAC | | GCCU | | AA | |
| | TACTGCCTC | | | | | |
| | CATCTAGTC | | | | | |
| NOX4_NM_ | TGTTTCAGG | 378 | GCUGAAGU | 428 | AAUUAGUUU | 478 |
| 016931 | AGGGCTGCT | | AUCAAACU | | GAUACUUCA | |
| 765 | GAAGTATCA | | AAUU | | GC | |
| | AACTAATTT | | | | | |
| | AGATACCCA | | | | | |
| NOX4_NM_ | GAATGTGGC | 379 | ACCGUCAC | 429 | UAAACAUGU | 1479 |
| 016931_ | AAGATGACC | | AUUACAUG | | AAUGUGACG | |
| 2024 | GTCACATTA | | UUUA | | GU | |
| | CATGTTTAA | | | | | |
| | TCTGGAAAC | | | | | |
| NOX4_NM_ | TTGCATAAC | 380 | UGGCAAGA | 430 | UCAGGUCUG | 480 |
| 016931_ | AAGTTTTGG | | GAACAGAC | | UUCUCUUGC | |
| 1579 | CAAGAGAA | | CUGA | | CA | |
| | CAGACCTGA | | | | | |
| | CTATGTCAA | | | | | |
| | C | | | | | |
| NOX4_NM_ | ATGTGTGTC | 381 | GACUGAAU | 431 | GAUAAAUCU | 481 |
| 016931_ | AGTAGGGAC | | AUAAGAUU | | UAUAUUCAG | |
| 2892 | TGAATATAA | | UAUC | | UC | |
| | GATTTATCT | | | | | |
| | CCTTTGCCA | | | | | |
| NOX4_NM_ | TTTGGTATC | 382 | UUCCAUAG | 432 | CCCAAUAGU | 482 |
| 016931_ | CTGAGTTTC | | GGAACUAU | | UCCCUAUGG | |
| 3078 | CATAGGGAA | | UGGG | | AA | |
| | CTATTGGGT | | | | | |
| | TTAAAGTCA | | | | | |
| NOX4_NM_ | AACTTAAGT | 383 | UCCAGAAU | 433 | UGCGACAGU | 483 |
| 016931_ | TTAGAATCC | | AUUACUGU | | AAUAUUCUG | |
| 3040 | AGAATATTA | | CGCA | | GA | |
| | CTGTCGCAC | | | | | |
| | TTTTTGGTA | | | | | |
| NOX4_NM_ | TTCTCTGGG | 384 | UCUUAUAA | 434 | UUAAGGCUU | 484 |
| 016931_ | GATCATTCT | | UACAAGCC | | GUAUUAUAA | |
| 4212 | TATAATACA | | UUAA | | GA | |
| | AGCCTTAAA | | | | | |
| | TCACTAATT | | | | | |
| NOX4_NM_ | ATGTTTCAG | 385 | UGCUGAAG | 435 | AUUAGUUUG | 485 |
| 016931_ | GAGGGCTGC | | UAUCAAAC | | AUACUUCAG | |
| 764 | TGAAGTATC | | UAAU | | CA | |
| | AAACTAATT | | | | | |
| | TAGATACCC | | | | | |
| NOX4_NM_ | CTAACTATG | 386 | UCUGGUAU | 436 | GAGGUUAUG | 486 |
| 016931_ | ATATCTTCT | | ACUCAUAA | | AGUAUACCA | |
| 698 | GGTATACTC | | CCUC | | GA | |
| | ATAACCTCT | | | | | |
| | TCTTTGTCT | | | | | |
| NOX4_NM_ | ATTTGTGAA | 387 | UAUGGAAG | 437 | GGAAUCUGG | 487 |
| 016931_ | GATTTGTAT | | AGCCCAGA | | GCUCUUCCA | |
| 939 | GGAAGAGC | | UUCC | | UA | |
| | CCAGATTCC | | | | | |
| | AAGCTAATT | | | | | |
| | T | | | | | |
| NOX4_NM_ | TTTCAGCTG | 388 | UUUGCCAU | 438 | GAGUCCUGC | 488 |
| 016931_ | AAAACTTTT | | GAAGCAGG | | UUCAUGGCA | |
| 1872 | GCCATGAAG | | ACUC | | AA | |
| | CAGGACTCT | | | | | |
| | AAAGAAGG | | | | | |
| | A | | | | | |
| NOX4_NM_ | TATTATTAT | 389 | UUUCCCAG | 439 | GACAAACCC | 489 |
| 016931_ | TGTTTTTTTC | | GUAGGGUU | | UACCUGGGA | |
| 2799 | CCAGGTAGG | | UGUC | | AA | |
| | GTTTGTCTT | | | | | |
| | AGGCTGTA | | | | | |
| NOX4_NM_ | AAGAATAGT | 390 | UUCUUAUU | 440 | UAAAUAAUC | 490 |
| 016931_ | AAGGTTTTC | | UAUGAUUA | | AUAAAUAAG | |
| 1975 | TTATTTATG | | UUUA | | AA | |
| | ATTATTTAA | | | | | |
| | AATGGAAAT | | | | | |
| NOX4_NM_ | ATGCAATAA | 391 | CUAACUAU | 441 | CCAGAAGAU | 491 |
| 016931_ | GAGTTTCTA | | GAUAUCUU | | AUCAUAGUU | |
| 683 | ACTATGATA | | CUGG | | AG | |
| | TCTTCTGGT | | | | | |
| | ATACTCATA | | | | | |
| NOX4_NM_ | TATGCAATA | 392 | UCUAACUA | 442 | CAGAAGAUA | 492 |
| 016931_ | AGAGTTTCT | | UGAUAUCU | | UCAUAGUUA | |
| 682 | AACTATGAT | | UCUG | | GA | |
| | ATCTTCTGG | | | | | |
| | TATACTCAT | | | | | |
| NOX4_NM_ | ATCATTCTT | 393 | CAAGCCUU | 443 | AAUUAGUGA | 493 |
| 016931_ | ATAATACAA | | AAAUCACU | | UUUAAGGCU | |
| 4222 | GCCTTAAAT | | AAUU | | UG | |
| | CACTAATTT | | | | | |
| | TAGTAGCAA | | | | | |
| NOX4_NM_ | TTACCTTTT | 394 | GAGGAAAU | 444 | UAAGAAUAU | 494 |
| 016931_ | AAGTAAGA | | AUGAUAUU | | CAUAUUUCC | |
| 3001 | GGAAATATG | | CUUA | | UC | |
| | ATATTCTTA | | | | | |
| | TTCAAACTT | | | | | |
| | A | | | | | |
| NOX4_NM_ | ATTTCTCTG | 395 | AUUCUUAU | 445 | AAGGCUUGU | 495 |
| 016931_ | GGGATCATT | | AAUACAAG | | AUUAUAAGA | |
| 4210 | CTTATAATA | | CCUU | | JAU | |
| | CAAGCCTTA | | | | | |
| | AATCACTAA | | | | | |
| NOX4_NM_ | ATCACAGCC | 396 | UAUGCAAU | 446 | UUAGAAACU | 496 |
| 016931_ | TCTACATAT | | AAGAGUUU | | CUUAUUGCA | |
| 667 | GCAATAAGA | | CUAA | | UA | |
| | GTTTCTAAC | | | | | |
| | TATGATATC | | | | | |
| NOX4_NM_ | TTACATAAA | 397 | CAUGAUAG | 447 | AGGCCUUAG | 497 |
| 016931_ | ACATAACAT | | AUGCUAAG | | CAUCUAUCA | |
| 3636 | GATAGATGC | | GCCU | | UG | |
| | TAAGGCCTT | | | | | |
| | TTTTTGCTA | | | | | |
| NOX4_NM_ | GGTCAAAGA | 398 | UAAAGCAA | 448 | ACUGACCAG | 498 |
| 016931_ | AAATTTTAA | | GACCUGGU | | GUCUUGCUU | |
| 1122 | AGCAAGACC | | CAGU | | UA | |
| | TGGTCAGTA | | | | | |
| | TATTACTCT | | | | | |
| NOX4_NM_ | ATTCTAGAT | 399 | CCUUGCUU | 449 | UGGAACUUA | 499 |
| 016931_ | GAGCCTCCT | | AUUUAAGU | | AAUAAGCAA | |
| 4076 | TGCTTATTT | | UCCA | | GG | |
| | AAGTTCCAG | | | | | |
| | TAGGTGTTC | | | | | |
| NOX4_NM_ | GTCCCTTTC | 400 | AUUUAAAU | 450 | CAAUAGUGA | 500 |
| 016931_ | ATCTTCATT | | CUAUCACU | | UAGAUUUAA | |
| 2595 | TAAATCTAT | | AUUG | | AU | |
| | CACTATTGC | | | | | |
| | ATGGTAATG | | | | | |
| NOX4_NM_ | TAGATTAAA | 401 | UUUCAUGG | 451 | UGUUAAAUG | 501 |
| 016931_ | AAGAATTTT | | AAACAUUU | | UUUCCAUGA | |
| 3876 | CATGGAAAC | | AACA | | AA | |
| | ATTTAACAG | | | | | |
| | AAAGAACTA | | | | | |
| NOX4_NM_ | TGAGTAACC | 402 | ACUCAUAU | 452 | AAAUCUUGU | 502 |
| 016931_ | AGAACAACT | | GGGACAAG | | CCCAUAUGA | |
| 1820 | CATATGGGA | | AUUU | | GU | |
| | CAAGATTTG | | | | | |
| | AATACAATA | | | | | |
| NOX4_NM_ | CGAAATTCT | 403 | CAUUCAAU | 453 | GAUAAUUUC | 503 |
| 016931_ | GCCCTTCAT | | CUAGAAAU | | UAGAUUGAA | |
| 1323 | TCAATCTAG | | UAUC | | UG | |
| | AAATTATCC | | | | | |
| | CAAGCTGTA | | | | | |
| NOX4_NM_ | CTGAATGAT | 404 | UUGUAGAU | 454 | AAAGGAAGA | 504 |
| 016931_ | ATTTACTTG | | AUUUCUUC | | AAUAUCUAC | |
| 3717 | TAGATATTT | | CUUU | | AA | |
| | CTTCCTTTC | | | | | |
| | CTTGAAATT | | | | | |
| NOX4_NM_ | AGACTATAT | 405 | UGAACCUU | 455 | CAACAUUUG | 505 |
| 016931_ | GCTAACTGA | | CCUCAAAU | | AGGAAGGUU | |
| 2464 | ACCTTCCTC | | GUUG | | CA | |
| | AAATGTTGT | | | | | |
| | TATAGTATC | | | | | |
| NOX4_NM_ | TATGGGACA | 406 | GAAUACAA | 456 | AAAGACUCU | 506 |
| 016931_ | AGATTTGAA | | UAAAGAGU | | UUAUUGUAU | |
| 1840 | TACAATAAA | | CUUU | | UC | |
| | GAGTCTTTC | | | | | |
| | AGCTGAAAA | | | | | |
| NOX4_NM_ | ATCGTCTCC | 407 | UAUGCUAA | 457 | CAAAUAGAG | 507 |
| 016931_ | ACTACTTAT | | UACCUCUA | | GUAUUAGCA | |
| 2421 | GCTAATACC | | UUUG | | UA | |
| | TCTATTTGA | | | | | |
| | TATCTGAAG | | | | | |

TABLE 11
Sequences targeting mouse NOX4 gene target regions.

| Sequence | Target | SEQ ID | | SEQ ID | Reverse | SEQ ID |
|---|---|---|---|---|---|---|
| ID | Region | NO | Sequence | NO | complement | NO |
| Nox4_NM_ | CATACTAAA | 508 | AGCAUCAA | 558 | ACAGGUGGU | 608 |
| 001285833_ | ACATTTAGC | | AUAACCAC | | UAUUUGAUG | |
| 3301 | ATCAAATAA | | CUGU | | CU | |
| | CCACCTGTA | | | | | |
| | TATTTAGTT | | | | | |
| Nox4_NM_ | GTTTATCAA | 509 | UUGGUAUA | 559 | CAUUAAUAA | 609 |
| 001285833_ | CTTAATTTG | | UAAUUAUU | | UUAUAUACC | |
| 3139 | GTATATAAT | | AAUG | | AA | |
| | TATTAATGC | | | | | |
| | TATCAAATA | | | | | |
| Nox4_NM_ | ATGATTACA | 510 | CCUGUUAU | 560 | GAAAUCCAA | 610 |
| 001285833_ | ATAAATCCT | | UAUUUGGA | | AUAAUAACA | |
| 3241 | GTTATTATT | | UUUC | | GG | |
| | TGGATTTCA | | | | | |
| | TGTGATTCA | | | | | |
| Nox4_NM_ | TTTTGTATA | 511 | GAUUACAA | 561 | UAACAGGAU | 611 |
| 001285833_ | TCACATGAT | | UAAAUCCU | | UUAUUGUAA | |
| 3228 | TACAATAAA | | GUUA | | UC | |
| | TCCTGTTAT | | | | | |
| | TATTTGGAT | | | | | |
| Nox4_NM_ | AAGACTTAG | 512 | AGCUGAAU | 562 | CACAGCUGU | 612 |
| 001285833_ | AAACTCAGC | | CAAACAGC | | UUGAUUCAG | |
| 2112 | TGAATCAAA | | UGUG | | CU | |
| | CAGCTGTGC | | | | | |
| | TATGCCAAA | | | | | |
| Nox4_NM_ | AGATTTAAG | 513 | GUUCAUGU | 563 | ACUGAUUGG | 613 |
| 001285833_ | TCCCCTGTT | | GAUCCAAU | | AUCACAUGA | |
| 3032 | CATGTGATC | | CAGU | | AC | |
| | CAATCAGTT | | | | | |
| | TATCACCTA | | | | | |
| Nox4_NM_ | CCTGTCACC | | UCAGUCAU | 564 | GAGGGAUGA | 614 |
| 001285833_ | ATCATCTCA | | CAAUCAUC | | UUGAUGACU | |
| 1130 | GTCATCAAT | | CCUC | | GA | |
| | CATCCCTCT | | | | | |
| | GATGTAATG | | | | | |
| | (SEQ ID NO: | | | | | |
| | 72) | | | | | |
| Nox4_NM_ | AAAGAATAC | 515 | UUUGCUAU | 565 | AAAUAAUCA | 615 |
| 001285833_ | CAAGGGTTT | | UUAUGAUU | | UAAAUAGCA | |
| 2154 | GCTATTTAT | | AUUU | | AA | |
| | GATTATTTA | | | | | |
| | AAATGAGAA | | | | | |
| Nox4_NM_ | AAGCATACT | 516 | UUUAGCAU | 566 | GGUGGUUAU | 616 |
| 001285833_ | AAAACATTT | | CAAAUAAC | | UUGAUGCUA | |
| 3298 | AGCATCAAA | | CACC | | AA | |
| | TAACCACCT | | | | | |
| | GTATATTTA | | | | | |
| Nox4_NM_ | ATGCTTGGT | 517 | UUGUAUAU | 567 | UGUAAUCAU | 617 |
| 001285833_ | AATCTTTTG | | CACAUGAU | | GUGAUAUAC | |
| 3215 | TATATCACA | | UACA | | AA | |
| | TGATTACAA | | | | | |
| | TAAATCCTG | | | | | |
| Nox4_NM_ | ATTTGGAAG | 518 | UGAGGAGU | 568 | CAUAGUUCA | 618 |
| 001285833_ | CCCATTTGA | | CACUGAAC | | GUGACUCCU | |
| 1561 | GGAGTCACT | | UAUG | | CA | |
| | GAACTATGA | | | | | |
| | AGTTAGTCT | | | | | |
| Nox4_NM_ | ATTATTTTT | 519 | UUUGAUAU | 569 | UAACUCACC | 619 |
| 001285833_ | GCACTGTTT | | CACGGUGA | | GUGAUAUCA | |
| 2995 | GATATCACG | | GUUA | | AA | |
| | GTGAGTTAC | | | | | |
| | TAGATTTAA | | | | | |
| Nox4_NM_ | ATCATAACT | 520 | UAUGCCUA | 570 | GAUGAAUUC | 620 |
| 001285833_ | TATTTTTAT | | UGUGAAUU | | ACAUAGGCA | |
| 3577 | GCCTATGTG | | CAUC | | UA | |
| | AATTCATCA | | | | | |
| | CATTTCAAA | | | | | |
| Nox4_NM_ | TGTGGAGAC | 521 | UAUGACAU | 571 | AGCAUUGCU | 621 |
| 001285833_ | ATAAACTAT | | CAUAGCAA | | AUGAUGUCA | |
| 3424 | GACATCATA | | UGCU | | UA | |
| | GCAATGCTG | | | | | |
| | TCTAGATTA | | | | | |
| Nox4_NM_ | TAAGAAAGA | 522 | UCAUACAG | 572 | UAAGUUGAU | 622 |
| 001285833_ | AGATATTCA | | UUUAUCAA | | AAACUGUAU | |
| 3117 | TACAGTTTA | | CUUA | | GA | |
| | TCAACTTAA | | | | | |
| | TTTGGTATA | | | | | |
| Nox4_NM_ | AGTTTATCA | 523 | UUUGGUAU | 573 | AUUAAUAAU | 623 |
| 001285833_ | ACTTAATTT | | AUAAUUAU | | UAUAUACCA | |
| 3138 | GGTATATAA | | UAAU | | AA | |
| | TTATTAATG | | | | | |
| | CTATCAAAT | | | | | |
| Nox4_NM_ | ACTTTAAAG | 524 | CUGGCCAG | 574 | GAGAAUAAU | 624 |
| 001285833_ | CAAGACCTG | | UAUAUUAU | | AUACUGGCC | |
| 1197 | GCCAGTATA | | UCUC | | AG | |
| | TTATTCTCC | | | | | |
| | ATTGCCCCA | | | | | |
| Nox4_NM_ | TTATACCCA | 525 | UGUAGUAU | 575 | AAACAUAAG | 625 |
| 001285833_ | TGTGGATGT | | CUACUUAU | | UAGAUACUA | |
| 2644 | AGTATCTAC | | GUUU | | CA | |
| | TTATGTTTG | | | | | |
| | CATCTTATC | | | | | |
| Nox4_NM_ | AGAAGCACA | 526 | CUCUCCUU | 576 | CAAAGUGAG | 626 |
| 001285833_ | AGACCTCTC | | UGACUCAC | | UCAAAGGAG | |
| 2854 | TCCTTTGAC | | UUUG | | AG | |
| | TCACTTTGA | | | | | |
| | TTTCAGATA | | | | | |
| Nox4_NM_ | ATTATTTAA | 527 | UGUUUGAU | 577 | AACAAAUCG | 627 |
| 001285833_ | TAGAACTGT | | CACCGAUU | | GUGAUCAAA | |
| 2734 | TTGATCACC | | UGUU | | CA | |
| | GATTTGTTC | | | | | |
| | AATTTAGTC | | | | | |
| Nox4_NM_ | GTTACAGCT | | UACGCAAU | 578 | UUAGAAACU | 628 |
| 001285833_ | TCTACCTAC | | AAGAGUUU | | CUUAUUGCG | |
| 731 | GCAATAAGA | | CUAA | | UA | |
| | GTTTCTAAT | | | | | |
| | TATGATATC | | | | | |
| | (SEQ ID NO: | | | | | |
| | 73) | | | | | |
| Nox4_NM_ | GTGCAATAC | 529 | CUUACCAA | 579 | AAUAAGUUA | 629 |
| 001285833_ | TGATTCCTT | | UCAUAACU | | UGAUUGGUA | |
| 3555 | ACCAATCAT | | UAUU | | AG | |
| | AACTTATTT | | | | | |
| | TTATGCCTA | | | | | |
| Nox4_NM_ | AGCTTTATC | 530 | AUAGAAGA | 580 | GAAAUGAAG | 630 |
| 001285833_ | ACACATATA | | CAGCUUCA | | CUGUCUUCU | |
| 3766 | GAAGACAGC | | UUUC | | AU | |
| | TTCATTTCTT | | | | | |
| | TTTAGATT | | | | | |
| Nox4_NM_ | AAGAATACC | 531 | UUGCUAUU | 581 | UAAAUAAUC | 631 |
| 001285833_ | AAGGGTTTG | | UAUGAUUA | | AUAAAUAGC | |
| 2155 | CTATTTATG | | UUUA | | AA | |
| | ATTATTTAA | | | | | |
| | AATGAGAAT | | | | | |
| Nox4_NM_ | GAGCCAAAG | 532 | UGAAGAAU | 582 | ACCACAUCA | 632 |
| 001285833_ | GGGCCCTGA | | AUCUGAUG | | GAUAUUCUU | |
| 2235 | AGAATATCT | | UGGU | | CA | |
| | GATGTGGTG | | | | | |
| | ATTCACATT | | | | | |
| Nox4_NM_ | GTTTATCAC | 533 | UUUAAUAU | 583 | UCUGAUUCC | 633 |
| 001285833_ | CTATTTTTTA | | UCAGGAAU | | UGAAUAUUA | |
| 3065 | ATATTCAGG | | CAGA | | AA | |
| | AATCAGAAG | | | | | |
| | GAAAATTT | | | | | |
| Nox4_NM_ | TTTCCTATC | 534 | AUGGGCAA | 584 | GAGCAUUGC | 634 |
| 001285833_ | CCTCCTATG | | UGUGCAAU | | ACAUUGCCC | |
| 3376 | GGCAATGTG | | GCUC | | AU | |
| | CAATGCTCA | | | | | |
| | TGTAGACTG | | | | | |
| Nox4_NM_ | TGAGATTCT | 535 | CAUUCACU | 585 | GGUAAUUUC | 635 |
| 001285833_ | GCCCTTCAT | | CUAGAAAU | | UAGAGUGAA | |
| 1504 | TCACTCTAG | | UACC | | UG | |
| | AAATTACCC | | | | | |
| | TAAGTTATA | | | | | |
| Nox4_NM_ | TTTATCTGG | 536 | AGAGACAU | 586 | UGGAAUGAU | 636 |
| 001285833_ | GTGTGCAGA | | CCAAUCAU | | UGGAUGUCU | |
| 1700 | GACATCCAA | | UCCA | | CU | |
| | TCATTCCAG | | | | | |
| | TGGTTTGCA | | | | | |
| Nox4_NM_ | TTATTTTTAT | 537 | UGUGAAUU | 587 | UGAAAUGUG | 637 |
| 001285833_ | GCCTATGTG | | CAUCACAU | | AUGAAUUCA | |
| 3585 | AATTCATCA | | UUCA | | CA | |
| | CATTTCAAA | | | | | |
| | AATTCAGG | | | | | |
| Nox4_NM_ | TGCAATACT | 538 | UUACCAAU | 588 | AAAUAAGUU | 638 |
| 001285833_ | GATTCCTTA | | CAUAACUU | | AUGAUUGGU | |
| 3556 | CCAATCATA | | AUUU | | AA | |
| | ACTTATTTTT | | | | | |
| | ATGCCTAT | | | | | |
| Nox4_NM_ | AACTCCAGT | 539 | UCAAGGAU | 589 | UUUAACAUA | 639 |
| 001285833_ | GCATGTTCA | | CUAUAUGU | | UAGAUCCUU | |
| 3700 | AGGATCTAT | | UAAA | | GA | |
| | ATGTTAAAC | | | | | |
| | ATGGTATAT | | | | | |
| Nox4_NM_ | CCTCTTTAA | 540 | UUCGGUUG | 590 | CUUGAAUGU | 640 |
| 001285833_ | ATTGTTTTC | | AACACAUU | | GUUCAACCG | |
| 2395 | GGTTGAACA | | CAAG | | AA | |
| | CATTCAAGA | | | | | |
| | TTGAACAAA | | | | | |
| Nox4_NM_ | TTGTATATC | 541 | UUACAAUA | 591 | AAUAACAGG | 641 |
| 001285833_ | ACATGATTA | | AAUCCUGU | | AUUUAUUGU | |
| 3230 | CAATAAATC | | UAUU | | AA | |
| | CTGTTATTA | | | | | |
| | TTTGGATTT | | | | | |
| Nox4_NM_ | GATTTCATG | 542 | AGUACUAA | 592 | UAUGCUUGU | 642 |
| 001285833_ | TGATTCAGT | | CAGACAAG | | CUGUUAGUA | |
| 3270 | ACTAACAGA | | CAUA | | CU | |
| | CAAGCATAC | | | | | |
| | TAAAACATT | | | | | |
| Nox4_NM_ | ACTTGTGAA | 543 | CCUGGAAG | 593 | GGAACUUGG | 643 |
| 001285833_ | GATTTGCCT | | AACCCAAG | | GUUCUUCCA | |
| 1003 | GGAAGAACC | | UUCC | | GG | |
| | CAAGTTCCA | | | | | |
| | AGCTCATTT | | | | | |
| Nox4_NM_ | AGATATTCA | 544 | UUAUCAAC | 594 | AUACCAAAU | 644 |
| 001285833_ | TACAGTTTA | | UUAAUUUG | | UAAGUUGAU | |
| 3126 | TCAACTTAA | | GUAU | | AA | |
| | TTTGGTATA | | | | | |
| | TAATTATTA | | | | | |
| Nox4_NM_ | TACTTTATC | 545 | UGCAGAGA | 595 | AAUGAUUGG | 645 |
| 001285833_ | TGGGTGTGC | | CAUCCAAU | | AUGUCUCUG | |
| 1697 | AGAGACATC | | CAUU | | CA | |
| | CAATCATTC | | | | | |
| | CAGTGGTTT | | | | | |
| Nox4_NM_ | CCATTTTCT | 546 | UGCAUAAG | 596 | AAAGCUACU | 646 |
| 001285833_ | AGGTTGTGC | | UGGAGUAG | | CCACUUAUG | |
| 2466 | ATAAGTGGA | | CUUU | | CA | |
| | GTAGCTTTC | | | | | |
| | ATTTGGATA | | | | | |
| Nox4_NM_ | GAAAATTTA | 547 | UAAGAAAG | 597 | UAUGAAUAU | 647 |
| 001285833_ | CTACCTTAA | | AAGAUAUU | | CUUCUUUCU | |
| 3102 | GAAAGAAG | | CAUA | | UA | |
| | ATATTCATA | | | | | |
| | CAGTTTATC | | | | | |
| | A | | | | | |
| Nox4_NM_ | CACTCTACT | 548 | CUGGAAAC | 598 | UUAACUUGU | 648 |
| 001285833_ | GGATGACTG | | CAUACAAG | | AUGGUUUCC | |
| 1654 | GAAACCATA | | UUAA | | AG | |
| | CAAGTTAAG | | | | | |
| | AAGACTGTA | | | | | |
| Nox4_NM__ | CTAATTATG | 549 | UCUGGUAU | 599 | GAGGUUGUG | 649 |
| 001285833_ | ATATCTTCT | | ACUCACAA | | AGUAUACCA | |
| 762 | GGTATACTC | | CCUC | | GA | |
| | ACAACCTCT | | | | | |
| | TCTTTGTCT | | | | | |
| Nox4_NM_ | ATTTAATAG | 550 | UUGAUCAC | 600 | UUGAACAAA | 650 |
| 001285833_ | AACTGTTTG | | CGAUUUGU | | UCGGUGAUC | |
| 2737 | ATCACCGAT | | UCAA | | AA | |
| | TTGTTCAAT | | | | | |
| | TTAGTCCCA | | | | | |
| Nox4_NM_ | CTTTTTAGA | 551 | GGUGGUGG | 601 | AUACAACAG | 651 |
| 001285833_ | TTCTATGGT | | UUACUGUU | | UAACCACCAC | |
| 3800 | GGTGGTTAC | | GUAU | | C | |
| | TGTTGTATA | | | | | |
| | AATCTTAAA | | | | | |
| Nox4_NM_ | TCTACATGC | 552 | UGUUGCAU | 602 | ACCACCUGA | 652 |
| 001285833_ | TGCTGCTGT | | GUUUCAGG | | AACAUGCAA | |
| 807 | TGCATGTTT | | UGGU | | CA | |
| | CAGGTGGTT | | | | | |
| | TGTTGAAGT | | | | | |
| Nox4_NM_ | TGGTATATA | 553 | AAUGCUAU | 603 | GUGUCCUAU | 653 |
| 001285833_ | ATTATTAAT | | CAAAUAGG | | UUGAUAGCA | |
| 3155 | GCTATCAAA | | ACAC | | UU | |
| | TAGGACACA | | | | | |
| | TTGCTGAAA | | | | | |
| Nox4_NM_ | GATATTCAT | 554 | UAUCAACU | 604 | UAUACCAAA | 654 |
| 001285833_ | ACAGTTTAT | | UAAUUUGG | | UUAAGUUGA | |
| 3127 | CAACTTAAT | | UAUA | | UA | |
| | TTGGTATAT | | | | | |
| | AATTATTAA | | | | | |
| Nox4_NM_ | CACATTGCT | 555 | AGUACCUU | 605 | CAAGCAUAA | 655 |
| 001285833_ | GAAATTAGT | | AACUUAUG | | GUUAAGGUA | |
| 3187 | ACCTTAACT | | CUUG | | CU | |
| | TATGCTTGG | | | | | |
| | TAATCTTTT | | | | | |
| Nox4_NM_ | GTATTTATT | 556 | UCAGUUUA | 606 | UAUUAGUGA | 656 |
| 001285833_ | CAAATTTCA | | GAAUCACU | | UUCUAAACU | |
| 2961 | GTTTAGAAT | | AAUA | | GA | |
| | CACTAATAT | | | | | |
| | TATTTTTGC | | | | | |
| Nox4_NM_ | ACTTCCAAG | 557 | CAUGCCUU | 607 | GGACACAGU | 657 |
| 001285833_ | ATGAACCAT | | CUUACUGU | | AAGAAGGCA | |
| 1370 | GCCTTCTTA | | GUCC | | UG | |
| | CTGTGTCCT | | | | | |
| | ACTGAAACC | | | | | |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
1. Younossi, Z. M., Blissett, D., Blissett, R., Henry, L., Stepanova, M., Younossi, Y., Racila, A., Hunt, S., and Beckerman, R. (2016). The economic and clinical burden of nonalcoholic fatty liver disease in the United States and Europe. Hepatology. John Wiley and Sons Inc.
2. Le, M. H., Yeo, Y. H., Zou, B., Barnet, S., Henry, L., Cheung, R., and Nguyen, M. H. (2022). Forecasted 2040 global prevalence of nonalcoholic fatty liver disease using hierarchical bayesian approach. Clin Mol Hepatol 28, 841-850. 10.3350/cmh.2022.0239.
3. Younossi, Z. M., Stepanova, M., Ong, J., Trimble, G., AlQahtani, S., Younossi, I., Ahmed, A., Racila, A., and Henry, L. (2021). Nonalcoholic Steatohepatitis Is the Most Rapidly Increasing Indication for Liver Transplantation in the United States. Clin Gastroenterol Hepatol 19, 580-589.e585. 10.1016/j.cgh.2020.05.064.
4. Younossi, Z. M., Koenig, A. B., Abdelatif, D., Fazel, Y., Henry, L., and Wymer, M. (2016). Global epidemiology of nonalcoholic fatty liver disease-Meta-analytic assessment of prevalence, incidence, and outcomes. Hepatology. John Wiley and Sons Inc.
5. M, N., and AJ, S. (2018). Pathogenesis of NASH: The Impact of Multiple Pathways. Current hepatology reports. Curr Hepatol Rep.
6. Lomonaco, R., Ortiz-Lopez, C., Orsak, B., Webb, A., Hardies, J., Darland, C., Finch, J., Gastaldelli, A., Harrison, S., Tio, F., and Cusi, K. (2012). Effect of adipose tissue insulin resistance on metabolic parameters and liver histology in obese patients with nonalcoholic fatty liver disease. Hepatology. Hepatology.
7. Esler, W. P., and Bence, K. K. (2019). Metabolic Targets in Nonalcoholic Fatty Liver Disease. Cellular and Molecular Gastroenterology and Hepatology. Elsevier.
8. Diehl, A. M., and Day, C. (2017). Cause, Pathogenesis, and Treatment of Nonalcoholic Steatohepatitis. In D. L. Longo, ed. New England Journal of Medicine. New England Journal of Medicine (NEJM/MMS).
9. Alexander, M., Loomis, A. K., Van Der Lei, J., Duarte-Salles, T., Prieto-Alhambra, D., Ansell, D., Pasqua, A., Lapi, F., Rijnbeek, P., Mosseveld, M., et al. (2019). Risks and clinical predictors of cirrhosis and hepatocellular carcinoma diagnoses in adults with diagnosed NAFLD: Real-world study of 18 million patients in four European cohorts. BMC Medicine. BioMed Central Ltd.
10. Ganz, M., and Szabo, G. (2013). Immune and inflammatory pathways in NASH. Hepatology International 2014 7:2. Springer.
11. Friedman, S. L., Neuschwander-Tetri, B. A., Rinella, M., and Sanyal, A. J. (2018). Mechanisms of NAFLD development and therapeutic strategies. Nature Medicine. Nature Publishing Group.
12. Llovet, J. M., Willoughby, C. E., Singal, A. G., Greten, T. F., Heikenwalder, M., El-Serag, H. B., Finn, R. S., and Friedman, S. L. (2023). Nonalcoholic steatohepatitis-related hepatocellular carcinoma: pathogenesis and treatment. Nature Reviews Gastroenterology & Hepatology. 10.1038/s41575-023-00754-7.
13. Muthiah, M. D., and Sanyal, A. J. (2020). Current management of non-alcoholic steatohepatitis. Liver International. Blackwell Publishing Ltd.
14. Tilg, H., Adolph, T. E., and Moschen, A. R. (2021). Multiple Parallel Hits Hypothesis in Nonalcoholic Fatty Liver Disease: Revisited After a Decade. Hepatology 73, 833-842. 10.1002/hep.31518.
15. Wong, V. W. S., and Singal, A. K. (2019). Emerging medical therapies for non-alcoholic fatty liver disease and for alcoholic hepatitis. Translational Gastroenterology and Hepatology. AME Publishing Company.
16. Valenti, L., Al-Serri, A., Daly, A. K., Galmozzi, E., Rametta, R., Dongiovanni, P., Nobili, V., Mozzi, E., Roviaro, G., Vanni, E., et al. (2010). Homozygosity for the patatin-like phospholipase-3/adiponutrin i148m polymorphism influences liver fibrosis in patients with nonalcoholic fatty liver disease. Hepatology. Hepatology.
17. Speliotes, E. K., Yerges-Armstrong, L. M., Wu, J., Hernaez, R., Kim, L. J., Palmer, C. D., Gudnason, V., Eiriksdottir, G., Garcia, M. E., Launer, L. J., et al. (2011). Genome-Wide Association Analysis Identifies Variants Associated with Nonalcoholic Fatty Liver Disease That Have Distinct Effects on Metabolic Traits. PLOS Genetics. Public Library of Science.
18. S, R., A, S., and L, V. (2020). Leveraging Human Genetics to Identify Potential New Treatments for Fatty Liver Disease. Cell metabolism. Cell Metab.
19. Mancina, R. M., Dongiovanni, P., Petta, S., Pingitore, P., Meroni, M., Rametta, R., Borén, J., Montalcini, T., Pujia, A., Wiklund, O., et al. (2016). The MBOAT7-TMC4 Variant rs641738 Increases Risk of Nonalcoholic Fatty Liver Disease in Individuals of European Descent. Gastroenterology. Elsevier.
20. Kozlitina, J., Stender, S., Hobbs, H. H., and Cohen, J. C. (2018). HSD17B13 and Chronic Liver Disease in Blacks and Hispanics. https://doi.org/10.1056/NEJMc1804027. Massachusetts Medical Society.
21. Kozlitina, J., Smagris, E., Stender, S., Nordestgaard, B. G., Zhou, H. H., Tybjærg-Hansen, A., Vogt, T. F., Hobbs, H. H., and Cohen, J. C. (2014). Exome-wide association study identifies a TM6SF2 variant that confers susceptibility to nonalcoholic fatty liver disease. Nature Genetics 2014 46:4. Nature Publishing Group.
22. Koo, B. K., Joo, S. K., Kim, D., Bae, J. M., Park, J. H., Kim, J. H., and Kim, W. (2018). Additive effects of PNPLA3 and TM6SF2 on the histological severity of non-alcoholic fatty liver disease. Journal of Gastroenterology and Hepatology. John Wiley & Sons, Ltd.
23. Eslam, M., and George, J. (2019). Genetic contributions to NAFLD: leveraging shared genetics to uncover systems biology. Nature Reviews Gastroenterology & Hepatology 2019 17:1. Nature Publishing Group.
24. Yenilmez, B., Wetoska, N., Kelly, M., Echeverria, D., Min, K., Lifshitz, L., Alterman, J. F., Hassler, M. R., Hildebrand, S., DiMarzio, C., et al. An RNAi therapeutic targeting hepatic DGAT2 in a genetically obese mouse model of nonalcoholic steatohepatitis. Molecular Therapy. 10.1016/j.ymthe.2021.11.007.
25. Yu, X. X., Murray, S. F., Pandey, S. K., Booten, S. L., Bao, D., Song, X. Z., Kelly, S., Chen, S., McKay, R., Monia, B. P., and Bhanot, S. (2005). Antisense oligonucleotide reduction of DGAT2 expression improves hepatic steatosis and hyperlipidemia in obese mice. Hepatology. Hepatology.
26. Yamaguchi, K., Yang, L., McCall, S., Huang, J., Xing, X. Y., Pandey, S. K., Bhanot, S., Monia, B. P., Li, Y. X., and Diehl, A. M. (2007). Inhibiting triglyceride synthesis improves hepatic steatosis but exacerbates liver damage and fibrosis in obese mice with nonalcoholic steatohepatitis. Hepatology. Hepatology.
27. Loomba, R., Morgan, E., Watts, L., Xia, S., Hannan, L. A., Geary, R. S., Baker, B. F., and Bhanot, S. (2020). Novel antisense inhibition of diacylglycerol O-acyltransferase 2 for treatment of non-alcoholic fatty liver disease: a multicentre, double-blind, randomised, placebo-controlled phase 2 trial. The Lancet Gastroenterology and Hepatology. Elsevier Ltd.
28. Choi, C. S., Savage, D. B., Kulkarni, A., Yu, X. X., Liu, Z.-X., Morino, K., Kim, S., Distefano, A., Samuel, V. T., Neschen, S., et al. (2007). Suppression of Diacylglycerol Acyltransferase-2 (DGAT2), but Not DGAT1, with Antisense Oligonucleotides Reverses Diet-induced Hepatic Steatosis and Insulin Resistance*. Journal of Biological Chemistry 282, 22678-22688. https://doi.org/10.1074/jbc.M704213200.
29. Calle, R. A., Amin, N. B., Carvajal-Gonzalez, S., Ross, T. T., Bergman, A., Aggarwal, S., Crowley, C., Rinaldi, A., Mancuso, J., Aggarwal, N., et al. (2021). ACC inhibitor alone or co-administered with a DGAT2 inhibitor in patients with non-alcoholic fatty liver disease: two parallel, placebo-controlled, randomized phase 2a trials. Nature Medicine 27, 1836-1848. 10.1038/s41591-021-01489-1.
30. Amin, N. B., Carvajal-Gonzalez, S., Purkal, J., Zhu, T., Crowley, C., Perez, S., Chidsey, K., Kim, A. M., and Goodwin, B. (2019). Targeting diacylglycerol acyltransferase 2 for the treatment of nonalcoholic steatohepatitis. Science Translational Medicine. American Association for the Advancement of Science.
31. Zammit, V. A. (2013). Hepatic triacylglycerol synthesis and secretion: DGAT2 as the link between glycaemia and triglyceridaemia. Biochemical Journal. Biochem J.
32. Yen, C. L. E., Stone, S. J., Koliwad, S., Harris, C., and Farese, R. V. (2008). DGAT enzymes and triacylglycerol biosynthesis. Journal of Lipid Research. Elsevier.
33. S, C., and A M, D. (2005). Role of inflammation in nonalcoholic steatohepatitis. Current opinion in gastroenterology. Curr Opin Gastroenterol.
34. Loomba, R., Noureddin, M., Kowdley, K. V., Kohli, A., Sheikh, A., Neff, G., Bhandari, B. R., Gunn, N., Caldwell, S. H., Goodman, Z., et al. (2021). Combination Therapies Including Cilofexor and Firsocostat for Bridging Fibrosis and Cirrhosis Attributable to NASH. Hepatology. Wiley.
35. Bates, J., Vijayakumar, A., Ghoshal, S., Marchand, B., Yi, S., Kornyeyev, D., Zagorska, A., Hollenback, D., Walker, K., Liu, K., et al. (2020). Acetyl-CoA carboxylase inhibition disrupts metabolic reprogramming during hepatic stellate cell activation. Journal of Hepatology 73, 896-905. https://doi.org/10.1016/j.jhep.2020.04.037.
36. O'Farrell, M., Duke, G., Crowley, R., Buckley, D., Martins, E. B., Bhattacharya, D., Friedman, S. L., and Kemble, G. (2022). FASN inhibition targets multiple drivers of NASH by reducing steatosis, inflammation and fibrosis in preclinical models. Scientific Reports 12, 15661. 10.1038/s41598-022-19459-z.
37. Loomba, R., Mohseni, R., Lucas, K. J., Gutierrez, J. A., Perry, R. G., Trotter, J. F., Rahimi, R. S., Harrison, S. A., Ajmera, V., Wayne, J. D., et al. (2021). TVB-2640 (FASN Inhibitor) for the Treatment of Nonalcoholic Steatohepatitis: FASCINATE-1, a Randomized, Placebo-Controlled Phase 2a Trial. Gastroenterology 161, 1475-1486. 10.1053/j.gastro.2021.07.025.
38. Syed-Abdul, M. M., Parks, E. J., Gaballah, A. H., Bingham, K., Hammoud, G. M., Kemble, G., Buckley, D., McCulloch, W., and Manrique-Acevedo, C. (2020). Fatty Acid Synthase Inhibitor TVB-2640 Reduces Hepatic de Novo Lipogenesis in Males With Metabolic Abnormalities. Hepatology 72, 103-118. 10.1002/hep.31000.
39. Afify, H., Ghoneum, A., Almousa, S., Abdulfattah, A. Y., Warren, B., Langsten, K., Gonzalez, D., Casals, R., Bharadwaj, M., Kridel, S., and Said, N. (2021). Metabolomic credentialing of murine carcinogen-induced urothelial cancer. Sci Rep 11, 22085. 10.1038/s41598-021-99746-3.
40. Chu, J., Xing, C., Du, Y., Duan, T., Liu, S., Zhang, P., Cheng, C., Henley, J., Liu, X., Qian, C., et al. (2021). Pharmacological inhibition of fatty acid synthesis blocks SARS-CoV-2 replication. Nat Metab 3, 1466-1475. 10.1038/s42255-021-00479-4.
41. Friedman, S. L. (2008). Hepatic stellate cells: protean, multifunctional, and enigmatic cells of the liver. Physiol Rev 88, 125-172. 10.1152/physrev.00013.2007.
42. Fondevila, M. F., Fernandez, U., Heras, V., Parracho, T., Gonzalez-Rellan, M. J., Novoa, E., Porteiro, B., Alonso, C., Mayo, R., da Silva Lima, N., et al. (2022). Inhibition of carnitine palmitoyltransferase 1A in hepatic stellate cells protects against fibrosis. J Hepatol 77, 15-28. 10.1016/j.jhep.2022.02.003.
43. Brown, K. E., Broadhurst, K. A., Mathahs, M. M., Brunt, E. M., and Schmidt, W. N. (2005). Expression of HSP47, a collagen-specific chaperone, in normal and diseased human liver. Laboratory Investigation 85, 789-797. 10.1038/labinvest.3700271.
44. Prestigiacomo, V., and Suter-Dick, L. (2018). Nrf2 protects stellate cells from Smad-dependent cell activation. PLoS One 13, e0201044. 10.1371/journal.pone.0201044.
45. Nisimoto, Y., Diebold, B. A., Cosentino-Gomes, D., and Lambeth, J. D. (2014). Nox4: a hydrogen peroxide-generating oxygen sensor. Biochemistry 53, 5111-5120. 10.1021/bi500331y.

46. Sena-Esteves, M., and Gao, G. (2020). Introducing Genes into Mammalian Cells: Viral Vectors. Cold Spring Harb Protoc 2020, 095513. 10.1101/pdb.top095513.
47. O'Shea, J., Theile, C. S., Das, R., Babu, I. R., Charisse, K., Manoharan, M., Maier, M. A., and Zlatev, I. (2018). An efficient deprotection method for 5′-[O,O-bis(pivaloyloxymethyl)]-(E)-vinylphosphonate containing oligonucleotides. Tetrahedron 74, 6182-6186. https://doi.org/10.1016/j.tet.2018.09.008.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A combination of oligonucleotides comprising:
a first oligonucleotide comprising a 5′ end, a 3′ end, and a first sequence substantially complementary to a Diacylglycerol O-Acyltransferase 2 (DGAT2) nucleic acid sequence; and
a second oligonucleotide comprising a 5′ end, a 3′ end, and a second sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence.

2. The combination of claim 1, wherein the DGAT2 nucleic acid sequence is any one of SEQ ID NOs: 1-5.

3. The combination of claim 1, wherein the DGAT2 nucleic acid sequence is any one of SEQ ID NOs: 6-10.

4-6. (canceled)

7. The combination of claim 1, wherein the FASN nucleic acid sequence is any one of Table 5.

8-10. (canceled)

11. The combination of claim 1, wherein the OPN nucleic acid sequence is any one of Table 8.

12-14. (canceled)

15. The combination of claim 1, wherein the NOX4 nucleic acid sequence is any one of Table 10.

16-18. (canceled)

19. The combination of claim 1, wherein each of the first and the second oligonucleotides is independently an antisense oligonucleotide (ASO) or a double stranded RNA (dsRNA) moiety, wherein the dsRNA moiety comprises a sense strand and an antisense strand, each strand comprising separately a 3′ end and a 5′ end.

20. The combination of claim 19, wherein each of the first and the second oligonucleotides is the dsRNA moiety, and wherein the antisense strand comprises about 15 nucleotides to about 25 nucleotides in length.

21-39. (canceled)

40. The combination of claim 19, wherein each of the first and the second oligonucleotides is the dsRNA moiety, and wherein the dsRNA moiety comprises at least one modified nucleotide, optionally wherein the at least one modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

41. (canceled)

42. The combination of claim 19, wherein each of the first and the second oligonucleotides is the dsRNA moiety, and wherein the dsRNA moiety comprises at least one modified internucleotide linkage, optionally wherein the at least one modified internucleotide linkage comprises a phosphorothioate internucleotide linkage.

43-45. (canceled)

46. The combination of claim 19, wherein each of the first and the second oligonucleotides is the dsRNA moiety, and wherein the dsRNA moiety comprises at least one modified internucleotide linkage of Formula I: 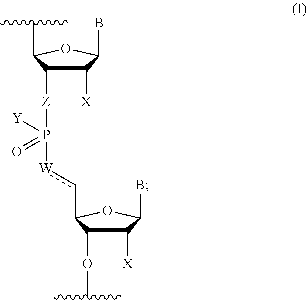
wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
R is a protecting group; and [symbol]
is an optional double bond.

47-58. (canceled)

59. The combination of claim 1, wherein:
A:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; or
B:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; or
C:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; or
D:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 80% 2′-O-methyl modifications;
(3) the nucleotides at positions 2, 4, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 80% 2′-O-methyl modifications; and
(7) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages;
E:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, 14, 16, and 20 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7 and 9-11 from the 3′ end of the sense strand are not 2′-methoxy-ribonucleotides; and
(8) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages; or
F:
the first oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a DGAT2 nucleic acid sequence of any one of SEQ ID NOs: 1-10;
(2) the antisense strand comprises at least 70% 2′-O-methyl modifications;
(3) the nucleotides at positions 2, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7, 10, and 11 from the 3′ end of the sense strand are not 2′-methoxy-ribonucleotides; and
(8) the nucleotides at positions 1-2 from the 5′ end of the sense strand are connected to each other via phosphorothioate internucleotide linkages; and
the second oligonucleotide is a double stranded RNA (dsRNA) moiety comprising a sense strand and an antisense strand, each strand comprising separately a 5′ end and a 3′ end, wherein:
(1) the antisense strand comprises a sequence substantially complementary to a FASN nucleic acid sequence of any one of Table 5, an OPN nucleic acid sequence of any one of Table 8, or a NOX4 nucleic acid sequence of any one of Table 10;
(2) the antisense strand comprises at least 70% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

60-65. (canceled)

66. The combination of claim 19, wherein each of the first and the second oligonucleotides is the dsRNA moiety, and wherein a functional moiety is linked to the 5′ end and/or the 3′ end of the antisense strand.

67-68. (canceled)

69. The combination of claim 66, wherein the functional moiety comprises an N-acetylgalactosamine (GalNAc) moiety.

70. The combination of claim 66, wherein the functional moiety comprises a hydrophobic moiety.

71. The combination of claim 70, wherein the hydrophobic moiety is selected from the group consisting of fatty acids, steroids, secosteroids, lipids, gangliosides, nucleoside analogs, endocannabinoids, vitamins, and a mixture thereof, optionally wherein:
the steroids are selected from the group consisting of cholesterol and lithocholic acid (LCA); or
the fatty acids are selected from the group consisting of eicosapentaenoic acid (EPA), docosahexaenoic acid (DHA), and docosanoic acid (DCA).

72-82. (canceled)

83. A pharmaceutical composition for inhibiting the expression of a Diacylglycerol O-Acyltransferase 2 (DGAT2) gene and a Fatty Acid Synthase (FASN) gene, an Osteopontin (OPN) gene, or a Nicotinamide Adenine Dinucleotide Phosphate Oxidase (NOX4) gene in an organism, wherein the pharmaceutical composition comprises the combination of claim 1 and a pharmaceutically acceptable carrier.

84-85. (canceled)

86. A method of treating or managing a disease associated with Diacylglycerol O-Acyltransferase 2 (DGAT2) and Fatty Acid Synthase (FASN), Osteopontin (OPN), or Nicotinamide Adenine Dinucleotide Phosphate Oxidase (NOX4), the method comprising administering to a patient in need of such treatment a therapeutically effective amount of a combination of oligonucleotides comprising:
a first oligonucleotide comprising a 5′ end, a 3′ end, and a first sequence substantially complementary to a DGAT2 nucleic acid sequence; and
a second oligonucleotide comprising a 5′ end, a 3′ end, and a second sequence substantially complementary to a FASN nucleic acid sequence, an OPN nucleic acid sequence, or a NOX4 nucleic acid sequence,
wherein the first oligonucleotide inhibits a DGAT2 gene expression by at least about 50% for four weeks post administration, and
wherein the second oligonucleotide inhibits a FASN gene, an OPN gene, or a NOX4 gene expression by at least about 50% for four weeks post administration.

87-90. (canceled)

91. A method for delivering the combination of claim 1 to a liver of a patient, the method comprising administering the combination to the patient.

92. A vector comprising:
a first regulatory sequence operably linked to a first nucleotide sequence that encodes a first oligonucleotide substantially complementary to a Diacylglycerol O-Acyltransferase 2 (DGAT2) nucleic acid sequence; and
a second regulatory sequence operably linked to a second nucleotide sequence that encodes a second oligonucleotide substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence.

93. (canceled)

94. A cell comprising the vector of claim 92.

95. A recombinant adeno-associated virus (rAAV) comprising the vector of claim 92 and an AAV capsid.

96. A branched compound comprising:
a first oligonucleotide comprising a 5′ end, a 3′ end, and a first sequence substantially complementary to a Diacylglycerol O-Acyltransferase 2 (DGAT2) nucleic acid sequence; and
a second oligonucleotide comprising a 5′ end, a 3′ end, and a second sequence substantially complementary to a Fatty Acid Synthase (FASN) nucleic acid sequence, an Osteopontin (OPN) nucleic acid sequence, or a Nicotinamide Adenine Dinucleotide Phosphate Oxidase (NOX4) nucleic acid sequence,
wherein the first and the second oligonucleotide are linked together by a linker.

97-102. (canceled)
